SEQUENCE LISTING (148 sequences)

SEQ ID NO: 1 - protein, 533 aa, Staphylococcus aureus
MLQVTDVSLRFGDRKLFEDVNIKFTEGNCYGLIGANGAGKSTFLKILSGELDSQTGHVSLGKNERLAVLKQDHYAYEDERVLDVVIKGHERLYEVMKEKDEIYMKPDFSDEDGIRAAELEGEFAEMNGWNAEADAANLLSGLGIDPTLHDKKMAELENNQKIKVLLAQSLFGEPDVLLLDEPTNGLDIPAISWLEDFLINFDNTVIVVSHDRHFLNNVCTHIADLDFGKIKVYVGNYDFWYQSSQLAQKMAQEQNKKKEEKMKELQDFIARFSANASKSKQATSRKKQLEKIELDDIQPSSRRYPFVKFTPEREIGNDLLIVQNLSKTIDGEKVLDNVSFTMNPNDKAILIGDSEIAKTTLLKILAGEMEPDEGSFKWGVTTSLSYFPKDNSEFFEGVNMNLVDWLRQYAPEDEQTETFLRGFLGRMLFSGEEVKKKASVLSGGEKVRCMLSKMMLSSANVLLLDEPTNHLDLESITAVNDGLKSFKGSIIFTSYDFEFINTIANRVIDLNKQGGVSKEIPYEEYLQEIGVLK

SEQ ID NO: 2 - protein, 535 aa, Staphylococcus epidermidis
MLQVTDVSLRFGDRKLFEDVNIKFTEGNCYGLIGANGAGKSTFLKILSGEIDSQTGHVSLGKDERLAVLKQDHFAYEDERVLDVVIKGHERLYQVMKEKDEIYMKPDFSDEDGIRAAELEGEFAEMNGWNAEADAANLLSGLGIEPDLHDKNMSELENNQKVKVLLAQSLFGDPDVLLLDEPTNGLDIPAISWLEDFLINFENTVIVVSHDRHFLNNVCTHIADLDFGKIKLYVGNYDFWYQSSQLAQKMAQEQNKKKEEKMKELQDFIARFSANASKSKQATSRKKQLEKIELDDIQPSSRRYPYVKFTPEREIGNDLLTVENLSKTIDGEKVLDNVSFTMNPNDKAILVGDSEIAKTTLLKILAGEMEPDEGTFKWGVTTSLSYFPKDNSEFFDGVDMNLVEWLRQYAPEDEQTETFLRGFLGRMLFSGEEVKKKASVLSGGEKVRCMLSKMMLSSANVLLLDEPTNHLDLESITAVNDGLKSFKGSIIFTSYDFEFINTIANRVIDLNQAGALSKEVPYEEYLQEIGVLQNN

SEQ ID NO: 3 - protein, 434 aa, Staphylococcus aureus [sequence omitted]

SEQ ID NO: 4 - protein, 434 aa, Staphylococcus epidermidis
MPIITDVYAREVLDSRGNPTVEVEVLTESGAFGRALVPSGASTGEHEAVELRDGDKSRYLGKGVTKAVENVNEMIAPEIVEGEFSVLDQVSIDKMMIQLDGTHNKGKLGANAILGVSIAVARAAADLLGQPLYKYLGGFNGKQLPVPMMNIVNGGSHSDAPIAFQEFMILPVGAESFKESLRWGAEIFHNLKSILSERGLETAVGDEGGFAPRFEGTEDAVETIIKAIEKAGYKPGEDVFLGFDCASSEFYENGVYDYTKFEGEHGAKRSAAEQVDYLEELIGKYPIITIEDGMDENDWEGWKQLTDRIGDKVQLVGDDLFVTNTEILSKGIEQGIGNSILIKVNQIGTLTETFDAIEMAQKAGYTAVVSHRSGETEDTTIADIAVATNAGQIKTGSLSRTDRIAKYNQLLRIEDELYETAKFEGIKSFYNLDK

SEQ ID NO: 5 - protein, 255 aa, Staphylococcus aureus
MKKIVTATIATAGLATIAFAGHDAQAAEQNNNGYNSNDAQSYSYTYTIDAQGNYHYTWTGNWNPSQLTQNNTYYYNNYNTYSYNNASYNNYYNHSYQYNNYTNNSQTATNNYYTGGSGASYSTTSNNVHVTTTAAPSSNGRSISNGYASGSNLYTSGQCTYYVFDRVGGKIGSTWGNASNWANAAASSGYTVNNTPKVGAIMQTTQGYYGHVAYVEGVNSNGSVRVSEMNYGHGAGVVTSRTISANQAGSYNFIH

SEQ ID NO: 6 - protein, 267 aa, Staphylococcus aureus
MKKIATATIATAGFATIAIASGNQAHASEQDNYGYNPNDPTSYSYTYTIDAQGNYHYTWKGNWHPSQLNQDNGYYSYYYYNGYNNYNNYNNGYSYNNYSRYNNYSNNNQSYNYNNYNSYNTNSYRTGGLGASYSTSSNNVQVTTTMAPSSNGRSISSGYTSGRNLYTSGQCTYYVFDRVGGKIGSTWGNASNWANAAARAGYTVNNTPKAGAIMQTTQGAYGHVAYVESVNSNGSVRVSEMNYGYGPGVVTSRTISASQAAGYNFIH

SEQ ID NO: 7 - protein, 257 aa, Staphylococcus epidermidis
MKKIATATIATAGIATFAFAHHDAQAAEQNNDGYNPNDPYSYSYTYTIDAEGNYHYTWKGNWSPDRVNTSYNYNNYNNYNYYGYNNYSNYNNYSNYNNYNNYQSNNTQSQRTTQPTGGLGASYSTSSSNVHVTTTSAPSSNGVSLSNARSASGNLYTSGQCTYYVFDRVGGKIGSTWGNANNWANAAARSGYTVNNSPAKGAILQTSQGAYGHVAYVEGVNSNGSIRVSEMNYGHGAGVVTSRTISASQAASYNYIH

SEQ ID NO: 8 - protein, 309 aa, Staphylococcus aureus
MKKLVPLLLALLLLVAACGTGGKQSSDKSNGKLKVVTTNSILYDMAKNVGGDNVDIHSIVPVGQDPHEYEVKPKDIKKLTDADVILYNGLNLETGNGWFEKALEQAGKSLKDKKVIAVSKDVKPIYLNGEEGNKDKQDPHAWLSLDNGIKYVKTIQQTFIDNDKKHKADYEKQGNKYIAQLEKLNNDSKDKFNDIPKEQRAMITSEGAFKYFSKQYGITPGYIWEINTEKQGTPEQMRQAIEFVKKHKLKHLLVETSVDKKAMESLSEETKKDIFGEVYTDSIGKEGTKGDSYYKMMKSNIETVHGSMK

SEQ ID NO: 9 - protein, 309 aa, Staphylococcus epidermidis
MKKILALAIAFLIILAACGNHSNHEHHSHEGKLKVVTTNSILYDMVKRVGGNKVDVHSIVPVGQDPHEYEVKPKDIKALTDADVVFYNGLNLETGNGWFEKALDQAGKSTKDKNVIAASNNVKPIYLNGEEGNKNKQDPHAWLSLENGIKYVKTIQKSLEHHDKKDKSTYEKQGNAYISKLEELNKDSKNKFDDIPKNQRAMMTSEGAFKYFAQQFDVKPGYIWEINTEKQGTPGQMKQAIKFVKDNHLKHLLVETSVDKKAMQSLSEETKKDIYGEVFTDSIGKEGTKGDSYYKMMKSNIDTIHGSMK

SEQ ID NO: 10 - protein, 233 aa, Staphylococcus aureus
MKKTIMASSLAVALGVTGYAAGTGHQAHAAEVNVDQAHLVDLAHNHQDQLNAAPIKDGAYDIHFVKDGFQYNFTSNGTTWSWSYEAANGQTAGFSNVAGADYTTSYNQGSDVQSVSYNAQSSNSNVEAVSAPTYHNYSTSTTSSSVRLSNGNTAGATGSSAAQIMAQRTGVSASTWAAIIARESNGQVNAYNPSGASGLFQTMPGWGPTNTVDQQINAAVKAYKAQGLGAWGF

SEQ ID NO: 11 - protein, 235 aa, Staphylococcus epidermidis
MKKTVIASTLAVSLGIAGYGLSGHEAHASETTNVDKAHLVDLAQHNPEELNAKPVQAGAYDIHFVDNGYQYNFTSNGSEWSWSYAVAGSDADYTESSSNQEVSANTQSSNTNVQAVSAPTSSESRSYSTSTTSYSAPSHNYSSHSSSVRLSNGNTAGSVGSYAAAQMAARTGVSASTWEHIIARESNGQLHARNASGAAGLFQTMPGWGSTGSVNDQINAAYKAYKAQGLSAWGM

SEQ ID NO: 12 - protein, 3890 aa, Staphylococcus aureus
MNYRDKIQKFSIRKYTVGTFSTVIATLVFLGFNTSQAHAAETNQPASVVKQKQQSNNEQTENRESQVQNSQNSQNSQSLSATHENEQPNNSQANLVNQKVAQSSTTNDEQPASQNVNTKKDSATAATTQPDKEESKHKQNESQSANKNGNDNRAAHVENHEANVVTASDSSDNGNVQHDRNELQAFFDANYHDYRFIDRENADSGTFNYVKGIFDKINTLLGSNDPINNKDLQLAYKELEQAVALIRTMPQRQQTSRRSNRIQTRSVESRAAEPRSVSDYQNANSSYYVENANDGSGYPVGTYINASSKGAPYNLPTTPWNTLKASDSKEIALMTAKQTGDGYQWVIKFNKGHAPHQNMIFWFALPADQVPVGRTDFVTVNSDGTNVQWSHGAGAGANKPLQQMWEYGVNDPDRSHDFKIRNRSGQVIYSWPTVHVYSLEDLSRASDYFSEAGATPATKAFGRQNFEYINGQKPAESPGVPKVYTFIGQGDASYTISFKTQGPTVNKLYYAAGGRALEYNQLFMYSQLYVESTQDHQQRLNGLRQVVNRTYRIGTTKRVEVSQGNVQTKKVLESTNLNIDDFVDDPLSYVKTPSNKVLGFYPTNANTNAFRPGGVQELNEYQLSQLFTDQKLQEAARTRNPIRLMIGFDYPDGYGNSETLVPVNLTVLPEIQHNIKFFKNDDTQNIAEKPFSKQAGHPVFYVYAGNQGNASVNLGGSVTSIQPLRINLTSNENFTDKDWQITGIPRTLHIENSTNRTNNARERNIELVGNLLPGDYFGTIRFGRKEQLFEIRVKPHTPTITTTAEQLRGTALQKVPVNISGIPLDPSALVYLVAPTNQTTNGGSEADQIPSGYTILATGTPDGVHNTITIRPQDYVVFIPPVGKQIRAVVYYNKVVASNMSNAVTILPDDIPPTINNPVGINAKYYRGDEVNFTMGVSDRHSGIKNTTITTLPSGWTSNLTKSDNKNGSLAITGRVSMNQAFNSDITFKVSATDNVNNTTNDSQSKHVSIHVGKISEDAHPIVLGNTEKVVVVNPTAVSNDEKQSIITAFMNKNQNIRGYLASTDPVTVDNNGNVTLHYRDGSSTTLDATNVMTYEPVVKSEYQTANAAKTATVTIAKGQSFNIGDIKQYFTLSNGQAIPNGTFTNITSDRTIPTAQEVSQMNAGTQLYHIVASNAYHKDTEDFYISLKIVDVKQPEGDQRVYRTSTYDLTTDEISKVKQAFINANRDVITLAEGDISVTNTPNGANVSTITVNINKGRLTKSFASNLANMNFLRWVNFPQDYTVTWTNAKIANRPTDGGLSWSDDHKSLIYRYDATLGTQITTNDILTMLKATTTVPGLRNNITGNEKAQAEAGGRPNYRTTGYSQSNATTDGQRQFTLNGQVIQILDIINPSNGYGGQPVTNSNTRANHSNSTVVNVNEPAANGAGAFTIDHVVKSNSTHNASDAVYKAQLYLTPYGPKQYVEHLNQNTGNTTDAINIYFVPSDLVNPTISVGNYTNHQVFSGETFTNTITANDNFGVQSVTVPNTSQITGTVDNNHQHVSATAPNVTSATSKTINLLATDTSGNTATTSFNVTVKPLRDKYRVGTSSTAANPVRIANISNNATVSQADQTTIINSLTFTSNAPNRNYATASANEITSKTVSNVSRTGNNANVTVTVTHQDGTTSTVTVPVKHVIPEIVAHSHYTVQGQDFPAGNGSSAADYFKLSNGSAIPDATITWVSGQAPNKDNTRIGEDITVTAHILIDGETTPITKTATYKVVRTVPKHVFETARGVLYPGVSDMYDAKQYVKPVNNSWSTNAQHMNFQFVGTYGPNKDVVGISTRLIRVTYDNRQTEDLTILSKVKPDPPRIDANSVTYKAGLTNQEIKVNNVLNNSSVKLFKADNTPLNVTNITHGSGFSSVVTVSDALPNGGIKAKSSISMNNVTYTTQDEHGQVVTVTRNESVDSNDSASVTVTPQLQATTEGAVFIKGGDGFDFGHVERFIQNPPHGATVAWHDSPDTWKNTVGNTHKTAVVTLPSGQGTRNVEVPVKVYPVANAKAPSRDVKGQNLTHGTNAIDYITFDPNTNTNGITAAWANRQQPNNQQAGVQHLNVDVTYPGISAAKRVPVTVNVYQFEFPQTTYTTTVGGTLASGTQASGYAHMQNASGLPTDGFTYKWNRDTTGTNDANWAAMNKPNTAQVVNAKYDVIYNGHTFATSLPAKFVVKDVQPAKPTVTETAAGAITIAPGANQTVNTHAGNVTTYADKLVIKRNGNVVTTFTRRNNTSPWVKEASADNVTGIVGTNNGITVAAGTFNPADTIQVVATQGSGETISDEQRSDDFTVVAPQPNQATTKIWQNGHIDITPNNPSGHLINPTQAMDIAYTEKVGNGAEHSKTINVVRGQNNQWTIANKPDYVTLDAQTGKVTFNANTIKPNSSITITPKAGTGHSVSSNPSTLTAPAAHTVNTTEIVKDYGSNVTAAEINNAVQVANKRTATIKNGTAMPTNLAGGSTTTIPVTVTYNDGSTEEVQESIFTKADKRELITAKNHLDDPVSTEGKKPGTITQYNNAMHNAQQQINTAKTEAQQVINNERATPQQVSDALTKVRAAQTKIDQAKALLQNKEDNSQLVTSKNNLQSSVNQVPSTAGMTQQSIDNYNAKKREAETEITAAQRVIDNGDATAQQISDEKHRVDNALTALNQAKHDLTADTHALEQAVQQLNRTGTTTGKKPASITAYNNSIRALQSDLTSAKNSANAIIQKPIRTVQEVQSALTNVNRVNERLTQAINQLVPLADNSALRTAKTKLDEEINKSVTTDGMTQSSIQAYENAKRAGQTETTNAQNVINNGDATDQQIAAEKTKVEEKYNSLKQAIAGLTPDLAPLQTAKTQLQNDIDQPTSTTGMTSASVAAFNDKLSAARTKIQEIDRVLASHPDVATIRQNVTAANAAKTALDQARNGLTVDKAPLENAKNQLQHSIDTQTSTTGMTQDSINAYNAKLTAARNKVQQINQVLAGSPTVDQINTNTSAANQAKSDLDHARQALTPDKAPLQNAKTQLEQSINQPTDTTGMTTASLNAYNQKLQAARQKLTEINQVLNGNPTVQNINDKVAEANQAKDQLNTARQGLTLDRQPALTTLHGASNLNQAQQNNFTQQINAAQNHAALETIKSNITALNTAMTKLKDSVADNNTIKSGQNYTDATPANKQAYDNAVNAAKGVIGETTNPTMDVNTVNQKAASVKSTKDALDGQQNLQRAKTEATNAITHASDLNQAQKNALTQQVNSAQNVQAVNDIKQTTQSLNTAMTGLKRGVANHNQVVQSDNYVNADTNKKNDYNNAYNHANDIINGNAQHPVITPSDVNNALSNVTSKEHALNGEAKLNAAKQEANTALGHLNNLNNVQRQNLQSQINGAHQIDAVNTIKQNATNLNSAMGNLRQAVADKDQVKRTEDYADADTAKQNAYNSAVSSAETIINQTANPTMSVDDVNRATSAVTTNKNALNGDEKLVQSKTDAARAIDALPHLNNAQKADVKSKINAASNIAGVNTVKQQGTDLNTAMGNLQGAINDEQTTLNSQNYQDATPSKKTAYTNAVQAAKDILNKSNGQNKTKDQVTEAMNQVNSAKNNLDGTRLLDQAKQTAKQQLNNMTHLTTAQKTNLTNQINSGTTVAGVHTVQSNANTLDQAMNTLRQSIANNDATKASEDYVDANNDKQTAYNNAVAAAETIINANSNPEMNPSTITQKAEQVNSSKTALNGDENLATAKQNAKTYLNTLTSITDAQKNNLISQISSATRVSGVDTVKQNAQHLDQAMANLQNGINNESQVKSSEKYRDADTNKQQEYDNAITAAKAILNKSTGPNTAQNAVEAALQRVNTAKDALNGDAKLIAAQNAAKQHLGTLTHITTAQRNDLTNQIS

SEQ ID NO: 13 - protein, 6713 aa, Staphylococcus aureus
MGNLQTAINDKSGTLASQNFLDADEQKRNAYNQAISAAETILNKQTGPNTAKTAVEQALNNVNSAKHALNGTQNLNNAKQAAITAINGASDLNQKQKDALKAQANGAQRVSNANDVQRNATELNTAMGQLQHAIADKTNTLASSKYVNADSTKQNAYTTKVTNAEHIISGTPTVVTTPSEVTAAANQVNSAKQELNGDERLRVAKQNANTAIDALTQLNTPQKAKLKEQVGQANRLEDVQSVQTNGQSLNNAMKGLRDSIANETTVKASQNYTDASPNNQSTYNSAVSNAKGIINQTNNPTMDTSAITQATTQVNNAKNGLNGAENLRNAQNTAKQNLNTLSHLTNNQKSAISSQIDRAGHVSEVTAAKNAATELNAQMGNLEQAIHDQNTVKQGVNFTDADKAKRDAYTNAVSRAETILNKTQGANTSKQDVEAAIQNVTSAKNALNGDQNVTNAKNAAKNALNNLTSINNAQKRDLTTKIDQATTVAGVEAVSNTGTQLNTAMANLQNGINDKANTLASENYHDADSDKKTAYTQAVTNAENILNKNSGSNLDKAAVENALSQVTNAKGALNGNHNLEQAKSNANTTINGLQHLTTAQKDKLKQQVQQAQNVAGVDTVKSSANTLNGAMGTLRNSIQDNTATKNGQNYLDATERNKTNYNNAVDSANGVINATSNPNMDANAINQIATQVTSTKNALDGTHNLTQAKQTATNAIDGATNLNKAQKDALKAQVTSAQRVANVTSIQQTANELNTAMGQLQHGIDDENATKQTQKYRDAEQSKKTAYDQAVAAAKAILNKQTGSNSDKAAVDRALQQVTSTKDALNGDAKLAEAKAAARQNLGTLNHITNAQRTALEGQINQATTVDGVNTVKTNANTLDGAMNSLQGAINDKDATLRNQNYLDADESKRNAYTQAVTAAEGILNKQTGGNTSKADVDNALNAVTRAKAALNGAENLRNAKTSATNTINGLPNLTQLQKDNLKHQVEQAQNVVGVNGVKDKGNTLNTAMGALRTSIQNDNTTKTSQNYLDASDSNKNNYNTAVNNANGVINATNNPNMDANAINDMANQVNTTKAALNGAQNLAQAKTNATNTINNAQDLNQKQKDALKTQVNNAQRVSDANNVQHTATELNGAMTALKAAIADKERTKASGNYVNADQEKRQAYDSKVTNAENIINGTPNATLTVNDVNSAASQVNAAKTALNGDNNLRVAKEHANNTIDGLAQLNNVQKAKLKEQVQSATTLDGVQTVKNSSQTLNTAMKGLRDSIANEATIKAGQNYTDASPNNRNEYDSAVTAAKAIINQTSNPTMEPNTITQATSQVTTKEHALNGAQNLAQAKTTAKNNLNNLTSINNAQKDALTRNIDGATTVAGVNQETAKATELNNAMHSLQNGINDETQTKQTQKYLDAEPSKKSAYDQAVNAAKAILTKASGQNVDKAAVEQALQNVNSTKTALNGDAKLNEAKAAAKQTLGTLTHINNAQRNALDNEITQATNVEGVNTVKAKAQQLDGAMGQLETSIRDKDTTLQSQNYQDADDAKRTAYSQAVNAAATILNKTAGGNTPKADVERAMQAVTQANTALNGIQNLERAKQAANTAITNASDLNTKQKEALKAQVTSAGRVSAANGVEHTATELNTAMTALKRAIADKADTKASGNYVNADANKRQAYDEKVTAAEHIVSGTPTPTLTPSDVTNAATQVTNAKTQLNGNHNLEVAKQNANTAIDGLTSLNGPQKAKLKEQVGQATTLPNVQTVRDNAQTLNTAMKGLRDSIANEATIKAGQNYTDASQNKQNDYNNAVTAAKAIIGQTTSPSMIAQEINQAKDQVTAKQQALNGQENLRTAQTNAKQHLNGLSDLTNAQKDAAKRQIEGATHVNEVTQAQNNADALNTAMTNLKNGIQDQNTIKQGVNFTDADEAKRNAYTNAVTQAEQILNKAQGPNTAKDGVETALQNVQRAKNELNGNQNVANAKTTAKNALNNLTSINNAQKAALKSQIEGATTVAGVNQVSTMASELNTAMSNLQRGINDEAATKAAQKYTEADRDKQTAYNDAVTAAKTLLDKTAGSNDNKVAVEQALQRVNTAKTALNGDARLNEAKNTAKQQLATMSHLTNAQKANLTEQIERGTTVAGVQGIQANAGTLNQAMNQLRQSIASKDATKSSEDYQDANADLQNAYNDAVTNAEGIISATNNPEMNPDTINQKASQVNSAKSALNGDEKLAAVKQTAKSDIGRLTDLNNAQRTAANAEVDQAPNLAAVTAAKNKATSLNTAMGNLKHALAEKDNTKRSVNYTDADQPKQQAYDTAVTQAEAITNANGSNANETQVQAALNQLNQAKNDLNGDNKVAQAKETAKRALASYSNLNNAQSTAATSQIDNATTVADVTAAQNTANELNTAMGQLQNGINDQNTVKQQVNFTDADQGKKDAYTNAVTNAQGILDKANGQNMTKAQVEAALNQVTTAKNALNGDANVRQAKSDAKANLGTLTHLNNAQKQDLTSQIEGATTVNGVNSVKTKAQDLDGAMQRLESAIANKDQTKASENYIDADPTKKTAFDNAITQAESYLNKDHGTNKDKQAVEQAIQSVTSTENALNGDANLQCAKTEATQAIDNLTQLNTPQKTALKQQVNAAQRVSGVTDLKNSATSLNNAMDQLKQAIGDHDTIVAGGNYTNASPDKQGAYTDAYNAAKNIVNGSPNVITNAADVTAATQRVNNAETSLNGDTNLATAKQQAKDALRQMTHLSDAQKQSITGQIDSATQVTGVQSVKDNATNLDNAMNQLRNSIANKDEVKASQPYVDADTDKQNAYNTAVTSAENIINATSQPTLDPSAVTQAANQVNTNKTALNGAQNLANKKQETTANINRLSHLNNAQKQDLNTQVTNAPNISTVNQVKTKAEQLDQAMERLINGIQDKDQVKQSVNFTDADPEKQTAYNNAVTAAENIINQANGTNANQSQVEAALSTVTTTKQALNGDRKVTDAKNNANQTLSTLDNLNNAQKGAVTGNINQAHTVAEVTQAIQTAQELNTAMGNLKNSLNDKDTTLGSQNFADADPEKKNAYNEAVRNAENILNKSTGTNVPKDQVEAAMNQVNTTKAALNGTQNLEKAKQHANTAIDGLSHLTNAQKEALKQLVQQSTTVAEAQGNEQKANNVDAAMDKLRQSIADNATTKQNQNYTDASPNKKDAYNNAVTTAQGIIDQTTNPSLDPTVINQAAGQVSTSKNALNGNENLEAAKQQATQSLGSLDNLNNAQKQAVTNQINGAHTVDEANQIKQNAQNLNTAMGNLKQAIADKDATKATVNFTDADQAKQQAYNTAVTNAENIISKANGGNATQTEVEQAIQQVNAAKQALNGNANVQHAKDEATALINNSNDLNQAQKDALKQQVQNATTVAGVNNVKQTAQELNNAMTQLKQGIADKEQTKADGNFVNADSDKQNAYNQAVAKAEALISGTPDVVVTPSEITAALNKVTQAKNDLNGNTNLATAKQNVQHAIDQLPNLNQAQRDEYSKQITQATLVPNVNAIQQAATTLNDAMTQLKQGIANKAQIKGSENYHDADTDKQTAYDNAVTKAEELLKQTTNPTMDPNTIQQALTKVNDTNQALNGNQKLADAKQDAKTTLGTLDHLNDAQKQALTTQVEQAPDIATVNNVKQNAQNLNNAMTNLNNALQDKTETLNSINFTDADQAKKDDYTNAVSHAEGILSKANGSNASQTEVEQAMQRVNEAKQALNGNDNVQRAKDAAKQVITNANDLNQAQKDALKQQVDAAQTVANVNTIKQTAQDLNQAMTQLKQGIADKDQTKANGNFVNADTDKQNAYNNAVAHAEQIISGTPNANVDPQQVAQALQQVNQAKGDLNGNHNLQVAKDNANTAIDQLPNLNQPQKTALKDQVSHAELVTGVNAIKQNADALNNAMGTLKQQIQANSQVPQSVDFTQADQDKQQAYNNAANQAQQIANGTPTPVLAPDTVTKAVTTMNQAKDALNGDEKLAQAKQDALANLDTLRDLNQPQRDALRNQINQAQALATVEQTKQNAQNVNTAMGNLKQGIANKDTVKASENYHDADVDKQTAYTNAVSQAEGIINQTTNPTLNPDDITRALTQVTDAKNSLNGEAKLATEKQNAKDAVSGMTHLNDAQKQALKGQIDQSPEIATVNQVKQTATSLDQAMDQLSQAINDKDQILADGNYLNADPDKQNAYKQAVAKAEALLNKQSGTNEVQAQVESITNEVNAAKQALNGNDNLANAKQQAKQQLANLTHLNDAQKQSFESQITQAPLVTDVTTINQKAQTLDHAMELLRNSVADNQTTLASEDYHDATAQRQNDYNKAVTAANNIINQTTSPTMNPDDVNGATTQVNNTKVALDGDENLAAAKQQANNRLDQLDHLNNAQKQQLQSQITQSSDIAAVNGHKQTAESLNTAMGNLINAIADHQAVEQRGNFINADTDKQTAYNTAVNEAAAMINKQTGQNANQTEVEQAITKVQTTLQALNGDHNLQVAKTNATQAIDVLTSLNDPQKTALKDQVTAATLVTAVHQIEQNANTLNQAMHGLRQSIQDNAATKANSKYINEDQPEQQNYDQAVQAANNIINEQTATLDNNAINQVAATVNTTKAALHGDVKLQNDKDHAKQTVSQLAHLNNAQKHMEDTLIDSETTRTAVKQDLTEVQALDQLMDALQQSIADKDATRASSAYVNAEPNKKQAYDEAVQNAESIIAGLNNPTINKGNVSSATQAVISSKNALDGVERLAQDKQTAGNSLNHLDQLTPAQQQALENQINNATTCDKVAEIIAQAQALNEAMKALKESIKDQPQTEASSKFINEDQAQKDAYTQAVQHAKDLINKTTDPTLAKSIIDQATQAVTDAKNNLHGDQKLAQDKQRATETLNNLSNLNTPQRQALENQINNAATRGEVAQKLTEAQALNQAMEALRNSIQDQQQTESGSKFINEDKPQKDAYQAAVQNAKDLINQTGNPTLDKAQVEQLTHAFKQAKDNLHGDQKLADDKQHAVTDLNQLNGLNNPQRQALESQINNAATRGEVAQKLAEAKALDQAMQALRNSIQDQQQTEAGSKFINEDKPQKDAYQAAVQNAKDLINQTGNPTLDKSQVEQLTQAVTTAKDNLHGDQKLARDQQQAVTTVNALPNLNHAQQQTLTDAINAAPTRTEVAQHVQTATELDHAMETLKNKVDQVNTDKAQPNYTEASTDKKEAVDQALQAAQSITDPTNGSNANKDAVEQALTKLQEKVNELNGNERVAEAKTQAKQTIDQLTHLNADQIATAKQNIDQATKLQPIAELVDQATQLNQSMDQLQQAVNEHANVEQTIDYTQADSDKQKAYKQAIADAENVLKQNANKQQVDQALQNILNAKQALNGDERVALAKTNGKHDIDQLNALNNAQQDGFKGRIDQSNDLNQIQQIVDEAKALNRAMDQLSQEITGNEGRTKGSTNYVNADTQVKQVYDEAVDKAKQALDKSSGQNLTAEQVIKLNDAVTAAKKALNGEERLNNRKAEALQRLDQLTHLNNAQRQLAIQQINNAETLNKASRAINRATKLDNAMGAVQQYIDEQHLGVISSTNYINADDNLKANYDNAIANAAHELDKVQGNAIAKAEAEQLKQNIIDAQNALNGDQNLANAKDKANAFVNSLNGLNQQQQDLAHKAINNADTVSDVTDIVNNQIDLNDAMETLKHLVDNEIPNAEQTVNYQNADDNAKTNFDDAKRLANTLLNSDNTNVNDINGAIQAVNDAIHNLNGDQRLQDAKDKAIQSINQALANKLKEIEASNATDQDKLIAKNKAEELANSIINNINKATSNQAVSQVQTAGNHAIEQVHANEIPKAKIDANKDVDKQVQALIDEIDRNPNLTDKEKQALKDRINQILQQGHNDINNALTKEEIEQAKAQLAQALQDIKDLVKAKEDAKQDVDKQVQALIDEIDQNPNLTDKEKQALKDRINQILQQGHNGINNAMTKEEIEQAKAQLAQALKEIKDLVKAKENAKQDVDKQVQALIDEIDQNPNLTDKEKQALKDRINQILQQGHNDINNAMTKEEIEQAKAQLAQALQDIKDLVKAKEDAKNAIKALANAKRDQINSNPDLTPEQKAKALKEIDEAEKRALQNVENAQTIDQLNRGLNLGLDDIRNTHVWEVDEQPAVNEIFEATPEQILVNGELIVHRDDIITEQDILAHINLIDQLSAEVIDTPSTATISDSLTAKVEVTLLDGSKVIVNVPVKVVEKELSVVKQQAIESIENAAQQKIDEINNSVTLTLEQKEAAIAEVNKLKQQAIDHVNNAPDVHSVEEIQQQEQAYIEQFNPEQFTIEQAKSNAIKSIEDAIQHMIDEIKARTDLTDKEKQEAIAKLNQLKEQAIQAIQRAQSISEITEQLEQFKAQMKAANPTAKELAKRKQEAISRIKDFSNEKINSIRNSEIGTADEKQAAMNQINEIVLETIRDINNAHTLQQVEAALNNGIARISAVQIVISDRAKQSSSTGNESNSHLTIGYGTANHPFNSSTIGHKKKLDEDDDIDPLHMRHFSNNFGNVIKNAIGVVGISGLLASFWFFIAKRRRKEDEEEELEIRDNNKDSIKETLDDTKHLPLLFAKRRRKEDEEDVTVEEKDSLNNGESLDKVKHTPFFLPKRRRKEDEEDVEVTNENTDEKVLKDNEHSPLLFAKRRKDKEEDVETTTSIESKDEDVPLLLAKKKNQKDNQSKDKKSASKNTSKKVAAKKKKKKSKKNKK

SEQ ID NO: 14 - protein, 701 aa, Staphylococcus epidermidis
MNNRDKLQKFSIRKYAIGTFSTVIATLVFMGINTNHASADELNQNQKLIKQLNQTDDDDSNTHSQEIENNKQNSSGQTESLRSSTSQNQANARLSDQFKDTNETSQQLPTNVSDDSINQSHSEANMNNEPLKVDNSTMQAHSKIVSDSDGNASENKHHKLTENVLAESRASKNDKEKENLQEKDKSQQVHPPLDKNALQAFFDASYHNYRMIDRDRADATEYQKVKSTFDYVNDLLGNNQNIPSEQLVSAYQQLEKALELARTLPQQSTTEKRGRRSTRSVVENRSSRSDYLDARTEYYVSKDDDDSGFPPGTFFHASNRRWPYNLPRSRNILRASDVQGNAYITTKRLKDGYQWDILFNSNHKGHEYMYYWFGLPSDQTPTGPVTFTIINRDGSSTSTGGVGFGSGAPLPQFWRSAGAINSSVANDFKHGSATNYAFYDGVNNFSDFARGGELYFDREGATQTNKYYGDENFALLNSEKPDQIRGLDTIYSFKGSGDVSYRISFKTQGAPTARLYYAAGARSGEYKQATNYNQLYVEPYKNYRNRVQSNVQVKNRTLHLKRTIRQFDPTLQRTTDVPILDSDGSGSIDSVYDPLSYVKNVTGTVLGIYPSYLPYNQERWQGANAMNAYQIEELFSQENLQNAARSGRPIQFLVGFDVEDSHHNPETLLPVNLYVKPELKHTIELYHDNEKQDRKEFSVSK

SEQ ID NO: 15 - protein, 9439 aa, Staphylococcus epidermidis
MSGTLHNTVGSGILPYQQEIRIKLTSNEPIKDSEWSITGYPNTLTLQNAVGRTNNATEKNLALVGHIDPGNYFITVKFGDKVEQFEIRSKPTPPRIITTANELRGNPNHKPEIRVTDIPNDTTAKIKLVMGGTDGDHDPEINPYTVPENYTVVAEAYHDNDPSKNGVLTFRSSDYLKDLPLSGELKAIVYYNQYVQSNFSKSVPFSSDTTPPTINEPAGLVHKYYRGDHVEITLPVTDNTGGSGLRDVNVNLPQGWTKTFTINPNNNTEGTLKLIGNIPSNEAYNTTYHFNITATDNSGNTTNPAKTFILNVGKLADDLNPVGLSRDQLQLVTDPSSLSNSEREEVKRKISEANANIRSYLLQNNPILAGVNGDVTFYYRDGSVDVIDAENVITYEPERKSIFSENGNTNKKEAVITIARGQNYTIGPNLRKYFSLSNGSDLPNRDFTSISAIGSLPSSSEISRLNVGNYNYRVNAKNAYHKTQQELNLKLKIVEVNAPTGNNRVYRVSTYNLTNDEINKIKQAFKAANSGLNLNDNDITVSNNFDHRNVSSVTVTIRKGDLIKEFSSNLNNMNFLRWVNIRDDYTISWTSSKIQGRNTDGGLEWSPDHKSLIYKYDATLGRQINTNDVLTLLQATAKNSNLRSNINSNEKQLAERGSNGYSKSIIRDDGEKSYLLNSNPIQVLDLVEPDNGYGGRQVSHSNVIYNEKNSSIVNGQVPEANGASAFNIDKVVKANAANNGIMGVIYKAQLYLAPYSPKGYIEKLGQNLSNTNNVINVYFVPSDKVNPSITVGNYDHHTVYSGETFKNTINVNDNYGLNTVASTSDSAITMTRNNNELVGQAPNVTNSINKIVKVKATDKSGNESIVSFTVNIKPLNEKYRITTSSSNQTPVRISNIQNNANLSIEDQNRVKSSLSMTKILGTRNYVNESNNDVRSQVVSKVNRSGNNATVNVTTTFSDGTTNTITVPVKHVLLEVVPTTRTTVRGQQFPTGKGTSPNDFFSLRTGGPVDARIVWVNNQGPDINSNQIGRDLTLHAEIFFDGETTPIRKDTTYKLSQSIPKQIYETTINGRFNSSGDAYPGNFVQAVNQYWPEHMDFRWAQGSGTPSSRNAGSFTKTVTVVYQNGQTENVNVLFKVKPNKPVIDSNSVISKGQLNGQQILVRNVPQNAQVTLYQSNGTVIPNTNTTIDSNGIATVTIQGTLPTGNITAKTSMTNNVTYTKQNSSGIASNTTEDISVFSENSDQVNVTAGMQAKNDGIKIIKGTNYNFNDFNSFISNIPAHSTLTWNEEPNSWKNNIGTTTKTVTVTLPNHQGTRTVDIPITIYPTVTAKNPVRDQKGRNLTNGTDVYNYIIFENNNRLGGTASWKDNRQPDKNIAGVQNLIALVNYPGISTPLEVPVKVWVYNFDFTQPIYKIQVGDTFPKGTWAGYYKHLENGEGLPIDGWKFYWNQQSTGTTSDQWQSLAYTRTPFVKTGTYDVVNPSNWGVWQTSQSAKFIVTNAKPNQPTITQSKTGDVTVTPGAVRNILISGTNDYIQASADKIVINKNGNKLTTFVKNNDGRWTVETGSPDINGIGPTNNGTAISLSRLAVRPGDSIEAIATEGSGETISTSATSEIYIVKAPQPEQVATHTYDNGTFDILPDNSRNSLNPTERVEINYTEKLNGNETQKSFTITKNNNGKWTINNKPNYVEFNQDNGKVVFSANTIKPNSQITITPKAGQGNTENTNPTVIQAPAQHTLTINEIVKEQGQNVTNDDINNAVQVPNKNRVAIKQGNALPTNLAGGSTSHIPVVIYYSDGSSEEATETVRTKVNKTELINARRRLDEEISKENKTPSSIRNFDQAMNRAQSQINTAKSDADQVIGTEFATPQQVNSALSKVQAAQNKINEAKALLQNKADNSQLVRAKEQLQQSIQPAASTDGMTQDSTRNYNNKRQAAEQAIQHANSVINNGDATSQQINDAKNTVEQAQRDYVEAKSNLRADKSQLQSAYDTLNRDVLTNDKKPASVRRYNEAISNIRKELDTAKADASSTLRNTNPSVEQVRDALNKINTVQPKVNQAIALLQPKENNSELVQAKKRLQDAVNDIPQTQGMTQQTINNYNDKQREAERALTSAQRVIDNGDATTQEITSEKSKVEQAMQALTNAKSNLRADKNELQTAYNKLIENVSTNGKKPASIRQYETAKARIQNQINDAKNEAERILGNDNPQVSQVTQALNKIKAIQPKLTEAINMLQNKENNTELVNAKNRLENAVNDTDPTHGMTQETINNYNAKKREAQNEIQKANMIINNGDATAQDISSEKSKVEQVLQALQNAKNDLRADKRELQTAYNKLIQNVNTNGKKPSSIQNYKSARRNIENQYNTAKNEAHNVLENTNPTVNAVEDALRKINAIQPEVTKAINILQDKEDNSELVRAKEKLDQAINSQPSLNGMTQESINNYTTKRREAQNIASSADTIINNGDASIEQITENKIRVEEATNALNEAKQHLTADTTSLKTEVRKLSRRGDTNNKKPSSVSAYNNTIHSLQSEITQTENRANTIINKPIRSVEEVNNALHEVNQLNQRLTDTINLLQPLANKESLKEARNRLESKINETVQTDGMTQQSVENYKQAKIKAQNESSIAQTLINNGDASDQEVSTEIEKLNQKLSELTNSINHLTVNKEPLETAKNQLQANIDQKPSTDGMTQQSVQSYERKLQEAKDKINSINNVLANNPDVNAIRTNKVETEQINNELTQAKQGLTVDKQPLINAKTALQQSLDNQPSTTGMTEATIQNYNAKRQKAEQVIQNANKIIENAQPSVQQVSDEKSKVEQALSELNNAKSALRADKQELQQAYNQLIQPTDLNNKKPASITAYNQRYQQFSNELNSTKTNTDRILKEQNPSVADVNNALNKVREVQQKLNEARALLQNKEDNSALVRAKEQLQQAVDQVPSTEGMTQQTKDDYNSKQQAAQQEISKAQQVIDNGDATTQQISNAKTNVERALEALNNAKTGLRADKEELQNAYNQLTQNIDTSGKTPASIRKYNEAKSRIQTQIDSAKNEANSILTNDNPQVSQVTAALNKIKAVQPELDKAIAMLKNKENNNALVQAKQQLQQIVNEVDPTQGMTTDTANNYKSKKREAEDEIQKAQQIINNGDATEQQITNETNRVNQAINAINKAKNDLRADKSQLENAYNQLIQNVDTNGKKPASIQQYQAARQAIETQYNNAKSEAHQILENSNPSVNEVAQALQKVEAVQLKVNDAIHILQNKENNSALVTAKNQLQQSVNDQPLTTGMTQDSINNYEAKRNEAQSAIRNAEAVINNGDATAKQISDEKSKVEQALAHLNDAKQQLTADTTELQTAVQQLNRRGDTNNKKPRSINAYNKAIQSLETQITSAKDNANAVIQKPIRTVQEVNNALQQVNQLNQQLTEAINQLQPLSNNDALKAARLNLENKINQTVQTDGMTQQSIEAYQNAKRVAQNESNTALALINNGDADEQQITTETDRVNQQTTNLTQAINGLTVNKEPLETAKTALQNNIDQVPSTDGMTQQSVANYNQKLQIAKNEINTINNVLANNPDVNAIKTNKAEAERISNDLTQAKNNLQVDTQPLEKIKRQLQDEIDQGTNTDGMTQDSVDNYNDSLSAAIIEKGKVNKLLKRNPTVEQVKESVANAQQVIQDLQNARTSLVPDKTQLQEAKNRLENSINQQTDTDGMTQDSLNNYNDKLAKARQNLEKISKVLGGQPTVAEIRQNTDEANAHKQALDTARSQLTLNREPYINHINNESHLNNAQKDNFKAQVNSAPNHNTLETIKNKADTLNQSMTALSESIADYENQKQQENYLDASNNKRQDYDNAVNAAKGILNQTQSPTMSADVIDQKAEDVKRTKTALDGNQRLEVAKQQALNHLNTLNDLNDAQRQTLTDTINHSPNINSVNQAKEKANTVNTAMTQLKQTIANYDDELHDGNYINADKDKKDAYNNAVNNAKQLINQSDANQAQLDPAEINKVTQRVNTTKNDLNGNDKLAEAKRDANTTIDGLTYLNEAQRNKAKENVGKASTKTNITSQLQDYNQLNIAMQALRNSVNDVNNVKANSNYINEDNGPKEAYNQAVTHAQTLINAQSNPEMSRDVVNQKTQAVNTAHQNLHGQQKLEQAQSSANTEIGNLPNLTNTQKAKEKELVNSKQTRTEVQEQLNQAKSLDSSMGTLKSLVAKQPTVQKTSVYINEDQPEQSAYNDSITMGQTIINKTADPVLDKTLVDNAISNISTKENALHGEQKLTTAKTEAINALNTLADLNTPQKEAIKTAINTAHTRTDVTAEQSKANQINSAMHTLRQNISDNESVTNESNYINAEPEKQHAFTEALNNAKEIVNEQQATLDANSINQKAQAILTTKNALDGEEQLRRAKENADQEINTLNQLTDAQRNSEKGLVNSSQTRTEVASQLAKAKELNKVMEQLNHLINGKNQMINSSKFINEDANQQQAYSNAIASAEALKNKSQNPELDKVTIEQAINNINSAINNLNGEAKLTKAKEDAVASINNLSGLTNEQKTKENQAVNGAQTRDQVANKLRDAEALDQSMQTLRDLVNNQNAIHSTSNYFNEDSTQKNTYDNAIDNGSTYITGQHNPELNKSTIDQTISRINTAKNDLHGVEKLQRDKGTANQEIGQLGYLNDPQKSGEESLVNGSNTRSEVEEHLNEAKSLNNAMKQLRDKVAEKTNVKQSSDYINDSTEHQRGYDQALQEAENIINEIGNPTLNKSEIEQKLQQLTDAQNALQGSHLLEEAKNNAITGINKLTALNDAQRQKAIENVQAQQTIPAVNQQLTLDREINTAMQALRDKVGQQNNVHQQSNYFNEDEQPKHNYDNSVQAGQTIIDKLQDPIMNKNEIEQAINQINTTQTALSGENKLHTDQESTNRQIEGLSSLNTAQINAEKDLVNQAKTRTDVAQKLAAAKEINSAMSNLRDGIQNKEDIKRSSAYINADPTKVTAYDQALQNAENIINATPNVELNKATIEQALSRVQQAQQDLDGVQQLANAKQQATQTVNGLNSLNDGQKRELNLLINSANTRTKVQEELNKATELNHAMEALRNSVQNVDQVKQSSNYVNEDQPEQHNYDNAVNEAQATINNNAQPVLDKLAIERLTQTVNTTKDALHGAQKLTQDQQAAETGIRGLTSLNEPQKNAEVAKVTAATTRDEVRNIRQEATTLDTAMLGLRKSIKDKNDTKNSSKYINEDHDQQQAYDNAVNNAQQVIDETQATLSSDTINQLANAVTQAKSNLHGDTKLQHDKDSAKQTIAQLQNLNSAQKHMEDSLIDNESTRTQVQHDLTEAQALDGLMGALKESIKDYTNIVSNGNYINAEPSKKQAYDAAVQNAQNIINGTNQPTINKGNVTTATQTVKNTKDALDGDHRLEEAKNNANQTIRNLSNLNNAQKDAEKNLVNSASTLEQVQQNLQTAQQLDNAMGELRQSIAKKDQVKADSKYLNEDPQIKQNYDDAVQRVETIINETQNPELLKANIDQATQSVQNAEQALHGAEKLNQDKQTSSTELDGLTDLTDAQREKLREQINTSNSRDDIKQKIEQAKALNDAMKKLKEQVAQKDGVHANSDYTNEDSAQKDAYNNALKQAEDIINNSSNPNLNAQDITNALNNIKQAQDNLHGAQKLQQDKNTTNQAIGNLNHLNQPQKDALIQAINGATSRDQVAEKLKEAEALDEAMKQLEDQVNQDDQISNSSPFINEDSDKQKTYNDKIQAAKEIINQTSNPTLDKQKIADTLQNIKDAVNNLHGDQKLAQSKQDANNQLNHLDDLTEEQKNHFKPLINNADTRDEVNKQLEIAKQLNGDMSTLHKVINDKDQIQHLSNYINADNDKKQNYDNAIKEAEDLIHNHPDTLDHKALQDLLNKIDQAHNELNGESRFKQALDNALNDIDSLNSLNVPQRQTVKDNINHVTTLESLAQELQKAKELNDAMKAMRDSIMNQEQIRKNSNYTNEDLAQQNAYNHAVDKINNIIGEDNATMDPQIIKQATQDINTAINGLNGDQKLQDAKTDAKQQITNFTGLTEPQKQALENIINQQTSRANVAKQLSHAKFLNGKMEELKVAVAKASLVRQNSNYINEDVSEKEAYEQAIAKGQEIINSENNPTISSTDINRTIQEINDAEQNLHGDNKLRQAQEIAKNEIQNLDGLNSAQITKLIQDIGRTTTKPAVTQKLEEAKAINQAMQQLKQSIADKDATLNSSNYLNEDSEKKLAYDNAVSQAEQLINQLNDPTMDISNIQAITQKVIQAKDSLHGANKLAQNQADSNLIINQSTNLNDKQKQALNDLINHAQTKQQVAEIIAQANKLNNEMGTLKTLVEEQSNVHQQSKYINEDPQVQNIYNDSIQKGREILNGTTDDVLNNNKIADAIQNIHLTKNDLHGDQKLQKAQQDATNELNYLTNLNNSQRQSEHDEINSAPSRTEVSNDLNHAKALNEAMRQLENEVALENSVKKLSDFINEDEAAQNEYSNALQKAKDIINGVPSSTLDKATIEDALLELQNARESLHGEQKLQEAKNQAVAEIDNLQALNPGQVLAEKTLVNQASTKPEVQEALQKAKELNEAMKALKTEINKKEQIKADSRYVNADSGLQANYNSALNYGSQIIATTQPPELNKDVINRATQTIKTAENNLNGQSKLAEAKSDGNQSIEHLQGLTQSQKDKQHDLINQAQTKQQVDDIVNNSKQLDNSMNQLQQIVNNDNTVKQNSDFINEDSSQQDAYNHAIQAAKDLITAHPTIMDKNQIDQAIENIKQALNDLHGSNKLSEDKKEASEQLQNLNSLTNGQKDTILNHIFSAPTRSQVGEKIASAKQLNNTMKALRDSIADNNEILQSSKYFNEDSEQQNAYNQAVNKAKNIINDQPTPVMANDEIQSVLNEVKQTKDNLHGDQKLANDKTDAQATLNALNYLNQAQRGNLETKVQNSNSRPEVQKVVQLANQLNDAMKKLDDALTGNDAIKQTSNYINEDTSQQVNFDEYTDRGKNIVAEQTNPNMSPTNINTIADKITEAKNDLHGVQKLKQAQQQSINTINQMTGLNQAQKEQLNQEIQQTQTRSEVHQVINKAQALNDSMNTLRQSITDEHEVKQTSNYINETVGNQTAYNNAVDRVKQIINQTSNPTMNPLEVERATSNVKISKDALHGERELNDNKNSKTFAVNHLDNLNQAQKEALTHEIEQATIVSQVNNIYNKAKALNNDMKKLKDIVAQQDNVRQSNNYINEDSTPQNMYNDTINHAQSIIDQVANPTMSHDEIENAINNIKHAINALDGEHKLQQAKENANLLINSLNDLNAPQRDAINRLVNEAQTREKVAEQLQSAQALNDAMKHLRNSIQNQSSVRQESKYINASDAKKEQYNHAVREVENIINEQHPTLDKEIIKQLTDGVNQANNDLNGVELLDADKQNAHQSIPTLMHLNQAQQNALNEKINNAVTRTEVAAIIGQAKLLDHAMENLEESIKDKEQVKQSSNYINEDSDVQETYDNAVDHVTEILNQTVNPTLSIEDIEHAINEVNQAKKQLRGKQKLYQTIDLADKELSKLDDLTSQQSSSISNQIYTAKTRTEVAQAIEKAKSLNHAMKALNKVYKNADKVLDSSRFINEDQPEKKAYQQAINHVDSIIHRQTNPEMDPTVINSITHELETAQNNLHGDQKLAHAQQDAANVINGLIHLNVAQREVMINTNTNATTREKVAKNLDNAQALDKAMETLQQVVAHKNNILNDSKYLNEDSKYQQQYDRVIADAEQLLNQTTNPTLEPYKVDIVKDNVLANEKILFGAEKLSYDKSNANDEIKHMNYLNNAQKQSIKDMISHAALRTEVKQLLQQAKILDEAMKSLEDKTQVVITDTTLPNYTEASEDKKEKVDQTVSHAQAIIDKINGSNVSLDQVRQALEQLTQASENLDGDQRVEEAKVHANQTIDQLTHLNSLQQQTAKESVKNATKLEEIATVSNNAQALNKVMGKLEQFINHADSVENSDNYRQADDDKIIAYDEALEHGQDIQKTNATQNETKQALQQLIYAETSLNGFERLNHARPRALEYIKSLEKINNAQKSALEDKVTQSHDLLELEHIVNEGTNLNDIMGELANAIVNNYAPTKASINYINADNLRKDNFTQAINNARDALNKTQGQNLDFNAIDTFKDDIFKTKDALNGIERLTAAKSKAEKLIDSLKFINKAQFTHANDEIMNTNSIAQLSRIVNQAFDLNDAMKSLRDELNNQAFPVQASSNYINSDEDLKQQFDHALSNARKVLAKENGKNLDEKQIQGLKQVIEDTKDALNGIQRLSKAKAKAIQYVQSLSYINDAQRHIAENNIHNSDDLSSLANTLSKASDLDNAMKDLRDTIESNSTSVPNSVNYINADKNLQIEFDEALQQASATSSKTSENPATIEEVLGLSQAIYDTKNALNGEQRLATEKSKDLKLIKGLKDLNKAQLEDVTNKVNSANTLTELSQLTQSTLELNDKMKLLRDKLKTLVNPVKASLNYRNADYNLKRQFNKALKEAKGVLNKNSGTNVNINDIQHLLTQIDNAKDQLNGERRLKEHQQKSEVFIIKELDILNNAQKAAIINQIRASKDIKIINQIVDNAIELNDAMQGLKEHVAQLTATTKDNIEYLNADEDHKLQYDYAINLANNVLDKENGTNKDANIIIGMIQNMDDARALLNGIERLKDAQTKAHNDIKDTLKRQLDEIEHANATSNSKAQAKQMVNEEARKALSNINDATSNDLVNQAKDEGQSAIEHIHADELPKAKLDANQMIDQKVEDINHLISQNPNLSNEEKNKLISQINKLVNGIKNEIQQAINKQQIENATTKLDEVIETTKKLIIAKAEAKQMIKELSQKKRDAINNNTDLTPSQKAHALADIDKTEKDALQHIENSNSIDDINNNKEHAFNTLAHIIIWDTDQQPLVFELPELSLQNALVTSEVVVHRDETISLESIIGAMTLTDELKVNIVSLPNTDKVADHLTAKVKVILADGSYVTVNVPVKVVEKELQIAKKDAIKTIDVLVKQKIKDIDSNNELTSTQREDAKAEIERLKKQAIDKVNHSKSIKDIETVKRTDFEEIDQFDPKRFTLNKAKKDIITDVNTQIQNGFKEIETIKGLTSNEKTQFDKQLTALQKEFLEKVEHAHNLVELNQLQQEFNNRYKHILNQAHLLGEKHIAEHKLGYVVVNKTQQILNNQSASYFIKQWALDRIKQIQLETMNSIRGAHTVQDVHKALLQGIEQILKVNVSIINQSFNDSLHNFNYLHSKFDARLREKDVANHIVQTETFKEVLKGTGVEPGKINKETQQPKLHKNDNDSLFKHLVDNFGKTVGVITLTGLLSSFWLVLAKRRKKEEEEKQSIKNHHKDIRLSDTDKIDPIVITKRKIDKEEQIQNDDKHSIPVAKHKKSKEKQLSEEDIHSIPVVKRKQNSDNKDTKQKKVTSKKKKTPQSTKKVVKTKKRSKK

SEQ ID NO: 16 - protein, 1115 aa, Staphylococcus aureus
MRDKKGPVNKRVDFLSNKLNKYSIRKFTVGTASILIGSLMYLGTQQEAEAAENNIENPTTLKDNVQSKEVKIEEVTNKDTAPQGVEAKSEVTSNKDTIEHEASVKAEDISKKEDTPKEVANVAEVQPKSSVTHNAEAPKVRKARSVDEGSFDITRDSKNVVESTPITIQGKEHFEGYGSVDIQKNPTDLGVSEVTRFNVGNESNGLIGALQLKNKIDFSKDFNFKVRVANNHQSNTTGADGWGFLFSKGNAEEYLTNGGILGDKGLVNSGGFKIDTGYIYTSSMDKTEKQAGQGYRGYGAFVKNDSSGNSQMVGENIDKSKTNFLNYADNSTNTSDGKFHGQRLNDVILTYVASTGKMRAEYAGKTWETSITDLGLSKNQAYNFLITSSQRWGLNQGINANGWMRTDLKGSEFTFTPEAPKTITELEKKVEEIPFKKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPINELTEYGPETIAPGHRDEFDPKLPTGEKEEVPGKPGIKNPETGDVVRPPVDSVTKYGPVKGDSIVEKEEIPFKKERKFNPDLAPGTEKVTREGQKGEKTITTPTLKNPLTGEIISKGESKEEITKDPINELTEYGPETITPGHRDEFDPKLPTGEKEEVPGKPGIKNPETGDVVRPPVDSVTKYGPVKGDSIVEKEEIPFEKERKFNPDLAPGTEKVTREGQKGEKTITTPTLKNPLTGEIISKGESKEEITKDPVNELTEFGGEKIPQGHKDIFDPNLPTDQTEKVPGKPGIKNPDTGKVIEEPVDDVIKHGPKTGTPETKTVEIPFETKREFNPKLQPGEERVKQEGQPGSKTITTPITVNPLTGEKVGEGQPTEEITKQPVDKIVEFGGEKPKDPKGPENPEKPSRPTHPSGPVNPNNPGLSKDRAKPNGPVHSMDKNDKVKKSKIAKESVANQEKKRAELPKTGLESTQKGLIFSSIIGIAGLMLLARRRKN

SEQ ID NO: 17 - protein, 1469 aa, Staphylococcus epidermidis
MGKRRQGPINKKVDFLPNKLNKYSIRKFTVGTASILLGSTLIFGSSSHEAKAAEEKQVDPITQANQNDSSERSLENTNQPTVNNEAPQMSSTLQAEEGSNAEAPNVPTIKANSDNDTQTQFSEAPTRNDLARKEDIPAVSKNEELQSSQPNTDSKIEPTTSEPVNLNYSSPFMSLLSMPADSSSNNTKNTIDIPPTTVKGRDNYDFYGRVDIQSNPTDLNATNLTRYNYGQPPGTTTAGAVQFKNQVSFDKDFDFNIRVANNRQSNTTGADGWGFMFSKKDGDDFLKNGGILREKGTPSAAGFRIDTGYYNNDPLDKIQKQAGQGYRGYGTFVKNDSQGNTSKVGSGTPSTDFLNYADNTTNDLDGKFHGQKLNNVNLKYNASNQTFTATYAGKTWTATLSELGLSPTDSYNFLVTSSQYGNGNSGTYADGVMRADLDGATLTYTPKAVDGDPITSTKEIPFNKKREFDPNLAPGTEKVVQKGEPGIETTTTPTYVNPNTGEKVGEGTPTTKITKQPVDEIVHYGGEEIKPGHKDEFDPNAPKGSQTTQPGKPGVKNPDTGEVVTPPVDDVTKYGPVDGDPITSTEEIPFDKKREFNPDLKPGEERVKQKGEPGTKTITTPTTKNPLTGEKVGEGEPTEKITKQPVDEITEYGGEEIKPGHKDEFDPNAPKGSQEDVPGKPGVKNPDTGEVVTPPVDDVTKYGPVDGDPITSTEEIPFDKKREFDPNLAPGTEKVVQKGEPGTKTITTPTTKNPLTGEKVGEGEPTEKITKQPVDEIVHYGGEEIKPGHKDEFDPNAPKGSQEDVPGKPGVKNPDTGEVVTPPVDDVTKYGPVDGDPITSTEEIPFDKKREFNPDLKPGEERVKQKGEPGTKTITTPTTKNPLTGEKVGEGEPTEKVTKQPVDEIVHYGGEEIKPGHKDEFDPNAPKGSQEDVPGKPGVKNPDTGEVVTPPVDDVTKYGPVDGDPITSTEEIPFDKKREFDPNLAPGTEKVVQKGEPGTKTITTPTTKNPLTGEKVGEGEPTEKITKQPVDEIVHYGGEEIKPGHKDEFDPNAPKGSQTTQPGKPGVKNPDTGEVVTPPVDDVTKYGPVDGDPITSTEEIPFDKKREFDPNLAPGTEKVVQKGEPGTKTITTPTTKNPLTGEKVGEGEPTEKITKQPVDEIVHYGGEQIPQGHKDEFDPNAPVDSKTEVPGKPGVKNPDTGEVVTPPVDDVTKYGPKVGNPITSTEEIPFDKKRVFNPDLKPGEERVKQKGEPGTKTITTPILVNPITGEKVGEGKSTEKVTKQPVDEIVEYGPTKAEPGKPAEPGKPAEPGKPAEPGKPAEPGTPAEPGKPAEPGKPAEPGKPAEPGKPAEPGKPAEPGTPAEPGKPAEPGKPAEPGKPAEPGTPAEPGKPAEPGTPAEPGKPAEPGTPTQSGAPEQPNRSMHSTDNKNQLPDTGENRQANEGTLVGSLLAIVGSLFIFGRRKKGNEK

SEQ ID NO: 18 - protein, 167 aa, Staphylococcus aureus
MKKLYTSYGTYGFLHQIKINNPTHQLFQFSASDTSVIFEETDGETVLKSPSIYEVIKEIGEFSEHHFYCAIFIPSTEDHAYQLEKKLISVDDNFRNFGGFKSYRLLRPAKGTTYKIYFGFADRHAYEDFKQSDAFNDHFSKDALSHYFGSSGQHSSYFERYLYPIKE

SEQ ID NO: 19 - protein, 167 aa, Staphylococcus epidermidis
MYLYTSYGTYQFLNQIKLNHQERSLFQFSTNDSSIILEESEGKSILKHPSAYQVIDSTGEFNEHHFYSAIFVPTSEDHRQQLEKKLLLVDVPLRNFGGFKSYRLLKPTEGSTYKIYFGFANRTAYEDFKASDIFNENFSKDALSQYFGASGQHSSYFERYLYPIEDH

SEQ ID NO: 20 - protein, 1141 aa, Staphylococcus aureus
MINRDNKKAITKKGMISNRLNKFSIRKYTVGTASILVGTTLIFGLGNQEAKAAENTSTENAKQDDATTSDNKEVVSETENNSTTENDSTNPIKKETNTDSQPEAKEESTTSSTQQQQNNVTATTETKPQNIEKENVKPSTDKTATEDTSVILEEKKAPNYTNNDVTTKPSTSEIQTKPTTPQESTNIENSQPQPTPSKVDNQVTDATNPKEPVNVSKEELKNNPEKLKELVRNDNNTDRSTKPVATAPTSVAPKRLNAKMRFAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTVKPEEKLYKIGDYVWEDVDKDGVQGTDSKEKPMANVLVTLTYPDGTTKSVRTDANGHYEFGGLKDGETYTVKFETPAGYLPTKVNGTTDGEKDSNGSSITVKINGKDDMSLDTGFYKEPKYNLGDYVWEDTNKDGIQDANEPGIKDVKVTLKDSTGKVIGTTTTDASGKYKFTDLDNGNYTVEFETPAGYTPTVKNTTAEDKDSNGLTTTGVIKDADNMTLDSGFYKTPKYSLGDYVWYDSNKDGKQDSTEKGIKDVKVTLLNEKGEVIGTTKTDENGKYRFDNLDSGKYKVIFEKPAGLTQTVTNTTEDDKDADGGEVDVTITDHDDFILDNGYFEEDTSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKALPETGSENNGSNNATLFGGLFAALGSLLLFGRRKKQNK

SEQ ID NO: 21 - protein, 1056 aa, Staphylococcus epidermidis
MINKKNNLLTKKKPIANKSNKYAIRKFTVGTASIVIGATLLFGLGHNEAKAEENSVQDVKDSNTDDELSDSNDQSSDEEKNDVINNNQSINTDDNNQIIKKEETNNYDGIEKRSEDRTESTTNVDENEATFLQKTPQDNTHLTEEEVKESSSVESSNSSIDTAQQPSHTTINREESVQTSDNVEDSHVSDFANSKIKESNTESGKEENTIEQPNKVKEDSTTSQPSGYTNIDEKISNQDELLNLPINEYENKARPLSTTSAQPSIKRVTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNDVNINFGNIDSPYIIKVISKYDPNKDDYTTIQQTVTMQTTINEYTGEFRTASYDNTIAFSTSSGQGQGDLPPEKTYKIGDYVWEDVDKDGIQNTNDNEKPLSNVLVTLTYPDGTSKSVRTDEDGKYQFDGLKNGLTYKITFETPEGYTPTLKHSGTNPALDSEGNSVWVTINGQDDMTIDSGFYQTPKYSLGNYVWYDTNKDGIQGDDEKGISGVKVTLKDENGNIISTTTTDENGKYQFDNLNSGNYIVHFDKPSGMTQTTTDSGDDDEQDADGEEVHVTITDHDDFSIDNGYYDDESDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDNDSDLGNSSDKSTKDKLPDTGANEDYGSKGTLLGTLFAGLGALLLGKRRKNRKNKN

SEQ ID NO: 22 - protein, 486 aa, Staphylococcus aureus
MSNNFKDDFEKNRQSIDTNSHQDHTEDVEKDQSELEHQDTIENTEQQFPPRNAQRRKRRRDLATNHNKQVHNESQTSEDNVQNEAGTIDDRQVESSHSTESQEPSHQDSTPQHEEEYYNKNAFAMDKSHPEPIEDNDKHETIKDAENNTEHSTVSDKSIAEQSQQPKPYFATGANQANTSKDKHDDVTVKQDKDESKDHHSGKKGAAIGAGTAGVAGAAGAMGVSKAKKHSNDAQNKSNSDKSNNSTEDKASQDKSKDHHNGKKGAAIGAGTAGLAGGAASKSASAASKPHASNNASQNHDEHDNHDRDKERKKGGMAKVLLPLIAAVLIIGALAIFGGMALNNHNNGTKENKIANTNKNNADESKDKDTSKDASKDKSKSTDSDKSKEDQDKATKDESDNDQNNANQANNQAQNNQNQQQANQNQQQQQQRQGGGQRHTVNGQENLYRIAIQYYGSGSPENVEKIRRANGLSGNNIRNGQQIVIP

SEQ ID NO: 23 - protein, 472 aa, Staphylococcus epidermidis
MIELIKMEGMIVVSNNNFKDDFEKNRQSINPDEQQTELKEDDKTNENKKEADSQNSLSNNSNQQFPPRNAQRRKRRRETATNQSKQQDDKHQKNSDAKTTEGSLDDRYDEAQLQQQHDKSQQQNKTEKQSQDNRMKDGKDAAIVNGTSESPEHKSKSTQNRPGPKAQQQKRKSESTQSKPSTNKDKKAATGAGIAGAAGVAGAAETSKRHHNKKDKQDSKHSNHENDEKSVKNDDQKQSKKGKKAAVGAGAAAGVGAAGVAHHNNQNKHHNEEKNSNQNNQYNDQSEGKKKGGFMKILLPLIAAILILGAIAIFGGMALNNHNDSKSDDQKIANQSKKDSDKKDGAQSEDNKDKKSDSNKDKKSDSDKNADDDSDNSSSNPNATSTNNNDNVANNNSNYTNQNQQDNANQNSNNQQATQGQQSHTVYGQENLYRIAIQYYGEGTQANVDKIKRANGLSSNNIHNGQTLVIPQ

SEQ ID NO: 24 - protein, 165 aa, Staphylococcus aureus
MKNKLIAKSLLTIAAIGITTTTIASTADASEGYGPREKKPVSINHNIVEYNDGTFKYQSRPKFNSTPKYIKFKHDYNILEFNDGTFEYGARPQFNKPAAKTDATIKKEQKLIQAQNLVREFEKTHTVSAHRKAQKAVNLVSFEYKVKKMVLQERIDNVLKQGLVR

SEQ ID NO: 25 - protein, 319 aa, Staphylococcus aureus
MKTRIVSSVTTTLLLGSILMNPVANAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAAENFLDPNKASSLLSSGFSPDFATVITMDRKASKQQTNIDVIYERVRDDYQLHWTSTNWKGTNTKDKWTDRSSERYKIDWEKEEMTN

SEQ ID NO: 26 - protein, 428 aa, Staphylococcus aureus
MHMKNKYISKLLVGAATITLATMISNGEAKASENTQQTSTKHQTTQNNYVTDQQKAFYQVLHLKGITEEQRNQYIKTLREHPERAQEVFSESLKDSKNPDRRVAQQNAFYNVLKNDNLTEQEKNNYIAQIKENPDRSQQVWVESVQSSKAKERQNIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLDALVAQKDAEKKVAPKVEAPQIQSPQIEKPKAESPKVEVPQSKLLGYYQSLKDSFNYGYKYLTDTYKSYKEKYDTAKYYYNTYYKYKGAIDQTVLTVLGSGSKSYIQPLKVDDKNGYLAKSYAQVRNYVTESINTGKVLYTFYQNPTLVKTAIKAQETASSIKNTLSNLLSFWK

SEQ ID NO: 27 - protein, 350 aa, Staphylococcus aureus
MTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 28 - protein, 645 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 29 - protein, 953 aa, Staphylococcus aureus
MNNKKTATNRKGMIPNRLNKFSIRKYSVGTASILVGTTLIFGLSGHEAKAAEHTNGELNQSKNETTAPSENKTTEKVDSRQLKDNTQTATADQPKVTMSDSATVKETSSNMQSPQNATASQSTTQTSNVTTNDKSSTTYSNETDKSNLTQAKNVSTTPKTTTIKQRALNRMAVNTVAAPQQGTNVNDKVHFTNIDIAIDKGHVNKTTGNTEFWATSSDVLKLKANYTIDDSVKEGDTFTFKYGQYFRPGSVRLPSQTQNLYNAQGNIIAKGIYDSKTNTTTYTFTNYVDQYTNVSGSFEQVAFAKRENATTDKTAYKMEVTLGNDTYSKDVIVDYGNQKGQQLISSTNYINNEDLSRNMTVYVNQPKKTYTKETFVTNLTGYKFNPDAKNFKIYEVTDQNQFVDSFTPDTSKLKDVTGQFDVIYSNDNKTATVDLLNGQSSSDKQYIIQQVAYPDNSSTDNGKIDYTLETQNGKSSWSNSYSNVNGSSTANGDQKKYNLGDYVWEDTNKDGKQDANEKGIKGVYVILKDSNGKELDRTTTDENGKYQFTGLSNGTYSVEFSTPAGYTPTTANAGTDDAVDSDGLTTTGVIKDADNMTLDSGFYKTPKYSLGDYVWYDSNKDGKQDSTEKGIKGVKVTLQNEKGEVIGTTETDENGKYRFDNLDSGKYKVIFEKPAGLTQTGTNTTEDDKDADGGEVDVTITDHDDFTLDNGYYEEETSDSDSDSDSDSDSDSDSDSDSDSDSDSDSDSDSDSDSDSDSDSDSDSDSDSDSDSESDSDSDSDSDSDSDSDSDSDSDSDSDSDSDSDSDSDSDSDSDSDSDSDSDNDSDSDSDSDSDSDSDSDSDSDSDSDSDSDSDSDSDSDSDSDSDSDSDSDSDSDSDSDSDSDSDSDSDSDAGKHTPTKPMSTVKDQHKTAKALPETGSENNNSNNGTLFGGLFAALGSLLLFGRRKKQNK

SEQ ID NO: 30 - protein, 935 aa, Staphylococcus aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQNTDASNKDVVSQAVNPSTPRMRAFSLAAVAADAPAAGTDITNQLTDVKVTIDSGTTVYPHQAGYVKLNYGFSVPNSAVKGDTFKITVPKELNLNGVTSTAKVPPIMAGDQVLANGVIDSDGNVIYTFTDYVDNKENVTANITMPAYIDPENVTKTGNVTLTTGIGTNTASKTVLIDYEKYGQFHNLSIKGTIDQIDKTNNTYRQTIYVNPSGDNVVLPALTGNLIPNTKSNALIDAKNTDIKVYRVDNANDLSESYYVNPSDFEDVTNQVRISFPNANQYKVEFPTDDDQITTPYIVVVNGHIDPASTGDLALRSTFYGYDSNFIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSASDSDSASDSDSASDSDSASDSDSASDSDSASDSDSASDSDSASDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSESDSDSDSDSDSDSDSDSDSDSDSESDSDSDSDSDSESDSDSDSDSDSDSASDSDSGSDSDSSSDSDSDSTSDTGSDNDSDSDSNSDSESGSNNNVVPPNSPKNGTNASNKNEAKDSKEPLPDTGSEDEANTSLIWGLLASLGSLLLFRRKKENKDKK

SEQ ID NO: 31 - protein, 1038 aa, Staphylococcus aureus
MKNNLRYGIRKHKLGAASVFLGTMIVVGMGQDKEAAASEQKTTTVEENGNSATDNKTSETQTTATNVNHIEETQSYNATVTEQPSNATQVTTEEAPKAVQAPQTAQPANVETVKEEEKPQVKETTQPQDNSGNQRQVDLTPKKVTQNQGTETQVEVAQPRTASESKPRVTRSADVAEAKEASDVSEVKGTDVTSKVTVESGSIEAPQGNKVEPHAGQRVVLKYKLKFADGLKRGDYFDFTLSNNVNTYGVSTARKVPEIKNGSVVMATGEILGNGNIRYTFTNEIEHKVEVTANLEINLFIDPKTVQSNGEQKITSKLNGEETEKTIPVVYNPGVSNSYTNVNGSIETFNKESNKFTHIAYIKPMNGNQSNTVSVTGTLTEGSNLAGGQPTVKVYEYLGKKDELPQSVYANTSDTNKFKDVTKEMNGKLSVQDNGSYSLNLDKLDKTYVIHYTGEYLQGSDQVNFRTELYGYPERAYKSYYVYGGYRLTWDNGLVLYSNKADGNGKNGQIIQDNDFEYKEDTAKGTMSGQYDAKQIIETEENQDNTPLDIDYHTAIDGEGGYVDGYIETIEETDSSAIDIDYHTAVDSEVGHVGGYTESSEESNPIDFEESTHENSKHHADVVEYEEDTNPGGGQVTTESNLVEFDEESTKGIVTGAVSDHTTIEDTKEYTTESNLIELVDELPEEHGQAQGPIEEITENNHHISHSGLGTENGHGNYGVIEEIEENSHVDIKSELGYEGGQNSGNQSFEEDTEEDKPKYEQGGNIVDIDFDSVPQIHGQNKGDQSFEEDTEKDKPKYEHGGNIIDIDFDSVPQIHGFNKHNEIIEEDTNKDKPNYQFGGHNSVDFEEDTLPKVSGQNEGQQTIEEDTTPPTPPTPEVPSEPETPMPPTPEVPSEPETPTPPTPEVPSEPETPTPPTPEVPSEPETPTPPTPEVPSEPETPTPPTPEVPAEPGKPVPPAKEEPKKPSKPVEQGKVVTPVIEINEKVKAVAPTKKAQSKKSELPETGGEESTNKGMLFGGLFSILGLALLRRNKKNNKA

SEQ ID NO: 32 - protein, 877 aa, Staphylococcus aureus
MKKRIDYLSNKQNKYSIRRFTVGTTSVIVGATILFGIGNHQAQASEQSNDTTQSSKNNASADSEKNNMIETPQLNTTANDTSDISANTNSANVDSTTKPMSTQTSNTTTTEPASTNETPQPTAIKNQATAAKMQDQTVPQEANSQVDNKTTNDANSIATNSELKNSQTLDLPQSSPQTISNAQGTSKPSVRTRAVRSLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVNPKDPTPGPPVDPEPSPDPEPEPTPDPEPSPDPEPEPSPDPDPDSDSDSDSGSDSDSGSDSDSESDSDSDSDSDSDSDSDSESDSDSESDSDSDSDSDSDSDSDSESDSDSDSDSDSDSDSDSESDSDSESDSESDSDSDSDSDSDSDSDSDSDSDSDSDSDSDSDSDSDSESDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSRVTPPNNEQKAPSNPKGEVNHSNKVSKQHKTDALPETGDKSENTNATLFGAMMALLGSLLLFRKRKQDHKEKA

SEQ ID NO: 33 - protein, 658 aa, Staphylococcus aureus
MKKQIISLGALAVASSLFTWDNKADAIVTKDYSKESRVNEKSKKGATVSDYYYWKIIDSLEAQFTGAIDLLEDYKYGDPIYKEAKDRLMTRVLGEDQYLLKKKIDEYELYKKWYKSSNKNTNMLTFHKYNLYNLTMNEYNDIFNSLKDAVYQFNKEVKEIEHKNVDLKQFDKDGEDKATKEVYDLVSEIDTLVVTYYADKDYGEHAKELRAKLDLILGDTDNPHKITNERIKKEMIDDLNSIIDDFFMETKQNRPNSITKYDPTKHNFKEKSENKPNFDKLVEETKKAVKEADESWKNKTVKKYEETVTKSPVVKEEKKVEEPQLPKVGNQQEVKTTAGKAEETTQPVAQPLVKIPQETIYGETVKGPEYPTMENKTLQGEIVQGPDFLTMEQNRPSLSDNYTQPTTPNPILEGLEGSSSKLEIKPQGTESTLKGIQGESSDIEVKPQATETTEASQYGPRPQFNKTPKYVKYRDAGTGIREYNDGTFGYEARPRFNKPSETNAYNVTTNQDGTVSYGARPTQNKPSETNAYNVTTHANGQVSYGARPTQKKPSKTNAYNVTTHANGQVSYGARPTQKKPSKTNAYNVTTHANGQVSYGARPTYKKPSETNAYNVTTHANGQVSYGARPTQKKPSETNAYNVTTHADGTATYGPRVTK

SEQ ID NO: 34 - DNA, 1602 bp, Staphylococcus aureus
atgttacaagtaactgatgtgagtttacgttttggagatcgtaaactatttgaagatgtaaatattaaatttacagaaggtaattgttatggattaattggtgcgaatggtgcaggtaaatcaacattcttaaaaatattatctggtgaattagattctcaaacaggacatgtttcattaggtaaaaatgaacgtctagctgttttaaaacaggaccactatgcttatgaagatgaacgcgtgcttgatgttgtaattaaaggtcacgaacgtctttatgaggttatgaaagaaaaagatgaaatctatatgaagccagatttcagtgatgaagatggtatccgtgctgctgaacttgaaggtgaatttgcagaaatgaatggttggaatgctgaagctgatgctgctaaccttttatctggtttaggtatcgatccaactttacacgataaaaaaatggctgaattagaaaacaaccaaaaaattaaagtattattagcgcaaagtttattcggtgaaccagacgtactattactggatgagcctactaacggtctcgatattccagcaatcagttggttagaagatttcttaattaactttgataatactgttatcgtagtatcgcatgaccgtcatttcttaaataatgtatgtactcatatcgctgatttagacttcggtaaaattaaagtttatgttggtaactatgatttttggtatcaatctagtcagttagctcaaaagatggctcaagaacaaaacaagaaaaaagaagaaaaaatgaaagagttacaggactttattgcacgtttctcagctaacgcttctaaatctaaacaagcaacaagtcgtaaaaaacaacttgagaaaattgaattagatgatattcaaccatcatcaagaagatatcctttcgttaaattcacgcctgagcgtgagattggtaacgacttattaatcgttcaaaatctttctaaaacaattgacggcgaaaaagtattagataatgtatcattcacaatgaatccaaatgataaagcgattttaattggagatagtgaaattgcaaaaacaacattacttaaaatattagctggcgaaatggaaccagacgaaggttcatttaaatggggtgttactacatcattaagttacttccctaaagataactcagagttctttgagggtgtaaatatgaatctcgttgattggttaagacaatatgctcctgaagatgaacaaacagaaacatttttacgtggtttcttaggtcgtatgttatttagtggtgaagaagttaagaaaaaagctagtgtgctttcaggtggagaaaaagtacgttgtatgctaagtaaaatgatgttatcaagtgcgaatgtacttttacttgacgaacctactaaccacttagacttagaaagtattactgctgtcaatgatggtcttaaatcatttaaaggttctatcatctttacttcttatgacttcgaatttatcaacacgattgcaaaccgtgttatcgatttaaataaacaaggcggcgtttcaaaagaaattccatatgaagaatacttgcaagaaatcggcgttttaaaataa

SEQ ID NO: 35 - DNA, 1608 bp, Staphylococcus epidermidis
atgttacaagtaactgatgtaagtttacgttttggtgatcgtaaactatttgaagatgtaaatataaaatttacagagggtaattgttatggattaattggtgcaaatggtgctgggaaatctacattcttgaagattttatcaggcgaaattgattcacagactggtcatgtatctctaggtaaagatgagcgtttggctgtgttaaaacaagatcattttgcttatgaagatgaacgtgttttagatgttgtgattaaaggacatgaacgtttgtatcaagtgatgaaagagaaagatgaaatttatatgaaacctgatttcagcgatgaggacggtattcgcgctgcagaacttgaaggagaatttgcagaaatgaacggttggaatgctgaagctgatgctgctaacttattatcaggattaggcatagaacctgacttacatgataaaaatatgtctgaacttgaaaataatcaaaaagttaaggtattgttagctcaaagtttatttggtgatcctgacgttcttttactagatgagcctaccaatggtttagatataccagcaataagttggttagaagactttttaattaattttgaaaatactgtcattgtcgtttcgcatgaccgtcacttcttaaataatgtttgtactcatattgctgatttagactttggcaaaattaaactttatgttggtaactatgatttttggtatcaatcaagtcaattagcacaaaaaatggcacaagaacaaaataagaaaaaagaagaaaaaatgaaagagttacaggatttcatcgcacgcttctcagcaaatgcttctaaatctaaacaggcaacaagtcgtaagaaacaattagaaaaaattgaattagatgatatccagccatcatctcgtagatacccttacgtgaaatttactcctgaacgtgaaattggaaatgatttacttacagtagaaaatctttctaaaacaattgacggcgaaaaagtactagacaatgtttcattcactatgaatcctaatgataaagctattttagttggtgatagcgaaattgctaaaacaacattgttaaaaattttagctggagaaatggaaccagatgaaggtacatttaaatggggtgtaacgacatctttaagttacttccctaaagataactctgagttctttgatggtgtcgatatgaatttagttgaatggttacgtcaatacgctccagaagatgaacaaactgaaacatttttacgtggtttcttaggtcgcatgttatttagtggtgaggaagttaagaaaaaagcaagcgtgctttcaggtggagaaaaagtacgttgcatgttaagtaaaatgatgttatcaagtgctaacgtacttttacttgatgagccaacaaaccatttagatttggaaagtatcactgctgtaaatgacggattaaaatcatttaaaggttctatcatcttcacttcttatgattttgaatttattaatacaatcgcaaatcgagtgattgacttgaatcaagctggtgccctttctaaagaagtaccttatgaggaatacttacaagaaattggtgtattacaaaataattaa

SEQ ID NO: 36 - DNA, 1305 bp, Staphylococcus aureus
atgccaattattacagatgtttacgctcgcgaagtcttagactctcgtggtaacccaactgttgaagtagaagtattaactgaaagtggcgcatttggtcgtgcattagtaccatcaggtgcttcaactggtgaacacgaagctgttgaattacgtgatggagacaaatcacgttatttaggtaaaggtgttactaaagcagttgaaaacgttaatgaaatcatcgcaccagaaattattgaaggtgaattttcagtattagatcaagtatctattgataaaatgatgatcgcattagacggtactccaaacaaaggtaaattaggtgcaaatgctattttaggtgtatctatcgcagtagcacgtgcagcagctgacttattaggtcaaccactttacaaatatttaggtggatttaatggtaagcagttaccagtaccaatgatgaacatcgttaatggtggttctcactcagatgctccaattgcattccaagaattcatgattttacctgtaggtgctacaacgttcaaagaatcattacgttggggtactgaaattttccacaacttaaaatcaattttaagcaaacgtggtttagaaactgcagtaggtgacgaaggtggtttcgctcctaaatttgaaggtactgaagatgctgttgaaacaattatccaagcaatcgaagcagctggttacaaaccaggtgaagaagtattcttaggatttgactgtgcatcatcagaattctatgaaaatggtgtatatgactacagtaagttcgaaggcgaacacggtgcaaaacgtacagctgcagaacaagttgactacttagaacaattagtagacaaatatcctatcattacaattgaagacggtatggacgaaaacgactgggatggttggaaacaacttacagaacgtatcggtgaccgtgtacaattagtaggtgacgatttattcgtaacaaacactgaaattttagcaaaaggtattgaaaacggaattggtaactcaatcttaattaaagttaaccaaatcggtacattaactgaaacatttgatgcaatcgaaatggctcaaaaagctggttacacagcagtagtttctcaccgttcaggtgaaacagaagatacaacaattgctgatattgctgttgctacaaacgctggtcaaattaaaactggttcattatcacgtactgaccgtattgctaaatacaatcaattattacgtatcgaagatgaattatttgaaactgctaaatatgacggtatcaaatcattctataacttagataaataa

SEQ ID NO: 37 - DNA, 1305 bp, Staphylococcus epidermidis
atgccaattattacagatgtttacgctcgcgaagtcttagactcacgtggtaacccaacagttgaagttgaagtattaactgaaagcggtgctttcggacgtgcattagtaccttctggtgcttctactggtgaacatgaagcagttgaattacgtgatggagataaatcacgttatttaggtaaaggtgtgactaaagcggtagaaaatgttaacgaaatgatcgcaccagaaatcgttgaaggtgaattttcagttttagatcaagtatctattgataaaatgatgattcaattagacggtacacacaacaaaggtaaattaggtgcaaatgccattttaggtgtttctattgccgtagctcgtgcagctgctgacttattaggtcaaccattatataaatatttaggtggatttaatggtaaacaattgccagtacctatgatgaatattgttaatggtggttctcactcagatgcaccaattgctttccaagagttcatgattttacctgtaggtgctgagtcattcaaagaatcattacgttggggtgcagaaatcttccataaccttaaatcaatcttaagtgaacgtggtttagaaactgcagtaggtgatgaaggtggtttcgctcctagatttgaaggcactgaagacgctgtagaaactattattaaagctatcgaaaaagcaggatacaaaccaggtgaagatgtattcttaggatttgactgtgcttcttctgaattctatgaaaatggtgtttatgattacactaaattcgaaggtgaacacggtgctaaacgtagtgcagcagagcaagttgactacttagaagaattaattggtaaatatccaatcatcactattgaagatggtatggatgaaaacgattgggaaggttggaaacaattaactgatcgtatcggtgataaagttcaattagttggtgatgatttattcgtaactaacactgaaattttatctaaaggtatcgaacaaggtattggtaactcaatcttaatcaaagtaaaccaaatcggtacattaactgaaacattcgatgctattgaaatggctcaaaaagctggatatactgcggttgtatctcaccgttctggtgaaactgaagatactacaattgctgatatcgcagttgctacaaatgcaggccaaattaaaacaggttcattatctagaactgaccgtattgctaaatacaatcaattattacgtattgaagatgaattatacgaaacagctaaatttgaaggaattaaatctttctacaatttagataaataa

SEQ ID NO: 38 - DNA, 768 bp, Staphylococcus aureus
atgaaaaaaatcgttacagctacaatcgctacagcaggacttgccactatcgcatttgcaggacatgatgcacaagccgcagaacaaaataacaatggatataattctaatgacgctcaatcatacagctatacgtatacaattgatgcacaaggtaattatcattacacttggacaggaaattggaatccaagtcaattaacgcaaaacaacacatactactacaacaactacaatacttatagttataacaatgcatcttacaataactactataatcattcatatcaatacaataactatacaaacaatagccaaacagcaacaaataactattatactggtggttcaggtgcaagttatagcacaacaagtaataatgttcatgtgactacaactgcagcgccatcttcaaatggtcgttcaatttctaatggttatgcatcaggaagtaacttatatacttcaggacaatgtacttattatgtatttgatcgtgttggtgggaaaattggttcaacatggggtaacgcaagtaattgggctaacgcagctgcatcatctggctatacagtgaacaatacaccaaaagttggtgctatcatgcaaacaacacaaggctattacggtcatgttgcttacgttgaaggcgttaacagcaacggttctgttcgtgtttcagaaatgaactatggacatggtgctggtgtggttacgtctcgtacaatttcagcaaaccaagcaggttcatataatttcattcattaa

SEQ ID NO: 39 - DNA, 804 bp, Staphylococcus aureus
atgaagaaaatcgctacagctactatcgcaactgcaggattcgctacaatcgcaattgcatcaggaaatcaagctcatgcttctgagcaagataactacggttataatccaaacgacccaacatcatatagctatacttacactattgatgcacaaggtaactaccattacacatggaaaggtaactggcatccaagtcaattaaaccaagataatggctactacagctattactactacaatggctacaataactacaacaattacaacaatggttatagctacaataattacagccgttacaacaactactcaaataataatcaatcatataactacaataactataatagttacaacacaaacagctaccgtactggtggtttaggtgcaagctacagcacttcaagcaacaatgttcaagtaactacaactatggctccatcatcaaatggccgttcaatctcaagtggttatacttcaggacgtaacttatacacttctggtcaatgtacatactacgtatttgatcgtgtaggtggtaaaatcggttcaacttggggcaatgcaagtaactgggctaacgcagctgcaagagctggttacacagtgaacaatacaccaaaagctggtgcaattatgcaaacaactcaaggtgcatacggtcacgttgcatacgttgaaagtgttaacagcaatggttcagtaagagtttcagaaatgaactatggttatggcccaggtgttgtaacttcacgtacaatctcagctagccaagctgctggttataacttcattcactaa

SEQ ID NO: 40 - DNA, 774 bp, Staphylococcus epidermidis
atgaaaaaaatcgctacagctacaattgcaactgcaggaatcgctactttcgcatttgcacaccatgacgcacaagcagcagaacaaaataatgatgggtacaatccaaacgacccttattcatatagctacacttacacaatcgatgctgaaggtaactaccactacacttggaaaggtaactggagtccagatcgtgtaaatacttcatataactataataattataataactacaactactatggttacaataactatagcaactacaataactacagtaattacaacaattacaacaactatcaatcaaacaacacgcaatcacaaagaacaactcaaccgactggtggtttaggcgcaagctattcaacatcaagtagtaatgttcacgttacaacaacttctgcgccatcatcaaacggtgtatctttatcaaacgctcgctcagcatctggtaacttatacacttcaggtcaatgtacatattatgtatttgacagagtaggtggcaaaatcggttcaacgtggggtaacgcaaacaactgggcaaacgctgcagcacgttctggttacacagtaaacaattcgcctgctaaaggtgcaatcttacaaacgtcacaaggtgcatacggacacgtagcatacgttgaaggtgtaaacagcaatggttcaatcagagtttcagaaatgaactacggtcacggtgcaggtgttgtcacttcacgtacaatctctgcgagccaagctgcttcatataactatattcactaa

SEQ ID NO: 41 - DNA, 930 bp, Staphylococcus aureus
atgaaaaaattagtacctttattattagccttattacttctagttgctgcatgtggtactggtggtaaacaaagcagtgataagtcaaatggcaaattaaaagtagtaacgacgaattcaattttatatgatatggctaaaaatgttggtggagacaacgtcgatattcatagtattgtacctgttggtcaagatcctcatgaatatgaagttaaacctaaagatattaaaaagttaactgacgctgacgttattttatacaacggattaaatttagagactggtaacggttggtttgaaaaagccttagaacaggctggtaaatcattaaaagataaaaaagttatcgcagtatcaaaagatgttaaacctatctatttaaacggtgaagaaggcaacaaagataaacaagatccacacgcatggttaagtttagataatggtattaaatacgtaaaaacaattcaacaaacatttatcgataacgacaaaaaacataaagcagattatgaaaagcaaggtaacaaatacattgctcaattggaaaaattaaataatgacagtaaagacaaatttaatgacattccaaaagaacaacgtgccatgattacaagtgaaggtgccttcaagtacttctcaaaacaatacggtattacaccaggttatatttgggaaattaacactgaaaaacaaggtacacctgaacaaatgagacaagctattgagtttgttaaaaagcacaaattaaaacacttattagtagaaacaagtgttgataagaaagcaatggaaagtttatctgaagaaacgaagaaagatatctttggtgaagtgtacacagattcaatcggtaaagaaggcactaaaggtgactcttactacaaaatgatgaaatcaaatattgaaactgtacacggaagcatgaaataa

SEQ ID NO: 42 - DNA, 930 bp, Staphylococcus epidermidis
gtgaaaaaaattctcgctttagcaatagcatttttaattatccttgccgcatgtgggaatcacagtaaccatgaacatcactcacatgaaggaaaattaaaagttgtaactacaaactctattctctatgacatggttaaacgtgtcggtggaaataaggtcgatgttcatagcatcgttccagtaggacaagacccacatgaatatgaggttaaacctaaagatattaaagcattaacagatgctgacgttgtattttataacggtttaaacctagaaactggaaatggttggtttgaaaaagcacttgaccaagcaggaaaatcaacaaaagataaaaatgtgatagcagcatcaaataatgttaaaccaatatacttaaatggtgaggaaggtaacaaaaacaaacaagatccacatgcatggttaagtttagagaatggaattaaatacgtaaaaacaatacaaaaatcactagaacatcatgataaaaaagataagtctacatatgaaaaacaagggaatgcatatatatcaaaattagaagaacttaataaagatagtaaaaataaatttgatgacatacccaaaaatcaacgtgccatgatgacaagtgaaggtgcatttaaatattttgctcaacaattcgatgttaaaccaggttatatttgggagataaacacagaaaaacaaggtacacctggtcaaatgaaacaagccattaaatttgttaaagataatcatttaaaacatttattagtcgaaacaagcgtagataaaaaagctatgcaaagtttatcagaagaaactaagaaagatatttatggtgaagtatttaccgactctataggtaaggaaggtactaaaggtgactcatactataaaatgatgaaatctaatattgatacaatacatggtagtatgaaataa

SEQ ID NO: 43 - DNA, 702 bp, Staphylococcus aureus
atgaaaaagacaattatggcatcatcattagcagtggcattaggtgtaacaggttacgcagcaggtacaggacatcaagcacacgctgctgaagtaaacgttgatcaagcacacttagttgacttagcgcataatcaccaagatcaattaaatgcagctccaatcaaagatggtgcatatgacatccactttgtaaaagatggtttccaatataactttacttcaaatggtactacatggtcatggagctatgaagcagctaatggtcaaactgctggtttctcaaacgttgcaggtgcagactacactacttcatacaaccaaggttcagatgtacaatcagtaagctacaatgcacaatcaagtaactcaaacgttgaagctgtttcagctccaacttaccataactacagcacttcaactacttcaagttcagtgagattaagcaatggtaatactgcaggtgctactggttcatcagcagctcaaatcatggctcaacgtactggtgtttcagcttctacatgggctgcaatcatcgctcgtgaatcaaatggtcaagtaaatgcttacaacccatcaggtgcttcaggtttattccaaactatgccaggttggggtccgacaaacactgttgaccaacaaatcaacgcagctgttaaagcatacaaagcacaaggtttaggtgcttggggattctaa

SEQ ID NO: 44 - DNA, 708 bp, Staphylococcus epidermidis
atgaaaaaaacagttatcgcttctacattagcagtatctttaggaattgcaggttacggtttatcaggacatgaagcacacgcttcagaaactacaaacgttgataaagcacacttagtagatttagcacaacataatcctgaagaattaaatgctaaaccagttcaagctggtgcttacgatattcatttcgtagacaatggataccaatacaacttcacttcaaatggttctgaatggtcatggagctacgctgtagctggttcagatgctgattacacagaatcatcatcaaaccaagaagtaagtgcaaatacacaatctagtaacacaaatgtacaagctgtttcagctccaacttcttcagaaagtcgtagctacagcacatcaactacttcatactcagcaccaagccataactacagctctcacagtagttcagtaagattatcaaatggtaatactgctggttctgtaggttcatatgctgctgctcaaatggctgcacgtactggtgtatctgcttcaacatgggaacacatcattgctagagaatcaaatggtcaattacatgcacgtaatgcttcaggtgctgctggattattccaaactatgccaggttggggttcaactggttcagtaaatgatcaaatcaatgccgcttataaagcatataaagcacaaggtttatctgcttggggtatgtaa

SEQ ID NO: 45 - DNA, 11670 bp, Staphylococcus aureus
gtgaattatcgtgataaaattcaaaagtttagtattcgtaaatatacagttggtacattttcaactgtcattgcgacattggtatttttaggattcaatacatcacaagcacatgctgctgaaacaaatcaaccagcaagcgtggttaaacagaaacaacaaagtaataatgaacagactgagaatcgagaatctcaagtacaaaattctcaaaattcacaaaatagtcaatcattatccgctactcatgaaaatgagcaaccaaataatagtcaagctaatttagtaaatcaaaaagtagcgcaatcatctactactaatgatgaacaaccagcatctcaaaatgtaaatacaaagaaagattcggcaacggctgcgacaacacaaccagataaagaagaaagtaagcataaacaaaacgaaagtcaatctgctaataaaaatggaaacgacaatagagcggctcatgtagaaaatcatgaagcaaatgtagtaacagcttcagattcatctgataatggtaacgtacaacatgaccgaaatgaattacaagcattttttgatgcaaattatcatgattatcgctttattgaccgtgaaaatgcagattctggcacatttaactatgtaaaaggcatttttgacaagattaatactttattaggcagtaatgatccaattaacaataaagacttgcaacttgcatacaaagaattggaacaagctgttgctttaattcgtacaatgcctcaacgtcaacaaactagccgtcgatcaaacagaattcaaacgcgttctgttgagtctagagctgcagagcctagatcagtatcagactatcaaaatgcaaattcatcatattatgttgaaaatgctaatgatggttcaggatatcctgtaggtacatatatcaatgcttctagtaaaggggcgccatataatttaccaactacaccatggaatacattgaaggcctctgactcaaaggaaattgctcttatgacagcgaaacaaactggagatggctaccaatgggttattaagtttaataaaggacatgctccacatcaaaatatgattttctggtttgcattaccagcagaccaagtgccagtaggaagaactgactttgtaacagttaattcagatggaacaaatgtacaatggagtcatggagcaggagcaggtgcaaataaaccacttcaacaaatgtgggaatatggagtaaatgatcctgatcgttcacatgactttaaaataagaaatagaagtggccaagtaatatatagctggccaactgtccatgtttattctttagaagatttatctagagcgagtgattattttagtgaagctggagcgacacctgctactaaagcatttggtagacaaaattttgaatatattaatggtcaaaaacctgctgaatcaccgggtgttcctaaagtttatactttcatcggtcaaggtgatgcaagttatacaatttcatttaaaacacaaggtccaactgttaataaattgtattatgcagcaggtgggcgtgctttagagtacaatcaattatttatgtacagtcaactatacgtcgaatcaacgcaagaccatcaacaacgtcttaatggtttaagacaagtggttaatcgtacatatcgcataggtacaactaaacgtgtagaagtgagtcaaggaaatgtacaaacgaaaaaggtattagaaagtacaaacctaaatatagatgattttgttgatgatcctttaagttatgttaagacgccgagtaataaagtgttaggtttttacccaactaatgcaaatactaacgcttttagaccggggggcgttcaagaattaaatgaatatcaattaagtcaattatttactgatcaaaaattacaagaagcagcaagaactagaaacccaataagattaatgattggtttcgactatcctgatggttatggtaatagtgaaactttagttcctgttaacttaacggtattacctgaaatccaacataatattaaattctttaaaaatgacgatactcaaaatattgctgaaaaaccattttcaaaacaagctgggcatccagttttctatgtatatgcaggtaaccaagggaatgcttccgtgaatttaggtggtagcgtaacatctattcaaccattacgtattaatttaacaagtaatgagaattttacagataaagattggcaaattacaggtattccgcgtacattacacattgaaaactcgacaaatagaactaataatgctagagaacgtaacattgaacttgttggtaatttattaccaggggattactttggtacgatacgttttggacgtaaagaacaattatttgaaattcgtgttaaaccacatacaccaacaattacaacgacagctgagcaattaagaggtacagcattacaaaaagtgcctgttaatatttcgggaataccgttggatccatcggcattggtttatttagttgcaccaacaaatcaaactacgaatggtggtagtgaggcagatcaaataccatctggttatacgatacttgcgactggtacacctgatggggtgcataatacaattactatacgaccgcaagattatgttgtattcataccacctgtaggtaaacaaattagagcagtagtttattataataaagtagttgcatctaatatgagtaatgctgttactattttgccagatgacattccaccaacaatcaataatcctgttggaataaatgccaaatactatcgaggcgacgaagtcaactttacaatgggagtctctgatagacattctggtataaaaaatacaactattactactttgccaagtggttggacatcaaatttaactaaatccgacaacaaaaacggctcattagctattacaggtagagtctctatgaatcaggcatttaacagtgatattacatttaaagtatcagcgacagacaatgtcaataatacgacaaatgatagtcaatctaaacatgtgtcaattcatgtaggtaaaattagtgaagatgctcatccgattgtattaggaaatactgagaaagttgtagtagtcaatccgactgctgtatctaatgatgaaaagcaaagcataattactgcctttatgaataaaaaccaaaatataagaggatatttagcatcaactgatccagtaactgtcgataataatggtaacgtcacattacattaccgtgatggctcatcaacaacgcttgatgctacaaatgtgatgacatacgaaccagttgtgaaatctgaatatcaaactgccaatgctgctaaaacagcaacggtaacgattgctaaaggacaatcatttaatattggtgatattaaacaatattttactttaagtaatggacaagctattccaaatggcacatttacaaatattacatctgatagaactattccaactgcacaagaagttagtcaaatgaatgcaggtacgcagttatatcatatagttgcttcaaatgcatatcataaagacactgaagatttctatattagtttaaaaatcgttgatgtgaaacaacctgaaggcgatcaacgtgtctatcgtacgtcaacatatgatttaaccactgatgaaatctcaaaagtaaaacaagcttttattaatgcaaatagagatgtaattacgcttgccgaaggtgatatttcagttacaaatacacctaatggtgctaatgtaagtactattacagtaaatattaataaaggtcgattaacgaaatcattcgcgtctaacctagctaatatgaatttcttgcgttgggttaatttcccacaagattatacagtgacatggacgaatgcaaaaattgcaaacagaccaacagatggtggtttatcatggtccgatgaccataaatctttaatttatcgttatgatgctacattaggcacacaaattacaactaatgatattttaacgatgctaaaagcgactactacagtgcctggattgcgtaataatattactggtaatgaaaaagcacaagcagaagcaggtggaagaccaaactatagaacaactggttattcacaatcaaatgcgacaactgatggtcaacgtcaatttacgttgaatggtcaagtgattcaaatattagacatcatcaacccttcaaacggttatggtgggcaacctgttacaaattcaaatactcgtgcaaaccatagtaactcaactgttgttaacgtaaacgaaccggcagctaatggtgctggcgcatttacaattgaccacgttgtaaaaagtaattctacacataatgcaagtgatgcagtttataaagcgcagttatacttaacgccatatggtccaaaacaatatgttgaacatttaaatcaaaatacaggaaatactactgacgctattaacatttattttgtaccaagtgacttagtgaatccaacaatttcagtaggtaattacactaatcatcaagtgttctcaggtgaaacatttacaaatacgattacagcgaatgataactttggtgtgcaatcggtaactgtaccaaatacatcacaaattacaggtactgttgataataaccatcaacatgtttctgcaacggcaccaaatgtgacatcagcaactagtaagacaatcaatttattagcaactgatacaagtggtaatacagctacaacttcattcaatgtaacagtgaaacctttgcgtgataaatatcgagttggtacttcatcaacggctgctaatcctgttagaattgccaatatttcgaataatgcgacagtatcacaagctgatcaaacgacaattattaattcgttaacgtttacaagtaatgcaccaaatagaaactatgcaacagcaagcgcaaatgaaatcactagtaaaacagttagtaatgtcagtcgtactggaaataatgccaatgtcacagtaactgttactcatcaagatggaacaacatcaacagtgactgtacctgtaaagcatgtcattccagaaatcgttgcacattcgcattacactgtacaaggccaagacttcccagcaggtaatggttctagtgcagcagattactttaagttatctaatggtagtgccattccagatgcaacgattacatgggtaagtggacaagcgccaaataaagataatacacgtattggtgaagatataacagtaactgcacatatcttaattgatggcgaaacaacgccgattacgaaaacagcaacatataaagtagtaagaactgtaccgaaacatgtctttgaaacagccagaggtgttttatacccaggtgtttcagatatgtatgatgcgaaacaatatgttaagccagtaaataattcttggtcgacaaatgcgcaacatatgaattttcaatttgttggaacatatggtcctaacaaagatgttgtaggtatatcaacgcgtcttattagagtgacttatgataatagacaaactgaagatttaactattttatctaaagttaaacctgacccaccaagaattgacgcaaactctgtgacatataaagcaggtcttacaaaccaagaaattaaagttaataacgtattaaataactcgtcagtaaaattatttaaagcagataatacaccattaaatgtcacaaatattactcatggtagtggttttagttcggttgtgacagtaagtgacgcgttaccaaatggcggaattaaagcaaaatcttcaatttcaatgaacaatgtgacgtatacgacgcaagacgaacatggtcaagttgttacagtaacaagaaatgaatctgttgattcaaatgatagtgcttctgttacagtaacaccacaattacaagcaactactgaaggcgctgtatttattaaaggtggcgacggttttgatttcggtcatgtagaacgatttattcaaaatccgccacatggggcaacggtcgcatggcatgatagtccagatacatggaagaatacagtcggcaacacacataaaactgcggttgtaacattacctagtggtcaaggtacgcgtaatgttgaagttccagtcaaagtttatccagttgctaatgctaaggcgccatcacgtgatgtgaaaggtcaaaatttgacacatggtacaaacgctattgattacattacatttgatccaaatactaatacgaatggtattacagcagcatgggcaaatagacaacaaccaaataaccagcaagcaggcgttcaacatttaaatgtcgatgtcacatatccaggtatttcagctgctaaacgagttcctgtaactgtgaacgtatatcaatttgaattccctcaaactacttatacaacaacagttggtggcactttagcaagtggtacgcaagcatcaggatatgcacatatgcaaaacgcttcaggtttaccaacagatggatttacgtataaatggaatcgtgatactacgggtacaaacgatgcaaactgggcagcaatgaataaaccaaatactgcacaagtcgttaatgcaaaatatgatgtcatctataatggacatacatttgcaacatctttaccagcgaaatttgtagtaaaagatgttcaaccagcgaaaccaactgtcactgaaacagcggcaggagcgattacaattgcacctggtgcgaaccaaacagtcaatactcatgctggtaatgttacgacatatgctgacaaattagttattaaacgtaatggaaatgttgtaacgacatttacacgtcgtaataatacgagcccatgggtgaaagaagcatcagcagataatgtaacaggtattgttggaactaataatggtattactgtggcagcaggtactttcaatcctgctgatacaattcaagttgttgcaacacaaggtagtggcgaaacaatcagtgacgagcaacgtagtgatgatttcacagttgtcgcaccacaaccgaaccaagcgactacgaaaatttggcaaaatggtcatattgatatcacgcctaataatccatcaggacatttaattaatccaacacaagcaatggatattgcttacactgaaaaagtgggtaatggtgcagaacatagtaagacaattaatgttgttcgtggtcaaaataatcaatggacaattgcgaataagcctgactatgtaacgttagatgcacaaactggtaaagtgacgttcaatgccaatactataaaaccaaattcatcaatcacaattactccgaaagcaggtacaggtcactcagtaagtagtaatccaagtacattaactgcaccggcagctcatactgtcaacacaactgaaattgtgaaagattatggttcaaatgtaacagcagctgaaattaacaatgcagttcaagttgctaataaacgtactgcaacgattaaaaatggcacagcaatgcctactaatttagctggtggtagcacaacgacgattcctgtgacagtaacttacaatgatggtagtactgaagaagtacaagagtccattttcacaaaagcggataaacgtgagttaatcacagctaaaaatcatttagatgatccagtaagcactgaaggtaaaaagccaggtacaattacgcagtacaataatgcaatgcataatgcgcaacaacaaatcaataccgcgaaaacagaagcacaacaagtgattaataatgagcgtgcaacaccacaacaagtttctgacgcactaactaaagttcgtgcagcacaaactaagattgatcaagctaaagcattacttcaaaataaagaagataatagccaattagtaacgtctaaaaataacttacaaagttctgtgaaccaagtaccatcaactgctggtatgacgcaacaaagtattgataactataatgcgaagaagcgtgaagcagaaactgaaataactgcagctcaacgtgttattgacaatggcgatgcaactgcacaacaaatttcagatgaaaaacatcgtgtcgataacgcattaacagcattaaaccaagcgaaacatgatttaactgcagatacacatgccttagagcaagcagtgcaacaattgaatcgcacaggtacaacgactggtaagaagccggcaagtattactgcttacaataattcgattcgtgcacttcaaagtgacttaacaagtgctaaaaatagcgctaatgctatcattcagaagccaataagaacagtgcaagaggtacaatctgcgttaacaaatgtaaatcgtgtcaatgagcgattaacgcaagcaattaatcaattagtacctttagctgataatagtgctttaagaactgctaagacgaaacttgatgaagaaatcaataaatcagtaactactgatggtatgacacaatcatcaatccaagcatatgaaaatgctaaacgtgcaggtcaaacagaaacaacaaatgcacaaaatgttattaacaatggtgacgcgacagaccaacaaattgccgcagaaaaaacaaaagtagaagaaaaatataatagcttaaaacaagcaattgctggattaacaccagacttggcaccattacaaactgcaaaaactcagttgcaaaatgatattgatcagccaacgagtacgactggtatgacaagcgcatctgttgctgcatttaatgacaaactttcagcagctagaactaaaattcaagaaattgatcgcgtactagcatctcatccagatgtagcaacgattcgtcaaaacgtgacagcagcgaatgctgctaaaacagcacttgatcaagcgcgcaatggcttaacagtcgataaagcacctttagaaaatgcgaaaaatcaactacaacatagtattgatacgcaaacaagtacaactggtatgacacaagactctataaatgcatacaatgcgaagttaacagctgcacgtaataaggttcaacaaatcaatcaagtattagcaggttcacctactgtagatcaaattaatacaaatacgtctgcagcaaatcaagcgaaatctgatttagatcatgcacgtcaagcgttaacaccagataaagcgccgcttcaaaatgcgaaaacgcaattagaacaaagcattaatcaaccaacagatacaacaggtatgacaaccgcttcgttaaatgcatacaaccaaaaattacaagcagcacgtcaaaagttaactgaaattaatcaagtgttgaatggcaacccaactgtccaaaatatcaatgataaagtggcagaggcaaaccaagctaaggatcaattaaatacagcacgtcaaggtttaacattagatagacagccagcgttaacaacattacatggtgcatctaacttaaaccaagcacaacaaaataatttcacgcaacaaattaatgctgctcaaaatcatgctgcgcttgaaacaattaagtctaacattacggctttaaatactgcgatgacgaaattaaaagacagtgttgcggataataatacaattaaatcaggtcaaaattacactgacgcaacaccagctaataaacaagcctatgataatgcagttaatgcggctaaaggtgtcattggagaaacgactaatccaacgatggatgttaacacagtgaaccaaaaagcagcatctgttaaatcgacgaaagatgctttagatggtcaacaaaacttacaacgtgcgaaaacagaagcaacaaatgcgattacgcatgcaagtgatttaaaccaagcacaaaagaatgcattaacacaacaagtgaatagtgcacaaaacgtgcaagcagtaaatgatattaaacaaacgactcaaagcttaaatactgctatgacaggtttaaaacgtggcgttgctaatcataaccaagtcgtacaaagtgataattatgtcaacgcagatactaataagaaaaatgattacaacaatgcatacaaccatgcgaatgacattattaatggtaatgcacaacatccagttataacaccaagtgatgttaacaatgctttatcaaatgtcacaagtaaagaacatgcattgaatggtgaagctaagttaaatgctgcgaaacaagaagcgaatactgcattaggtcatttaaacaatttaaataatgtacaacgtcaaaacttacaatcgcaaattaatggtgcgcatcaaattgatgcagttaatacaattaagcaaaatgcaacaaacttgaatagtgcaatgggtaacttaagacaagctgttgcagataaagatcaagtgaaacgtacagaagattatgcggatgcagatacagctaaacaaaatgcatataacagtgcagtttcaagtgctgaaacaattattaatcaaacagctaatccgacaatgtctgttgatgatgttaatcgtgcaacttcagctgttactactaataaaaatgcattaaatggtgatgaaaaattagtacaatctaaaacagatgctgcaagagcaattgatgcattaccacatttaaataatgcacaaaaagcagatgttaaatctaaaattaatgctgcatcaaatattgctggtgtaaataccgttaaacaacaaggtacagatttaaatacagcgatgggtaacttgcagggtgcaatcaatgatgaacaaacgacgcttaatagtcaaaattatcaagatgcgacacctagtaagaaaacagcatacacaaatgcggtgcaagctgcgaaagatattttaaataaatcaaatggtcaaaataaaacgaaagatcaagttactgaagcgatgaatcaagtgaattcggctaaaaataacttagatggtacgcgtttattagatcaagcgaagcaaacagcgaaacagcagttaaataatatgacgcatttaacaactgcacaaaaaacgaatttaacaaatcaaattaatagtggtactactgttgctggtgttcatacggttcaatcaaatgccaacacattagatcaagcgatgaatacgttaagacaaagtattgctaacaatgatgcgactaaagcaagtgaagattacgtagatgctaataatgataagcaaacagcatataacaacgcggtagctgctgctgaaacgattattaatgcgaatagtaatccagaaatgaatccaagtacgattacacaaaaagcagagcaagtgaatagttctaaaacggcacttaacggtgatgaaaacttagctacggcaaaacaaaatgcgaaaacgtacttaaacacattaacgagtattacagatgctcaaaagaacaatttgattagtcaaattagtagtgcgacaagagtgagtggtgttgatactgtaaaacaaaatgcacaacatttagatcaagctatggctaacttacaaaatggtattaacaacgaatctcaagtgaaatcatctgagaaatatcgtgatgctgatacaaataaacaacaagagtatgataatgctattactgcagcgaaagcgattttaaataaatcgacaggtccaaacactgcgcaaaatgcagttgaagcagcattgcaacgtgttaatactgcgaaagatgcattgaatggtgatgcaaaattaattgcagctcaaaacgcagcgaaacaacatttaggtactttaacgcatatcactacagcacaacgcaatgatttaacaaatcaaatttca

SEQ ID NO: 46 - DNA, 20139 bp, Staphylococcus aureus
atgggtaacttacaaacggctatcaacgataagtcaggaacattagcgagccaaaacttcttggatgctgatgagcaaaaacgtaatgcttacaatcaagctatatcagctgccgaaaccattttaaataaacaaactggaccgaatacagcgaaaacagcggttgaacaagcacttaataatgttaatagtgcgaaacatgcattaaatggtacgcaaaacttaaataatgcgaaacaagcagcgattacagcaattaatggcgcatctgatttaaatcaaaaacaaaaagatgcattaaaagcacaagctaatggtgctcaacgcgtatctaatgcaaatgatgtacaacgtaatgcgactgaactgaacacggcaatgggtcaattacaacatgccatcgcagataagacgaatacgttagcaagcagtaaatatgtcaacgccgatagcactaaacaaaatgcttacacaactaaagttaccaatgctgaacatattattagcggtacgccaacggttgttacaacaccttcagaagtaacagctgcagctaatcaagtaaacagcgcgaaacaagaattaaatggtgacgaaagattacgtgttgcaaaacaaaacgccaatactgctattgatgcattaacgcaattaaatactcctcaaaaagctaaattaaaagaacaagtgggacaagccaatagattagaagacgtacaatctgttcaaacaaatggacaatcattgaacaatgcaatgaaaggcttaagagatagtattgctaacgaaacaacagtcaaagcaagtcaaaactatacagacgcaagtccgaataaccaatcaacatataatagcgctgtgtcaaatgcgaaaggtatcattaatcaaactaacaatccaactatggatactagtgcgattacccaagctacaacacaagtgaataatgctaaaaatggtttaaacggtgctgaaaacttaagaaatgcacaaaacactgctaagcaaaacttaaatacgttatcacacttaacaaataaccaaaaatctgcaatctcatcacaaattgatcgtgcaggtcatgtgagtgaggtaacagctgctaaaaatgcagcaactgagttaaacgcgcaaatgggcaacttggaacaagctatccatgatcaaaacacagttaaacaaggtgttaacttcactgatgcagataaagctaaacgtgatgcttatacaaatgcggtaagcagagcagaaacaattctgaataaaacgcaaggtgcaaatacgtctaaacaagatgttgaagcggctattcaaaatgttacaagtgctaaaaatgcattgaatggtgatcaaaacgttacaaatgcgaagaatgcagctaaaaatgcattaaataacttaacgtcaattaataatgcacaaaaacgtgacttaacaactaaaattgatcaagcaacaacagtagctggtgttgaagcggtatctaatacaggtacacaattgaatacagcgatggctaacttgcaaaatggtattaatgataaagcgaatactttagcgagcgaaaactatcatgatgctgattcagataagaaaactgcttatactcaagccgttacgaacgcagaaaatattttaaataaaaatagtggatcaaatttagataaagctgccgttgaaaacgcgttgtcacaagtgacaaatgcgaaaggtgccctaaatggtaaccataatttagagcaagctaaatcaaatgcaaacactactataaacggccttcaacatttaacaacagcacaaaaagataaattgaaacaacaagtgcaacaagcacaaaatgttgcaggtgtagatactgttaaatcaagtgccaacacattaaatggtgctatgggtacgttaagaaatagcatacaagataacacagctacgaaaaatggccaaaactatcttgatgctacagaacgtaacaaaacaaactataacaatgctgttgatagtgctaatggtgtcattaatgcaacaagcaatccaaatatggatgctaatgcaattaaccaaatcgctacacaagtgacatcaacgaaaaatgcattagatggtacacataatttaacgcaagcgaaacaaacagcaacaaatgccatcgatggtgctactaacttaaataaagcgcaaaaagatgcgttaaaagcacaagttacaagtgcgcaacgtgttgcaaatgtaacaagtatccaacaaactgcaaatgaacttaatacagctatgggtcaattacaacatggtattgatgatgaaaatgcaacaaaacaaactcaaaaatatcgtgacgctgaacaaagtaagaaaactgcttatgatcaagctgtagctgctgcgaaagcaattttaaataaacaaacaggttccaattcagataaagcagcagttgaccgtgcattacaacaagtaacaagtacgaaagatgcattgaatggggatgctaaactggcagaagcgaaagcggcagctagacaaaacttaggtactttaaaccatattacgaatgcacaacgtactgcgttagaaggtcaaatcaatcaagcgacgactgttgatggcgttaatactgtaaaaacaaatgccaatacattagacggcgctatgaatagcttacaaggtgcaatcaatgataaagatgcgacattaagaaatcaaaattatcttgatgcagatgaatcaaaacgaaatgcatatacgcaagctgtcacagcggctgaaggcattttaaataaacaaacaggtggtaacacatctaaagcagacgttgataatgcattaaatgcagttacaagagcgaaagcggctttaaatggtgctgaaaacttaagaaatgcgaaaacttcagcaacaaatacgattaatggtttacctaacttaacacaattacaaaaagacaacttgaagcatcaagttgaacaagcgcaaaatgtagttggtgtaaatggtgttaaagataaaggtaatacattaaatactgccatgggtgcattacgtacaagtatccaaaatgataatacgacgaaaacaagtcaaaattatcttgatgcatctgatagcaacaaaaataattacaatactgctgtaaataatgcaaatggtgttattaatgcaacgaacaatccaaatatggatgctaatgcgattaatgacatggcaaatcaagtcaatacaacaaaagcagcgttaaatggtgcacaaaacttagctcaagctaaaacaaatgcgacgaacacaattaacaacgcgcaagacttaaaccaaaaacaaaaagatgcattaaaaacacaagttaacaatgcacaacgtgtatctgatgcaaataacgttcaacatacagctactgaattgaacggtgcgatgacagcacttaaagcagctattgcggataaagaaagaacaaaagcaagcggtaattatgtcaatgctgatcaagaaaaacgtcaagcgtatgattcaaaagtgactaacgctgaaaatatcattaatggtacaccaaatgcgacattaacagtcaatgacgtaaatagtgcggcatcacaagtcaatgcggctaaaacagcattaaatggtgataacaacttacgtgtagcgaaagagcatgctaacaatacaattgacggcttagcacaattgaataatgtacaaaaagcaaaattaaaagaacaagttcaaagtgcaactacattagatggtgttcaaactgttaaaaatagttctcaaacgttgaatacagcgatgaaaggcttaagagatagtattgcgaatgaagcaacgattaaagcaggtcaaaactacactgacgcaagtccaaataatcgtaacgagtacgacagcgcagttactgcagcaaaagcaatcattaatcaaacatcgaacccaacgatggaaccaaatactattacgcaagcaacatcacaagtgacaactaaagaacatgcattaaatggtgcgcaaaacttagctcaagctaagacaacagcgaaaaacaacttgaataacttaacatcaattaacaatgcacaaaaagatgcgttaacgcgtaacattgatggtgcaactacagtagctggtgtaaatcaagaaactgcaaaagcaacagaattaaataacgcaatgcacagtttacaaaatggtatcaatgatgagacacaaacaaaacaaactcagaaatacctagatgctgagccaagtaagaaatcagcttatgatcaagcagtaaatgcagcaaaagcaattttaacaaaagctagtggtcaaaatgtagacaaagcagcagttgaacaagcattacaaaatgtgaacagtacgaagacggcgttgaacggtgatgcgaaattaaatgaagctaaagctgctgcgaaacaaacgttaggtacattaacacacattaataatgcacaacgtaatgcgttagataatgaaattacacaagcaacaaatgttgaaggtgttaatacagttaaagccaaagcgcaacaattagatggtgctatgggtcaattagaaacatcaattcgtgataaagacacgacgttacaaagtcaaaattatcaagatgctgatgatgctaaacgaacggcttattctcaagcagtaaatgcagcagcaactattttaaataaaacagctggaggaaatacacctaaagcagatgtcgaaagagcaatgcaagctgttacacaagccaatactgcattaaacggtattcaaaacttagaacgtgcgaaacaggctgcgaacacagcgattacaaatgcttcggacttaaatacaaaacaaaaagaagcattgaaagcacaagtaacaagtgcaggacgcgtatctgcagcaaatggtgttgaacatactgcgactgaattaaatactgcgatgacagctttaaaacgtgccattgctgataaagctgacacaaaagctagtggtaattatgtcaatgctgatgcgaataaacgccaagcatatgatgaaaaagtgacagctgcagaacatatcgttagtggtacaccaacaccaacgttaacaccatcagatgttacaaatgcagcaacgcaagtaacgaatgcgaagacgcagttaaacggtaatcataatttagaagtagcgaaacaaaatgctaacacagcaattgatggtttaacttctttaaatggtccgcaaaaagcaaaacttaaagaacaagtgggtcaagcgacgacgttgccaaatgttcaaactgttcgtgataatgcacaaacattaaacactgcaatgaaaggtctacgagatagcattgcgaatgaagcaacgattaaagcaggtcaaaactacacagatgcaagtcaaaacaaacaaaatgactacaacaatgcagtcactgcagcaaaagcaatcattggtcaaacaactagtccatcaatgattgcgcaagaaattaatcaagcgaaagaccaagtgacagctaaacaacaagcgttaaacggtcaagaaaacttaagaactgcgcaaacaaatgcgaagcaacatttgaatggcttaagtgacttaactaatgcacaaaaagatgcagcgaaacgccaaatcgaaggtgcaacgcatgttaatgaagtaacacaagcgcaaaataatgcggacgcattaaatacagctatgacgaacttgaaaaatggtattcaagatcaaaatacgattaagcaaggtgttaacttcactgatgcagatgaagcgaaacgtaatgcatatacaaatgcagtgacgcaagctgaacaaattttaaataaagcacaaggtccaaatactgcaaaagacggtgtcgaaactgcgttacaaaatgtacaacgtgctaaaaacgaattgaacggtaatcaaaatgttgcgaacgctaagacaactgcgaaaaatgcattgaataaccttacatcaattaataatgcacaaaaagcagcattgaaatcacaaattgaaggtgcgacaacagttgcaggtgtaaatcaagtgtctacaatggcatctgaattaaatactgcaatgagcaacttacaacgtggtattaatgacgaagcagctacaaaagcagctcagaaatatactgaagcagatagagataaacaaactgcatacaatgatgctgtaacagcagctaaaacgttattagataaaacagctggttcaaatgacaataaagtagccgttgaacaagcattacaacgtgtgaatactgctaaaacagcattaaatggtgacgcgcgattaaatgaagcgaagaacacagctaaacaacaattagcgacaatgtcacatttaactaatgctcaaaaagcaaacttaacagaacaaattgaacgtggtacaactgttgctggtgttcaaggcatccaagcaaatgctggtactttaaatcaagcaatgaatcaattaagacaaagtattgcttctaaagatgcgactaaatcaagcgaagattatcaagacgcgaatgcagatttacaaaatgcatacaatgatgcggtaactaatgctgaaggtattattagtgcaacgaataaccctgaaatgaatcctgatacaattaaccaaaaagcgagccaagtgaacagtgcgaagtctgcattgaacggtgatgaaaaattagcagcagtaaaacaaactgcgaaatcagatatcggtcgtttgacagacttgaacaatgcacaacgaactgcggcaaatgctgaagtggatcaagcaccaaatcttgcagctgtcacagcggctaaaaataaagcaacatcgttaaacacagcgatgggtaatttgaaacatgcacttgctgaaaaggataatacgaaacgtagtgtcaattacacagatgcggatcaaccaaaacaacaagcgtatgatactgcagttacacaagcagaagcaattactaatgcaaatggcagtaacgcgaatgaaacacaagttcaagcagcgcttaaccaattgaatcaagctaaaaacgacttgaatggtgataataaagttgctcaagcgaaagaaacagcaaaacgtgcattagcttcatatagtaacttgaataacgcgcaatcaactgcagcaactagtcaaattgacaatgcaacgacagtagcagacgtaactgctgcacaaaatactgctaatgaattaaatacagcaatgggtcaacttcaaaatggtattaatgaccaaaacactgttaaacaacaagtgaactttacagatgctgaccaaggtaagaaagatgcttacacaaatgctgttacgaatgctcaaggtattttagataaagcaaacggtcaaaatatgacaaaagcacaagttgaagctgcattaaatcaagtaacgactgctaagaatgctttaaacggtgatgcaaatgtaagacaagcaaaatcagatgcgaaagcaaacttaggtacattaacacacttaaataatgcacaaaaacaagatttaacatcacaaatcgaaggtgcaacaacagtcaacggtgtaaatagtgttaaaacgaaagcacaagacttagatggtgcaatgcaacgattagagtcagcaatcgcaaataaagatcaaactaaagcgagcgaaaactacattgacgcagatccaactaagaaaacagcatttgataatgccatcacacaagctgaatcttacttaaataaagatcatggtacgaataaagataagcaagctgttgaacaagcaattcaaagtgtaacgtctactgaaaatgctttgaacggtgacgcgaacttacaatgcgctaaaactgaagctacacaagctatcgataacttgacacaattgaatacaccgcaaaaaacagcattgaaacaacaagtgaatgctgcacaacgcgtatcaggtgtaactgatctgaaaaatagtgctacatcacttaataatgcgatggatcaattaaaacaagcaattggtgatcatgacacaattgtagctggtggtaattacactaacgcaagtcctgataaacaaggtgcttacactgatgcatataatgctgcgaagaatatcgtaaatggttcacctaatgtgattacaaatgcagcagatgttactgcggcaacacaacgtgtcaataatgctgaaacaagtttaaatggtgatacaaacttagcaactgcgaagcaacaagctaaagatgcattacgtcaaatgacacatttatctgatgcacaaaaacaaagtattactggtcaaattgatagcgcgacacaagtaactggtgtacaaagtgtgaaagacaatgcaacaaatcttgacaatgcaatgaatcaacttcgaaatagtattgcgaataaagatgaagtaaaagcgagtcaaccatatgttgatgcagatacagataaacaaaatgcatacaatacagcagttacaagtgctgaaaatatcattaatgcaacgagtcagccaacacttgatccatctgcagtaacacaagcagctaatcaagtgaacactaacaaaactgcgcttaatggtgcgcaaaacttagcaaataaaaagcaagaaacaactgctaacatcaaccgattaagtcatttaaacaatgctcaaaagcaagatttaaatacacaagtgacaaatgcaccaaatattagcacagtaaatcaagtgaaaactaaagctgaacaattagatcaagcaatggaacgtttaatcaacggaatccaagacaaagatcaagtgaaacaaagtgttaactttacagatgcagatccagaaaaacaaacagcatacaacaatgcggtaactgctgctgaaaatattattaatcaagcaaatggtacaaatgcgaaccaatcacaagttgaagcagcactttcaactgtaacaactactaaacaagcgttgaatggtgatagaaaagtaacagatgctaaaaacaatgcaaaccaaacattatctacgttagataacttaaacaatgcacaaaaaggtgctgttactggaaacatcaatcaagcgcacactgtagctgaagtaacgcaagccattcaaaccgctcaggaactgaatacagcgatgggtaacttgaaaaatagcttgaatgataaagacactacacttggcagtcaaaactttgcagatgcagatccagagaagaaaaatgcatacaatgaagcggttcgtaatgctgaaaatattttaaataaatctacaggtacgaacgtgcctaaagatcaagttgaagcagctatgaatcaagtgaatactacaaaagcagcgcttaatggtactcaaaaccttgaaaaagcgaaacaacacgcaaatacagcaattgacggtttaagccatttaacaaatgcacaaaaagaggcattaaaacaattggtacaacaatcgactactgttgcagaagcacaaggtaatgaacaaaaagcaaacaatgttgatgcagcaatggacaaattacgtcaaagtattgcagataatgcgacaacaaaacaaaaccaaaattatactgatgcaagtccgaataaaaaggatgcgtacaataatgctgtcacaactgcacaaggtattattgatcaaactacaaacccttcattagatccgactgttatcaatcaagctgctggacaagtaagcacgtctaaaaatgctttaaatggtaatgaaaacttagaggcagcgaagcaacaagcaacgcaatctttaggttcattagacaacttaaataatgcgcaaaaacaagctgttactaatcaaattaatggcgcgcatactgttgatgaagcaaatcaaattaagcaaaatgcgcaaaacttaaatactgcgatgggtaacttgaaacaagcgatagctgataaagatgctacgaaagcaacagttaacttcactgatgcagatcaagcaaaacaacaagcatataacactgcagttacaaatgctgaaaatatcatttcaaaagctaatggtggtaatgcaacacaaactgaagttgaacaagcaatccaacaagtaaatgcagcaaaacaagcattaaatggtaatgccaacgttcaacatgcaaaagacgaagcaacagcattaattaataactctaatgatcttaaccaagcacagaaagatgcattaaaacaacaagtacaaaatgcaactactgtagctggtgtaaacaatgttaaacaaacggcgcaagagttaaacaatgcgatgacacaattaaaacaaggcattgcagataaagaacaaacaaaagctgatggtaactttgtcaatgcagattctgacaagcaaaatgcatataatcaagcagtagcgaaagctgaagcattaattagtggtacgcctgatgttgtcgttacacctagcgaaattactgcagcgttaaataaagttacgcaagctaaaaatgatttaaatggtaatacaaacttagcaacggcgaaacaaaatgttcaacatgctattgatcaattgccaaacttaaaccaagcgcaacgtgatgaatacagcaaacaaatcacgcaagcaacacttgtaccaaacgtcaatgctattcaacaagcggcaacaacgcttaatgacgcgatgacacaattgaaacaaggtattgcgaataaagcacaaattaaaggtagcgagaactatcacgatgctgatactgacaagcaaacagcatatgataatgcagtaacaaaagcagaagaattgttaaaacaaacaacaaatccaacaatggatccaaatacaattcaacaagcattaactaaagtgaatgacacaaatcaagcacttaacggtaatcaaaaattagctgatgccaaacaagatgctaagacaacacttggtacactagatcatttaaatgatgctcaaaaacaagcgctaacaactcaagttgaacaagcaccagatattgcaacagttaataatgttaagcaaaatgctcaaaatctgaataatgctatgactaacttaaacaatgcattacaagataaaactgagacattaaatagcattaactttactgatgcagatcaagctaagaaagatgattatactaatgcggtttcacatgcagaaggtattttatctaaagcaaatggcagcaatgcaagtcaaactgaagtggaacaagcgatgcaacgtgtgaacgaagcgaaacaagcattgaatggtaatgacaatgtacaacgtgcaaaagatgcagcgaaacaagtaattacaaatgcaaatgatttaaatcaagcgcaaaaagatgcattaaaacaacaagtcgatgctgcgcaaactgttgcaaatgtaaacacgattaagcaaacagcacaagatttaaatcaagcaatgacacaattgaaacaaggtattgcagataaagaccaaactaaagcaaatggtaactttgtcaatgctgatactgataagcaaaatgcatataacaatgcggtagcgcatgctgaacaaatcattagtggtacaccaaatgcaaacgtggatccacaacaagtggctcaagcgttacaacaagtgaatcaagctaagggtgatttaaacggtaaccacaacttacaagttgctaaagacaatgcaaatacagccattgatcagttaccaaacttaaatcaaccacaaaaaacagcattaaaagaccaagtgtcgcatgcagaacttgttacaggtgttaatgctattaagcaaaatgctgatgcgttaaataatgcaatgggtacgttgaaacaacaaattcaagcgaatagtcaagtaccacaatcagttgactttacacaagcggatcaagacaaacaacaagcttataacaatgcagctaaccaagcgcaacaaatcgcaaatggcacaccaacacctgtattggcgcctgatacagtaacaaaagcagttacaactatgaatcaagcgaaagatgcattaaacggtgatgaaaaattagcgcaagcgaaacaagatgctttagcaaatcttgatacgttacgtgacttaaatcaaccacaacgtgatgcattacgaaaccaaatcaatcaagcacaagctttagctacagttgaacaaactaaacaaaatgcacaaaatgtgaatacagcaatgggtaacttgaaacaaggtattgcaaataaagatactgtgaaagcaagtgagaactaccacgatgctgatgtcgataagcaaacagcatatacaaatgcagtgtctcaagcggaaggtattatcaatcaaacgacaaatccaacgcttaacccagatgacattactcgtgcattaactcaagtgactgatgctaaaaatagcttaaacggtgaagctaaattagccactgaaaagcaaaatgctaaagatgccgtaagtggaatgacgcatttaaacgatgctcaaaaacaagcattaaaaggtcaaatcgatcaatcgcctgaaattgctacagtgaaccaagttaaacaaacagcaacgagcctagatcaagcaatggatcaattatcacaagctattaatgataaagatcaaatattagcggacggtaattacttaaatgcagatcctgacaaacaaaatgcgtataaacaggcagtagcaaaagctgaagcattattgaataaacaaagtggtactaatgaagtacaagcacaagttgaaagcatcactaatgaagtgaacgcagcgaaacaagcattaaatggtaatgacaatttggcaaatgcaaaacaacaagcaaaacaacaattggcgaacttaacacacttaaatgatgcacaaaaacaatcatttgaaagtcaaattacacaagcgccacttgttacagatgtcactacgattaatcaaaaagcacaaacgttagatcatgcgatggaattattaagaaatagtgttgcggataatcaaacgacattagcgtctgaagattatcatgatgcaactgcgcaaagacaaaatgactataacaaagctgtaacagctgctaataatatcattaatcaaactacatcgcctacgatgaatccagatgatgttaatggtgcaacgacacaagtgaataatacgaaagttgcattagatggtgatgaaaaccttgcagcagctaaacaacaagcaaacaacagacttgatcaattagatcatttgaataatgcgcaaaagcaacagttacaatcacaaattacgcaatcatctgatattgctgcagttaatggtcacaaacaaacagcagaatctttaaatactgcgatgggtaacttaattaatgcgattgcagatcatcaagccgttgaacaacgtggtaacttcatcaatgctgatactgataaacaaactgcttataatacagcggtaaatgaagcagcagcaatgattaacaaacaaactggtcaaaatgcgaaccaaacagaagtagaacaagctattactaaagttcaaacaacacttcaagcgttaaatggagatcataatttacaagttgctaaaacaaatgcgacgcaagcaattgatgttttaacaagcttaaatgatcctcaaaaaacagcattaaaagaccaagttacagctgcaactttagtaactgcagttcatcaaattgaacaaaatgcgaatacgcttaaccaagcaatgcatggtttaagacagagcattcaagataacgcagcaactaaagcaaatagcaaatatatcaacgaagatcaaccagagcaacaaaactatgatcaagctgttcaagccgcaaataatattatcaatgaacaaactgcaacattagataataatgcgattaatcaagtagcggcaactgtgaatacaacgaaagcagcattacatggtgatgtgaaattacaaaatgataaagatcatgctaaacaaacggttagccaattagcacatctaaacaatgcacaaaaacatatggaagatacgttaattgatagtgaaacaactagaacagcagttaagcaagatttgactgaagtacaagcattagatcaacttatggatgcattacaacaaagtattgctgacaaagatgcaacacgtgcgagcagtgcatatgtcaatgcagaaccgaataaaaaacaagcctatgatgaagcagttcaaaatgctgagtctatcattgcaggattaaataatccaactatcaataaaggtaatgtatcaagtgcgactcaagcagtaatatcatctaaaaatgcattagatggtgttgaacgattagctcaagataagcaaactgctggaaattctctaaatcatttagatcaattaacaccagctcaacaacaagcgctagaaaatcaaattaataatgcaacaacttgtgataaagtggctgaaatcattgcacaagcgcaagcattaaatgaagcgatgaaagcattaaaagaaagtattaaggatcaaccacaaactgaagcaagtagtaaatttattaacgaggatcaagcgcaaaaagatgcatatacgcaagcagtacaacacgcgaaagatttgattaacaaaacaactgatcctacattagctaaatcaatcattgatcaagcgacacaggcagtgactgatgctaaaaacaatttacatggtgatcaaaaactagctcaagataagcaacgtgcaacagaaacgttaaataacttgtctaacttgaatacaccacaacgtcaagcacttgaaaatcaaatcaataatgcagcaactcgtggtgaagtagcacaaaaattaactgaagcacaagcacttaaccaagcaatggaagctttacgtaatagcattcaagatcaacaacaaacagaatctggtagcaagtttattaatgaagataaaccgcaaaaagatgcttaccaagcagcagttcaaaatgcaaaagatttaattaaccaaacaggtaatccaacgcttgataaagcacaagttgaacaattgacacatgcttttaaacaagctaaagataacctacacggtgatcaaaaacttgcagacgataaacaacatgcggttactgatttaaatcaattaaatggtttgaataatccgcaacgtcaagcacttgaaagccaaataaacaacgcagcaactcgtggcgaagtagcgcaaaaattagctgaagcaaaagcgcttgatcaagcaatgcaagcattacgaaatagtattcaagatcaacaacaaacggaagcgggtagcaagtttatcaatgaagataaaccgcaaaaagatgcttaccaagcagcagttcaaaatgcaaaagatttaattaaccaaacaggtaatccaacactcgacaaatcacaagtagaacaattaacacaagcagtaacaactgcaaaagataatctacatggtgatcaaaaacttgctcgtgatcaacaacaagcagtaacaactgtaaatgcattgccaaacttaaatcatgcacaacaacaaacattaactgatgctataaatgcagcgcctacaagaacagaggttgcacaacatgttcaaactgctactgaacttgatcacgcgatggaaacattgaaaaataaagttgatcaagtgaatacagataaggctcaaccaaattacactgaagcgtcaactgataaaaaagaagcagtagatcaagcgttacaagctgcacaaagcattacagatccaactaatggttcaaatgcgaataaagacgctgtagaacaagcattaactaagcttcaagaaaaagtgaatgagttaaatggtaatgagagagtcgctgaagctaaaacacaagcgaaacaaactattgaccaattaacacatttaaatgctgatcaaattgcaactgctaaacaaaatattgatcaagcgacgaaacttcaaccaatcgctgaattagtagatcaagcaacgcaattgaaccaatcaatggatcaattacaacaagcagttaatgaacatgctaacgttgagcaaactatagattacacacaagcagattcagataagcaaaaggcttataaacaagcgattgctgatgctgaaaatgtattgaaacaaaatgcgaataagcaacaagtggatcaagcacttcaaaatattttaaatgcaaaacaagcattaaatggtgatgaacgtgtagcacttgctaaaacaaatggtaaacatgacatcgaccaattgaatgcattaaacaatgctcaacaagatggatttaaaggtcgcatcgatcaatcaaacgatttaaatcaaatccaacaaattgtagatgaggctaaggcacttaatcgtgcaatggatcaattgtcacaagaaatcactggcaatgaaggacgcacgaaaggtagcacgaactatgtcaatgcagatacacaagtcaaacaagtatatgatgaagcggttgataaagcgaaacaagcacttgataaatcgtctgggcaaaacttaactgcagaacaagttatcaaattaaatgatgcagtcactgcagctaagaaagcattaaatggtgaagaaagacttaataatcgtaaagctgaagcattacaaagattggatcaattaacacatctaaacaatgctcaaagacaattagcaatccaacaaattaataatgctgaaacgctaaataaagcatctcgagcaattaatagagcaactaaattagataatgcaatgggtgcagtacaacaatatattgacgaacagcaccttggtgttatcagcagcacaaattacatcaatgcagatgacaatttgaaagcaaattatgataatgcaattgcgaatgcagcacatgagttagataaagtgcaaggtaatgcaattgcaaaagctgaagcagagcaattgaaacaaaatattatcgatgctcaaaatgcattaaatggagaccaaaaccttgcaaatgccaaagataaagcaaatgcgtttgttaattcgttaaatggattaaatcaacagcaacaagatcttgcacataaagcaattaacaatgccgatactgtatcagatgtaacagatattgttaataatcaaattgacttaaatgatgcaatggaaacattgaaacatttagttgacaatgaaattccaaatgcagagcaaactgtcaattaccaaaacgctgacgataatgctaaaacaaacttcgatgatgccaaacgtctagcaaatacattgctaaatagtgataacacaaatgtgaatgatatcaatggcgcaatccaagcagtcaatgatgcaatccataatcttaatggtgatcaacgactacaagatgctaaagacaaggcaattcaatcaattaatcaagctttagctaataagctaaaagaaatcgaagcttcaaatgcgacggatcaagacaagcttattgcgaaaaataaagcagaagaattggcaaacagcatcatcaacaacattaataaagcaacaagtaatcaggctgtatctcaagttcaaacagcaggcaaccacgcgattgaacaagtgcatgctaatgaaataccaaaagcaaaaattgatgccaataaagacgttgataagcaagttcaagcattaattgacgaaattgatcgaaatccaaatctaacagataaggaaaaacaagcacttaaagatcgtattaatcaaatacttcaacaaggtcataacgacattaacaatgcgctgactaaagaagaaattgaacaagctaaagcacaacttgcgcaagcattacaagacatcaaagatttagtgaaagctaaagaagatgcgaaacaagatgttgataaacaagttcaagcattaattgacgaaatcgatcaaaatccaaatctaacagataaggaaaaacaagcacttaaagatcgtattaatcaaatacttcaacaaggtcataacggcattaacaatgcgatgactaaagaagaaattgaacaagccaaagcacaacttgcacaagcattaaaagaaattaaagatttagtgaaagctaaagaaaatgcgaaacaagatgttgataaacaagttcaagcattaattgacgaaatcgatcaaaatccaaatctaacagataaggaaaaacaagcgcttaaagatcgaatcaatcaaatactgcaacaaggtcataacgacattaacaatgcgatgactaaagaagaaattgaacaagccaaagcacaacttgcacaagcattacaagacatcaaagatttagtgaaagctaaagaagatgcgaaaaatgcaataaaagccttagctaatgcgaagcgtgatcaaatcaattcaaatccagatttaacacctgagcaaaaagcaaaagcgctcaaagaaattgacgaagctgaaaaacgagcactacaaaacgttgagaatgctcaaactatagatcaattaaatcgaggattaaacttaggtttagatgacattagaaatacacatgtatgggaggttgatgaacaacctgctgtaaatgaaatttttgaagcaacacctgagcaaatcctagttaatggtgaactcattgtacatcgtgatgacatcattacagaacaagatattcttgcacacataaacttaattgatcagctttcagcagaagttattgatacaccatcaactgcaacgatttctgatagcttaacagcaaaagttgaagttacattgcttgatggatcaaaagtgattgttaatgttcctgtaaaagttgtagaaaaagaattgtcagtagtcaaacaacaggcaattgaatcaatcgaaaatgcggcacaacaaaagattgatgaaatcaataatagtgtgacattaacactggaacaaaaagaagctgcaattgcagaagttaataagcttaaacaacaagcaattgatcatgttaacaatgcacctgatgttcattcagttgaagaaattcaacaacaagaacaagcgtatattgaacaatttaatccagaacaatttacgattgaacaagcaaaatcaaatgcaattaaatcgattgaagatgcaattcaacatatgattgatgaaatcaaagctcgtactgatctaacagataaagagaagcaagaagctattgctaagttaaatcaattaaaagaacaagcaattcaagcgattcaacgtgcgcaaagcatcagtgaaataactgagcaattggaacaatttaaagctcaaatgaaagcagctaatccaacagcaaaagaactagctaaacgcaagcaagaagctattagtagaattaaagacttttcaaatgaaaaaataaatagtattcgaaatagtgaaattggcacagctgatgaaaaacaagcagcaatgaatcaaattaacgaaattgtgcttgaaacaattagagatattaataatgcgcatacattacagcaagttgaggctgcattgaacaatggtattgctcgaatttcagcagtacaaattgtaatatctgatcgtgctaaacaatcgtcaagtactggaaatgaatctaatagccatttaacaattggttatggaactgcaaatcatccatttaacagttcgactattggacataaaaagaaacttgatgaagatgatgacattgatccacttcatatgcgtcactttagtaataatttcggtaatgttattaaaaacgctattggtgtggtgggtatctctggcttactagctagtttctggttcttcattgccaaacgtcgtcgtaaagaagatgaagaggaagaattagaaataagagataataataaagattcaataaaagagactttagacgatacaaaacatttaccacttttatttgcgaaacgtcgcagaaaagaagatgaagaagatgttactgttgaagaaaaagattcgctaaataatggcgagtcactcgataaagttaaacatacgccgttcttcttaccaaaacgtcgtcgtaaagaagatgaagaagatgtggaagttacaaatgaaaacacagatgaaaaagtgttgaaagataacgaacattcaccactcttattcgcaaaacgacgcaaagataaagaggaagatgttgaaacaacaactagtattgaatctaaagatgaggacgttcctttattattggctaaaaagaaaaatcaaaaagataaccaatccaaagacaaaaagtcagcatcaaaaaatacttctaaaaaggtagcagctaaaaagaagaaaaagaaatctaagaaaaataaaaaa

SEQ ID NO: 47 - DNA, 2103 bp, Staphylococcus epidermidis
ttgaataatcgtgataaattacaaaaatttagtattcgaaaatacgcaattggaacattttctactgtgattgcaacacttgtgttcatgggtatcaatacaaaccatgcaagtgccgacgagttgaatcaaaatcaaaagttaattaaacaattaaatcaaacagatgatgatgattcgaatacgcatagtcaagaaatcgaaaataacaaacaaaattctagtgggcagactgaatcattacgttcatcaactagtcaaaatcaagcaaatgcacgactgtcggatcaattcaaagacactaatgaaacatcgcaacaattacctacaaatgtttcggatgatagtatcaatcaatcgcatagtgaagcaaatatgaataacgaaccattgaaagttgataatagtactatgcaagcacatagtaaaatagtaagcgatagcgatgggaatgcttctgaaaataaacatcataaactaacagaaaatgtacttgcagaaagccgagcaagtaaaaatgacaaagagaaagagaatctacaagagaaagataaatcgcagcaagtacatccaccattagataaaaatgcattacaagctttttttgacgcatcatatcacaattacagaatgattgatagagatcgtgcggatgcaacagaatatcaaaaagtcaaatctacttttgactacgtcaatgacttactaggtaataatcaaaatattccttcagaacagcttgtttcggcatatcaacaattagagaaagcattagaacttgcacgtacgttaccacaacaatctactacagaaaaacgtggtagaagaagtacgagaagtgttgttgagaatcgttcatcaagaagcgattacttagatgctagaactgaatattatgtttcaaaagacgatgatgattctggtttccctcctggtactttcttccatgcttcaaatagaagatggccttataatttaccaagatctaggaacatcttacgtgcttctgatgtacaaggtaatgcttatatcactacaaaacgacttaaagatggatatcaatgggatattttatttaatagtaatcataaagggcatgaatatatgtactattggtttggacttccaagtgatcaaacaccaactggtccagtaactttcactattatcaaccgtgatggttcaagtacatctactggtggcgttggatttggatcaggtgcaccactacctcaattttggagatcagcaggtgctattaattctagcgtagcgaatgattttaaacatggctccgctacaaattatgcattttatgatggtgttaataatttttctgactttgctagagggggagaattatacttcgacagagaaggcgctacacaaactaataaatattatggcgatgaaaacttcgcattgctaaatagtgagaaaccagatcaaataagaggattagatacaatatatagttttaaaggtagtggtgatgtaagttatcgtatttcatttaaaactcaaggagctccaactgcaagattgtattatgctgctggcgcgcgttctggtgaatataaacaagcaacgaactataaccaactctatgtcgaaccttataagaattatcgaaatcgagtacagtcaaatgtccaagttaaaaatcgtacacttcatttaaaaagaacaatcagacaattcgatcctacattacagagaactactgatgttcctattttggatagtgacggttccggaagtattgattcggtatacgacccattaagttatgtaaagaatgtgactggtacagtcctaggtatttatccatcttatcttccttataatcaggaaagatggcagggagctaatgcaatgaatgcctatcaaattgaagaacttttttcacaagaaaatcttcaaaatgcagcacgttcaggccgtccaattcaatttcttgtaggttttgatgttgaagatagccatcataaccctgaaactcttttaccagtaaatttatatgtaaaacctgagttaaaacatacaattgagttatatcacgataatgaaaaacaagatagaaaggaattttcagtatcgaaa

SEQ ID NO: 48 - DNA, 28317 bp, Staphylococcus epidermidis
atgagtggaacgcttcataacactgtaggatcaggaatattaccttatcaacaagagatacgtatcaaacttactagtaatgaaccaattaaagatagtgaatggtctattacaggatatcctaacacgcttacattacaaaacgctgtgggtagaacaaataatgctactgaaaaaaacttagctcttgttggtcatattgatccaggaaattatttcatcactgttaagtttggtgataaagtagaacaatttgaaattagatcaaaaccaactccaccaagaatcattacaactgctaatgaattacgtggaaatcctaaccataagcctgaaataagagtaacagatataccaaatgatactactgctaaaatcaaacttgtgatgggcggaaccgatggcgatcatgatccagaaataaatccatatactgtccctgaaaactacacagtagttgcagaagcataccatgataatgatccaagtaaaaatggggtcttaacattccgttcatcagactaccttaaagatctaccattaagcggtgaattaaaggcaattgtttattacaatcaatatgtacaatcaaactttagtaaaagcgttccgtttagtagcgatacaacaccacctacaattaatgaaccggcaggactagttcataagtattacaggggagatcatgtagaaattactcttccagtcactgataatactggcggttcaggtttaagagatgtaaacgtcaatttacctcaaggttggacaaaaacctttacaatcaatcctaataataatactgagggtacgcttaagttaattggtaatatacctagtaatgaagcatataatacgacatatcatttcaatattactgcaaccgataattctggaaatacaacaaatccagctaaaacctttattttaaatgttggtaagttggctgatgatttaaatccagtcggattatctagagatcaactacaattagtgacagacccttcttcattatctaattccgaacgagaagaggtaaaaagaaaaataagtgaagcaaatgctaatataagatcatatttattacaaaataacccaatactcgctggagtaaacggcgatgttacattttattatagagatggttctgtagatgttattgatgctgaaaatgtaatcacatatgagcccgaaagaaaatccattttcagtgaaaatggtaatacaaataaaaaagaagcagtaatcactattgctagaggacaaaactataccattggtccaaacttaagaaaatatttctcattaagtaatggttcggatttacctaatagagatttcacctctatatcagctattggatctttaccttcatcgagtgaaattagtcgactcaatgttggaaattataactatagagttaatgctaaaaatgcttatcataagactcaacaagaacttaatttaaaacttaaaatagtagaggttaatgcacctactggtaataatcgtgtatatagagttagtacttataatttaactaatgatgaaatcaataaaatcaaacaagcatttaaagcagctaattctggacttaatttaaacgataacgatatcactgtttcgaataactttgaccatagaaatgttagtagtgtgacagtaactatacgtaagggcgatttgataaaagagttttcatcaaatctcaataatatgaatttcttacgttgggttaatataagggatgattataccatttcgtggacttctagtaagattcaaggtagaaatacagatggtggattagaatggtcaccagatcataaatcacttatttataaatatgatgcaacattaggtagacaaataaatactaatgacgtgttaactttacttcaagcaacagctaaaaactcaaatttacgttcaaatatcaatagtaatgaaaaacagttagcagaacgagggtctaatgggtattctaaatctataattagagatgatggcgagaaatcttatttacttaactcaaatcctattcaagtattagacttagtagaaccagataatggttacggtggacgtcaagtcagtcattctaacgttatatataatgaaaaaaattcttctatcgtaaatggtcaagttccagaagctaatggggcatccgcttttaatattgataaagttgttaaagctaatgcggcaaataatggtattatgggtgttatctataaggcacaattatacttagcaccatacagtccaaaaggttacattgaaaaattaggccaaaatttaagcaataccaataacgtgattaatgtttattttgtgccttctgataaagtaaatcctagtataactgtaggtaattacgaccatcatacggtatattctggtgaaacatttaaaaatactatcaatgtaaatgataattatggattaaatacagtagcttctacaagtgatagtgcaattactatgaccagaaacaacaacgagttagtaggtcaggctcctaatgttactaatagcataaataaaattgtaaaagttaaagccacagataaaagtggaaatgaaagtattgtttctttcacagtaaatataaaaccattaaacgagaaatatagaataacaacttcatcaagtaatcaaacaccagtgagaattagtaatattcaaaacaatgctaacctttcaattgaagatcaaaatagagtaaaatcttcactcagcatgactaaaattttaggtacaagaaattatgtcaatgagtcaaataatgacgttcgtagtcaagttgtaagtaaagtaaatagaagtgggaacaatgctacagttaatgttacaactacattttctgatggtacaactaatacaataaccgttccagttaaacatgtgttattagaagttgtacctactactagaacaacagtaagaggacaacaatttccaaccggcaaaggaacttccccaaatgatttctttagtttaagaacgggaggtccagttgatgcgagaatagtttgggttaataatcagggacccgatataaatagtaatcaaattggtagagatttaacattacacgctgaaatattctttgatggtgaaacaacaccaattagaaaagatactacttacaaacttagtcaatctattccaaagcaaatatatgaaacaactatcaatggtcgatttaattcatcaggtgatgcatatccaggaaattttgttcaagcagtaaatcaatattggccagaacatatggacttcagatgggcccaaggatcaggcacaccaagttctcgtaatgcaggttcatttactaaaacagttacggtagtttatcaaaacggccaaactgaaaacgttaatgtactattcaaagtcaaaccaaataaacctgttattgatagtaatagtgtgatttcaaaaggacaattaaatggtcaacaaattttagttcgaaatgttccacaaaatgcacaagtcactctatatcaatcaaatggaactgttattcctaatacaaatacaactatagattctaatggtatagctactgtaacaattcaaggcactctaccaaccggaaatattactgctaaaacctcaatgacaaataatgtaacgtacactaaacaaaatagtagtggaattgcttcaaatacaactgaagatataagtgttttttcagaaaacagtgatcaagtaaatgttaccgctggcatgcaagctaaaaatgatggtattaaaataattaaaggtacaaactataattttaatgacttcaatagtttcataagtaatataccagcccattctactcttacatggaacgaggagcctaatagttggaaaaacaacatcggtactacaacaaaaactgttacagttactctacctaatcatcaaggtacgagaactgtagatattccaataacaatctatccaacagttacagctaagaatccagtaagagatcaaaaaggacgaaacttaaccaatggtactgacgtttataattatattatttttgaaaataataaccgtcttggaggaacagcttcttggaaagacaatcgtcaacctgataaaaacatagccggtgtacaaaatttaattgcacttgttaattatcctggcatatctacaccattagaagttcctgttaaagtgtgggtatataattttgatttcactcaacctatctacaaaattcaagtaggagatacattccctaaaggaacatgggcaggctattacaaacatcttgaaaatggagagggattaccaatagatggttggaaattttattggaaccagcaaagtacaggaactactagtgatcaatggcaatcattagcatatactagaactccttttgttaaaactggtacttatgatgtcgttaatcctagcaactggggtgtttggcaaacatcacaatcagctaaatttatagttacaaatgctaaacctaatcaaccaaccataactcagtctaaaactggtgatgtaacagtaacacctggtgctgtgcgtaatatactaataagtgggacaaatgattatatccaagcatctgcagataagattgttattaataaaaatggaaataaattaactacatttgttaaaaataatgatggtcgttggactgttgaaactgggtcacctgacataaatggtatcggaccaacaaataacggaactgctatatctttaagtcgattagcagttagacctggggattcaatagaagcaatagcgactgaaggttccggagaaactataagtacttcagcaactagtgaaatttatattgtcaaagctccacaacctgaacaagtagcaactcatacttatgataatggaacattcgatatattacctgacaattcacgtaattctttaaatccaactgaacgtgtcgaaattaattacactgaaaaattaaatggcaatgaaacacaaaaatcattcactattactaaaaataacaacggcaaatggacgataaataataaaccaaattatgtcgagttcaatcaggataatggtaaagttgtattttcggccaatacaattaaacctaattctcaaattacaataactcctaaagcaggtcagggtaacactgaaaacacaaatcctactgtaattcaagcacctgcgcaacatactttaacaatcaatgaaattgttaaagaacagggtcaaaatgtgactaatgatgatattaataatgcggttcaagtgccaaataaaaatagagttgcgattaaacaaggaaacgctcttccaacaaatttagctggtggtagtacatcacatattccagtagttatttattacagtgatggaagttctgaagaagctactgagactgttagaactaaagttaataaaaccgaattaatcaatgctcgtcgtcgactagatgaagaaattagtaaagagaacaaaacaccatcaagtatcagaaactttgatcaagctatgaatcgtgctcaatcacaaattaatacagctaaaagtgatgctgaccaagttataggcacagaatttgcaacacctcaacaagtaaattcagctttatctaaagttcaagcggcacaaaataaaataaatgaagctaaagcattattacaaaacaaggctgataatagtcaacttgtgagagcaaaagaacaattacaacaatcgattcaaccagccgcttcaactgatggtatgactcaagatagcacaaggaactacaacaataaacgccaagcagctgaacaagcaatacaacatgcaaatagcgttataaataatggagatgcaacatcccaacaaattaatgatgctaaaaacacagttgaacaggcacagagagattatgttgaagctaaaagcaacttacgtgctgataagtcacagttacaaagcgcttatgatacgttaaatagagatgttttaacaaatgataaaaagccagcatctgtaagacgctataatgaagccatttcaaatattagaaaagaattagatacagctaaagcggatgcaagtagtactttgcgaaacaccaatccttccgttgaacaagttagagacgctttaaataaaataaatactgttcaacctaaagtgaatcaagcaattgctttacttcaaccaaaagaaaataattcagaacttgtacaagctaaaaaacgtttacaagacgctgtaaatgacatacctcaaacacaaggtatgacacaacaaacaattaataattataatgacaaacaacgtgaagctgaaagagcacttacatctgcacaaagagtgattgataatggggatgctacaactcaagaaattacttctgaaaaatctaaagtagagcaagcaatgcaagctttaactaatgctaaaagtaatctgagagctgataagaatgagttacagactgcatataacaaattaattgagaacgtatctaccaatggtaaaaaaccggcgagtatacgtcaatacgaaacagccaaagccagaatacaaaatcaaattaatgatgctaaaaatgaagcggagcgaattttaggtaatgataatccacaagtatcacaagtaactcaagcattgaacaaaatcaaagctattcaaccaaaattaacagaagctatcaacatgcttcaaaacaaagaaaataatacagaattagtcaatgctaaaaacagacttgaaaatgcagtaaatgatacagatccaacacacggtatgactcaagaaacaattaataattacaacgctaaaaagcgagaagctcaaaatgaaatacaaaaagcgaacatgattattaataatggagatgctactgctcaagatatttcttctgaaaaatctaaagtagagcaagtattacaagcattacaaaatgctaagaatgacttaagagctgataaaagagaattacagactgcatacaataaacttatacaaaatgttaataccaatggtaaaaaaccatctagtattcaaaactataagtctgcaagacgaaatatcgaaaaccaatataataccgctaaaaatgaagcacataatgttcttgaaaatacaaaccctactgtaaatgcagtagaagatgctttacgtaagataaatgcaattcaaccagaggttacaaaagctattaatatacttcaagataaagaagataatagcgaacttgttagagcaaaagaaaaattagatcaagcgattaatagtcaaccatcactaaatggtatgactcaagaatctattaataattacacaacaaaacgtagagaagcacaaaatatagcaagttctgctgacactattattaataatggggatgcatctattgaacaaataacagaaaataaaattcgagttgaagaggcaactaatgcacttaacgaagcaaaacaacatttaacggcagatacaacttctttaaaaactgaagtacggaaattaagtaggagaggcgacacaaacaacaaaaagcctagcagtgttagtgcttataacaatactattcattcgctacaatctgaaattacacagactgaaaatagagcaaatactatcatcaataagcctattcgttctgttgaagaagtaaataatgcattgcatgaagtaaaccaattgaaccaacgcttaacagatacaattaacttattacaacctttagcgaataaagaaagcttaaaagaagctcgtaatcgacttgaaagtaaaattaatgaaaccgttcaaacagacggtatgactcaacaatctgttgagaattataagcaagctaaaataaaagctcaaaatgaatctagtattgcacaaactcttattaataatggtgatgcatctgatcaagaagtttctacagaaatagaaaaattaaatcaaaagctgtctgaattaacaaattcaatcaatcacttaacagttaataaagaacctttagaaactgccaaaaatcagttacaagcaaatattgaccaaaaacctagcactgatggtatgacgcaacaatctgtacaaagctatgaacgtaaactacaagaagccaaagataaaataaactcaattaataatgtcttagctaacaatccagatgttaatgctatcagaacaaacaaagttgagacggaacaaatcaataatgaattaacacaggcgaaacaaggtcttactgttgataaacaaccattgattaatgcaaaaactgctttgcaacaaagtctagataatcaaccaagtactactggtatgactgaagcaacaattcaaaattataacgctaaacgtcaaaaagcagagcaagttatacaaaatgcaaataaaattattgaaaacgctcaacctagtgtacaacaagtgtctgatgagaaatctaaggtagagcaagcactcagtgaattgaacaacgccaaatcagcgcttagagctgataaacaagaattacagcaagcatataatcagttgattcaaccaacggatttaaataataagaaaccagcttctatcactgcgtacaatcaaagatatcaacaatttagtaacgaattgaacagcactaaaacaaatacagatcgcattttaaaagagcaaaatccaagtgtagctgatgtcaacaatgcactaaataaagtaagagaagtacaacaaaaattaaacgaagccagagcacttttacaaaataaagaagataatagtgcactagttcgagccaaagaacaacttcaacaggcagttgaccaagtcccttcaacagaaggtatgacgcaacaaactaaagatgattacaattcaaaacaacaagctgctcaacaagaaatatcaaaagcacaacaagttatcgataatggcgatgcgactacacaacaaatttctaacgccaaaacaaatgttgaacgcgctttagaagcattaaataatgcaaaaactggtttaagagcagataaagaggaacttcaaaatgcatataatcaattaactcaaaatattgatacgagcggtaaaacgcctgcaagtatcaggaaatacaatgaagctaagtcacgtattcaaactcaaattgattcagctaaaaatgaagcaaacagtattttaacaaatgacaatcctcaagtatcacaagtgactgctgcgttaaacaaaataaaagctgttcaacctgaattagataaagcgatagcaatgcttaaaaataaagagaataataatgcattggttcaagcgaaacaacaacttcaacaaattgttaatgaagtagatccaacacaaggcatgacaacagatactgctaataactataaatcaaaaaaacgtgaagctgaagatgaaatacaaaaagctcaacaaatcattaacaatggcgatgccactgagcaacaaattactaacgaaacaaatagagtaaatcaagcgattaatgcaataaacaaagccaaaaacgatttacgtgctgataagtctcaattggaaaatgcttataaccaattaatacaaaatgttgatacaaatggtaaaaaacctgctagtattcaacaataccaagctgctcgacaagctattgagacgcaatacaataacgctaaatcagaagcacatcaaattcttgaaaatagtaacccttcagttaatgaagtagcacaagcattacaaaaagttgaagctgtacaacttaaagttaatgacgcgattcatatacttcaaaataaagagaataatagtgcacttgtcacagctaaaaatcaacttcagcaatcagttaatgatcaaccattaacaacaggtatgactcaagattctattaataactatgaagctaagagaaatgaggctcaaagtgctatcagaaatgcagaagctgtcatcaacaatggcgatgcaactgcaaaacaaatttcagacgagaaatctaaagttgaacaagcactagcacatttgaatgatgctaaacagcaattaactgcagatactactgaattacaaacagcagttcaacaattaaacagaagaggcgatacaaataataaaaagccaagaagtatcaatgcatataataaagcaattcaatcattagaaacacaaattacttctgctaaagataatgccaacgctgtgatacaaaaacctatacgtactgttcaagaggtaaataatgcattacaacaagtaaatcagttgaatcaacaattaactgaagcaattaatcaacttcaaccgctatcaaataatgatgcattaaaagctgcaagattaaatttagaaaataaaattaatcaaactgtacaaactgatggtatgacacaacaatctatagaggcttatcaaaacgctaaacgcgtagcccaaaatgaatctaacactgctttagcattaattaataacggcgatgccgatgaacaacaaattacaactgaaacagaccgagtcaatcagcaaactacaaacttaactcaagcaattaacgggttaacagttaataaagaaccattagaaaccgctaaaacagcgttacaaaataacatcgaccaggtacctagtacagatggtatgactcagcaatctgttgcaaattataatcaaaaactacaaatagctaaaaacgaaattaacacaattaataacgttttagcgaacaatccagatgttaatgcaatcaaaacgaataaagcagaagcggaacgaatcagtaacgatttaacacaagctaagaataacttacaagttgatactcaacctttagaaaaaataaaaagacaacttcaagatgaaattgatcaaggtactaacacagatggaatgactcaagattcagtggataattacaatgatagcttaagtgcagcaattatagaaaaaggcaaagtaaataaattacttaaacgtaatccgacagtagaacaagttaaagagagcgttgctaatgcacaacaagtcatacaagatttacaaaatgctcgaacttcacttgttccagacaaaactcaacttcaagaagctaaaaatagattagaaaacagtattaaccaacaaacagatactgacggcatgactcaagattcgcttaacaattataatgataaattagcaaaagctagacaaaaccttgaaaaaatatctaaagttttaggtggtcaacctactgtagctgaaattagacaaaatacagatgaagcaaatgcacataaacaagcattagacactgcacgttctcaacttacattaaatagagagccatatatcaatcatattaataatgaaagtcatttaaataacgcgcaaaaagataattttaaagctcaagttaactcagcacctaatcataatactttagaaacgattaaaaataaggctgatactttaaatcaatctatgacagcattaagtgaaagtattgcagattacgaaaatcaaaaacaacaagaaaattatttagatgcatctaacaataaacgtcaagactatgacaatgcagtcaatgcggctaaaggtattttaaaccaaactcaaagtccgacaatgagtgctgatgtgattgatcaaaaagctgaagatgttaaacgtacgaaaactgcgttagatggaaatcaaagattagaagttgctaaacaacaagcacttaatcatttaaataccttaaatgatttaaacgatgctcagcgacaaactttaactgatactataaatcactctccaaacatcaattcagtgaatcaagctaaagaaaaagctaatactgttaacacagcaatgactcaactgaaacaaactattgctaactatgacgatgaattgcatgacggcaattacattaatgcagataaagacaaaaaagatgcttataataacgctgttaacaatgctaaacaactgattaatcaatctgatgctaatcaagcacaacttgatccagctgaaattaataaagttacacaaagagtcaatacgactaaaaatgatctaaatggtaatgacaaattggctgaagctaaaagagatgctaatacaaccattgatggtttaacttatctaaatgaagctcaacgtaacaaagctaaagaaaatgtaggcaaagcttctacaaaaacaaatattacgagtcagttacaagattacaatcaattgaatattgctatgcaagcattacgtaacagtgtgaacgacgttaacaatgttaaagcaaatagcaattatataaatgaagataatggtccaaaagaagcttacaatcaagccgttactcatgctcaaacattgataaatgcacaatctaaccctgaaatgagccgtgacgtagtaaatcaaaaaacacaagcagtaaatactgcccatcagaatttacatggacaacaaaagttagaacaagcacaaagtagtgctaatacagaaatcggtaacttaccaaacttaactaatactcaaaaagctaaagaaaaggaactggtaaatagtaaacaaactcgtacggaagtacaagaacaacttaaccaagctaagtcactagatagttctatgggcacgttaaaatcattagttgctaaacaacctacagtacaaaaaacaagtgtttatattaacgaagatcaacctgagcaatctgcctacaatgattccattacaatgggacaaactataattaataaaacagctgatccagtacttgataaaactttagttgataacgcaatcagtaacatttcaactaaagagaatgcactgcatggtgaacaaaaattaacaactgctaaaacggaagcaattaatgcacttaatacattagctgatttaaacacacctcagaaagaggctattaaaacagctattaacactgctcatacaagaactgatgtaactgcagagcaaagtaaggctaatcaaataaatagtgcaatgcacacgttgagacaaaacatttctgacaacgaatcagtaacaaacgaaagtaattatattaacgctgaacccgaaaaacaacatgcctttactgaggctctaaataatgctaaagaaatagttaatgaacaacaagccactcttgatgccaattcaattaaccaaaaagcacaagcgattcttactactaaaaatgctttagatggtgaagaacaattacgtcgtgctaaagaaaatgccgatcaagaaatcaatacgttaaatcaattgactgatgcgcaaagaaatagtgaaaaaggtttagtcaacagttctcaaactagaacagaagttgcttctcaattagcaaaagctaaagaactaaataaggtgatggaacaactgaatcaccttatcaatggtaaaaaccaaatgataaatagcagtaaatttatcaatgaagatgcgaaccaacaacaagcatattcaaatgcgattgcaagtgcagaagcgcttaaaaacaaatcacaaaaccctgaattagataaagtaacaattgaacaagcaattaataatattaattctgcaattaacaatctaaacggtgaagctaaactgactaaagctaaagaagatgctgttgcttcaataaacaacctaagcggattaacaaacgagcaaaaaacaaaagaaaatcaagccgttaatggcgctcaaactagagaccaagttgctaataaattacgtgatgctgaagcattagatcaatcaatgcaaacattacgtgacttagttaacaatcaaaatgcaatacattcaacaagtaattattttaacgaggattcaactcaaaagaatacttatgataatgcaattgataatggctcgacatatataactggtcaacacaatccagaattaaataaatctactattgatcaaacgattagccgaattaacacagctaaaaatgatttacatggtgtagaaaagttacaaagagataagggaactgctaatcaagaaattggacaattaggttatttaaatgaccctcaaaaatctggtgaggaatccttagtcaacggttcaaatacacgttctgaagtagaagagcatcttaatgaagctaaatcattaaataatgcaatgaaacaattaagagataaagtagctgaaaagactaatgtcaaacaaagtagcgattacattaatgattcaactgaacatcaacgtgggtatgatcaagcacttcaagaagcagaaaatattattaatgaaatcggtaatccaacattaaataaatcggaaattgaacaaaagttacaacaattgactgacgctcaaaatgcgttacaaggttcacatctattagaagaagctaaaaataatgcgattactggaatcaataaacttacagcattaaatgatgcacaacgtcaaaaagcaattgaaaatgttcaagcacagcagacaatcccagcagttaatcaacaattaactttggatagagaaataaatactgcaatgcaagctttacgagataaagtaggccaacaaaataacgttcaccaacaaagtaattatttcaatgaagatgaacaaccaaaacataactatgataattctgtacaagccggtcaaactattattgataaacttcaagatccaatcatgaacaaaaatgaaattgagcaggctattaatcaaatcaatacgactcaaacagcgttaagtggagaaaataaattacacactgaccaagaaagcacaaatagacaaatagaaggtttatctagtttgaacacagctcaaatcaacgccgaaaaagatttagtcaatcaagctaaaacaagaacagatgttgctcaaaagttagctgcagctaaagaaataaattctgctatgagtaatttaagagatggcattcaaaataaagaggacatcaaacgtagcagtgcatatatcaacgcagatccgactaaagttacagcttacgatcaagcactacagaacgcagaaaatatcatcaatgccacaccaaacgtagagcttaataaagctacaattgaacaagcgctatcacgcgttcaacaagcacaacaagatcttgatggtgttcaacaattagctaatgctaaacaacaagctacacaaactgtcaatgggttaaatagcttaaatgacggtcaaaagcgtgaattaaatctattaattaattcagctaatacccgtacaaaagtacaagaagaattaaacaaagcaactgaattgaaccatgcgatggaagctttaagaaacagtgttcaaaacgttgatcaagtaaaacaaagtagcaattatgtcaatgaagatcaacctgaacagcacaattatgataatgctgtcaatgaagctcaagctacaatcaacaacaatgctcaacctgttctagacaaattagctatagaacgtttaactcaaactgttaacactacaaaagatgcattacatggtgctcaaaaactgacacaagaccaacaagctgctgaaactggaatacgtggtttaacgagtctcaatgaacctcagaaaaatgctgaagtagctaaagtaactgcagcaacaacacgtgatgaagtgagaaatattcgtcaagaagcaacaacattagatactgcaatgcttggtttacgtaaaagcattaaagataaaaacgatactaaaaatagtagtaaatatattaatgaggatcatgaccaacaacaagcttatgacaatgctgtaaataatgctcaacaagttatcgatgaaactcaagcaacgttaagctcagatacaatcaatcaattggcaaatgccgtaactcaagctaaatctaatcttcatggagatactaaactacaacacgataaagatagtgctaaacaaacgattgctcaattacagaatttgaattcagctcaaaaacatatggaagattctttaattgataatgaatctacacgtacgcaagtccaacacgatttaacagaagctcaagctttagatggtttaatgggtgccttaaaagaaagtattaaagattatactaatattgtttcaaacggtaattacatcaatgcggaaccatctaagaaacaagcatatgatgcagctgtacaaaatgctcaaaatataataaatggaacgaatcaaccaacaattaataaaggtaatgtcactacagcaacacaaaccgtgaaaaatactaaagatgccttagacggtgatcatagattagaggaagctaaaaataatgccaatcaaacaatcagaaatctatctaatttgaacaatgcccaaaaagatgcagagaaaaatctagttaatagcgcatcaacattagaacaagttcaacaaaacttacaaaccgctcaacaattagataatgctatgggtgagttacgacaaagtattgctaaaaaagatcaagtgaaagcagatagtaaatatctaaatgaagatcctcaaattaagcaaaactatgatgatgcagttcaacgtgttgaaactattattaacgaaactcaaaaccctgaattacttaaagcaaacattgaccaagcaactcaatccgttcaaaatgcagaacaagctttacatggtgctgaaaaattaaatcaagacaaacaaacgtcttcgacagaactagatggattaacagatttaacagatgcacaacgtgaaaaactcagagaacaaattaacacttctaatagtagagatgatattaagcaaaaaattgagcaagcaaaagcactaaatgacgcaatgaaaaaacttaaagaacaagttgcgcaaaaagatggtgttcatgctaacagtgattatacaaatgaagattctgcacaaaaagatgcgtataataatgcacttaaacaagcggaagacattattaataacagctcaaatcctaacttaaatgcacaagacattactaatgctttaaataatattaaacaagcacaagataaccttcatggagctcaaaaattacagcaagacaaaaatacaactaatcaagccattggtaacttaaatcatcttaatcaacctcaaaaagatgcgcttatacaagctattaatggagctacatctagggaccaagttgcagaaaaacttaaagaggccgaagcgcttgatgaagctatgaaacaacttgaagatcaagtgaatcaagatgatcaaatttcaaatagcagcccattcataaatgaagactcagacaaacaaaaaacttataatgataaaatccaagctgcaaaagaaataattaatcaaacatctaatccaaccttagataaacaaaaaattgctgatacacttcaaaatattaaagatgcagtgaataatttacatggtgatcaaaaattagctcaatctaaacaagatgctaataatcaattaaatcatttagatgacttaaccgaagaacaaaaaaaccattttaaaccgttaattaataatgctgatactcgagatgaggtaaataaacaactagagattgctaaacaattaaatggtgatatgagtacacttcataaagtcataaatgataaagatcaaattcaacatttaagcaattacattaatgctgataatgataaaaaacaaaattatgataatgctattaaagaagctgaggatttaattcataatcatccagatacattagatcataaagcattacaagatttattaaacaagatagaccaagcgcataacgaattaaatggagaatccagatttaaacaggctttagacaatgctttaaacgacatagatagcttaaacagtctcaatgttccacaacgccaaactgttaaggataacatcaaccatgtgacaactctagaaagtttagctcaagaattgcagaaagcaaaagagcttaatgatgctatgaaagcaatgagagatagcattatgaatcaagagcaaattcgtaaaaatagcaattatactaatgaagacttagctcaacaaaatgcctataatcatgcagtagataaaataaataacattattggtgaagacaatgcgacgatggatcctcaaataatcaaacaagcaactcaagatataaatacagctataaatggattaaatggagatcaaaaacttcaagatgcaaagacagatgctaaacaacaaattactaactttactggtttaactgaaccacaaaaacaagcattggaaaacatcattaaccaacaaacaagcagagcaaatgttgctaaacagttaagtcatgctaaattcttaaatggaaaaatggaagaattaaaagttgcagtagccaaagcgtcattagtaagacaaaatagtaactatattaatgaagatgtctctgaaaaagaagcatatgaacaagctatcgcaaaaggtcaggaaataattaattcagaaaataatccaacaataagtagtactgatatcaatcgtaccattcaagaaattaatgatgctgaacaaaatcttcatggtgataataaattaagacaagcacaggaaattgcaaagaatgaaatacaaaatctagacggattaaattcagctcaaataacaaaattaatccaagatataggcagaacaacaactaaacctgcagtaactcagaaactagaagaagcaaaagcaataaaccaagctatgcaacaacttaaacaaagtatagccgataaggatgctactctaaattctagtaactatctcaatgaagattctgagaaaaagttagcgtacgataatgctgtaagccaagctgaacaactcataaatcaacttaacgacccaactatggatataagtaatattcaagctattactcaaaaggtcattcaagcaaaagattcattgcacggtgcgaataaacttgcacaaaatcaagcagattcaaatttaataataaatcaatcaacaaatttaaatgataaacaaaagcaagcattaaatgacttaattaatcatgctcaaactaaacagcaagtggcagaaataattgcacaagctaataagttaaataacgaaatgggcacactaaaaacactcgtagaagaacagtcaaacgttcatcaacaaagtaaatatattaatgaagatccgcaagttcaaaatatttataatgactccattcaaaaaggtcgagaaatattaaacggcactacagatgatgttttaaacaacaataaaatagcagatgccattcaaaacattcatttaactaaaaacgatttacatggtgatcaaaaattacaaaaagcacaacaagatgcaaccaatgaattaaactatttaacaaatctaaacaattctcaaagacaaagcgagcatgatgagattaactctgctccttcaagaactgaagtttctaatgatttaaatcatgctaaagcacttaatgaagctatgcgtcaacttgagaatgaagttgctcttgaaaacagtgttaaaaaattaagcgactttatcaatgaagatgaagcggcacaaaatgaatatagtaatgcacttcaaaaagctaaagacattatcaacggcgttccaagtagcactttagataaagctacaattgaagatgctttattagaattgcaaaatgctagagaaagtttacatggtgagcaaaaacttcaagaggctaaaaatcaagctgttgctgaaattgataatttacaagcattaaatcctggacaggttcttgctgaaaaaacattagttaaccaagcatcaaccaaaccagaagttcaagaagccttacaaaaagcaaaagaacttaatgaagctatgaaagcactgaaaactgaaataaataaaaaagaacaaatcaaggctgatagtagatatgtaaatgctgacagtggtcttcaagcaaattacaattctgcgttaaattatggttctcaaattattgcaactacccaaccaccagagcttaataaagatgtaataaatagagcaactcaaacgattaaaactgctgaaaataatttaaatgggcaatctaaattagcagaggctaagtcagacggaaatcaaagcatcgaacatttgcaaggattaacacaatcacaaaaagataaacaacatgatttaattaatcaagctcaaactaaacaacaggtagatgatatcgtaaataactctaaacaattagataactctatgaatcaactacaacaaattgttaacaatgacaatacagtaaaacaaaatagtgatttcattaatgaagattccagccaacaggatgcttataatcatgcaattcaagcagcaaaagatttgataactgctcatccaactatcatggataaaaatcaaatagatcaagctattgaaaatatcaaacaagcacttaatgatttacacggtagtaataaactatcagaagataaaaaagaagcttcagaacaactacaaaaccttaatagcttgacgaacgggcaaaaagatacgattttaaatcatattttcagtgcaccaacaagaagccaagtaggagaaaaaattgcaagtgctaaacaattaaataatacaatgaaagcacttagagattctattgctgataataatgaaattttacaaagtagtaagtacttcaatgaagattctgaacaacaaaatgcttataatcaagccgtaaataaagctaaaaatataattaatgatcaaccaacaccagtaatggcaaatgatgagattcaaagtgtcctaaatgaagttaaacaaactaaagataatttacatggtgatcaaaaacttgctaacgacaagacagatgctcaagcaacattaaatgcgttaaattacttaaatcaagcgcaaagaggtaatcttgaaactaaagttcaaaactctaattctagaccagaagtacaaaaagtagttcaattagcaaatcaacttaatgatgcgatgaaaaaattagatgatgctttaactggtaatgacgcaataaaacaaacgagtaattatattaatgaagatacttctcaacaagttaactttgatgagtatacagatagaggtaaaaacatagttgctgaacaaacaaatccaaatatgtctccaactaatattaacactattgctgataaaattactgaagctaaaaacgatttacatggcgtacaaaaactaaaacaagctcaacaacagtccatcaatactattaatcaaatgactggtctaaaccaagctcaaaaagaacaattaaatcaagaaattcaacaaactcaaacccgttctgaagtacatcaagtaattaataaagcacaagctttaaatgattcaatgaatactttacgtcaaagtattactgatgaacatgaagttaaacaaacaagtaactacatcaatgaaactgttggtaatcaaactgcatataacaatgccgttgatcgtgtaaaacaaataatcaatcaaacatctaatccaactatgaatcctttagaggtggaacgtgcaacatcaaatgtaaaaatttctaaagatgcacttcatggtgaacgtgaattgaatgacaataaaaattcaaaaacttttgcagtcaatcacttagataacctcaatcaagctcaaaaagaagcattaactcatgaaattgaacaagcaactatagtttcacaagtaaataatatctataacaaagcgaaagctttaaataatgatatgaaaaaacttaaagatatcgttgctcaacaagataatgtgagacaatcaaacaattatataaacgaggatagtacacctcaaaatatgtacaacgatacaattaatcatgcacaatcaatcattgatcaagtagcaaaccctacgatgtctcatgacgaaatagagaatgcaatcaataacataaagcatgccatcaatgcactcgatggagaacataaattacaacaagcaaaagaaaatgcaaacttattgattaatagtttaaacgatttaaatgcaccacaaagagatgccataaatagattggttaatgaagctcaaacaagagaaaaagtagctgaacaacttcaaagtgctcaagctttaaatgacgctatgaagcatttaagaaacagcattcaaaatcaatcatccgtaagacaagagagcaaatatattaatgcaagtgatgctaaaaaagagcaatataatcacgcagttagagaagtcgaaaatattatcaatgaacaacatccaacattggataaagaaataattaagcaactaacggatggtgtaaatcaagcgaataatgacttaaatggcgttgaattattagatgctgataagcaaaacgcacatcaatcgatacctacattgatgcacttaaatcaagcacaacaaaacgcattaaatgaaaaaattaataacgcagttaccagaactgaagttgcggctattattggccaagcaaaactactcgatcatgctatggagaatttagaagaaagtatcaaagataaagagcaagtcaaacagtcaagtaactatattaatgaagattctgatgttcaagaaacatacgataacgccgttgatcatgtgacagaaatacttaatcaaacagtaaatccaactttatctattgaagatatagagcatgctatcaacgaagttaatcaagcgaaaaaacaactcagaggtaaacaaaaactttatcaaactatcgatttagctgataaagaattaagtaaattggatgatttaacatcacaacaaagcagttcaatatctaatcaaatatatactgctaaaacgagaacagaagttgcccaagcaattgaaaaagcaaaatcattaaatcatgcaatgaaagcacttaacaaagtatataaaaatgcagataaagtgttagatagtagtcgattcattaacgaagatcaacctgaaaaaaaggcgtatcaacaagctataaatcatgttgattcaatcattcatagacaaacaaatcctgaaatggatccaacagtaatcaatagcataactcatgaactcgaaacagctcaaaataacttacatggtgatcagaaacttgctcatgcacaacaagatgccgctaatgtaattaatggtctaattcatcttaatgttgctcaacgtgaggtaatgataaatacgaatacaaatgctacaacacgcgaaaaagttgcaaagaacttagataatgctcaagctcttgataaagctatggaaacactacaacaagtagttgctcataaaaataatatattgaacgatagtaaatatttaaatgaagattcaaaatatcaacaacaatacgatcgagttattgctgatgccgaacaactacttaatcagacaacaaatccaacattagaaccttataaagtcgatattgttaaggataatgtcctagctaacgaaaaaatactatttggcgcagaaaaactatcatatgacaaatcaaatgcaaatgatgaaattaaacatatgaattatcttaataatgcacaaaagcaatctataaaagatatgatttctcacgcagcattaagaactgaagttaaacaacttctgcaacaagctaaaatccttgatgaagccatgaaatcacttgaagataaaactcaagtagtgattacagatactactttgcctaattacactgaagcttcagaggataaaaaggaaaaagtagaccaaactgtatcacatgctcaagcgattattgataaaataaatggctcaaatgtaagtttagatcaagtacgacaagcactagaacaattaactcaagcatcagaaaacctcgatggtgatcagcgagttgaagaagctaaagttcatgctaatcaaacaattgatcaattaacacatcttaattcattacaacaacaaactgcgaaagaaagtgttaaaaacgcaacaaaactagaagaaatcgctactgttagtaacaatgctcaggcattaaacaaagtaatgggtaaattagaacaattcattaatcatgctgattctgttgaaaatagtgataattatagacaagccgacgacgacaaaatcatcgcttatgatgaagcacttgaacatggacaagatatacaaaaaactaacgcaacccaaaatgaaacaaaacaagcgttacaacaattaatatatgcagaaacatcgttaaatggtttcgaaagattaaatcatgctagaccacgagctttagaatatatcaaatcactagaaaaaataaacaatgctcaaaagtctgctttagaggataaagtaacgcaatcgcatgatttattagaattagaacatattgtcaacgagggcacaaacctcaatgacattatgggtgaattagctaacgcaatcgttaataactatgctccaaccaaagcaagtataaattatattaacgccgataacctacgcaaagataactttactcaagctatcaacaatgcacgtgatgcactcaacaaaactcaaggtcagaacttagatttcaatgcaattgatacatttaaagatgatatattcaaaactaaagatgcacttaacggtattgaacgtttaacagctgcaaaatcaaaagcagaaaaactaattgatagtttaaaatttattaataaagctcaattcacacatgcaaatgatgaaattatgaatactaattctattgcacaattgtctagaatcgtgaatcaagcatttgatttaaatgatgcaatgaaatctttaagagatgaacttaataatcaagcttttcctgtccaagcaagctcaaattatataaattcagatgaagatttaaaacaacaatttgaccatgctttaagtaatgctcgaaaagttcttgcaaaagaaaatggtaaaaatttagatgaaaaacaaattcagggactcaaacaagtgattgaggatactaaagatgctttaaatggtatccaacgtttatcaaaagctaaagctaaagcaattcaatacgtacaatctttatcttatatcaatgatgcacagcgtcatattgctgaaaataatattcacaactctgatgatttatcatctttagcaaatacattatctaaagctagtgatttagataatgcaatgaaagacttacgagatactatagaaagtaattcaacttctgttccaaatagtgtgaattatattaatgctgataagaatttacaaattgaatttgatgaggcgctacaacaagcaagtgcaacaagttctaaaacttcagaaaatccagcaacgattgaagaagtattaggtcttagtcaagccatttacgatacaaaaaatgcattaaatggtgaacaacgacttgcaactgagaagagcaaagatctaaaattaataaaaggattaaaagatttaaataaagcacaacttgaagatgtcacaaacaaggtaaattcagcaaatactttaacagagttatctcagctcactcaatcaacgttagaattaaacgataaaatgaaattattgagagataagcttaaaactttagtaaatcctgttaaagcaagtttaaattatagaaacgctgattataatttaaaacgtcaatttaacaaagctttaaaagaagctaaaggcgtattaaataaaaatagcggtacaaatgtcaatatcaatgacattcaacatcttttaacacaaatagataatgctaaagaccaattaaatggtgaacgacgtctaaaagaacatcaacaaaaatctgaagtatttattattaaagaattagatatacttaataatgctcaaaaagctgcaataattaatcagattagagcgtctaaagacattaaaataattaatcaaatcgttgataatgcaatagaattaaatgatgctatgcaaggtttaaaagaacatgtagctcaattaacagcaactacaaaagacaacattgaatatttaaatgctgatgaagaccataaattacaatatgattacgctatcaacttagcgaataatgttcttgacaaagaaaacggtacaaataaagacgctaatatcataattggaatgattcaaaacatggatgatgctagagcacttctaaatggaattgaaagacttaaagatgctcaaacaaaagcacataatgacattaaagatacgctcaaacgtcaacttgatgaaattgaacacgctaatgcaacatcaaattctaaagctcaagctaaacaaatggtaaatgaggaagctagaaaagcgctttctaatattaatgacgcaacatcaaatgatttagttaatcaagcaaaagatgaagggcaatctgcaattgaacacatacatgcagatgaattacctaaagcaaaactagatgctaatcaaatgattgaccaaaaagttgaagatataaatcacttaattagtcaaaatccaaacttatcaaatgaagaaaaaaataaactaatatctcaaattaataagttagtaaatggaattaagaatgaaattcaacaagctataaacaaacaacaaatagaaaatgctacaacaaaactagatgaagtcattgaaactactaaaaaattaattatcgccaaagcagaagctaaacaaatgataaaagagttatcacaaaagaaacgagatgcaataaataacaacactgatttaacaccttctcaaaaggcacatgctttagcagatattgataaaacagaaaaagatgcacttcaacatatcgaaaattctaattcaattgatgatatcaataacaataaagagcatgcatttaatactttagctcatatcattatttgggatactgatcagcaaccattagtttttgaactacctgaattgagccttcaaaatgctctagtaacaagtgaggtggttgttcacagagatgaaactatttcattagaatctataattggagctatgactttaactgatgaacttaaagtcaatattgtttcattaccgaacactgataaagtagctgatcacctaaccgctaaagttaaggttattttagctgatggctcatatgtcactgtaaatgttccagtcaaagttgtagaaaaagaattacaaatagctaaaaaggatgctataaaaacaattgatgttctggtaaaacaaaaaatcaaagatatagattctaataacgaattaacgtctactcaacgtgaagatgcaaaagctgaaattgaaagattgaaaaagcaagccatcgataaagtgaatcattcaaaatcgattaaagatattgaaacagtaaaacgaactgattttgaagaaatagatcagtttgatcctaaacgctttacgctaaataaagctaaaaaggatatcattactgatgttaatactcaaatccaaaatggtttcaaagaaattgaaacaataaaaggtttaacttctaatgaaaaaactcagtttgataaacaattaactgcactacaaaaagaatttttagaaaaagtcgagcatgctcataatttagtagaattaaatcaattacaacaagagtttaataatagatataaacatattttaaaccaagcacatttactaggtgaaaaacatatagcagaacataaattaggatatgttgtagtaaacaaaactcagcaaatactaaataatcaatctgcttcttactttataaaacaatgggcacttgatagaattaaacaaattcaactagaaacgatgaattcaattcgtggtgcgcataccgtacaagatgtacacaaagcattattacaaggtatagagcaaatcttgaaagtaaatgtaagtattataaatcaatctttcaacgattccttgcataactttaattatcttcattcaaaatttgatgctagattaagagaaaaggatgttgcaaaccatatcgtacaaactgaaacattcaaagaagttctaaaaggaacgggtgttgaaccaggtaaaatcaacaaagaaacacagcaaccaaaacttcataagaatgataatgatagcctattcaaacatttagttgataatttcggcaaaactgtaggtgttattacattaactggtttactttctagtttctggttagttttggctaaaagacgtaaaaaagaagaagaagaaaaacaatcgataaaaaatcatcacaaagatattcgtctttcagatactgataaaatagatccaattgtaataactaagcgtaaaatagataaagaagaacaaattcaaaacgatgacaaacattcaattccagttgctaaacataagaaatctaaagaaaagcaattgagtgaagaggatattcattcaatccccgtcgttaagcgtaaacaaaacagtgataacaaagatacaaaacagaagaaagttacttctaaaaagaagaaaacgcctcagtcaactaaaaaagttgtaaaaaccaaaaagcgttctaaaaag

SEQ ID NO: 49 - DNA, 3348 bp, Staphylococcus aureus
atgagagataagaaaggaccggtaaataaaagagtagattttctatcaaataaattgaataaatattcaataagaaaatttacagttggaacagcatctattttaattggctcactaatgtatttgggaactcaacaagaagcagaagcagctgaaaacaatattgagaatccaactacattaaaagataatgtccaatcaaaagaagtgaagattgaagaagtaacaaacaaagacactgcaccacaaggtgtagaagctaaatctgaagtaacttcaaacaaagacacaatcgaacatgaagcatcagtaaaagctgaagatatatcaaaaaaggaggatacaccaaaagaagtagctaatgttgctgaagttcagccgaaatcgtcagtcactcataacgcagaggcacctaaggttagaaaagctcgttctgttgatgaaggctcttttgatattacaagagattctaaaaatgtagttgaatctaccccaattacaattcaaggtaaagaacattttgaaggttacggaagtgttgatatacaaaaaaacccaacagatttaggggtatcagaggtaaccaggtttaatgttggtaatgaaagtaatggtttgataggagctttacaattaaaaaataaaatagattttagtaaggatttcaattttaaagttagagtggcaaataaccatcaatcaaataccacaggtgctgatggttgggggttcttatttagtaaaggaaatgcagaagaatatttaactaatggtggaatccttggggataaaggtctggtaaattcaggcggatttaaaattgatactggatacatttatacaagttccatggacaaaactgaaaagcaagctggacaaggttatagaggatacggagcttttgtgaaaaatgacagttctggtaattcacaaatggttggagaaaatattgataaatcaaaaactaattttttaaactatgcggacaattcaactaatacatcagatggaaagtttcatgggcaacgtttaaatgatgtcatcttaacttatgttgcttcaactggtaaaatgagagcagaatatgctggtaaaacttgggagacttcaataacagatttaggtttatctaaaaatcaggcatataatttcttaattacatctagtcaaagatggggccttaatcaagggataaatgcaaatggctggatgagaactgacttgaaaggttcagagtttacttttacaccagaagcgccaaaaacaataacagaattagaaaaaaaagttgaagagattccattcaagaaagaacgtaaatttaatccggatttagcaccagggacagaaaaagtaacaagagaaggacaaaaaggtgagaagacaataacaacaccaacactaaaaaatccattaactggagaaattattagtaaaggtgaatcgaaagaagagatcacaaaagatccgattaatgaattaacagaatacggaccagaaacgatagcaccaggtcatcgagacgaatttgatccgaagttaccaacaggagagaaagaagaagttccaggtaaaccaggaattaagaatccagaaacaggagacgtagttagaccaccggtcgatagtgtaacaaaatatggacctgtaaaaggagactcgattgtagaaaaagaagaaattccattcgagaaagaacgtaaatttaatcctgatttagcaccaggaacagaaaaagtaacaagagaaggacaaaaaggtgagaagacaataacgacaccaacactaaaaaatccattaactggagaaattattagtaaaggtgaatcgaaagaagagatcacaaaagatccgattaatgaattaacagaatacggacctgaaacaatagcgccaggtcatcgagacgaatttgatccgaagttaccaacaggagagaaagaagaagttccaggtaaaccaggaattaagaatccagaaacaggagacgtagttagaccgccggtcgatagcgtaacaaaatatggacctgtaaaaggagactcgattgtagaaaaagaagaaattccattcaagaaagaacgtaaatttaatcctgatttagcaccagggacagaaaaagtaacaagagaaggacaaaaaggtgagaagacaataacgacgccaacactaaaaaatccattaactggagaaattattagtaaaggtgaatcgaaagaagaaatcacaaaagatccgattaatgaattaacagaatacggaccagaaacgataacaccaggtcatcgagacgaatttgatccgaagttaccaacaggagagaaagaggaagttccaggtaaaccaggaattaagaatccagaaacaggagatgtagttagaccaccggtcgatagcgtaacaaaatatggacctgtaaaaggagactcgattgtagaaaaagaagaaattccattcgagaaagaacgtaaatttaatcctgatttagcaccagggacagaaaaagtaacaagagaaggacaaaaaggtgagaagacaataacgacgccaacactaaaaaatccattaactggagaaattattagtaaaggtgaatcgaaagaagaaatcacaaaagatccagttaatgaattaacagaattcggtggcgagaaaataccgcaaggtcataaagatatctttgatccaaacttaccaacagatcaaacggaaaaagtaccaggtaaaccaggaatcaagaatccagacacaggaaaagtgatcgaagagccagtggatgatgtgattaaacacggaccaaaaacgggtacaccagaaacaaaaacagtagagataccgtttgaaacaaaacgtgagtttaatccaaaattacaacctggtgaagagcgagtgaaacaagaaggacaaccaggaagtaagacaatcacaacaccaatcacagtgaacccattaacaggtgaaaaagttggcgagggtcaaccaacagaagagatcacaaaacaaccagtagataagattgtagagttcggtggagagaaaccaaaagatccaaaaggacctgaaaacccagagaagccgagcagaccaactcatccaagtggcccagtaaatcctaacaatccaggattatcgaaagacagagcaaaaccaaatggcccagttcattcaatggataaaaatgataaagttaaaaaatctaaaattgctaaagaatcagtagctaatcaagagaaaaaacgagcagaattaccaaaaacaggtttagaaagcacgcaaaaaggtttgatctttagtagtataattggaattgctggattaatgttattggctcgtagaagaaagaattaa

SEQ ID NO: 50 - DNA, 4410 bp, Staphylococcus epidermidis
atgggcaaacgtagacaaggtcctattaataaaaaagtggattttttacctaacaaattaaacaagtattctataagaaaattcactgttggtacggcctcaatattacttggttcgacacttatttttggaagtagtagccatgaagcgaaagctgcagaagaaaaacaagttgatccaattacacaagctaatcaaaatgatagtagtgaaagatcacttgaaaacacaaatcaacctactgtaaacaatgaagcaccacagatgtcttctacattgcaagcagaagaaggaagcaatgcagaagcaccgaatgttccaactatcaaagctaattcagataatgatacacaaacacaattttcagaagcccctacaagaaatgacctagctagaaaagaagatatccctgctgtttctaaaaacgaggaattacaatcatcacaaccaaacactgacagtaaaatagaacctacaacttcagaacctgtgaatttaaattatagttctccgtttatgtccttattaagcatgcctgctgatagttcatccaataacactaaaaatacaatagatataccgccaactacggttaaaggtagagataattacgatttttacggtagagtagatatccaaagtaatcctacagatttaaatgcgacaaatttaacgagatataattatggacagccacctggtacaacaacagctggtgcagttcaatttaaaaatcaagttagttttgataaagatttcgactttaacattagagtagcaaacaatcgtcaaagtaatacaactggtgcagatggttggggctttatgttcagcaagaaagatggggatgatttcctaaaaaacggtggtatcttacgtgaaaaaggtacacctagtgcagctggtttcagaattgatacaggatattataataacgatccattagataaaatacagaaacaagctggtcaaggctatagagggtatgggacatttgttaaaaatgactcccaaggtaatacttctaaagtaggatcaggtactccatcaacagattttcttaactacgcagataatactactaatgatttagatggtaaattccatggtcaaaaattaaataatgttaatttgaaatataatgcttcaaatcaaacttttacagctacttatgctggtaaaacttggacggctacgttatctgaattaggattgagtccaactgatagttacaattttttagttacatcaagtcaatatggaaatggtaatagtggtacatacgcagatggcgttatgagagctgatttagatggtgcaacattgacatatactcctaaagcagtcgatggagacccaattacatcaactaaggaaataccatttaataaaaaacgcgaatttgatccaaacttagcgccaggtacagaaaaagtcgttcaaaaaggtgaaccaggaattgaaacaacaacaacaccaacttatgtcaatcctaatactggagaaaaagtaggtgaaggcacacctacaacaaagatcactaaacaaccagtggatgaaatcgttcattatggtggcgaagaaatcaagccaggacataaagatgaatttgatccaaatgcaccgaaaggtagtcaaacaacgcaaccaggtaagccaggagttaaaaatcctgatacaggcgaagtagtcacaccaccagtggatgatgtgacaaaatatggtccagttgatggagatccgattacgtcaacggaagaaattccattcgacaagaaacgtgaattcaatcctgatttaaaaccaggtgaagagcgtgttaaacaaaaaggtgaaccaggaacaaaaacaattacaacaccaacaactaagaacccattaacaggggaaaaagttggcgaaggtgaaccaacagaaaaaataacaaaacaaccagtagatgaaatcacagaatatggtggcgaagaaatcaagccaggccataaggatgaatttgatccgaacgcaccgaaaggtagccaagaggacgttccaggtaaaccaggagttaaaaatcctgatacaggcgaagtagtcacaccaccagtggatgatgtgacaaaatatggtccagttgatggagatccgattacgtcaacggaagaaattccgtttgataaaaaacgcgaatttgatccaaacttagcgccaggtacagagaaagtcgttcaaaaaggtgaaccaggaacaaaaacaattacaacaccaacaactaagaacccattaacaggagaaaaagttggcgaaggtgaaccaacagaaaaaataacaaaacaaccagtggatgaaatcgttcattatggtggcgaagaaatcaagccaggccataaggatgaatttgatccgaacgcaccgaaaggtagccaagaggacgttccaggtaagccaggagttaaaaatcctgatacaggcgaagtagtcacaccaccagtggatgatgtgacaaaatatggtccagttgatggagatccgattacgtcaacggaagaaattccattcgacaagaaacgtgaattcaatcctgatttaaaaccaggtgaagagcgtgttaaacaaaaaggtgaaccaggaacaaaaacaattacaacaccaacaactaagaacccattaacaggggaaaaagttggcgaaggtgaaccaacagaaaaagtaacaaaacaaccagtggatgaaatcgttcattatggtggcgaagaaatcaagccaggccataaggatgaatttgatccaaatgcaccgaaaggtagccaagaagacgttccaggtaaaccaggagttaaaaaccctgatacaggcgaagtagttactccaccagtggatgatgtgacaaaatatggtccagttgatggagatccgattacgtcaacggaagaaattccgtttgataaaaaacgcgaatttgatccaaacttagcgccaggtacagagaaagtcgttcaaaaaggtgaaccaggaacaaaaacaattacaacaccaacaactaagaacccattaacaggagaaaaagttggcgaaggtgaaccaacagaaaaaataacaaaacaaccagtggatgagatcgttcattatggtggcgaagaaatcaagccaggccataaggatgaatttgatccgaacgcaccgaaaggtagtcaaacaacgcaaccaggtaagccaggagttaaaaatcctgatacaggcgaagtagtcacaccaccagtggatgatgtgacaaaatatggtccagttgatggagatccgattacgtcaacggaagaaattccgtttgataaaaaacgcgaatttgatccaaacttagcgccaggtacagagaaagtcgttcaaaaaggtgaaccaggaacaaaaacaattacaacgccaacaactaagaacccattaacaggagaaaaagttggcgaaggtgaaccaacagaaaaaataacaaaacaaccagtggatgagattgttcattatggtggtgaacaaataccacaaggtcataaagatgaatttgatccaaatgcacctgtagatagtaaaactgaagttccaggtaaaccaggagttaaaaatcctgatacaggtgaagttgttaccccaccagtggatgatgtgacaaaatatggtccgaaagttggtaatccaatcacatcaacggaagagattccatttgataagaaacgtgtatttaatcctgatttaaaaccaggtgaagagcgcgttaaacaaaaaggtgaaccaggaacaaaaacaattacaacaccaatattagttaatcctattacaggagaaaaagttggcgaaggtaaatcaacagaaaaagtcactaaacaacctgttgacgaaattgttgagtatggtccaacaaaagcagaaccaggtaaaccagcggaaccaggtaaaccagcggaaccaggtaaaccagcggaaccaggtaaaccagcggaaccaggtacgccagcagaaccaggtaaaccagcggaaccaggtaaaccagcggaaccaggtaaaccagcggaaccaggtaaaccagcggaaccaggtaaaccagcggaaccaggtacgccagcagaaccaggtaaaccagcggaaccaggtaaaccagcggaaccaggtaaaccagcggaaccaggtacgccagcagaaccaggtaaaccagcggaaccaggtacgccagcagaaccaggtaaaccagcggaaccaggtacgccaacacaatcaggtgcaccagaacaaccaaatagatcaatgcattcaacagataataaaaatcaattacctgatacaggtgaaaatcgtcaagctaatgagggaactttagtcggatctctattagcaattgtcggatcattgttcatatttggtcgtcgtaaaaaaggtaatgaaaaataa

SEQ ID NO: 51 - DNA, 504 bp, Staphylococcus aureus
atgaagaaactatatacatcttatggcacttatggatttttacatcaaataaaaatcaataacccgacccatcaactattccaattttcagcatcagatacttcagttatttttgaagaaactgatggtgagactgttttaaaatcaccttcaatatatgaagttattaaagaaattggtgaattcagtgaacatcatttctattgtgcaatcttcattccttcaacagaagatcatgcatatcaacttgaaaagaaactgattagtgtagacgataatttcagaaactttggtggctttaaaagctatcgtttgttaagacctgctaaaggtacaacatataaaatttatttcggatttgctgatcgacatgcatacgaagactttaagcaatctgatgcctttaatgaccatttttcaaaagacgcattaagtcattactttggttcaagcggacaacattcaagttattttgaaagatatctatacccaataaaagaatag

SEQ ID NO: 52 - DNA, 504 bp, Staphylococcus epidermidis
atgtatttatatacatcttatgggacttaccaatttttaaatcaaattaaacttaatcatcaagaacgtagtttatttcaattttccactaatgattcctcaataatcttagaagagtctgagggaaaatcaatcttaaaacatcctagtgcatatcaagtgattgatagcacaggtgaatttaacgaacatcatttttatagtgctatttttgtccctacatctgaagatcatcgtcaacagctagagaaaaaattattactcgtagacgtacctttaagaaattttggtggttttaaaagctatcgtttattaaaacccactgaggggtctacctacaaaatttactttggttttgcaaatcgaacagcatatgaagatttcaaagcttctgatatatttaatgaaaacttttcaaaagatgcattgagccaatactttggtgctagtggtcaacattctagctactttgaaagatatttatatccaatagaagatcattaa

SEQ ID NO: 53 - DNA, 3426 bp, Staphylococcus aureus
atgattaacagggataataaaaaggcaataacaaaaaagggtatgatttcaaatcgcttaaacaaattttcgattagaaagtatactgtaggaactgcatcgattttagtaggtacgacattgatttttggtctagggaaccaagaagctaaagctgctgaaaacactagtacagaaaatgcgaaacaagatgatgcaacgactagtgataataaagaagtagtgtcggaaactgaaaataattcgacaacagaaaatgattcaacaaatccaattaagaaagaaacaaatactgattcacaaccagaagctaaagaagaatcaactacatcaagtactcaacaacagcaaaataacgttacagctacaactgaaactaagcctcaaaacattgaaaaagaaaatgttaaaccttcaactgataaaactgcgacagaagatacatctgttattttagaagagaagaaagcaccaaattatacaaataacgatgtaactacaaaaccatctacaagtgaaattcaaacaaaaccaactacacctcaagaatctacaaatattgaaaattcacaaccgcaaccaacgccttcaaaagtagacaatcaagttacagatgcaactaatccaaaagaaccagtaaatgtgtcaaaagaagaacttaaaaataatcctgagaaattaaaagaattagttagaaatgataacaatacagatcgttcaactaaaccagttgctacagctccaacaagtgttgcaccaaaacgattaaatgcgaaaatgcgttttgcagttgcacaaccagcagcagttgcttcaaataatgtaaatgacttaattacagttacgaaacagacgatcaaagttggcgatggtaaagataatgtggcagcagcgcatgacggtaaagatattgaatatgatacagagtttacaattgacaataaagtcaaaaaaggcgatacaatgacgattaattatgataagaatgtaattccttcggatttaacagataaaaatgatcctatcgatattactgatccatcaggagaggtcattgccaaaggaacatttgataaagcgactaagcaaatcacatatacatttacagattatgtagataaatatgaagatataaaagcacgtttaactttatactcatatattgataagcaagcagtacctaatgaaactagtttgaatttaacgtttgcaacagcaggtaaagaaactagccaaaacgtttctgttgattatcaagacccaatggttcatggtgattcaaacattcaatctatctttacaaagttagatgaaaacaaacaaactattgaacaacaaatttatgttaatcctttgaaaaaaacagcaactaacactaaagttgatatagctggtagtcaagtagatgattatggaaatattaaactaggaaatggtagtaccattattgaccaaaatacagaaataaaagtttataaagttaaccctaatcaacaattgcctcaaagtaatagaatctatgattttagtcaatacgaagatgtaacaagtcaatttgataataaaaaatcatttagtaataatgtagcaacattggattttggtgatattaattcagcctatattatcaaagttgttagtaaatatacacctacatcagatggcgaactagatattgctcaaggtactagtatgagaacaactgataaatatggttattataattatgcaggatattcaaacttcatcgtaacttctaatgacactggcggtggcgacggtactgttaaacctgaagaaaagttatacaaaattggtgactatgtatgggaagacgttgataaagacggtgtccaaggtacagattcgaaagaaaagccaatggcaaacgttttagttacattaacttacccggacggtactacaaaatcagtaagaacagatgctaacggtcattatgaattcggtggtttgaaagacggagaaacttatacagttaaattcgaaacgccagctggatatcttccaacaaaagtaaatggaacaactgatggtgaaaaagactcaaatggtagttctataactgttaaaattaatggtaaagatgatatgtctttagacactggtttttataaagaacctaaatataatcttggtgactatgtatgggaagatacaaataaagatggtatccaagatgctaatgaacctggtatcaaagatgttaaggttacattaaaagatagtactggaaaagttattggtacaactactactgatgcctcgggtaaatataaatttacagatttagataatggtaactatacagtagaatttgaaacaccagcaggttacacgccaacggttaaaaatactacagctgaagataaagattctaatggtttaacaacaacaggtgtcattaaagatgcagataatatgacattagacagtggtttctataaaacaccaaaatacagtttaggtgattatgtttggtacgacagtaataaagacggtaaacaagattcaactgaaaaaggtatcaaagatgttaaagttactttattaaatgaaaaaggcgaagtaattggaacaactaaaacagatgaaaatggtaaatatcgtttcgataatttagatagcggtaaatacaaagttatttttgaaaagcctgctggcttaacacaaacagttacaaatacaactgaagatgataaagatgccgatggtggcgaagttgacgtaacaattacggatcatgatgatttcatacttgataacggatacttcgaagaagatacatcagacagtgattcagactcagacagtgattcagactcagacagcgactcagattcagacagtgattcagactcagatagcgattcagattcagacagcgactcagactcagatagcgactcagactcagacagcgactcagactcagatagcgactcagattcggacagcgattcagactcagatagcgactcagattcagacagcgattcagactcagatagcgactcagattcagacagtgactcagactcagatagcgactcagactcagacagtgactcagactcagacagcgattcagattcagatagcgactcagattcggacagtgattcagactcagatagcgactcagattcagacagcgactcagactcagatagcgactcagactcagacagtgattcagactcagatagcgattcggactcggatgcaggaaaacatacacctgttaaaccaatgagtactactaaagaccatcacaataaagcaaaagcattaccagaaacaggtagtgaaaataacggctcaaataacgcaacgttatttggtggattatttgcagcattaggttcattattgttattcggtcgtcgcaaaaaacaaaacaaataa

SEQ ID NO: 54 - DNA, 3171 bp, Staphylococcus epidermidis
atgattaataaaaaaaataatttactaactaaaaagaaacctatagcaaataaatccaataaatatgcaattagaaaattcacagtaggtacagcgtctattgtaataggtgcaacattattgtttggtttaggtcataatgaggccaaagccgaggagaattcagtacaagacgttaaagattcgaatacggatgatgaattatcagacagcaatgatcagtctagtgatgaagaaaagaatgatgtgatcaataataatcagtcaataaacaccgacgataataaccaaataattaaaaaagaagaaacgaataactacgatggcatagaaaaacgctcagaagatagaacagagtcaacaacaaatgtagatgaaaacgaagcaacatttttacaaaagacccctcaagataatactcatcttacagaagaagaggtaaaagaatcctcatcagtcgaatcctcaaattcatcaattgatactgcccaacaaccatctcacacaacaataaatagagaagaatctgttcaaacaagtgataatgtagaagattcacacgtatcagattttgctaactctaaaataaaagagagtaacactgaatctggtaaagaagagaatactatagagcaacctaataaagtaaaagaagattcaacaacaagtcagccgtctggctatacaaatatagatgaaaaaatttcaaatcaagatgagttattaaatttaccaataaatgaatatgaaaataaggctagaccattatctacaacatctgcccaaccatcgattaaacgtgtaaccgtaaatcaattagcggcggaacaaggttcgaatgttaatcatttaattaaagttactgatcaaagtattactgaaggatatgatgatagtgaaggtgttattaaagcacatgatgctgaaaacttaatctatgatgtaacttttgaagtagatgataaggtgaaatctggtgatacgatgacagtggatatagataagaatacagttccatcagatttaaccgatagctttacaataccaaaaataaaagataattctggagaaatcatcgctacaggtacttatgataacaaaaataaacaaatcacctatacttttacagattatgtagataagtatgaaaatattaaagcacaccttaaattaacgtcatacattgataaatcaaaggttccaaataataataccaagttagatgtagaatataaaacggccctttcatcagtaaataaaacaattacggttgaatatcaaagacctaacgaaaatcggactgctaaccttcaaagtatgtttacaaacatagatacgaaaaatcatacagttgagcaaacgatttatattaaccctcttcgttattcagccaaggaaacaaatgtaaatatttcagggaatggtgatgaaggttcaacaattatagacgatagcacaataattaaagtttataaggttggagataatcaaaatttaccagatagtaacagaatttatgattacagtgaatatgaagatgtcacaaatgatgattatgcccaattaggaaataataatgatgtgaatattaattttggtaatatagattcaccatatattattaaagttattagtaaatatgaccctaataaggatgattacacgactatacagcaaactgtgacaatgcagacgactataaatgagtatactggtgagtttagaacagcatcctatgataatacaattgctttctctacaagttcaggtcaaggacaaggtgacttgcctcctgaaaaaacttataaaatcggagattacgtatgggaagatgtagataaagatggtattcaaaatacaaatgataatgaaaaaccgcttagtaatgtattggtaactttgacgtatcctgatggaacttcaaaatcagtcagaacagatgaagatgggaaatatcaatttgatggattgaaaaacggattgacttataaaattacattcgaaacacctgaaggatatacgccgacgcttaaacattcaggaacaaatcctgcactagactcagaaggtaattctgtatgggtaactattaatggacaagacgatatgacgattgatagtggattttatcaaacacctaaatacagcttagggaactatgtatggtatgacactaataaagatggtattcaaggtgatgatgaaaaaggaatctctggagttaaagtgacgttaaaagatgaaaacggaaatatcattagtacaactacaaccgatgaaaatggaaagtatcaatttgataatttaaatagtggtaattatattgttcattttgataaaccttcaggtatgactcaaacaacaacagattctggtgatgatgacgaacaggatgctgatggggaagaagttcatgtaacaattactgatcatgatgactttagtatagataacggatactatgatgacgaatcggattccgatagtgactcagacagcgactcagattccgatagtgattcagactccgatagcgactcggattcagacagcgactcagattcagacagcgactcggattctgatagcgactcggattcagacagcgactcagactcagacagtgattcagattcagacagcgactcagattccgatagtgattcagactcagacagcgactcagattctgatagtgattcagactcagacagtgattcagattcagacagcgactcagattccgatagtgattcagactcagacagcgactcagattccgatagtgattcagactcagacagcgactcagattctgatagtgattcagactcagacagtgattcagattccgatagtgattcagactccgatagcgactcagactcggatagtgactcagattctgatagtgattcagactcagacagtgattcggattccgatagtgattcagactcagacagcgactcagattctgatagtgattcagactcagacaacgactcagatttaggcaatagctcagataagagtacaaaagataaattacctgatacaggagctaatgaagattatggctctaaaggcacgttacttggaactctgtttgcaggtttaggagcgttattattagggaaacgtcgcaaaaatagaaaaaataaaaattaa

SEQ ID NO: 55 - DNA, 1461 bp, Staphylococcus aureus
atgtctaataattttaaagatgactttgaaaaaaatcgtcaatcgatagacacaaattcacatcaagaccatacggaagatgttgaaaaagaccaatcagaattagaacatcaggatacaatagagaatacggagcaacagtttccgccaagaaatgcccaaagaagaaaaagacgccgtgatttagcaacgaatcataataaacaagttcacaatgaatcacaaacatctgaagacaatgttcaaaatgaggctggcacaatagatgatcgtcaagtcgaatcatcacacagtactgaaagtcaagaacctagccatcaagacagtacacctcaacatgaagaggaatattataataagaatgcttttgcaatggataaatcacatccagaaccaatcgaagacaatgataaacacgagactattaaagatgcagaaaataacactgagcattcaacagtttctgataagagtatagctgaacaatctcagcaacctaaaccatattttgcaacaggtgctaaccaagcaaatacatcaaaagataaacatgatgatgtaactgttaagcaagacaaagatgaatctaaagatcatcatagtggtaaaaaaggcgcagcaattggtgctggaacagcgggtgttgcaggtgcagctggtgcaatgggtgtttctaaagctaagaaacattcaaatgacgctcaaaacaaaagtaattctgacaagtcgaataactcgactgaggataaagcgtctcaagataagtctaaagatcatcataatggcaaaaaaggtgcagcgatcggtgctggaacagcaggtttggctggaggcgcagcaagtaaaagtgcttctgccgcttcaaaaccacatgcctctaataatgcaagccaaaaccatgatgaacatgacaatcatgacagagataaagaacgtaaaaaaggtggcatggccaaagtattgttaccattaattgcagctgtactaattatcggtgcattagcgatatttggaggcatggcattaaacaatcataataatggtacaaaagaaaataaaatcgcgaatacaaataaaaataatgctgatgaaagtaaagacaaagacacatctaaagacgcttctaaagataaatcaaaatctacagacagtgataaatcaaaagaggatcaagacaaagcgactaaagatgaatctgataatgatcaaaacaacgctaatcaagcgaacaatcaagcacaaaataatcaaaatcaacaacaagctaatcaaaatcaacaacagcaacaacaacgtcaaggtggtggccaaagacatacagtgaatggtcaagaaaacttataccgtatcgcaattcaatactacggttcaggttcaccggaaaatgttgaaaaaattagacgtgccaatggtttaagtggtaacaatattagaaacggtcaacaaatcgttattccataa

SEQ ID NO: 56 - DNA, 1419 bp, Staphylococcus epidermidis
gtgattgaattaattaaaatggaagggatgatagttgtgtctaataataattttaaagatgatttcgaaaagaatcgtcaatctattaatccagacgaacagcaaacagaattaaaagaagatgataaaacaaatgaaaataaaaaagaagctgactctcaaaacagtttatctaataactcaaatcaacaatttcctccgagaaatgcccaacgacgaaaaagacgtagagagacagcaactaatcaaagcaaacaacaagacgacaaacatcaaaaaaatagtgacgctaaaactacagaaggttcattagatgaccgttatgacgaagcacagttacagcaacaacatgataaatcgcaacaacaaaataaaactgaaaaacaatcacaagataatagaatgaaagatggaaaagatgcagctattgtaaatggaacatctgagtcaccagaacataaatcaaaatcaacacaaaatagacccggccctaaagctcaacaacaaaagcgtaaatcagaaagtacgcaatcaaaaccgtcaacaaacaaagataaaaaagcagctacaggtgctggaatagctggtgcagctggtgttgctggtgcagcagaaacatccaaacgtcatcataataaaaaagataaacaagattctaaacactcaaaccatgagaatgacgaaaaatctgttaaaaatgatgaccaaaagcaatctaaaaaaggcaaaaaagcagcagtcggtgctggcgcagctgcaggagttggtgcggctggtgttgcgcatcataataatcaaaataaacatcataatgaggaaaaaaattctaatcaaaacaatcagtacaatgaccaatcagaaggtaagaaaaaaggtggtttcatgaaaatcttgttaccacttatagcagccattcttattctaggtgcaatagcaatattcggtggtatggctctaaataatcacaacgatagtaaaagtgatgaccaaaaaatagcgaatcaaagtaagaaagactcagataaaaaagatggtgcgcaatccgaagataacaaagacaaaaaatctgatagtaacaaagacaaaaaatctgattctgataagaacgcagatgatgactctgataatagttcctcaaatcctaacgctacttcaactaataataacgataatgtagccaataataactcaaattatacaaaccaaaatcaacaagataatgcaaaccaaaatagcaataatcaacaggcaactcaaggtcaacaatcacatacagtatacggtcaagaaaacttatatcgtatcgccatacaatattatggagaaggaactcaagctaacgtagataaaattaaacgtgcgaatggattaagcagtaataatattcataatggtcaaacattagttattcctcaataa

SEQ ID NO: 57 - DNA, 498 bp, Staphylococcus aureus
atgaaaaataaattgatagcaaaatctttattaacaatagcggcaattggtattactacaactacaattgcgtcaacagcagatgcgagcgaaggatacggtccaagagaaaagaaaccagtgagtattaatcacaatatcgtagagtacaatgatggtacttttaaatatcaatctagaccaaaatttaactcaacacctaaatatattaaattcaaacatgactataatattttagaatttaacgatggtacattcgaatatggtgcacgtccacaatttaataaaccagcagcgaaaactgatgcaactattaaaaaagaacaaaaattgattcaagctcaaaatcttgtgagagaatttgaaaaaacacatactgtcagtgcacacagaaaagcacaaaaggcagtcaacttagtttcgtttgaatacaaagtgaagaaaatggtcttacaagagcgaattgataatgtattaaaacaaggattagtgagataa

SEQ ID NO: 58 - DNA, 960 bp, Staphylococcus aureus
atgaaaacacgtatagtcagctcagtaacaacaacactattgctaggttccatattaatgaatcctgtcgctaatgccgcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggttctatgaaagcagcagagaacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcattggacttcaacaaattggaaaggtaccaatactaaagataaatggacagatcgttcttcagaaagatataaaatcgattgggaaaaagaagaaatgacaaattaa

SEQ ID NO: 59 - DNA, 1287 bp, Staphylococcus aureus
atacacatgaaaaataaatatatctcgaagttgctagttggggcagcaacaattactttagctacaatgatttcaaatggggaagcaaaagcgagtgaaaacacgcaacaaacttcaactaagcaccaaacaactcaaaacaactacgtaacagatcaacaaaaagctttttatcaagtattacatctaaaaggtatcacagaagaacaacgtaaccaatacatcaaaacattacgcgaacacccagaacgtgcacaagaagtattctctgaatcacttaaagacagcaagaacccagaccgacgtgttgcacaacaaaacgctttttacaatgttcttaaaaatgataacttaactgaacaagaaaaaaataattacattgcacaaattaaagaaaaccctgatagaagccaacaagtttgggtagaatcagtacaatcttctaaagctaaagaacgtcaaaatattgaaaatgcggataaagcaattaaagatttccaagataacaaagcaccacacgataaatcagcagcatatgaagctaactcaaaattacctaaagatttacgcgataaaaataaccgctttgtagaaaaagtttcaattgaaaaagcaatcgttcgtcatgatgagcgtgtgaaatcagcaaatgatgcaatctcaaaattaaatgaaaaagattcaattgaaaacagacgtttagcacaacgtgaagttaacaaagcacctatggatgtaaaagagcatttacagaaacaattagacgcattagtagctcaaaaagatgctgaaaagaaagtggcgccaaaagttgaggctcctcaaattcaatcaccacaaattgaaaaacctaaagcagaatcaccaaaagttgaagtccctcaatctaaattattaggttactaccaatcattaaaagattcatttaactatggttacaagtatttaacagatacttataaaagctataaagaaaaatatgatacagcaaagtactactataatacgtactataaatacaaaggtgcgattgatcaaacagtattaacagtactaggtagtggttctaaatcttacatccaaccattgaaagttgatgataaaaacggctacttagctaaatcatatgcacaagtaagaaactatgtaactgagtcaatcaatactggtaaagtattatatactttctaccaaaacccaacattagtaaaaacagctattaaagctcaagaaactgcatcatcaatcaaaaatacattaagtaatttattatcattctggaaataa

SEQ ID NO: 60 - DNA, 1053 bp, Staphylococcus aureus
atgacaaaacattatttaaacagtaagtatcaatcagaacaacgttcatcagctatgaaaaagattacaatgggtacagcatctatcattttaggttcccttgtatacataggcgcagacagccaacaagtcaatgcggcaacagaagctacgaacgcaactaataatcaaagcacacaagtttctcaagcaacatcacaaccaattaatttccaagtgcaaaaagatggctcttcagagaagtcacacatggatgactatatgcaacaccctggtaaagtaattaaacaaaataataaatattatttccaaaccgtgttaaacaatgcatcattctggaaagaatacaaattttacaatgcaaacaatcaagaattagcaacaactgttgttaacgataataaaaaagcggatactagaacaatcaatgttgcagttgaacctggatataagagcttaactactaaagtacatattgtcgtgccacaaattaattacaatcatagatatactacgcatttggaatttgaaaaagcaattcctacattagctgacgcagcaaaaccaaacaatgttaaaccggttcaaccaaaaccagctcaacctaaaacacctactgagcaaactaaaccagttcaacctaaagttgaaaaagttaaacctactgtaactacaacaagcaaagttgaagacaatcactctactaaagttgtaagtactgacacaacaaaagatcaaactaaaacacaaactgctcatacagttaaaacagcacaaactgctcaagaacaaaataaagttcaaacacctgttaaagatgttgcaacagcgaaatctgaaagcaacaatcaagctgtaagtgataataaatcacaacaaactaacaaagttacaaaacataacgaaacgcctaaacaagcatctaaagctaaagaattaccaaaaactggtttaacttcagttgataactttattagcacagttgccttcgcaacacttgcccttttaggttcattatctttattacttttcaaaagaaaagaatctaaataa

SEQ ID NO: 61 - DNA, 1938 bp, Staphylococcus aureus
atgaacaaacagcaaaaagaatttaaatcattttattcaattagaaagtcatcactaggcgttgcatctgtagcgattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagcagttaaaccagcagcaaaagccactaacaatacatatcctattttgaatcaggaacttagagaagcgattaaaaaccctgcaataaaagataaagatcatagcgcaccaaactctcgtccaattgattttgaaatgaaaaaagaaaatggtgagcaacaattttatcattatgccagctctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattacgcttacattcgcttctctgtttcaaatggaacaaaagccgttaaaattgtaagttcaactcacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaactaa

SEQ ID NO: 62 - DNA, 2862 bp, Staphylococcus aureus
atgaataataaaaagacagcaacaaatagaaaaggcatgataccaaatcgattaaacaaattttcgataagaaagtattctgtaggtactgcttcaattttagtagggacaacattgatttttgggttaagtggtcatgaagctaaagcggcagaacatacgaatggagaattaaatcaatcaaaaaatgaaacgacagccccaagtgagaataaaacaactgaaaaagttgatagtcgtcaactaaaagacaatacgcaaactgcaactgcagatcagcctaaagtgacaatgagtgatagtgcaacagttaaagaaactagtagtaacatgcaatcaccacaaaacgctacagctagtcaatctactacacaaactagcaatgtaacaacaaatgataaatcatcaactacatatagtaatgaaactgataaaagtaatttaacacaagcaaaaaacgtttcaactacacctaaaacaacgactattaaacaaagagctttaaatcgcatggcagtgaatactgttgcagctccacaacaaggaacaaatgttaatgataaagtacattttacgaacattgatattgcgattgataaaggacatgttaataaaacaacaggaaatactgaattttgggcaacttcaagtgatgttttaaaattaaaagcgaattacacaatcgatgattctgttaaagagggcgatacatttacttttaaatatggtcaatatttccgtccaggttctgtaagattaccttcacaaactcaaaatttatataatgcccaaggtaatattattgcaaaaggtatttacgatagtaaaacaaatacaacaacgtatacttttacgaattatgtagatcaatacacaaatgttagcggtagctttgaacaagtcgcatttgcgaaacgtgaaaatgcaacaactgataaaactgcttataaaatggaagtaactttaggtaatgatacatatagtaaagatgtcattgtcgattatggtaatcaaaaaggtcaacaacttatttcgagtacaaattatattaataatgaagatttgtcacgtaatatgactgtttatgtaaatcaacctaaaaagacctatacaaaagaaacatttgtaacaaatttaactggttataaatttaatccagatgctaaaaacttcaaaatttacgaagtgacagatcaaaatcaatttgtggatagtttcaccccagatacttcaaaacttaaagatgttactggtcaattcgatgttatttatagtaatgataataagacggcgacagtagatttattgaatggtcaatctagtagtgataaacagtacatcattcaacaagttgcttatccagataatagttcaacagataatgggaaaattgattatactttagaaacacaaaatggaaaaagtagttggtcaaacagttattcaaatgtgaatggctcatcaactgcaaatggcgaccaaaagaaatataatctaggtgactatgtatgggaagatacaaataaagatggtaaacaagatgccaatgaaaaagggattaaaggtgtttatgtcattcttaaagatagtaacggtaaagaattagatcgtacgacaacagatgaaaatggtaaatatcagttcactggtttaagcaatggaacttatagtgtagagttttcaacaccagccggttatacaccgacaactgcaaatgcaggtacagatgatgctgtagattctgatggactaactacaacaggtgtcattaaagacgctgacaacatgacattagatagtggattctacaaaacaccaaaatatagtttaggtgattatgtttggtacgacagtaataaagatggtaaacaagattcgactgaaaaaggaattaaaggtgttaaagttactttgcaaaacgaaaaaggcgaagtaattggtacaactgaaacagatgaaaatggtaaataccgctttgataatttagatagtggtaaatacaaagttatctttgaaaagcctgctggtttaactcaaacaggtacaaatacaactgaagatgataaagatgccgatggtggcgaagttgatgtaacaattacggatcatgatgatttcacacttgataatggctactacgaagaagaaacatcagatagtgactcagattcggacagcgattcagactcagatagcgactcagattcagatagtgactcagactcagatagcgactcagactcagatagcgactcagacagcgactcagactcagatagtgattcagattcggacagcgactcagattcagacagcgaatcagattcggatagcgactcagactcagatagcgactcagacagcgactcagattcagacagtgactcagactcagacagcgactcagattcagacagcgattcagattcggatagcgactcagattcagatagcgattcggactcagacaacgactcagattctgacagcgattcagactcagatagcgactcagattcagacagcgactcagattcagacagcgattcagattcagatagcgattcagattcagacagcgactcagattcagatagcgactcagactcagacagcgattcagactcagatagcgactcagacagcgattcagattcggatagcgattcagattcagatgcaggtaaacatactccgactaaaccaatgagtacggttaaagatcagcataaaacagctaaagcattaccagaaacaggtagtgaaaataataattcaaataatggcacattattcggtggattattcgcggcattaggatcattattgttattcggtcgtcgtaaaaaacaaaataaataa

SEQ ID NO: 63 - DNA, 2808 bp, Staphylococcus aureus
atgaatatgaagaaaaaagaaaaacacgcaattcggaaaaaatcgattggcgtggcttcagtgcttgtaggtacgttaatcggttttggactactcagcagtaaagaagcagatgcaagtgaaaatagtgttacgcaatctgatagcgcaagtaacgaaagcaaaagtaatgattcaagtagcgttagtgctgcacctaaaacagacgacacaaacgtgagtgatactaaaacatcgtcaaacactaataatggcgaaacgagtgtggcgcaaaatccagcacaacaggaaacgacacaatcatcatcaacaaatgcaactacggaagaaacgccggtaactggtgaagctactactacgacaacgaatcaagctaatacaccggcaacaactcaatcaagcaatacaaatgcggaggaattagtgaatcaaacaagtaatgaaacgacttctaatgatactaatacagtatcatctgtaaattcacctcaaaattctacaaatgcggaaaatgtttcaacaacgcaagatacttcaactgaagcaacaccttcaaacaatgaatcagctccacagaatacagatgcaagtaataaagatgtagttagtcaagcggttaatccaagtacgcctagaatgagagcatttagtttagcggcagtagctgcagatgcaccggcagctggcacagatattacgaatcagttgacagatgtgaaagttactattgactctggtacgactgtgtatccgcaccaagcaggttatgtcaaactgaattatggtttttcagtgcctaattctgctgttaaaggtgacacattcaaaataactgtacctaaagaattaaacttaaatggtgtaacttcaactgctaaagtgccaccaattatggctggagatcaagtattggcaaatggtgtaatcgatagtgatggtaatgttatttatacatttacagactatgttgataataaagaaaatgtaacagctaatattactatgccagcttatattgaccctgaaaatgttacaaagacaggtaatgtgacattgacaactggcataggaaccaatactgctagtaagacagtattaatcgactatgagaaatatggacaattccataatttatcaattaaaggtacgattgatcaaatcgataaaacaaataatacgtatcgccaaacaatttatgtcaatccaagcggagataacgttgtgttacctgccttaacaggtaatttaattcctaatacaaagagtaatgcgttaatagatgcaaaaaacactgatattaaagtttatagagtcgataatgctaatgatttatctgaaagttattatgtgaatcctagcgattttgaagatgtaactaatcaagttagaatttcatttccaaatgctaatcaatacaaagtagaatttcctacggacgatgaccaaattacaacaccgtatattgtagttgttaatggccatattgatcctgctagtacaggtgatttagcactacgttcgacattttatggttatgattctaattttatatggagatctatgtcatgggacaacgaagtagcatttaataacggatcaggttctggtgacggtatcgataaaccagttgttcctgaacaacctgatgagcctggtgaaattgaaccaattccagaggattcagattctgacccaggttcagattctggcagcgattctaattcagatagcggttcagattctggcagtgattctacatcagatagtggttcagattcagcgagtgattcagattcagcaagtgattcagactcagcgagtgattcagattcagcaagtgattcagattcagcaagtgattcagattcagcaagtgattcagactcagcaagtgattcagattcagcaagtgattcagattcagcaagcgattcagattcagcgagcgattcagattcagcgagcgattcagattcagcgagtgattccgactcagcgagcgattcagactcagatagtgactcagattccgatagcgattccgactcagatagcgactcagattcagacagcgattctgactcagacagcgattctgactcagacagtgactcagattccgatagcgattctgactcagacagtgactcagattccgatagcgattcagattcagacagtgattcagactcagatagcgattcagattccgacagtgactcagactcagacagcgattcagattccgatagcgattcagattccgacagtgactcagattccgatagtgactcggattcagcgagtgattcagattcagatagcgattcagaatcagatagtgactcagactcagacagtgattcagattcagatagtgactcagactcagacagcgattcagaatcagatagtgactccgattcagacagcgattcagaatcagatagtgactccgattcagatagcgattcggattcagcgagtgattcagactcaggtagtgactccgattcatcaagtgattcagattccgattcaacgagtgacacaggatcagacaacgactcagacagtgattcaaatagcgattccgagtcaggttctaacaataatgtagttccgcctaattcacctaaaaatggtactaatgcttctaataaaaatgaggctaaagatagtaaagaaccattaccagatacaggttctgaagatgaagcgaatacgtcactaatttggggattattagcatcattaggttcattactacttttcagaagaaaaaaagaaaataaagataagaaataa

SEQ ID NO: 64 - DNA, 3117 bp, Staphylococcus aureus
gtgaaaaacaatcttaggtacggcattagaaaacataaattgggagcagcatcagtattcttaggaacaatgatcgttgttgggatgggacaagataaagaagctgcagcatcagaacaaaagacaactacagtagaagaaaatgggaattcagctactgataataaaacaagtgaaacacaaacaactgctactaacgttaatcatatagaagaaactcaatcatataacgcaacagtaacagaacaaccgtcaaacgcaacacaagtaacaactgaagaagcaccaaaagcagtacaagcaccacaaactgcacaaccagcaaatgtagaaacagttaaagaagaagagaaacctcaagttaaggaaacgacacaacctcaagacaatagcggaaatcaaagacaagtagatttaacacctaaaaaggttacacaaaatcaagggacagaaacacaagttgaagtggcacagccaagaacggcatcagaaagtaagccacgtgtgacaagatcagcagatgtagcggaagctaaggaagctagtgacgtttcagaagttaaaggcacagatgttacaagtaaagttacagtagaaagtggttctattgaggcacctcaaggaaataaagtagagccacatgctggtcaacgtgtcgtattgaaatacaaattgaaattcgcagatggattaaaaagaggagattattttgattttacattatcaaataatgtaaatacttatggggtttcaacagctagaaaggtaccagagattaaaaatggctcagttgtaatggctacaggtgagatcttagggaatggtaacataagatatacatttactaacgaaattgaacacaaggtagaggtaacagctaatttagaaatcaacttatttattgaccctaaaactgtacaaagcaatggagaacaaaagattacttctaaattaaatggtgaagaaacagaaaaaacaataccagttgtttataatccaggtgttagcaatagttatacaaatgtaaatggatcaattgaaacatttaataaagaatctaataaatttacacatatagcttatattaagccaatgaatggaaaccagtcaaacactgtatcagtaacagggacgttgactgaaggtagtaatttagctggtggacaacctactgttaaagtatatgaatatctagggaaaaaagatgaattgccacaaagtgtttatgcaaatacatcagatactaacaaattcaaagatgtaacaaaggaaatgaatggaaaattgagtgtgcaagacaatggtagttactcattgaatttagataagttggataaaacgtatgtcattcattatacaggtgaatatttgcaagggtcagatcaggttaattttagaactgaattatatgggtatccagaacgagcatataaatcttactatgtttatgggggatatcgtttaacttgggataatggtttagttttatatagcaataaagctgacggcaatggtaaaaatggacaaattattcaagataatgattttgaatataaagaagatactgcaaaaggaactatgagcgggcagtacgatgccaagcaaattattgaaacagaagaaaatcaagacaatacaccgcttgacattgattaccacacagctatagatggtgagggtggttatgttgatgggtatattgaaacaatagaagaaacggattcatcagctattgatatcgattaccatactgctgtggatagtgaagtgggtcacgttggaggatacactgagtcctctgaggaatcaaatccaattgactttgaagaatcgacacatgaaaattcaaaacatcacgctgatgttgttgaatatgaagaggatacaaatccaggtggtggccaagtaacaactgagtctaacttagttgaatttgacgaagagtctacaaaaggtattgtaactggcgcagtgagcgaccatacaacaattgaagatacgaaagaatatacgactgaaagtaatctgattgaactagtagatgaactacctgaagaacatggtcaagcacaaggaccaatcgaggaaattactgaaaacaatcatcatatttctcattctggtttaggaactgaaaatggtcacggtaattatggcgtgattgaagaaatcgaagaaaatagccacgttgatattaagagtgaattaggttacgaaggtggccaaaatagcggtaaccagtcattcgaggaagacacagaagaagacaaacctaaatatgaacaaggtggcaatatcgtagatatcgatttcgacagtgtacctcaaattcatggtcaaaataaaggtgaccagtcattcgaagaagatacagagaaagacaagcctaaatatgaacatggcggtaatatcattgatatcgacttcgacagtgtgccacaaattcatggattcaataagcataatgaaattattgaagaagatacaaacaaagataaacctaattatcaattcggtggacacaatagtgttgactttgaagaagatacacttccaaaagtaagcggccaaaatgaaggtcaacaaacgattgaagaagatacaacgccgccaacgccaccgacaccagaagtaccgagtgagccggaaacaccaatgccaccgacaccagaagtaccgagtgagccggaaacaccaacgccaccaacaccagaggtaccaagtgagccggaaacaccaacaccaccgactccggaagtaccaagtgagccggaaacaccaacaccaccgacaccagaagtgccgagtgagccagaaacaccaacaccgccaacaccagaggtaccagctgaacctggtaaaccagtaccacccgcaaaagaagaacctaaaaagccttctaaaccagtggaacaaggtaaagtagtaacacctgttattgaaatcaatgaaaaggttaaagcagtggcaccaactaaaaaagcacaatctaagaaatctgaactacctgaaacaggtggagaagaatcaacaaacaaaggtatgttgttcggcggattattcagcattctaggtttagcattattacgcagaaataaaaagaataacaaagcataa

SEQ ID NO: 65 - DNA, 2634 bp, Staphylococcus aureus
ttgaaaaaaagaattgattatttgtcgaataagcagaataagtattcgattagacgttttacagtaggtaccacatcagtaatagtaggggcaactatactatttgggataggcaatcatcaagcacaagcttcagaacaatcgaacgatacaacgcaatcttcgaaaaataatgcaagtgcagattccgaaaaaaacaatatgatagaaacacctcaattaaatacaacggctaatgatacatctgatattagtgcaaacacaaacagtgcgaatgtagatagcacaacaaaaccaatgtctacacaaacgagcaataccactacaacagagccagcttcaacaaatgaaacacctcaaccgacggcaattaaaaatcaagcaactgctgcaaaaatgcaagatcaaactgttcctcaagaagcaaattctcaagtagataataaaacaacgaatgatgctaatagcatagcaacaaacagtgagcttaaaaattctcaaacattagatttaccacaatcatcaccacaaacgatttccaatgcgcaaggaactagtaaaccaagtgttagaacgagagctgtacgtagtttagctgttgctgaaccggtagtaaatgctgctgatgctaaaggtacaaatgtaaatgataaagttacggcaagtaatttcaagttagaaaagactacatttgaccctaatcaaagtggtaacacatttatggcggcaaattttacagtgacagataaagtgaaatcaggggattattttacagcgaagttaccagatagtttaactggtaatggagacgtggattattctaattcaaataatacgatgccaattgcagacattaaaagtacgaatggcgatgttgtagctaaagcaacatatgatatcttgactaagacgtatacatttgtctttacagattatgtaaataataaagaaaatattaacggacaattttcattacctttatttacagaccgagcaaaggcacctaaatcaggaacatatgatgcgaatattaatattgcggatgaaatgtttaataataaaattacttataactatagttcgccaattgcaggaattgataaaccaaatggcgcgaacatttcttctcaaattattggtgtagatacagcttcaggtcaaaacacatacaagcaaacagtatttgttaaccctaagcaacgagttttaggtaatacgtgggtgtatattaaaggctaccaagataaaatcgaagaaagtagcggtaaagtaagtgctacagatacaaaactgagaatttttgaagtgaatgatacatctaaattatcagatagctactatgcagatccaaatgactctaaccttaaagaagtaacagaccaatttaaaaatagaatctattatgagcatccaaatgtagctagtattaaatttggtgatattactaaaacatatgtagtattagtagaagggcattacgacaatacaggtaagaacttaaaaactcaggttattcaagaaaatgttgatcctgtaacaaatagagactacagtattttcggttggaataatgagaatgttgtacgttatggtggtggaagtgctgatggtgattcagcagtaaatccgaaagacccaactccagggccgccggttgacccagaaccaagtccagacccagaaccagaaccaacgccagatccagaaccaagtccagacccagaaccggaaccaagcccagacccggatccggattcggattcagacagtgactcaggctcagacagcgactcaggttcagatagcgactcagaatcagatagcgattcggattcagacagtgattcagattcagacagcgactcagaatcagatagcgattcagaatcagatagcgactcagattcagatagcgattcagattcagatagcgattcagaatcagatagcgattcggattcagacagtgattcagattcagacagcgactcagaatcagatagcgactcagaatcagatagtgagtcagattcagacagtgactcggactcagacagtgattcagactcagatagcgattcagactcagatagcgattcagactcagacagcgattcagattcagacagcgactcagaatcagacagcgactcagactcagatagcgactcagactcagacagcgactcagattcagatagcgattcagactcagacagcgactcagactcagacagcgactcagactcagatagcgattcagactcagacagcgactcagattcagatagcgattcggactcagacagcgattcagattcagacagcgactcagactcggatagcgattcagattcagacagcgactcagactcggatagcgactcggattcagatagtgactccgattcaagagttacaccaccaaataatgaacagaaagcaccatcaaatcctaaaggtgaagtaaaccattctaataaggtatcaaaacaacacaaaactgatgctttaccagaaacaggagataagagcgaaaacacaaatgcaactttatttggtgcaatgatggcattattaggatcattactattgtttagaaaacgcaagcaagatcataaagaaaaagcgtaa

SEQ ID NO: 66 - DNA, 1977 bp, Staphylococcus aureus
atgaaaaagcaaataatttcgctaggcgcattagcagttgcatctagcttatttacatgggataacaaagcagatgcgatagtaacaaaggattatagtaaagaatcaagagtgaatgagaaaagtaaaaagggagctactgtttcagattattactattggaaaataattgatagtttagaggcacaatttactggagcaatagacttattggaagattataaatatggagatcctatctataaagaagcgaaagatagattgatgacaagagtattaggagaagaccagtatttattaaagaaaaagattgatgaatatgagctttataaaaagtggtataaaagttcaaataagaacactaatatgcttactttccataaatataatctttacaatttaacaatgaatgaatataacgatatttttaactctttgaaagatgcagtttatcaatttaataaagaagttaaagaaatagagcataaaaatgttgacttgaagcagtttgataaagatggagaagacaaggcaactaaagaagtttatgaccttgtttctgaaattgatacattagttgtaacttattatgctgataaggattatggggagcatgcgaaagagttacgagcaaaactggacttaatccttggagatacagacaatccacataaaattacaaatgagcgtataaaaaaagaaatgatcgatgacttaaattcaattatagatgatttctttatggagactaaacaaaatagaccgaattctataacaaaatatgatccaacaaaacacaattttaaagagaagagtgaaaataaacctaattttgataaattagttgaagaaacaaaaaaagcagttaaagaagcagacgaatcttggaaaaataaaactgtcaaaaaatacgaggaaactgtaacaaaatctcctgttgtaaaagaagagaagaaagttgaagaacctcaattacctaaagttggaaaccagcaagaggttaaaactacggctggtaaagctgaagaaacaacacaaccagtggcacagccattagtaaaaattccacaagaaacaatctatggtgaaactgtaaaaggtccagaatatccaacgatggaaaataaaacgttacaaggtgaaatcgttcaaggtcccgattttctaacaatggaacaaaacagaccatctttaagcgataattatactcaaccgacgacaccgaaccctattttagaaggtcttgaaggtagctcatctaaacttgaaataaaaccacaaggtactgaatcaacgttgaaaggtattcaaggagaatcaagtgatattgaagttaaacctcaagcaactgaaacaacagaagcttctcaatatggtccgagaccgcaatttaacaaaacacctaagtatgtgaaatatagagatgctggtacaggtatccgtgaatacaacgatggaacatttggatatgaagcgagaccaagattcaacaagccaagtgaaacaaatgcatacaacgtaacgacaaatcaagatggcacagtatcatacggagctcgcccaacacaaaacaagccaagtgaaacaaacgcatataacgtaacaacacatgcaaatggtcaagtatcatacggtgctcgcccaacacaaaaaaagccaagcaaaacaaatgcatacaacgtaacaacacatgcaaatggtcaagtatcatatggcgctcgcccgacacaaaaaaagccaagcaaaacaaatgcatataacgtaacaacacatgcaaatggtcaagtatcatacggagctcgcccgacatacaagaagccaagcgaaacaaatgcatacaacgtaacaacacatgcaaatggtcaagtatcatatggcgctcgcccgacacaaaaaaagccaagcgaaacaaacgcatataacgtaacaacacatgcagatggtactgcgacatatgggcctagagtaacaaaataa

SEQ ID NO: 67 - protein, 961 aa, Staphylococcus aureus
MKSNLRYGIRKHKLGAASVFLGTMIVVGMGQEKEAAASEQNNTTVEESGSSATESKASETQTTTNNVNTIDETQSYSATSTEQPSKSTQVTTEEAPTTVQAPKVETEMKSQEDLPSEKVADKETTGTQVDIAQPSNVSEIKPRMKRSADVTAVSEKEVAEEAKATGTDVTNKVEVTESSLEGHNKDSNIVNPHNAQRVTLKYKWKFGEGIKAGDYFDFTLSDNVETHGISTLRKVPEIKSSTEDKVMANGQVINERTIRYTFTDYINNKKDLTAELNLNLFIDPTTVTKQGSQKVEVTLGQNKVSKEFDIKYLDGVKDRMGVTVNGRIDTLNKEEGKFSHFAYVKPNNQSLTSVTVTGQVTSGYKQSANNPTVKVYKHIGSDELAESVYAKLDDTSKFEDVTEKVNLSYTSNGGYTLNLGDLDNSKDYVIKYEGEYDQNAKDLNFRTHLSGYHKYYPYYPYYPYYPVQLTWNNGVAFYSNNAKGDGKDKPNDPIIEKSEPIDLDIKSEPPVEKHELTGTIEESNDSKPIDFEYHTAVEGAEGHAEGIIETEEDSIHVDFEESTHENSKHHADVVEYEEDTNPGGGQVTTESNLVEFDEESTKGIVTGAVSDHTTVEDTKEYTTESNLIELVDELPEEHGQAQGPIEEITENNHHISHSGLGTENGHGNYGVIDEIEENSHVDIKSELGYEGGQNSGNQSFEEDTEEDKPKYEQGGNIVDIDFDSVPQIHGQNNGNQSFEEDTEEDKPKYEQGGNIIDIDFDSVPQIHGFNKHNEIIEEDTNKDKPNYQFGGHNSVDFEEDTLPKVSGQNEGQQTIEEDTTPPTPPTPEVPSEPETPTPPTPEVPSEPGEPTPPKPEVPSEPETPVPPTPEVPSEPGKPVPPAKEEPKKPSKPVEQGKVVTPVIEINEKVKAVAPTKQKQSKKSELPETGGEESTNKGMLFGGLFSILGLVLLRRNKKNNKA

SEQ ID NO: 68 - protein, 578 aa, Staphylococcus aureus
MKFKSLITTTLALGVIASTGANFNTNEASAAAKPLDKSSSTLHHGHSNIQIPYTITVNGTSQNILSSLTFNKNQNISYKDIENKVKSVLYFNRGISDIDLRLSKQAEYTVHFKNGTKRVIDLKSGIYTADLINTSDIKAISVNVDTKKQPKDKAKANVQVPYTITVNGTSQNILSNLTFNKNQNISYKDLEGKVKSVLESNRGITDVDLRLSKQAKYTVNFKNGTKKVIDLKSGIYTANLINSSDIKSININVDTKKHIENKAKRNYQVPYSINLNGTSTNILSNLSFSNKPWTNYKNLTSQIKSVLKHDRGISEQDLKYAKKAYYTVYFKNGGKRILQLNSKNYTANLVHAKDVKRIEITVKTGTKAKADRYVPYTIAVNGTSTPILSKLKISNKQLISYKYLNDKVKSVLKSERGISDLDLKFAKQAKYTVYFKNGKKQVVNLKSDIFTPNLFSAKDIKKIDIDVKQYTKSKKKINKSNNVKFPVTINKFENIVSNEFVFYNASKITINDLSIKLKSAMANDQGITKHDIGLAERAVYKVYFKNGSSKYVDLKTEYKDERVFKATDIKKVDIELKF

SEQ ID NO: 69 - protein, 895 aa, Staphylococcus aureus
MNKHHPKLRSFYSIRKSTLGVASVIVSTLFLITSQHQAQAAENTNTSDKISENQNNNATTTQPPKDTNQTQPATQPANTAKNYPAADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKSDTNDAVVTNDQSSSVASNQTNTNTSNQNISTINNANNQPQATTNMSQPAQPKSSTNADQASSQPAHETNSNGNTNDKTNESSNQSDVNQQYPPADESLQDAIKNPAIIDKEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVDEETYNLQKLLAPYHKAKTLERQVYELEKLQEKLPEKYKAEYKKKLDQTRVELADQVKSAVTEFENVTPTNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGYEGQYHVRIINQDINTKDDDTSQNNTSEPLNVQTGQEGKVADTDVAENSSTATNPKDASDKADVIEPESDVVKDADNNIDKDVQHDVDHLSDMSDNNHFDKYDLKEMDTQIAKDTDRNVDKDADNSVGMSSNVDTDKDSNKNKDKVIQLNHIADKNNHTGKAAKLDVVKQNYNNTDKVTDKKTTEHLPSDIHKTVDKTVKTKEKAGTPSKENKLSQSKMLPKTGETTSSQSWWGLYALLGMLALFIPKFRKESK

SEQ ID NO: 70 - protein, 747 aa, Staphylococcus aureus
MAETTQDQTTNKNVLDSNKVKATTEQAKAEVKNPTQNISGTQVYQDPAIVQPKTANNKTGNAQVSQKVDTAQVNGDTRANQSATTNNTQPVAKSTSTTAPKTNTNVTNAGYSLVDDEDDNSENQINPELIKSAAKPAALETQYKTAAPKAATTSAPKAKTEATPKVTTFSASAQPRSVAATPKTSLPKYKPQVNSSINDYICKNNLKAPKIEEDYTSYFPKYAYRNGVGRPEGIVVHDTANDRSTINGEISYMKNNYQNAFVHAFVDGDRIIETAPTDYLSWGVGAVGNPRFINVEIVHTHDYASFARSMNNYADYAATQLQYYGLKPDSAEYDGNGTVWTHYAVSKYLGGTDHADPHGYLRSHNYSYDQLYDLINEKYLIKMGKVAPWGTQSTTTPTTPSKPTTPSKPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKPGTKLYTVPWGTSKQVAGSVSGSGNQTFKASKQQQIDKSIYLYGSVNGKSGWVSKAYLVDTAKPTPTPTPKPSTPTTNNKLTVSSLNGVAQINAKNNGLFTTVYDKTGKPTKEVQKTFAVTKEASLGGNKFYLVKDYNSPTLIGWVKQGDVIYNNAKSPVNVMQTYTVKPGTKLYSVPWGTYKQEAGAVSGTGNQTFKATKQQQIDKSIYLFGTVNGKSGWVSKAYLAVPAAPKKAVAQPKTAVK

SEQ ID NO: 71 - protein, 482 aa, Staphylococcus aureus
MAYTVTKPQTTQTVSKIAQVKPNNTGIRASVYEKTAKNGAKYADRTFYVTKERAHGNETYVLLNNTSHNIPLGWFNVKDLNVQNLGKEVKTTQKYTVNKSNNGLSMVPWGTKNQVILTGNNIAQGTFNATKQVSVGKDVYLYGTINNRTGWVNAKDLTAPTAVKPTTSAAKDYNYTYVIKNGNGYYYVTPNSDTAKYSLKAFNEQPFAVVKEQVINGQTWYYGKLSNGKLAWIKSTDLAKELIKYNQTGMTLNQVAQIQAGLQYKPQVQRVPGKWTDAKFNDVKHAMDTKRLAQDPALKYQFLRLDQPQNISIDKINQFLKGKGVLENQGAAFNKAAQMYGINEVYLISHALLETGNGTSQLAKGADVVNNKVVTNSNTKYHNVFGIAAYDNDPLREGIKYAKQAGWDTVSKAIVGGAKFIGNSYVKAGQNTLYKMRWNPAHPGTHQYATDVDWANINAKIIKGYYDKIGEVGKYFDIPQYK

SEQ ID NO: 72 - protein, 706 aa, Staphylococcus aureus
DRVLASHPDVATIRQNVTAANAAKSALDQARNGLTVDKAPLENAKNQLQHSIDTQTSTTGMTQDSINAYNAKLTAARNKIQQINQVLAGSPTVEQINTNTSTANQAKSDLDHARQALTPDKAPLQTAKTQLEQSINQPTDTTGMTTASLNAYNQKLQAARQKLTEINQVLNGNPTVQNINDKVTEANQAKDQLNTARQGLTLDRQPALTTLHGASNLNQAQQNNFTQQINAAQNHAALETIKSNITALNTAMTKLKDSVADNNTIKSDQNYTDATPANKQAYDNAVNAAKGVIGETTNPTMDVNTVNQKAASVKSTKDALDGQQNLQRAKTEATNAITHASDLNQAQKNALTQQVNSAQNVQAVNDIKQTTQSLNTAMTGLKRGVANHNQVVQSDNYVNADTNKKNDYNNAYNHANDIINGNAQHPVITPSDVNNALSNVTSKEHALNGEAKLNAAKQEANTALGHLNNLNNAQRQNLQSQINGAHQIDAVNTIKQNATNLNSAMGNLRQAVADKDQVKRTEDYADADTAKQNAYNSAVSSAETIINQTTNPTMSVDDVNRATSAVTSNKNALNGYEKLAQSKTDAARAIDALPHLNNAQKADVKSKINAASNIAGVNTVKQQGTDLNTAMGNLQGAINDEQTTLNSQNYQDATPSKKTAYTNAVQAAKDILNKSNGQNKTKDQVTEAMNQVNSAKNNLDGTRLLD

SEQ ID NO: 73 - protein, 241 aa, Staphylococcus aureus
ASTQHTVQSGESLWSIAQKYNTSVESIKQNNQLDNNLVFPGQVISVGGSDAQNTSNTSPQAGSASSHTVQAGESLNIIASRYGVSVDQLMAANNLRGYLIMPNQTLQIPNGGSGGTTPTATTGSNGNASSFNHQNLYTAGQCTWYVFDRRAQAGSPISTYWSDAKYWAGNAANDGYQVNNTPSVGSIMQSTPGPYGHVAYVERVNGDGSILISEMNYTYGPYNMNYRTIPASEVSSYAFIH

SEQ ID NO: 74 - protein, 995 aa, Staphylococcus aureus
MNNKKTATNRKGMIPNRLNKFSIRKYSVGTASILVGTTLIFGLSGHEAKAAEHTNGELNQSKNETTAPSENKTTKKVDSRQLKDNTQTATADQPKVTMSDSATVKETSSNMQSPQNATANQSTTKTSNVTTNDKSSTTYSNETDKSNLTQAKDVSTTPKTTTIKPRTLNRMAVNTVAAPQQGTNVNDKVHFSNIDIAIDKGHVNQTTGKTEFWATSSDVLKLKANYTIDDSVKEGDTFTFKYGQYFRPGSVRLPSQTQNLYNAQGNIIAKGIYDSTTNTTTYTFTNYVDQYTNVRGSFEQVAFAKRKNATTDKTAYKMEVTLGNDTYSEEIIVDYGNKKAQPLISSTNYINNEDLSRNMTAYVNQPKNTYTKQTFVTNLTGYKFNPNAKNFKIYEVTDQNQFVDSFTPDTSKLKDVTDQFDVIYSNDNKTATVDLMKGQTSSNKQYIIQQVAYPDNSSTDNGKIDYTLDTDKTKYSWSNSYSNVNGSSTANGDQKKYNLGDYVWEDTNKDGKQDANEKGIKGVYVILKDSNGKELDRTTTDENGKYQFTGLSNGTYSVEFSTPAGYTPTTANVGTDDAVDSDGLTTTGVIKDADNMTLDSGFYKTPKYSLGDYVWYDSNKDGKQDSTEKGIKGVKVTLQNEKGEVIGTTETDENGKYRFDNLDSGKYKVIFEKPAGLTQTGTNTTEDDKDADGGEVDVTITDHDDFTLDNGYYEEETSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDNDSDSDSDSDSDSDSDSDSDSDSDSDSDSDSDSDSDSDSDSDSDSDSDSDSDSDSDSDSDSDSDSDNDSDSDSDSDSDAGKHTPAKPMSTVKDQHKTAKALPETGSENNNSNNGTLFGGLFAALGSLLLFGRRKKQNK

SEQ ID NO: 75 - protein, 2186 aa, Staphylococcus aureus
MNLLKKNKYSIRKYKVGIFSTLIGTVLLLSNPNGAQALTTDNNVQSDTNQATPVNSQDKDVANNRGLANSAQNTPNQSATTNQATNQALVNHNNGSIVNQATPTSVQSSTPSAQNNNHTDGNTTATETVSNANNNDVVSNNTALNVPTKTNENGSGGHLTLKEIQEDVRHSSNKPELVAIAEPASNRPKKRSRRAAPADPNATPADPAAAAVGNGGAPVAITAPYTPTTDPNANNAGQNAPNEVLSFDDNGIRPSTNRSVPTVNVVNNLPGFTLINGGKVGVFSHAMVRTSMFDSGDNKNYQAQGNVIALGRIHGTDTNDHGDFNGIEKALTVNPNSELIFEFNTMTTKNGQGATNVIIKNADTNDTIAEKTVEGGPTLRLFKVPDNVRNLKIQFVPKNDAITDARGIYQLKDGYKYYSFVDSIGLHSGSHVFVERRTMDPTATNNKEFTVTTSLKNNGNSGASLDTNDFVYQVQLPEGVEYVNNSLTKDFPSNNSGVDVNDMNVTYDAANRVITIKSTGGGTANSPARLMPDKILDLRYKLRVNNVPTPRTVTFNETLTYKTYTQDFINSAAESHTVSTNPYTIDIIMNKDALQAEVDRRIQQADYTFASLDIFNGLKRRAQTILDENRNNVPLNKRVSQAYIDSLTNQMQHTLIRSVDAENAVNKKVDQMEDLVNQNDELTDEEKQAAIQVIEEHKNEIIGNIGDQTTDDGVTRIKDQGIQTLSGDTATPVVKPNAKKAIRDKATKQREIINATPDATEDEIQDALNQLATDETDAIDNVTNATTNADVETAKNNGINTIGAVVPQVTHKKAARDAINQATATKRQQINSNREATQEEKNAALNELTQATNHALEQINQATTNANVDNAKGDGLNAINPIAPVTVVKQAARDAVSHDAQQHIAEINANPDATQEERQAAIDKVNAAVTAANTNILNANTNADVEQVKTNAIQGIQAITPATKVKTDAKNAIDKSAETQHNTIFNNNDATLEEQQAAQQLLDQAVATAKQNINAADTNQEVAQAKDQGTQNIVVIQPATQVKTDTRNVVNDKAREAITNINATTGATREEKQEAINRVNTLKNRALTDIGVTSTTAMVNSIRDDAVNQIGAVQPHVTKKQTATGVLNDLATAKKQEINQNTNATTEEKQVALNQVDQELATAINNINQADTNAEVDQAQQLGTKAINAIQPNIVKKPAALAQINQHYNAKLAEINATPDATNDEKNAAINTLNQDRQQAIESIKQANTNAEVDQAATVAENNIDAVQVDVVKKQAARDKITAEVAKRIEAVKQTPNATDEEKQAAVNQINQLKDQAINQINQNQTNDQVDTTTNQAVNAIDNVEAEVVIKTKAIADIEKAVKEKQQQIDNSLDSTDNEKEVASQALAKEKEKALAAIDQAQTNSQVNQAATNGVSAIKIIQPETKVKPAAREKINQKANELRAKINQDKEATAEERQVALDKINEFVNQAMTDITNNRTNQQVDDTTSQALDSIALVTPDHIVRAAARDAVKQQYEAKKREIEQAEHATDEEKQVALNQLANNEKRALQNIDQAIANNDVKRVETNGIATLKGVQPHIVIKPEAQQAIKASAENQVESIKDTPHATVDELDEANQLISDTLKQAQQEIENTNQDAAVTDVRNQTIKAIEQIKPKVRRKRAALDSIEENNKNQLDAIRNTLDTTQDERDVAIDTLNKIVNTIKNDIAQNKTNAEVDRTETDGNDNIKVILPKVQVKPAARQSVGVKAEAQNALIDQSDLSTEEERLAAKHLVEQALNQAIDQINHADKTAQVNQDSINAQNIISKIKPATTVKATALQQIQNIATNKINLIKANNEATDEEQNIAIAQVEKELIKAKQQIASAVTNADVAYLLHDEKNEIREIEPVINRKASAREQLTTLFNDKKQAIEANIQATVEERNSILAQLQNIYDTAIGQIDQDRSNAQVDKTASLNLQTIHDLDVHPIKKPDAEKTINDDLARVTALVQNYRKVSNRNKADALKAITALKLQMDEELKTARTNADVDAVLKRFNVALSDIEAVITEKENSLLRIDNIAQQTYAKFKAIATPEQLAKVKVLIDQYVADGNRMIDEDATLNDIKQHTQFIVDEILAIKLPAEATKVSPKEIQPAPKVCTPIKKEETHESRKVEKELPNTGSEGMDLPLKEFALITGAALLARRRTKNEKES

SEQ ID NO: 76 - protein, 773 aa, Staphylococcus aureus
EENSVQDVKDSNTDDELSDSNDQSSDEEKNDVINNNQSINTDDNNQIIKKEETNNYDGIEKRSEDRTESTTNVDENEATFLQKTPQDNTHLTEEEVKESSSVESSNSSIDTAQQPSHTTINREESVQTSDNVEDSHVSDFANSKIKESNTESGKEENTIEQPNKVKEDSTTSQPSGYTNIDEKISNQDELLNLPINEYENKARPLSTTSAQPSIKRVTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNDVNINFGNIDSPYIIKVISKYDPNKDDYTTIQQTVTMQTTINEYTGEFRTASYDNTIAFSTSSGQGQGDLPPEKTYKIGDYVWEDVDKDGIQNTNDNEKPLSNVLVTLTYPDGTSKSVRTDEDGKYQFDGLKNGLTYKITFETPEGYTPTLKHSGTNPALDSEGNSVWVTINGQDDMTIDSGFYQTPKYSLGNYVWYDTNKDGIQGDDEKGISGVKVTLKDENGNIISTTTTDENGKYQFDNLNSGNYIVHFDKPSGMTQTTTDSGDDDEQDADGEEVHVTITDHDDFSIDNGYYDDE

SEQ ID NO: 77 - DNA, 2886 bp, Staphylococcus aureus
gtgaaaagcaatcttagatacggcataagaaaacacaaattgggagcggcctcagtattcttaggaacaatgatcgttgttggaatgggacaagaaaaagaagctgcagcatcggaacaaaacaatactacagtagaggaaagtgggagttcagctactgaaagtaaagcaagcgaaacacaaacaactacaaataacgttaatacaatagatgaaacacaatcatacagcgcgacatcaactgagcaaccatcaaaatcaactcaagtaacaacagaagaagcaccaacaactgtgcaagcaccaaaagtagaaaccgaaatgaaatcacaagaagatttaccatcagaaaaagttgctgataaggaaactacaggaactcaagttgacatagctcaaccaagtaacgtctcagaaattaaaccaagaatgaaaagatcagctgacgttacagcagtttcagagaaagaagtagcggaagaagctaaagcgacaggtacagatgtaacaaataaagtggaagttactgaaagctctttagaaggacataataaagattcgaatattgttaatccgcataatgctcaaagagtaactttaaaatacaaatggaaatttggagaaggaattaaggcaggagattattttgatttcacattaagtgataatgttgaaacacatggtatatcaacactgcgtaaagttccggagataaaaagttcaacagaagataaagttatggcaaatggtcaagttataaatgaacgtacaattcgctatacatttactgattatataaataacaaaaaagatttaactgctgaattaaacttaaacctattcattgacccaacaacagtgacaaagcaagggagtcaaaaagttgaagtaacactaggtcaaaataaagtctcaaaagaatttgatatcaaatatttagacggcgttaaagatagaatgggtgttactgttaatggtcgtattgatactttgaataaagaagagggtaaatttagccattttgcatatgtgaagcctaacaaccagtcgttaacttctgtcacagtaactggtcaagtaacatctggatataaacaaagtgctaataatccaacagtcaaagtatataaacacattggttcagatgaattagctgaaagtgtttatgcaaagcttgatgataccagtaaatttgaagatgtgactgaaaaagtaaatctatcttacacaagtaatggtgggtacacattgaaccttggcgatttagataattcgaaagactatgtaattaaatatgaaggtgaatatgatcaaaatgctaaggatctaaatttccgaacacatctttcaggatatcataaatactacccatactatccttattacccgtattatccagttcaattaacttggaacaacggtgttgcattttactctaataatgctaaaggcgatggtaaagataaaccaaatgatcctatcattgagaagagtgaaccaattgatttagacattaaatcagagccaccagtggagaagcatgaattgactggtacaatcgaagaaagtaacgattctaagccaattgattttgaatatcatacagctgttgaaggtgcagaaggtcatgcagaaggtattattgaaactgaagaagattctattcatgtggattttgaagaatctacacatgaaaattcaaaacatcacgctgatgttgttgaatatgaagaggatacaaacccaggtggtggccaagtaacaactgagtctaacttagttgaatttgacgaagagtctacaaaaggtattgtaactggcgcagtgagcgaccatacaacagttgaagatacgaaagaatatacaactgaaagtaatctgattgaattagtggatgaattacctgaagaacatggtcaagcacaagggccaatcgaggaaattactgaaaacaatcatcatatttctcattctggtttaggaactgaaaatggtcacggtaattatggcgtgattgatgaaatcgaagaaaatagccacgttgatattaagagtgaattaggttatgaaggtggccaaaatagcggtaatcagtcattcgaggaagacacagaagaagataaacctaaatatgaacaaggtggtaatatcgtagatatcgatttcgacagtgtacctcaaattcatggtcaaaataatggtaaccagtcattcgaggaagacacagaagaagacaagcctaagtatgaacaaggtggtaacatcattgatatcgacttcgacagtgtgccacaaattcatggattcaataagcataatgaaattattgaagaagatacaaacaaagataaacctaattatcaatttggtggacacaacagtgttgattttgaagaagatacacttccaaaagtaagtggtcaaaatgaaggtcaacaaacgattgaagaagatacaacgccgccaacaccgccaacaccagaggtaccaagtgagccggaaacaccaacaccaccaacaccagaagtaccgagtgagccaggcgaaccaacgccaccaaaaccggaagtaccaagtgagccggaaacaccagtaccaccaacaccagaggtaccatctgaacctggtaaaccagtaccacctgctaaagaagaacctaaaaaaccttctaaaccagtggaacaaggtaaggtagtaacacctgttattgaaatcaatgaaaaggttaaagcagtggcaccaactaaacaaaaacaatctaagaaatctgaactacctgaaacaggtggagaagaatcaacaaacaaaggtatgttgttcggcggattattcagcattctaggtttagtattattacgcagaaataaaaagaataacaaagcataa

SEQ ID NO: 78 - DNA, 1737 bp, Staphylococcus aureus
atgaaatttaagtcattgattacaacaacattagcattaggcgttatagcatcaacaggagcaaactttaatactaacgaagcatctgccgcagctaagccattagataaatcatcaagtacattacaccatggacattctaacatccagattccatatacaattactgtgaacggtacaagccaaaacattttatcaagcttaacatttaataagaatcaaaatattagttataaagatatagagaataaagttaaatcagttttatactttaatagaggtattagtgatatcgatttaagactttcaaagcaagcggaatatacggttcattttaaaaatggaacaaaaagagttatcgatttgaaatcaggtatctacacagctgacttaatcaatacaagtgacattaaagctatcagtgttaacgtagatactaaaaagcaacctaaagataaagctaaagcaaatgttcaagtgccatatacaatcacagtgaacggcacaagccaaaacattttatcaaacctaacatttaataaaaatcaaaatattagttacaaagatttagagggtaaagttaaatcagttttagaatcaaatagaggtattactgatgttgatttaagactttcgaagcaagcgaaatatacagttaattttaaaaatggaacgaagaaagttatcgatttgaaatcaggtatttacacagcgaatttaatcaattcaagtgatattaaaagtatcaatattaacgtagatacaaaaaaacatatcgaaaataaagctaaaagaaactatcaagttccatattcaattaatctaaatggtacatctacaaacattttatcgaatctttcattttcaaataaaccttggacaaattacaaaaatttaactagtcaaataaaatcagtactgaagcatgatagaggtattagtgaacaagatttaaaatatgctaagaaagcttattatactgtttattttaaaaatggtggtaaaagaatcttacagttaaattcaaaaaattacacagcaaacttagttcatgcgaaagatgttaagagaattgaaattactgttaaaacaggaactaaagcgaaagcagacagatatgtaccatacacaattgcagtaaatggcacatcaacaccaattttatcaaaactaaaaatttcgaataaacaattaattagttacaaatatttaaacgacaaagtgaaatctgtattaaaaagtgaaagaggtatcagtgatcttgacttaaaatttgcgaaacaagcaaaatatacagtatatttcaaaaatggaaagaaacaagtagtgaatttaaaatcagacatctttacacctaatttatttagtgccaaagatattaaaaagattgatattgatgtaaaacaatacactaaatcaaaaaaaaaaataaataaatctaataatgtgaaattcccagtaacaataaataaatttgaaaacatagtttcaaatgaatttgtgttctataatgcaagcaaaattacaattaatgatttaagtataaaacttaaatcagcaatggcaaatgatcaagggataactaaacatgacataggacttgctgaacgcgcagtgtataaagtgtattttaaaaatggttcgtcaaaatatgtagacttaaaaactgagtataaagatgaaagagtatttaaagcaactgacattaaaaaggtagatattgaacttaaattctaa

SEQ ID NO: 79 - DNA, 2688 bp, Staphylococcus aureus
atgaacaaacatcacccaaaattaaggtctttctattctattagaaaatcaactctaggcgttgcatcggtcattgtcagtacactatttttaattacttctcaacatcaagcacaagcagcagaaaatacaaatacttcagataaaatctcggaaaatcaaaataataatgcaactacaactcagccacctaaggatacaaatcaaacacaacctgctacgcaaccagcaaacactgcgaaaaactatcctgcagcggatgaatcacttaaagatgcaattaaagatcctgcattagaaaataaagaacatgatataggtccaagagaacaagtcaatttccagttattagataaaaacaatgaaacgcagtactatcactttttcagcatcaaagatccagcagatgtgtattacactaaaaagaaagcagaagttgaattagacatcaatactgcttcaacatggaagaagtttgaagtctatgaaaacaatcaaaaattgccagtgagacttgtatcatatagtcctgtaccagaagaccatgcctatattcgattcccagtttcagatggcacacaagaattgaaaattgtttcttcgactcaaattgatgatggagaagaaacaaattatgattatactaaattagtatttgctaaacctatttataacgatccttcacttgtaaaatcagatacaaatgatgcagtagtaacgaatgatcaatcaagttcagtcgcaagtaatcaaacaaacacgaatacatctaatcaaaatatatcaacgatcaacaatgctaataatcaaccgcaggcaacgaccaatatgagtcaacctgcacaaccaaaatcgtcaacgaatgcagatcaagcgtcaagccaaccagctcatgaaacaaattctaatggtaatactaacgataaaacgaatgagtcaagtaatcagtcggatgttaatcaacagtatccaccagcagatgaatcactacaagatgcaattaaaaacccggctatcatcgataaagaacatacagctgataattggcgaccaattgattttcaaatgaaaaatgataaaggtgaaagacagttctatcattatgctagtactgttgaaccagcaactgtcatttttacaaaaacaggaccaataattgaattaggtttaaagacagcttcaacatggaagaaatttgaagtttatgaaggtgacaaaaagttaccagtcgaattagtatcatatgattctgataaagattatgcctatattcgtttcccagtatctaatggtacgagagaagttaaaattgtgtcatctattgaatatggtgagaacatccatgaagactatgattatacgctaatggtctttgcacagcctattactaataacccagacgactatgtggatgaagaaacatacaatttacaaaaattattagctccgtatcacaaagctaaaacgttagaaagacaagtttatgaattagaaaaattacaagagaaattgccagaaaaatataaggcggaatataaaaagaaattagatcaaactagagtagagttagctgatcaagttaaatcagcagtgacggaatttgaaaatgttacacctacaaatgatcaattaacagatttacaagaagcgcattttgttgtttttgaaagtgaagaaaatagtgagtcagttatggacggctttgttgaacatccattctatacagcaactttaaatggtcaaaaatatgtagtgatgaaaacaaaggatgacagttactggaaagatttaattgtagaaggtaaacgtgtcactactgtttctaaagatcctaaaaataattctagaacgctgattttcccatatatacctgacaaagcagtttacaatgcgattgttaaagtcgttgtggcaaacattggttatgaaggtcaatatcatgtcagaattataaatcaggatatcaatacaaaagatgatgatacatcacaaaataacacgagtgaaccgctaaatgtacaaacaggacaagaaggtaaggttgctgatacagatgtagctgaaaatagcagcactgcaacaaatcctaaagatgcgtctgataaagcagatgtgatagaaccagagtctgacgtggttaaagatgctgataataatattgataaagatgtgcaacatgatgttgatcatttatccgatatgtcggataataatcacttcgataaatatgatttaaaagaaatggatactcaaattgccaaagatactgatagaaatgtggataaagatgccgataatagcgttggtatgtcatctaatgtcgatactgataaagactctaataaaaataaagacaaagtcatacagctgaatcatattgccgataaaaataatcatactggaaaagcagcaaagcttgacgtagtgaaacaaaattataataatacagacaaagttactgacaaaaaaacaactgaacatctgccgagtgatattcataaaactgtagataaaacagtgaaaacaaaagaaaaagccggcacaccatcgaaagaaaacaaacttagtcaatctaaaatgctaccaaaaactggagaaacaacttcaagccaatcatggtggggcttatatgcgttattaggtatgttagctttattcattcctaaattcagaaaagaatctaaataa

SEQ ID NO: 80 - DNA, 2238 bp, Staphylococcus aureus
gctgagacgacacaagatcaaactactaataaaaacgttttagatagtaataaagttaaagcaactactgaacaagcaaaagctgaggtaaaaaatccaacgcaaaacatttctggcactcaagtatatcaagaccctgctattgtccaaccaaaaacagcaaataacaaaacaggcaatgctcaagtaagtcaaaaagttgatactgcacaagtaaatggtgacactcgtgctaatcaatcagcgactacaaataatacgcagcctgttgcaaagtcaacaagcactacagcacctaaaactaacactaatgttacaaatgctggttatagtttagttgatgatgaagatgataattcagaaaatcaaattaatccagaattaattaaatcagctgctaaacctgcagctcttgaaacgcaatataaaaccgcagcacctaaagctgcaactacatcagcacctaaagctaaaactgaagcgacacctaaagtaactacttttagcgcttcagcacaaccaagatcagttgctgcaacaccaaaaacgagtttgccaaaatataaaccacaagtaaactcttcaattaacgattacatttgtaaaaataacttaaaagcacctaaaattgaagaagattatacatcttacttccctaaatacgcataccgtaacggcgtaggtcgtcctgaaggtatcgtagttcatgatacagctaatgatcgttcgacgataaatggtgaaattagttatatgaaaaataactatcaaaacgcattcgtacatgcatttgttgatggggatcgtataatcgaaacagcaccaacggattacttatcttggggtgtcggtgcagtcggtaaccctagattcatcaatgttgaaatcgtacacacacacgactatgcttcatttgcacgttcaatgaataactatgctgactatgcagctacacaattacaatattatggtttaaaaccagacagtgctgagtatgatggaaatggtacagtatggactcactacgctgtaagtaaatatttaggtggtactgaccatgccgatccacatggatatttaagaagtcataattatagttatgatcaattatatgacttaattaatgaaaaatatttaataaaaatgggtaaagtggcgccatggggtacgcaatctacaactacccctactacaccatcaaaaccaacaacaccgtcgaaaccatcaactggtaaattaacagttgctgcaaacaatggtgtcgcacaaatcaaaccaacaaatagtggtttatatactactgtatacgacaaaactggtaaagcaactaatgaagttcaaaaaacatttgctgtatctaaaacagctacattaggtaatcaaaaattctatcttgttcaagattacaattctggtaataaatttggttgggttaaagaaggcgatgtggtttacaacacagctaaatcacctgtaaatgtaaatcaatcatattcaatcaaacctggtacgaaactttatacagtaccttggggtacatctaaacaagttgctggtagtgtgtctggctctggaaaccaaacatttaaggcttcaaagcaacaacaaattgataaatcaatttatttatatggctctgtgaatggtaaatctggttgggtaagtaaagcatatttagttgatactgctaaacctacgcctacaccaacacctaagccatcaacacctacaacaaataataaattaacagtttcatcattaaacggtgttgctcaaattaatgctaaaaacaatggcttattcactacagtttatgacaaaactggtaagccaacgaaagaagttcaaaaaacatttgctgtaacaaaagaagcaagtttaggtggaaacaaattctacttagttaaagattacaatagtccaactttaattggttgggttaaacaaggtgacgttatttataacaatgcaaaatcacctgtaaatgtaatgcaaacatatacagtaaaaccaggcactaaattatattcagtaccttggggcacttataaacaagaagctggtgcagtttctggtacaggtaaccaaacttttaaagcgactaagcaacaacaaattgataaatctatctatttatttggaactgtaaatggtaaatctggttgggtaagtaaagcatatttagctgtacctgctgcacctaaaaaagcagtagcacaaccaaaaacagctgtaaaa

SEQ ID NO: 81 - DNA, 1443 bp, Staphylococcus aureus
gcttatactgttactaaaccacaaacgactcaaacagttagcaagattgctcaagttaaaccaaacaacactggtattcgtgcttctgtttatgaaaaaacagcgaaaaacggtgcgaaatatgcagaccgtacgttctatgtaacaaaagagcgtgctcatggtaatgaaacgtatgtattattaaacaatacaagccataacatcccattaggttggttcaatgtaaaagacttaaatgttcaaaacttaggcaaagaagttaaaacgactcaaaaatatactgttaataaatcaaataacggcttatcaatggttccttggggtactaaaaaccaagtcattttaacaggcaataacattgctcaaggtacatttaatgcaacgaaacaagtatctgtaggcaaagatgtttatttatacggtactattaataaccgcactggttgggtaaatgcaaaagatttaactgcaccaactgctgtgaaaccaactacatcagctgccaaagattataactacacttatgtaattaaaaatggtaatggttattactatgtaacaccaaattctgatacagctaaatactcattaaaagcatttaatgaacaaccattcgcagttgttaaagaacaagtcattaatggacaaacttggtactatggtaaattatctaacggtaaattagcatggattaaatcaactgatttagctaaagaattaattaagtataatcaaacaggtatgacattaaaccaagttgctcaaatacaagctggtttacaatataaaccacaagtacaacgtgtaccaggtaagtggacagatgctaaatttaatgatgttaagcatgcaatggatacgaagcgtttagctcaagatccagcattaaaatatcaattcttacgcttagaccaaccacaaaatatttctattgataaaattaatcaattcttaaaaggtaaaggtgtattagaaaaccaaggtgctgcatttaacaaagctgctcaaatgtatggcattaatgaagtttatcttatctcacatgccctattagaaacaggtaacggtacttctcaattagcgaaaggtgcagatgtagtgaacaacaaagttgtaactaactcaaacacgaaataccataacgtatttggtattgctgcatatgataacgatcctttacgtgaaggtattaaatatgctaaacaagctggttgggacacagtatcaaaagcaatcgttggtggtgctaaattcatcggcaactcatatgtaaaagctggtcaaaatacactttacaaaatgagatggaatcctgcacatccaggaacacaccaatatgctacagatgtagattgggctaacatcaatgctaaaatcatcaaaggctactatgataaaattggcgaagtcggcaaatacttcgacatcccacaatataaa

SEQ ID NO: 82 - DNA, 2118 bp, Staphylococcus aureus
gatcgtgtattagcctcacatccagatgttgcgacaatacgtcaaaacgtgacagcagcgaatgccgctaaatcagcacttgatcaagcacgtaatggcttaacagtcgataaagcgcctttagaaaatgcgaaaaatcaactacaacatagtattgacacgcaaacaagtacaactggtatgacacaagactctataaatgcatacaatgcgaagttaacagctgcacgtaataagattcaacaaatcaatcaagtattagcaggttcaccgactgtagaacaaattaatacaaatacgtctacagcaaatcaagctaaatctgatttagatcatgcacgtcaagctttaacaccagataaagcgccgcttcaaactgcgaaaacgcaattagaacaaagcattaatcaaccaacggatacaacaggtatgacgaccgcttcgttaaatgcgtacaaccaaaaattacaagcagcgcgtcaaaagttaactgaaattaatcaagtgttgaatggcaacccaactgtccaaaatatcaatgataaagtgacagaggcaaaccaagctaaggatcaattaaatacagcacgtcaaggtttaacattagatagacagccagcgttaacaacattacatggtgcatctaacttaaaccaagcacaacaaaataatttcacgcaacaaattaatgctgctcaaaatcatgctgcgcttgaaacaattaagtctaacattacggctttaaatactgcgatgacgaaattaaaagacagtgttgcggataataatacaattaaatcagatcaaaattacactgacgcaacaccagctaataaacaagcgtatgataatgcagttaatgcggctaaaggtgtcattggagaaacgactaatccaacgatggatgttaacacagtgaaccaaaaagcagcatctgttaaatcgacgaaagatgctttagatggtcaacaaaacttacaacgtgcgaaaacagaagcaacaaatgcgattacgcatgcaagtgatttaaaccaagcacaaaagaatgcattaacacaacaagtgaatagtgcacaaaacgtgcaagcagtaaatgatattaaacaaacgactcaaagcttaaatactgctatgacaggtttaaaacgtggcgttgctaatcataaccaagtcgtacaaagtgataattatgtcaacgcagatactaataagaaaaatgattacaacaatgcatacaaccatgcgaatgacattattaatggtaatgcacaacatccagttataacaccaagtgatgttaacaatgctttatcaaatgtcacaagtaaagaacatgcattgaatggtgaagctaagttaaatgctgcgaaacaagaagcgaatactgcattaggtcatttaaacaatttaaataatgcacaacgtcaaaacttacaatcgcaaattaatggtgcgcatcaaattgatgcagttaatacaattaagcaaaatgcaacaaacttgaatagtgcaatgggtaacttaagacaagctgttgcagataaagatcaagtgaaacgtacagaagattatgcggatgcagatacagctaaacaaaatgcatataacagtgcagtttcaagtgccgaaacaatcattaatcaaacaacaaatccaacgatgtctgttgatgatgttaatcgtgcaacttcagctgttacttctaataaaaatgcattaaatggttatgaaaaattagcacaatctaaaacagatgctgcaagagcaattgatgcattaccacatttaaataatgcacaaaaagcagatgttaaatctaaaattaatgctgcatcaaatattgctggcgtaaatactgttaaacaacaaggtacagatttaaatacagcgatgggtaacttgcaaggtgcaatcaatgatgaacaaacgacgcttaatagtcaaaactatcaagatgcgacacctagtaagaaaacagcatacacaaatgcggtacaagctgcgaaagatattttaaataaatcaaatggtcaaaataaaacgaaagatcaagttactgaagcgatgaatcaagtgaattctgctaaaaataacttagatggtacgcgtttattagat

SEQ ID NO: 83 - DNA, 726 bp, Staphylococcus aureus
gcttctacacaacatacagtacaatctggtgaatcattatggagtattgctcaaaaatacaacacttcagtagagagtattaaacaaaataaccaattagataacaacttggtattccctggtcaagttatctcagtaggtggaagtgatgcacaaaatacgtcaaacacttctccacaagctggttcagcatcatctcatactgtacaagctggtgaatcattaaatatcattgctagcagatatggtgtttcagttgatcaattaatggcagccaataacttacgtggttatttaattatgcctaaccaaacattacaaattcctaatggtggatcaggtggtacaacaccaacagctacaacaggtagcaatggcaatgcatcatcttttaatcaccaaaatttatacactgctggtcaatgtacatggtacgtatttgaccgtcgtgctcaagctggtagtccaattagcacatattggtcagacgctaagtattgggctggtaacgcagctaatgatggttaccaagtaaacaacacaccatcagttggttcaattatgcaaagcacacctggtccatatggtcatgttgcttatgttgaacgtgtcaatggtgatggtagtatcttgatttctgaaatgaattacacatatggtccatacaatatgaactaccgtacaattccagcttcagaagtttctagctatgcattcatccattaa

SEQ ID NO: 84 - DNA, 2988 bp, Staphylococcus aureus
atgaataataaaaagacagcaacaaatagaaaaggcatgataccaaatcgattaaacaaattttcgataagaaagtattctgtaggtactgcttcaattttagtagggacaacattgatttttgggttaagtggtcatgaagctaaagcggcagaacatacgaatggagaattaaatcaatcaaaaaatgaaacgacagccccaagtgagaataaaacaactaaaaaagttgatagtcgtcaactaaaagacaatacgcaaactgcaactgcagatcagcctaaagtgacaatgagtgatagtgcaacagttaaagaaactagtagtaacatgcaatcaccacaaaacgctacagctaatcaatctactacaaaaactagcaatgtaacaacaaatgataaatcatcaactacatatagtaatgaaactgataaaagtaatttaacacaagcaaaagatgtttcaactacacctaaaacaacgactattaaaccaagaactttaaatcgcatggcagtgaatactgttgcagctccacaacaaggaacaaatgttaatgataaagtacatttttcaaatattgacattgcgattgataaaggacatgttaatcagactactggtaaaactgaattttgggcaacttcaagtgatgttttaaaattaaaagcaaattacacaatcgatgattctgttaaagagggcgatacatttacttttaaatatggtcaatatttccgtccaggatcagtaagattaccttcacaaactcaaaatttatataatgcccaaggtaatattattgcaaaaggtatttatgatagtacaacaaacacaacaacatatacttttacgaactatgtagatcaatatacaaatgttagaggtagctttgaacaagttgcatttgcgaaacgtaaaaatgcaacaactgataaaacagcttataaaatggaagtaactttaggtaatgatacatatagcgaagaaatcattgtcgattatggtaataaaaaagcacaaccgcttatttcaagtacaaactatattaacaatgaagatttatcgcgtaatatgactgcatatgtaaatcaacctaaaaatacatatactaaacaaacgtttgttactaatttaactggatataaatttaatccaaatgcaaaaaacttcaaaatttacgaagtgacagatcaaaatcaatttgtggatagtttcacccctgatacttcaaaacttaaagatgttactgatcaattcgatgttatttatagtaatgataataaaacagctacagtcgatttaatgaaaggccaaacaagcagcaataaacaatacatcattcaacaagttgcttatccagataatagttcaacagataatggaaaaattgattatactttagacactgacaaaactaaatatagttggtcaaatagttattcaaatgtgaatggctcatcaactgctaatggcgaccaaaagaaatataatctaggtgactatgtatgggaagatacaaataaagatggtaaacaagatgccaatgaaaaagggattaaaggtgtttatgtcattcttaaagatagtaacggtaaagaattagatcgtacgacaacagatgaaaatggtaaatatcagttcactggtttaagcaatggaacttatagtgtagagttttcaacaccagccggttatacaccgacaactgcaaatgtaggtacagatgatgctgtagattctgatggactaactacaacaggtgtcattaaagacgctgacaacatgacattagatagtggattctacaaaacaccaaaatatagtttaggtgattatgtttggtacgacagtaataaagatggtaaacaagattcgactgaaaaaggaattaaaggtgttaaagttactttgcaaaacgaaaaaggcgaagtaattggtacaactgaaacagatgaaaatggtaaataccgctttgataatttagatagtggtaaatacaaagttatctttgaaaaacctgctggcttaactcaaacaggtacaaatacaactgaagatgataaagatgccgatggtggcgaagttgatgtaacaattacggatcatgatgatttcacacttgataatggctactacgaagaagaaacatcagatagcgactcagattctgacagcgattcagactcagatagcgactcagattcagatagcgactcagattcagacagcgattcagacagcgactcagactcagatagcgattcagattcagacagcgactcagactcagacagcgattcagactcggatagcgactcagactcagatagcgactcagattcggatagcgactcagactcagatagcgattcagattcagatagcgattcggactcagacagtgattcagattcagactcagatagcgactcagattctgacagcgattcagactcagacagcgactcagactcagacagtgattcagattcagacagcgactcagattcagatagcgactcagactcagatagcgactcagattcagatagcgattcggactcagacaacgactcagattcagatagcgattcagattcagatagcgactcagattcggacagcgattcagactcagatagcgattcagactcagacagcgattcagattcagatagcgactcagactcagatagcgactcagactcggatagcgattcagattcagacagcgactcagattcagatagcgattcggactcagacaacgactcagattcagatagcgattcagattcagatgcaggtaaacatactccggctaaaccaatgagtacggttaaagatcagcataaaacagctaaagcattaccagaaacaggtagtgaaaataataattcaaataatggcacattattcggtggattattcgcggcattaggatcattattgttattcggtcgtcgtaaaaaacaaaataaataa

SEQ ID NO: 85 - DNA, 6561 bp, Staphylococcus aureus
atgaatttgttaaagaaaaataaatatagtattaggaagtataaagtaggcatattctctactttaatcggaacagttttattactttcaaacccaaatggtgcacaagccttaactacggataataatgtacaaagcgatactaatcaagcaacacctgtaaattcacaagataaagatgttgctaataatagaggtttagcaaatagtgcgcagaatacacctaatcaatctgcaacaaccaatcaagcaacgaatcaagcattggttaatcataataatggtagtatagtaaatcaagctacgccaacatcagtgcaatcaagtacgccttcagcacaaaacaataatcatacagatggcaatacaacagcaactgagacagtgtcaaacgctaataataatgatgtagtgtcgaataataccgcattaaatgtaccaactaaaacaaatgaaaatggttcaggaggacatctaactttaaaggaaattcaagaagatgttcgtcattcttcaaataaaccagagctagttgcaattgctgaaccagcatctaatagaccgaaaaagagaagtagacgtgcggcaccggcagatcctaatgcaactccagcagatccagcggctgcagcggtaggaaacggtggtgcaccagttgcaattacagcgccatatacgccaacaactgatcctaatgccaataatgcaggacaaaatgcacctaacgaagtgctgtcatttgatgacaatggtattagaccaagtaccaaccgttctgtgccaacagtaaacgttgttaataacttgccgggcttcacactaatcaatggtggcaaagtaggggtgtttagtcatgcaatggtaagaacgagcatgtttgattcaggagataataagaactatcaagcacaaggaaatgtaattgcattaggtcgtatacatggaactgatacgaatgaccatggcgattttaatggtatcgagaaagcattaacagtaaatccgaattctgaattaatctttgaatttaatacaatgactactaaaaacggtcaaggcgcaacaaatgttattatcaaaaatgctgatactaatgatacgattgctgaaaagactgttgaaggcggtccaactttgcgtttatttaaagtacctgataatgtgagaaatctcaaaattcaatttgtacctaaaaatgacgcaataacagatgcgcgtggcatttatcaactaaaagatggttacaaatactatagctttgttgactctatcggacttcattctgggtcacatgtttttgttgaaagacgaacaatggatccaacagcaacaaataataaagagtttactgtaacaacatcattaaagaataatggtaattctggtgcttctctagatacaaatgactttgtatatcaagttcaattacctgaaggtgttgaatatgtgaacaattcattgactaaagattttccaagtaacaattcaggcgttgatgttaatgatatgaatgttacatatgatgcagcaaatcgtgtgataacaattaaaagtactggaggaggtacagcaaactctccggcacgacttatgcctgataaaatactcgatttaagatataaattacgtgtaaataatgtgccgacaccaagaacagtaacatttaacgagacattaacgtataaaacatatacacaagatttcattaattcagctgcagaaagtcatactgtaagtacaaatccatatactatcgatatcatcatgaataaagatgcattacaagccgaagttgacagacgtattcaacaagctgattatacatttgcgtcattagatatctttaatggtctgaaacgacgcgcacaaacgattttagatgaaaatcgtaacaatgtaccattaaataaaagagtttctcaagcatatattgattcattaactaatcaaatgcaacatacgttaattcgaagtgttgatgctgaaaatgcagttaataaaaaagttgaccaaatggaagatttagttaatcaaaatgatgaattgacagatgaagaaaaacaagcagcaatacaagttatcgaggaacataaaaatgaaataattggtaatattggtgaccaaacgactgatgatggcgttactagaatcaaagatcaaggtatacagaccttaagtggggatactgcaacaccggttgttaaaccaaatgctaaaaaagcaatacgtgataaagcaacgaaacaaagggaaattatcaatgcaacaccagatgctactgaagacgagattcaagatgcactaaatcaattagctacggatgaaacagatgctattgataatgttacgaatgctactacaaatgctgacgttgaaacagctaaaaataatggcatcaatactattggagcagttgttcctcaagtaactcataaaaaagctgcaagagatgcaattaaccaagcaacagcaacgaaaagacaacaaataaatagtaatagagaagcaactcaggaagagaaaaatgcagcattgaacgaattaactcaagcaaccaaccatgctttagaacaaatcaatcaagcaacaacaaatgctaatgttgataacgccaaaggagatggtctaaatgccattaatccaattgctcctgtaactgttgttaagcaagctgcaagggatgccgtatcacatgatgcacaacaacatatcgcagagatcaatgctaatcctgatgcgactcaagaagaaagacaagcagcaattgacaaagtgaatgctgctgtaactgcagcaaacacaaacattttaaacgctaataccaatgctgatgttgaacaagtaaagacaaatgcgattcaaggaatacaagcaattacaccagctacaaaagtaaaaacagatgcaaaaaatgccatcgataaaagtgcggaaacgcaacataatacgatatttaataataatgatgcgacgctcgaagaacaacaagcagcacaacaattacttgatcaagctgtagccacagcgaagcaaaatattaatgcagcagatacgaatcaagaagttgcacaagcaaaagatcagggcacacaaaatatagtagtgattcaaccggcaacacaagttaaaacggatactcgcaatgttgtaaatgataaagcgcgagaggcgataacaaatatcaatgctacaactggcgcgactcgagaagagaaacaagaagcgataaatcgtgtcaatacacttaaaaatagagcattaactgatattggtgtgacgtctactactgcgatggtcaatagtattagagacgatgcagtcaatcaaatcggcgcagttcaaccgcatgtaacgaagaaacaaactgctacaggtgtattaaatgatttagcaactgctaaaaagcaagaaattaatcaaaacacaaatgcaacaactgaagaaaagcaagtggctttaaatcaagtggatcaagagttagcaacggcaattaataatataaatcaagctgatacaaatgcggaagtagatcaagcgcaacaattaggtacaaaagcaattaatgcgattcagccaaatattgttaaaaaacctgcagcattagcacaaatcaatcagcattataatgctaaattagctgaaatcaatgctacaccagatgcaacgaatgatgagaaaaatgctgcgatcaatactttaaatcaagacagacaacaagctattgaaagtattaaacaagctaacacaaatgcagaagtagaccaagctgcgacagtagcagagaataatatcgatgctgttcaagttgatgtagtaaaaaaacaagcagcgcgagataaaatcactgctgaagtggcgaagcgtattgaagcggttaaacaaacacctaatgcaactgacgaagaaaagcaggctgctgttaatcaaatcaatcaacttaaagatcaagcaattaatcaaattaatcaaaaccaaacaaatgatcaggtagacacaactacaaatcaagcggtaaatgctatagataatgttgaagctgaagtagtaattaaaacaaaggcaattgcagatattgaaaaagctgttaaagaaaagcaacagcaaattgataatagtcttgattcaacagataatgagaaagaagttgcttcacaagcattagctaaagaaaaagaaaaagcacttgcagctattgaccaagctcaaacgaatagtcaggtgaatcaagcagcaacaaatggtgtatcagcgattaaaattattcaacctgaaacaaaagttaaaccagctgcacgtgaaaaaatcaatcaaaaagcgaatgaattacgtgctaagattaatcaggataaagaagcaacagcagaagaaagacaagtagcactagataaaatcaatgaatttgtaaatcaagccatgacagatattacgaataatagaacaaatcaacaagttgatgatacaacaagtcaagcgcttgatagcattgctttagtgacgcctgaccatattgttagagcagctgctagagatgcagttaagcaacaatatgaagctaaaaagcgcgaaattgagcaagcggaacatgcgactgatgaagaaaaacaagttgctttaaatcaattagcgaataatgaaaaacgtgcattacaaaacatcgatcaagcaatagcgaataatgatgtgaaacgtgttgaaacaaatggcattgctacactaaaaggtgtacaacctcatattgtaattaagcctgaagcacaacaagcaataaaagcaagtgcagaaaatcaagtagaatcaataaaagatacaccacatgcaacagttgatgaattagatgaagcgaatcaattaattagcgacacactcaaacaagcgcaacaagaaatagaaaatacaaatcaagatgctgctgttactgatgttagaaatcaaacaatcaaggcaatagagcaaataaaacctaaagtaagacgtaaacgagctgcgcttgatagcattgaagaaaataataaaaatcaactcgatgcaatccgaaatacgttggatactactcaagatgaaagagatgttgctattgatactttaaataaaattgtaaatacaattaaaaatgacattgcacaaaacaaaacgaatgcagaagtggatcgaactgagactgatggcaacgacaacatcaaagtgattttacctaaagttcaagttaaaccagcagcgcgtcaatctgttggtgtaaaagccgaagctcaaaatgcactaatcgatcaaagcgatttatcaactgaagaagaaagactagctgctaaacatttagtagaacaagcacttaatcaggctattgatcagatcaatcatgcagataagactgcccaagttaatcaagatagtataaatgctcaaaatattatttcaaaaattaaaccagcgacaacagttaaagcaacagcattacaacaaattcaaaatatcgctacaaataaaattaatttaattaaagcaaataacgaagcgacagatgaagaacaaaatattgcaatagcacaagttgaaaaagagttaattaaagctaaacaacaaattgctagtgcagtgactaatgcagatgtggcatatttattgcatgatgagaaaaacgaaattcgtgaaatcgaacctgttattaacagaaaggcgtctgctcgagaacaattgacaacattattcaacgataaaaaacaagcaattgaagcgaatattcaagcaacggtagaagaaagaaatagtatattagcacagttacaaaatatttatgacactgctattggacaaattgatcaagatcgtagcaatgcacaagttgataaaacagcatcattaaatctacaaacaatacatgatttagatgtacatcctattaaaaagccagatgctgaaaaaacgattaatgatgatcttgcacgcgtcactgctttagtgcaaaattatcgaaaagtaagtaatcgtaataaggctgatgcattaaaagctataactgctttaaaattacaaatggatgaagaattaaaaacagcacgcactaatgctgatgttgatgcagttttaaaacgatttaatgttgcattaagcgatatagaagcagtaattactgaaaaagaaaatagcttactgcgaattgataacattgctcaacaaacatatgcgaaattcaaagcgatcgcaacaccagaacaattagctaaagtaaaagtattaattgatcaatatgttgcagatggcaatagaatgattgatgaagatgcgacattaaatgacatcaaacaacacacgcaattcattgttgatgaaattttagcaattaaattaccagctgaagcgacgaaagtatcaccaaaagaaattcagccagctccaaaagtttgtacgcctattaaaaaagaagagacacatgaatcgcgcaaagttgaaaaagaacttccaaatacaggttctgaaggaatggatttaccattgaaagaatttgcactgattacaggtgcggctttgttagctagaagacgtactaaaaacgaaaaagaatcataa

SEQ ID NO: 86 - DNA, 2319 bp, Staphylococcus aureus
gaggagaattcagtacaagacgttaaagattcgaatacggatgatgaattatcagacagcaatgatcagtctagtgatgaagaaaagaatgatgtgatcaataataatcagtcaataaacaccgacgataataaccaaataattaaaaaagaagaaacgaataactacgatggcatagaaaaacgctcagaagatagaacagagtcaacaacaaatgtagatgaaaacgaagcaacatttttacaaaagacccctcaagataatactcatcttacagaagaagaggtaaaagaatcctcatcagtcgaatcctcaaattcatcaattgatactgcccaacaaccatctcacacaacaataaatagagaagaatctgttcaaacaagtgataatgtagaagattcacacgtatcagattttgctaactctaaaataaaagagagtaacactgaatctggtaaagaagagaatactatagagcaacctaataaagtaaaagaagattcaacaacaagtcagccgtctggctatacaaatatagatgaaaaaatttcaaatcaagatgagttattaaatttaccaataaatgaatatgaaaataaggctagaccattatctacaacatctgcccaaccatcgattaaacgtgtaaccgtaaatcaattagcggcggaacaaggttcgaatgttaatcatttaattaaagttactgatcaaagtattactgaaggatatgatgatagtgaaggtgttattaaagcacatgatgctgaaaacttaatctatgatgtaacttttgaagtagatgataaggtgaaatctggtgatacgatgacagtggatatagataagaatacagttccatcagatttaaccgatagctttacaataccaaaaataaaagataattctggagaaatcatcgctacaggtacttatgataacaaaaataaacaaatcacctatacttttacagattatgtagataagtatgaaaatattaaagcacaccttaaattaacgtcatacattgataaatcaaaggttccaaataataataccaagttagatgtagaatataaaacggccctttcatcagtaaataaaacaattacggttgaatatcaaagacctaacgaaaatcggactgctaaccttcaaagtatgtttacaaacatagatacgaaaaatcatacagttgagcaaacgatttatattaaccctcttcgttattcagccaaggaaacaaatgtaaatatttcagggaatggtgatgaaggttcaacaattatagacgatagcacaataattaaagtttataaggttggagataatcaaaatttaccagatagtaacagaatttatgattacagtgaatatgaagatgtcacaaatgatgattatgcccaattaggaaataataatgatgtgaatattaattttggtaatatagattcaccatatattattaaagttattagtaaatatgaccctaataaggatgattacacgactatacagcaaactgtgacaatgcagacgactataaatgagtatactggtgagtttagaacagcatcctatgataatacaattgctttctctacaagttcaggtcaaggacaaggtgacttgcctcctgaaaaaacttataaaatcggagattacgtatgggaagatgtagataaagatggtattcaaaatacaaatgataatgaaaaaccgcttagtaatgtattggtaactttgacgtatcctgatggaacttcaaaatcagtcagaacagatgaagatgggaaatatcaatttgatggattgaaaaacggattgacttataaaattacattcgaaacacctgaaggatatacgccgacgcttaaacattcaggaacaaatcctgcactagactcagaaggtaattctgtatgggtaactattaatggacaagacgatatgacgattgatagtggattttatcaaacacctaaatacagcttagggaactatgtatggtatgacactaataaagatggtattcaaggtgatgatgaaaaaggaatctctggagttaaagtgacgttaaaagatgaaaacggaaatatcattagtacaactacaaccgatgaaaatggaaagtatcaatttgataatttaaatagtggtaattatattgttcattttgataaaccttcaggtatgactcaaacaacaacagattctggtgatgatgacgaacaggatgctgatggggaagaagttcatgtaacaattactgatcatgatgactttagtatagataacggatactatgatgacgaa

SEQ ID NO: 87 - protein, 1141 aa, Staphylococcus aureus; identical to SEQ ID NO: 20

SEQ ID NO: 88 - DNA, 3426 bp, Staphylococcus aureus; identical to SEQ ID NO: 53

SEQ ID NO: 89 - protein, 1349 aa, Staphylococcus aureus
MLNRENKTAITRKGMVSNRLNKFSIRKYTVGTASILVGTTLIFGLGNQEAKAAESTNKELNEATTSASDNQSSDKVDMQQLNQEDNTKNDNQKEMVSSQGNETTSNGNKLIEKESVQSTTGNKVEVSTAKSDEQASPKSTNEDLNTKQTISNQEALQPDLQENKSVVNVQPTNEENKKVDAKTESTTLNVKSDAIKSNDETLVDNNSNSNNENNADIILPKSTAPKRLNTRMRIAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQEVYKIGNYVWEDTNKNGVQELGEKGVGNVTVTVFDNNTNTKVGEAVTKEDGSYLIPNLPNGDYRVEFSNLPKGYEVTPSKQGNNEELDSNGLSSVITVNGKDNLSADLGIYKPKYNLGDYVWEDTNKNGIQDQDEKGISGVTVTLKDENGNVLKTVTTDADGKYKFTDLDNGNYKVEFTTPEGYTPTTVTSGSDIEKDSNGLTTTGVINGADNMTLDSGFYKTPKYNLGNYVWEDTNKDGKQDSTEKGISGVTVTLKNENGEVLQTTKTDKDGKYQFTGLENGTYKVEFETPSGYTPTQVGSGTDEGIDSNGTSTTGVIKDKDNDTIDSGFYKPTYNLGDYVWEDTNKNGVQDKDEKGISGVTVTLKDENDKVLKTVTTDENGKYQFTDLNNGTYKVEFETPSGYTPTSVTSGNDTEKDSNGLTTTGVIKDADNMTLDSGFYKTPKYSLGDYVWYDSNKDGKQDSTEKGIKDVKVTLLNEKGEVIGTTKTDENGKYCFDNLDSGKYKVIFEKPAGLTQTVTNTTEDDKDADGGEVDVTITDHDDFTLDNGYFEEDTSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKALPETGSENNGSNNATLFGGLFAALGSLLLFGRRKKQNK

SEQ ID NO: 90 - DNA, 4050 bp, Staphylococcus aureus
atgctaaacagagaaaataaaacggcaataacaaggaaaggcatggtatccaatcgattaaataaattttcgattagaaagtacacagtgggaacagcatcaattttagtaggtacaacattaatttttggtctggggaaccaagaagcaaaggctgcagaaagtactaataaagaattgaacgaagcgacaacttcagcaagtgataatcaatcgagtgataaagttgatatgcagcaactaaatcaagaagacaatactaaaaatgataatcaaaaagaaatggtatcatctcaaggtaatgaaacgacttcaaatgggaataaattaatagaaaaagaaagtgtacaatctaccactggaaataaagttgaagtttcaactgccaaatcagatgagcaagcttcaccaaaatctacgaatgaagatttaaacactaaacaaactataagtaatcaagaagcgttacaacctgatttgcaagagaataaatcagtggtaaatgttcaaccaactaatgaggaaaacaaaaaggtagatgccaaaactgaatcaactacattaaatgttaaaagtgatgctatcaagagtaatgatgaaactcttgttgataacaatagtaattcaaataatgaaaataatgcagatatcattttgccaaaaagtacagcacctaaacgtttgaatacaagaatgcgtatagcagcagtacagccatcatcaacagaggctaaaaatgttaatgatttaatcacatcaaatacaacattaactgtcgttgatgcagataaaaacaataaaatcgtaccagcccaagattatttatcattaaaatcacaaattacagttgatgacaaagttaaatcaggtgattatttcacaattaaatactcagatacagtacaagtatatggattgaatccggaagatattaaaaatattggtgatattaaagatccaaataatggtgaaacaattgcgactgcaaaacatgatactgcaaataatttaattacatatacatttacagattatgttgatcgatttaattctgtacaaatgggaattaattattcaatttatatggatgctgatacaattcctgttagtaaaaacgatgttgagtttaatgttacgataggtaatactacaacaaaaacaactgctaacattcaatatccagattatgttgtaaatgagaaaaattcaattggatcagcgttcactgaaacagtttcacatgttggaaataaagaaaatccagggtactataaacaaacgatttatgtaaatccatcggaaaattctttaacaaatgccaaactaaaagttcaagcttaccactcaagttatcctaataatatcgggcaaataaataaagatgtaacagatataaaaatatatcaagttcctaaaggttatacattaaataaaggatacgatgtgaatactaaagagcttacagatgtaacaaatcaatacttgcagaaaattacatatggcgacaacaatagcgctgttattgattttggaaatgcagattctgcttatgttgtaatggttaatacaaaattccaatatacaaatagcgaaagcccaacacttgttcaaatggctactttatcttcaacaggtaataaatccgtttctactggcaatgctttaggatttactaataaccaaagtggcggagctggtcaagaagtatataaaattggtaactacgtatgggaagatactaataaaaacggtgttcaagaattaggagaaaaaggcgttggcaatgtaactgtaactgtatttgataataatacaaatacaaaagtaggagaagcagttactaaagaagatgggtcatacttgattccaaacttacctaatggagattaccgtgtagaattttcaaacttaccaaaaggttatgaagtaaccccttcaaaacaaggtaataacgaagaattagattcaaacggcttatcttcagttattacagttaatggcaaagataacttatctgcagacttaggtatttacaaacctaaatacaacttaggtgactatgtctgggaagatacaaataaaaatggtatccaagaccaagatgaaaaaggtatatctggcgtaacggtaacattaaaagatgaaaacggtaacgtgttaaaaacagttacaacagacgctgatggcaaatataaatttactgatttagataatggtaattataaagttgaatttactacaccagaaggctatacaccgactacagtaacatctggtagcgacattgaaaaagactctaatggtttaacaacaacaggtgttattaatggtgctgataacatgacattagatagtggattctacaaaacaccaaaatataatttaggtaattatgtatgggaagatacaaataaagatggtaagcaggattcaactgaaaaaggtatttcaggcgtaacagttacattgaaaaatgaaaacggtgaagttttacaaacaactaaaacagataaagatggtaaatatcaatttactggattagaaaatggaacttataaagttgaattcgaaacaccatcaggttacacaccaacacaagtaggttcaggaactgatgaaggtatagattcaaatggtacatcaacaacaggtgtcattaaagataaagataacgatactattgactctggtttctacaaaccgacttacaacttaggtgactatgtatgggaagatacaaataaaaacggtgttcaagataaagatgaaaagggcatttcaggtgtaacagttacgttaaaagatgaaaacgacaaagttttaaaaacagttacaacagatgaaaatggtaaatatcaattcactgatttaaacaatggaacttataaagttgaattcgagacaccatcaggttatacaccaacttcagtaacttctggaaatgatactgaaaaagattctaatggtttaacaacaacaggtgtcattaaagatgcagataacatgacattagacagtggtttctataaaacaccaaaatatagtttaggtgattatgtttggtacgacagtaataaagacggcaaacaagattcaactgaaaaaggtatcaaagatgttaaagttactttattaaatgaaaaaggcgaagtaattggaacaactaaaacagatgaaaatggtaaatactgctttgataatttagatagcggtaaatacaaagttatttttgaaaagcctgctggcttaacacaaacagttacaaatacaactgaagatgataaagatgcagatggtggcgaagttgacgtaacaattacggatcatgatgatttcacacttgataacggatacttcgaagaagatacatcagacagcgattcagactcagatagtgactcagacagcgactcagactcagacagcgactcagactcagacagtgattcagattcagacagcgactcagattcagatagcgactcagattcggacagcgattcagactcagatagcgactcagattcagatagcgattcagactcagacagcgactcagattcagatagcgattcggactcagacagcgattcagactcagatagcgactcagactcagacagcgactcagattcagatagcgattcggactcagatagcgactcagattcagacagcgattcagactcagatagcgactcagattcagacagcgattcagactcagatagcgactcagactcagacagtgattcagattcagacagcgactcagactcagatagcgactcagattcggacagcgactcagactctgatagcgactcagactcagacagtgattcagacagcgattcagactcggatgcaggaaaacatacacctgttaaaccaatgagtactactaaagaccatcacaataaagcaaaagcattaccagaaacaggtagtgaaaataacggctcaaataacgcaacgttatttggtggattatttgcagcattaggttcattattgttattcggtcgtcgcaaaaaacaaaacaaataa

SEQ ID NO: 91 - protein, 635 aa, Staphylococcus aureus
MAKYRGKPFQLYVKLSCSTMMATSIILTNILPYDAQAASEKDTEITKEILSKQDLLDKVDKAIRQIEQLKQLSASSKEHYKAQLNEAKTASQIDEIIKRANELDSKDNKSSHTEMNGQSDIDSKLDQLLKDLNEVSSNVDRGQQSGEDDLNAMKNDMSQTATTKHGEKDDKNDEAMVNKALEDLDHLNQQIHKSKDASKDTSEDPAVSTTDNNHEVAKTPNNDGSGHVVLNKFLSNEENQSHSNRLTDKLQGSDKINHAMIEKLAKSNASTQHYTYHKLNTLQSLDQRIANTQLPKNQKSDLMSEVNKTKERIKSQRNIILEELARTDDKKYATQSILESIFNKDEAVKILKDIRVDGKTDQQIADQITRHIDQLSLTTSDDLLTSLIDQSQDKSLLISQILQTKLGKAEADKLAKDWTNKGLSNRQIVDQLKKHFASTGDTSSDDILKAILNNAKDKKQAIETILATRIERQKAKLLADLITKIETDQNKIFNLVKSALNGKADDLLNLQKRLNQTKKDIDYILSPIVNRPSLLDRLNKNGKTTDLNKLANLMNQGSDLLDSIPDIPTPKPEKTLTLGKGNGLLSGLLNADGNVSLPKAGETIKEHWLPISVIVGAMGVLMIWLSRRNKLKNKA

SEQ ID NO: 92 - DNA, 1908 bp, Staphylococcus aureus
atggctaaatatcgagggaaaccgtttcaattatatgtaaagttatcgtgttcgacaatgatggcgacaagtatcattttaacgaatatcttgccgtacgatgcccaagctgcatctgaaaaggatactgaaattacaaaagagatattatctaagcaagatttattagacaaagttgacaaggcaattcgtcaaattgagcaattaaaacagttatcggcttcatctaaagaacattataaagcacaactaaatgaagcgaaaacagcatcgcaaatagatgaaatcataaaacgagctaatgagttggatagcaaagacaataaaagttctcacactgaaatgaacggtcaaagtgatatagacagtaaattagatcaattgcttaaagatttaaatgaggtttcttcaaatgttgataggggtcaacaaagtggcgaggacgatcttaatgcaatgaaaaatgatatgtcacaaacggctacaacaaaacatggagaaaaagatgataaaaatgatgaagcaatggtaaataaggcgttagaagacctagaccatttgaatcagcaaatacacaaatcgaaagatgcatcgaaagatacatcggaagatccagcagtgtctacaacagataataatcatgaagtagctaaaacgccaaataatgatggttctggacatgttgtgttaaataaattcctttcaaatgaagagaatcaaagccatagtaatcgactcactgataaattacaaggaagcgataaaattaatcatgctatgattgaaaaattagctaaaagtaatgcctcaacgcaacattacacatatcataaactgaatacgttacaatctttagatcaacgtattgcaaatacgcaacttcctaaaaatcaaaaatcagacttaatgagcgaagtaaataagacgaaagagcgtataaaaagtcaacgaaatattattttggaagaacttgcacgtactgatgataaaaagtatgctacacaaagcattttagaaagtatatttaataaagacgaggcagttaaaattctaaaagatatacgtgttgatggtaaaacagatcaacaaattgcagatcaaattactcgtcatattgatcaattatctctgacaacgagtgatgatttattaacgtcattgattgatcaatcacaagataagtcgctattgatttctcaaattttacaaacgaaattaggaaaagctgaagcagataaattggctaaagattggacgaataaaggattatcaaatcgccaaatcgttgaccaattgaagaaacattttgcatcaactggcgacacgtcttcagatgatatattaaaagcaattttgaataatgccaaagataaaaaacaagcaattgaaacgattttagcaacacgtatagaaagacaaaaggcaaaattactggcagatttaattactaaaatagaaacagatcaaaataaaatttttaatttagttaaatcggcattgaatggtaaagcggatgatttattgaatttacaaaagagactcaatcaaacgaaaaaagatatagattatattttatcaccaatagtaaatcgtccaagtttactagatcgattgaataaaaatgggaaaacgacagatttaaataagttagcaaatttaatgaatcaaggatcagatttattagacagtattccagatatacccacaccaaagccagaaaagacgttaacacttggtaaaggtaatggattgttaagtggattattaaatgctgatggtaatgtatctttgcctaaagcgggggaaacgataaaagaacattggttgccgatatctgtaattgttggtgcaatgggtgtactaatgatttggttatcacgacgcaataagttgaaaaataaagcataa

SEQ ID NO: 93 - protein, 241 aa, Staphylococcus aureus
MKKLATVGSLIVTSTLVFSSMPFQNAHADTTSMNVSNKQSQNVQNHRPYGGVVPQGMTQAQYTELEKALPQLSAGSNMQDYNMKLYDATQNIADKYNVIITTNVGVFKPHAVRDMNGHALPLTKDGNFYQTNVDANGVNHGGSEMVQNKTGHMSQQGHMNQNTHMNQQPHMQQGHMQSSNHQMMSPKANMHSSNHQMNQSNKKVLPAAGESMTSSILTASIAALLLVSGLFLAFRRRSTNK

SEQ ID NO: 94 - DNA, 726 bp, Staphylococcus aureus
atgaaaaaattagcaacagtaggttctttaattgtaacaagcactttagtattctcaagtatgccttttcaaaatgcgcatgccgacacaacttcaatgaatgtgtcgaataaacaaagccaaaatgtacaaaatcatcgtccttatggcggagtagtaccacaaggaatgacgcaagcacaatatactgaattagagaaagctttaccccaattaagcgctggcagtaatatgcaagactataatatgaaattgtatgatgcgacgcaaaatattgctgataaatacaatgtgataattacaactaatgtaggggtatttaaaccacatgctgttagagatatgaatggccatgcgttacctttaacaaaagatggcaatttttatcaaacgaatgtagatgcaaatggtgttaatcatggtggtagtgaaatggtgcaaaataaaacaggtcatatgagtcaacaaggccatatgaatcagaacacacacatgaaccaacagccacacatgcaacaaggtcatatgcaatcatcaaaccatcaaatgatgagtccaaaagcaaatatgcattcatcaaatcatcaaatgaaccaaagtaacaaaaaagttttaccagctgctggtgaaagtatgacatcaagtattcttactgcaagtattgccgcactactattagtatctgggttattcttagcatttagacgacgttcaacaaataaataa

SEQ ID NO: 95 - protein, 5 aa, Artificial Sequence
  VARIANT (1)...(5): Xaa = Any Amino Acid
  in silico derived motif
LPXTG

SEQ ID NO: 96 - protein, 774 aa, Staphylococcus epidermidis
MEENSVQDVKDSNTDDELSDSNDQSSDEEKNDVINNNQSINTDDNNQIIKKEETNNYDGIEKRSEDRTESTTNVDENEATFLQKTPQDNTHLTEEEVKESSSVESSNSSIDTAQQPSHTTINREESVQTSDNVEDSHVSDFANSKIKESNTESGKEENTIEQPNKVKEDSTTSQPSGYTNIDEKISNQDELLNLPINEYENKARPLSTTSAQPSIKRVTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNDVNINFGNIDSPYIIKVISKYDPNKDDYTTIQQTVTMQTTINEYTGEFRTASYDNTIAFSTSSGQGQGDLPPEKTYKIGDYVWEDVDKDGIQNTNDNEKPLSNVLVTLTYPDGTSKSVRTDEDGKYQFDGLKNGLTYKITFETPEGYTPTLKHSGTNPALDSEGNSVWVTINGQDDMTIDSGFYQTPKYSLGNYVWYDTNKDGIQGDDEKGISGVKVTLKDENGNIISTTTTDENGKYQFDNLNSGNYIVHFDKPSGMTQTTTDSGDDDEQDADGEEVHVTITDHDDFSIDNGYYDDE

SEQ ID NO: 97 - protein, 128 aa, Staphylococcus aureus
MDVNTVNQKAASVKSTKDALDGQQNLQRAKTEATNAITHASDLNQAQKNALTQQVNSAQNVHAVNDIKQTTQSLNTAMTGLKRGVANHNQVVQSDNYVNADTNKKNDYNNAYNHANDIINGNAQHPVI

SEQ ID NO: 98 - protein, 128 aa, Staphylococcus aureus; identical to SEQ ID NO: 97

SEQ ID NO: 99 - protein, 52 aa, Staphylococcus aureus
QTTQNNYVTDQQKAFYQVLHLKGITEEQRNQYIKTLREHPERAQEVFSESLK

SEQ ID NO: 100 - protein, 36 aa, Staphylococcus aureus
VKNNLRYGIRKHKLGAASVFLGTMIVVGMGQDKEAA

SEQ ID NO: 101 - protein, 6 aa, Staphylococcus aureus
DRHFLN

SEQ ID NO: 102 - protein, 4 aa, Staphylococcus aureus
GNYD

SEQ ID NO: 103 - protein, 5 aa, Staphylococcus aureus
RRYPF

SEQ ID NO: 104 - protein, 6 aa, Staphylococcus aureus
KTTLLK

SEQ ID NO: 105 - protein, 7 aa, Staphylococcus aureus
GVTTSLS

SEQ ID NO: 106 - protein, 5 aa, Staphylococcus aureus
VDWLR

SEQ ID NO: 107 - protein, 4 aa, Staphylococcus aureus
RGFL

SEQ ID NO: 108 - protein, 15 aa, Staphylococcus aureus
KIKVYVGNYDFWYQS

SEQ ID NO: 109 - protein, 16 aa, Staphylococcus aureus
TVIVVSHDRHFLYNNV

SEQ ID NO: 110 - protein, 15 aa, Staphylococcus aureus
TETFLRGFLGRMLFS

SEQ ID NO: 111 - DNA, 20 bp, Artificial Sequence
  CpG adjuvant
tccatgacgttcctgacgtt

SEQ ID NO: 112 - DNA, 18 bp, Artificial Sequence
  CpG adjuvant
tctcccagcgtgcgccat

SEQ ID NO: 113 - DNA, 30 bp, Artificial Sequence
  CpG adjuvant
accgatgacgtcgccggtgacggcaccacg

SEQ ID NO: 114 - DNA, 24 bp, Artificial Sequence
  CpG adjuvant
tcgtcgttttgtcgttttgtcgtt

SEQ ID NO: 115 - DNA, 20 bp, Artificial Sequence
  CpG adjuvant
tccatgacgttcctgatgct

SEQ ID NO: 116 - DNA, 22 bp, Artificial Sequence
  CpG adjuvant
tcgacgttttcggcgcgcgccg

SEQ ID NO: 117 - DNA, 35 bp, Staphylococcus aureus
cgcggatccgcagattctgatattaatattaaaac

SEQ ID NO: 118 - DNA, 33 bp, Staphylococcus aureus
cccaagcttttaatttgtcatttcttctttttc

SEQ ID NO: 119 - DNA, 34 bp, Staphylococcus aureus
cgcggatccgctgggtctaataattttaaagatg

SEQ ID NO: 120 - DNA, 30 bp, Staphylococcus aureus
cccaagcttttatggaataacgatttgttg

SEQ ID NO: 121 - DNA, 32 bp, Staphylococcus aureus
cgcggatccagtgaaaatagtgttacgcaatc

SEQ ID NO: 122 - DNA, 33 bp, Staphylococcus aureus
cccaagcttttactctggaattggttcaatttc

SEQ ID NO: 123 - DNA, 31 bp, Staphylococcus aureus
cgcggatccacacaaacaactgcaactaacg

SEQ ID NO: 124 - DNA, 35 bp, Staphylococcus aureus
cccaagcttttatgctttgtgattctttttcaaac

SEQ ID NO: 125 - DNA, 26 bp, Staphylococcus aureus
cgcggatccaacacgcaacaaacttc

SEQ ID NO: 126 - DNA, 36 bp, Staphylococcus aureus
ggaactgcagttatttccagaatgataataaattac

SEQ ID NO: 127 - DNA, 28 bp, Staphylococcus aureus
cgcggatccgcagaacatacgaatggag

SEQ ID NO: 128 - DNA, 32 bp, Staphylococcus aureus
cccaagcttttatgtttcttcttcgtagtagc

SEQ ID NO: 129 - DNA, 28 bp, Staphylococcus epidermidis
cgcggatccgaggagaattcagtacaag

SEQ ID NO: 130 - DNA, 31 bp, Staphylococcus aureus
cccaagcttttattcgtcatcatagtatccg

SEQ ID NO: 131 - DNA, 25 bp, Staphylococcus aureus
aaaagtactcaccaccaccaccacc

SEQ ID NO: 132 - DNA, 29 bp, Staphylococcus aureus
aaaagtactcacttgattcatcgcttcag

SEQ ID NO: 133 - DNA, 33 bp, Staphylococcus aureus
gcgcgccatggcacaagcttctacacaacatac

SEQ ID NO: 134 - DNA, 32 bp, Staphylococcus aureus
gcgcgctcgagatggatgaatgcatagctaga

SEQ ID NO: 135 - DNA, 48 bp, Staphylococcus aureus
gcatccatggcaccatcaccatcaccacgaagtaaacgttgatcaagc

SEQ ID NO: 136 - DNA, 34 bp, Staphylococcus aureus
agcactcgagttagaatccccaagcacctaaacc

SEQ ID NO: 137 - DNA, 29 bp, Staphylococcus aureus
gcacccatggcagaaaatacaaatacttc

SEQ ID NO: 138 - DNA, 31 bp, Staphylococcus aureus
ttttctcgagcattttagattgactaagttg

SEQ ID NO: 139 - DNA, 33 bp, Staphylococcus aureus
caagtcccatggctgagacgacacaagatcaac

SEQ ID NO: 140 - DNA, 31 bp, Staphylococcus aureus
cagtctcgagttttacagctgtttttggttg

SEQ ID NO: 141 - DNA, 30 bp, Staphylococcus aureus
agctcatatggcttatactgttactaaacc

SEQ ID NO: 142 - DNA, 29 bp, Staphylococcus aureus
gcgcctcgagtttatattgtgggatgtcg

SEQ ID NO: 143 - DNA, 34 bp, Staphylococcus aureus
caagtcccatggcaacagaagctacgaacgcaac

SEQ ID NO: 144 - DNA, 35 bp, Staphylococcus aureus
accagtctcgagtaattctttagctttagagcttg

SEQ ID NO: 145 - DNA, 33 bp, Staphylococcus aureus
tattctcgaggctttgagtgtgtccatcatttg

SEQ ID NO: 146 - DNA, 32 bp, Staphylococcus aureus
gaagccatggcagcagctgaagaaacaggtgg

SEQ ID NO: 147 - DNA, 30 bp, Staphylococcus aureus
gattacaccatggttaaacctcaagcgaaa

SEQ ID NO: 148 - DNA, 30 bp, Staphylococcus aureus
aggtgtctcgagtgcgattgtagcttcatt

(19) United States
(12) Patent Application Publication
(10) Pub. No.: US 2018/0104323 A1
(43) Pub. Date: Apr. 19, 2018

(54) IMMUNOGENIC COMPOSITION

(71) Applicant: GLAXOSMITHKLINE BIOLOGICALS, Rixensart (BE)

(72) Inventors: Philippe DENOEL, Rixensart (BE); Jan Poolman, Rixensart (BE)

(21) Appl. No.: 15/840,527

(22) Filed: Dec. 13, 2017

Related U.S. Application Data
(63) Continuation of application No. 12/294,774, filed on Sep. 26, 2008.
(60) Provisional application No. 60/787,249, filed on Mar. 30, 2006.
(60) Provisional application No. 60/787,587, filed on Mar. 30, 2006.

(30) Foreign Application Priority Data
Mar. 30, 2006 (GB) 0606416.6

(51) Int. Cl. A61K 39/085; A61K 31/70; A61K 39/00

(52) U.S. Cl. CPC A61K 39/085; A61K 31/70; A61K 2039/6037; A61K 47/646; A61K 47/61

(57) ABSTRACT

The present application relates to immunogenic compositions comprising Type 5 and/or 8 capular polysaccharide or oligosaccharide from S. aureus having between 30-100% O-acetylation. Vaccines, methods of treatment using and processes to make an immunogenic composition comprising Type 5 and/or 8 capsular polysaccharides with 30-100% O-acetylation are also described.

This application is filed pursuant to 35 U.S.C. § 371 as a United States National Phase Application of International Patent Application Serial No. PCT/EP2007/053057 filed Mar. 29, 2007, which claims priority from Great Britain Application No. 0606416.6 filed in the United Kingdom on Mar. 30, 2006, from U.S. Application No. 60/787,249 filed in the United States on Mar. 30, 2006, and from U.S. Application No. 60/787,587 filed in the United States on Mar. 30, 2006, the contents of which are incorporated herein by reference.

### TECHNICAL FIELD

The present invention relates to the field of Staphylococcal immunogenic compositions and vaccines, their manufacture and the use of such compositions in medicine. More particularly, it relates to vaccine compositions comprising type 5 and/or 8 polysaccharides from S. aureus in which the degree of O-acetylation is between 30-100%. Methods for the treatment or prevention of staphylococcal infections using such vaccines are also provided.

### BACKGROUND

The number of both community acquired and hospital acquired infections have increased over recent years with the increased use of intravascular devices. Hospital acquired (nosocomial) infections are a major cause of morbidity and mortality, more particularly in the US, where they affect more than 2 million patients annually. Following various studies, about 6 percent of the US patients will acquire an infection during their stay in hospital. The economic burden in the USA was estimated to be more than $4.5 billion in 1992 (Emori and Gaynes, 1993, Clin. Microbiol. Rev. 6; 428). The most frequent infections are urinary tract infections (UTI-33% of the infections), followed by pneumonia (15.5%), surgical site infections (14.8%) and primary bloodstream infections (13%) Emori and Gaynes, 1993, Clin. Microbiol. Rev. 6; 428).

Staphylococcus aureus, Coagulase-negative Staphylococci (mostly Staphylococcus epidermidis), enterococcus spp, Esherichia coli and Pseudomonas aeruginosa are the major nosocomial pathogens. Although those pathogens almost cause the same number of infections, the severity of the disorders they can produce combined with the frequency of antibiotic resistant isolates balance this ranking towards S. aureus and S. epidermidis as being the most significant nosocomial pathogens.

Staphylococcus aureus is the most common cause of nosocomial infections with a significant morbidity and mortality (Romero-Vivas et al 1995, Infect. Dis. 21; 1417). It is the cause of some cases of osteomyelitis, endocarditis, septic arthritis, pneumonia, abscesses and toxic shock syndrome.

S. epidermidis is a normal skin commensal which is also an important opportunistic pathogen responsible for infections of implanted medical devices and infections at sites of surgery. Medical devices infected by S. epidermidis include cardiac pacemakers, cerebrospinal fluid shunts, continuous ambulatory peritoneal dialysis catheters, orthopaedic devices and prosthetic heart valves.

S. aureus and S. epidermidis infections are treated with antibiotics, with penicillin being the drug of choice whereas vancomycin is used for methicillin resistant isolates. The percentage of staphylococcal strains exhibiting wide-spectrum resistance to antibiotics has become increasingly prevalent since the 1980's (Panlilo et al 1992, Infect. Control. Hosp. Epidemiol. 13; 582), posing a threat for effective antimicrobial therapy. In addition, the recent emergence of vancomycin resistant S. aureus strain has aroused fear that methicillin resistant S. aureus strains will emerge and spread for which no effective therapy is available.

An alternative approach of using antibodies against staphylococcal antigens in passive immunotherapy has been investigated. Therapy involving administration of polyclonal antisera are under development (WO 00/15238, WO 00/12132) as well as treatment with a monoclonal antibody against lipoteichoic acid (WO 98/57994).

An alternative approach would be use of active vaccination to generate an immune response against staphylococci. Several candidates for inclusion as vaccine components have been identified. These include Fibronectin binding protein (U.S. Pat. No. 5,840,846), MHC II analogue (U.S. Pat. No. 5,648,240), fibrinogen binding protein (U.S. Pat. No. 6,008,341), GehD (US 2002/0169288), collagen binding protein (U.S. Pat. No. 6,288,214), SdrF, SdrG and SdrH (WO 00/12689), mutant SEA and SEB exotoxins (WO 00/02523) and 52 kDa vitronectin binding protein (WO 01/60852).

The S. aureus genome has been sequenced and many of the coding sequences have been identified (EP786519, WO02/094868). The same is true for S. epidermidis (WO 01/34809). As a refinement of this approach, others have identified proteins that are recognised by hyperimmune sera from patients who have suffered staphylococcal infection (WO01/98499, WO 02/059148).

The first generation of vaccines targeted against S. aureus or against the exoproteins it produces have met with limited success (Lee 1996 Trends Microbiol. 4; 162). There remains a need to develop effective vaccines against staphylococcal infections.

### DESCRIPTION OF FIGURES

FIG. 1—Polypeptide sequences of proteins for inclusion in an immunogenic composition. Table 1 provides information on which protein is represented by each SEQ ID.

FIG. 2—Nucleotide sequences encoding proteins for inclusion in an immunogenic composition. Table 1 provides information on which protein is encoded by each SEQ ID.

FIG. 3—Purification of alpha toxin under native conditions. Panel A shows a COOMASSIE™ stained SDS-PAGE of samples prepared during the purification of alpha toxin. Lane 1—molecular weight markers, lane 2—soluble fraction containing over-expressed alpha toxin, lane 3—flow through from the Ni-NTA column, lane 4—fractions eluted with 10% buffer B, lane 5—fractions eluted with 20% buffer B, lane 6—fractions eluted with 30% buffer B, lane 7—fractions eluted with 50% buffer B, lane 8—fractions eluted with 75% buffer B, lane 9 and 10 fractions eluted with 100% buffer B, lane 11 bacteria at T=0 before induction, lane 12—bacteria at T=4 hours after induction, lane 13—cell lysate, lane 14—soluble fraction, lane 15—insoluble fraction.

Panel B shows a COOMASSIE™ stained SDS-PAGE of 10, 5, 2 and 1 μl of the purified alpha toxin.

FIG. 4—Purification of SdrC underdenaturing conditions. Panel A shows a COOMASSIE™ stained SDS-PAGE of samples prepared during the purification of alpha toxin. Lane M—molecular weight markers, lane Start—supernatant formed from the insoluble fraction containing over-expressed SdrC, lane FT1—flow through from the Ni-NTA column, lane C—fractions eluted with wash buffer C, lane D—fractions eluted with buffer D, lane E—fractions eluted with buffer E.

Panel B shows a COOMASSIE™ stained SDS-PAGE of 1, 2, 5 and 10 μl of the purified SdrC.

FIG. 5—ELISA results for antisera against staphylococcal proteins in plates coated with purified proteins.

Pool mice pre—result using pooled sera extracted from mice pre-innoculation. Pool mice Post III—result using pooled mouse sera extracted post-immunisation. Pool rabbit pre—result using pooled sera extracted from rabbits pre-innoculation. Pool rabbit Post III—result using pooled rabbit sera extracted post-immunisation. Blc—negative control.

FIG. 6—ELISA results for mouse antisera raised against staphylococcal proteins in plates coated with killed staphylococci.

Panel A uses plates coated with S. aureus serotype 5 killed whole cells. Panel B uses plates coated with S. aureus serotype 8 killed whole cells. Panel C uses plates coated with S. epidermidis killed whole cells.

The line marked with square signs shows the ELISA result using antisera from mice immunised three times with the indicated staphylococcal protein. The line marked with diamond signs shows the ELISA result for pre-immune mouse sera.

FIG. 7—ELISA results for rabbit antisera raised against staphylococcal proteins in plates coated with killed staphylococci.

Panel A uses plates coated with S. aureus serotype 5 killed whole cells. Panel B uses plates coated with S. aureus serotype 8 killed whole cells. Panel C uses plates coated with S. epidermidis killed whole cells.

The line marked with square signs shows the ELISA result using antisera from rabbits immunised three times with the indicated staphylococcal protein (except for HarA where only one immunisation was given). The line marked with diamond signs shows the ELISA result for pre-immune rabbit sera.

### DETAILED DESCRIPTION

The present invention discloses immunogenic compositions which comprise S. aureus polysaccharides Type 5 and/or 8 in which the Type 5 and/or Type 8 capsular polysaccharide or oligosaccharide is between 30% and 100% O-acetylated. In particular embodiments, the immunogenic composition of the invention additionally comprises PNAG or staphylococcal protein(s). PNAG is highly conserved among Gram positive bacteria and provides protection against a broad range of bacteria whereas Type 5 and 8 polysaccharides are potent immunogens that elicit an immune response against most strains of S. aureus which is the most common cause of nosocomial infection.

### Polysaccharides

The immunogenic compositions of the invention comprise PNAG and type 5 and 8 polysaccharides from S. aureus either or both of which are between 30% and 100% 0-acetylated.

### Poly N-Acetylated Glucosamine (PNAG)

PNAG is a polysaccharide intercellular adhesin and is composed of a polymer of β-(1→6)-linked glucosamine, optionally substituted with N-acetyl and/or O-succinyl constituents. This polysaccharide is present in both S. aureus and S. epidermidis and can be isolated from either source (Joyce et al 2003, Carbohydrate Research 338; 903; Maira-Litran et al 2002, Infect. Imun. 70; 4433). For example, PNAG may be isolated from S. aureus strain MN8m (WO 04/43407). The preparation of dPNAG is described in WO 04/43405.

The polysaccharide previously known as poly-N-succinyl-β-(1→6)-glucosamine (PNSG) was recently shown not to have the expected structure since the identification of N-succinylation was incorrect (Maira-Litran et al 2002, Infect. Imun. 70; 4433). Therefore the polysaccharide formally known as PNSG and now found to be PNAG is also encompassed by the term PNAG.

PNAG may be of different sizes varying from over 400 kDa to between 75 and 400 kDa to between 10 and 75 kDa to oligosaccharides composed of up to 30 repeat units (of β-(1→6)-linked glucosamine, optionally substituted with N-acetyl and O-succinyl constituents). Any size of PNAG polysaccharide or oligosaccharide may be use in an immunogenic composition of the invention, for example a size of over 40 kDa can be used.

Sizing may be achieved by any method known in the art, for instance by microfluidisation, ultrasonic irradiation or by chemical cleavage (WO 03/53462, EP497524, EP497525).

Size ranges of PNAG are for example 40-400 kDa, 50-350 kDa, 40-300 kDa, 60-300 kDa, 50-250 kDa and 60-200 kDa.

PNAG can have different degree of acetylation due to substitution on the amino groups by acetate. PNAG produced in vitro is almost fully substituted on amino groups (95-100%). Alternatively, a deacetylated PNAG can be used having less than 50%, 40%, 30%, 20%, 10% or 5% N-acetylation. Use of a deacetylated PNAG allows opsonic killing of Gram positive bacteria, optionally S. aureus and/or S. epidermidis (WO 04/43405). In an embodiment, the PNAG has a size between 40 kDa and 300 kDa and is deacetylated so that less than 50%, 40%, 30%, 20%, 10% or 5% of amino groups are N acetylated.

In an embodiment, the PNAG is not 0-succinylated or is O-succinylated on less than 25, 20, 15, 10, 5, 2, 1 or 0.1% of residues.

The term deacetylated PNAG (dPNAG) refers to a PNAG polysaccharide or oligosaccharide in which less than 50%, 40%, 30%, 20%, 10% or 5% of the amino groups are acetylated.

As used herein, the term PNAG encompasses both acetylated and deacetylated forms of the saccharide.

In an embodiment, PNAG is deacetylated to form dPNAG, by chemically treating the native polysaccharide. For example, the native PNAG is treated with a basic solution such that the pH rises to above 10. For instance the PNAG is treated with 0.1-5M, 0.2-4M, 0.3-3M, 0.5-2M, 0.75-1.5M or 1M NaOH, KOH or NH4OH. Treatment is for at least 10 or 30 minutes, or 1, 2, 3, 4, 5, 10, 15 or 20 hours at a temperature of 20-100, 25-80, 30-60 or 30-50 or 35-45° C. dPNAG may be prepared as described in WO 04/43405.

In an embodiment, the polysaccharide(s) included in the immunogenic composition of the invention are conjugated to a carrier protein as described below or alternatively unconjugated.

Type 5 and Type 8 Polysaccharides from S. aureus

Most strains of S. aureus that cause infection in man contain either Type 5 or Type 8 polysaccharides. Approximately 60% of human strains are Type 8 and approximately 30% are Type 5. The structures of Type 5 and Type 8 capsular polysaccharide antigens are described in Moreau et al Carbohydrate Res. 201; 285 (1990) and Fournier et al Infect. Immun. 45; 87 (1984). Both have FucNAcp in their repeat unit as well as ManNAcA which can be used to introduce a sulfhydryl group.

Recently (Jones Carbohydrate Research 340, 1097-1106 (2005)) NMR spectroscopy revised the structures of the capsular polysaccharides to:

Type 5

→4)-β-D-ManNAcA-(1→4)-α-L-FucNAc(3OAc)-(1→3)-β-D-FucNAc-(1→

Type 8

→3)-β-D-ManNAcA(4OAc)-(1→3)-α-L-FucNAc(1→3)-α-D-FucNAc(1→

Polysaccharides may be extracted from the appropriate strain of S. aureus using methods well known to the skilled man, for instance as described in U.S. Pat. No. 6,294,177 or Infection and Immunity (1990) 58(7); 2367. For example, ATCC 12902 is a Type 5 S. aureus strain and ATCC 12605 is a Type 8 S. aureus strain.

Polysaccharides are of native size or alternatively may be sized, for instance by microfluidisation, ultrasonic irradiation or by chemical treatment. The invention also covers oligosaccharides derived from the type 5 and 8 polysaccharides from S. aureus.

The type 5 and/or 8 capsular polysaccharide or oligosaccharides included in the immunogenic composition of the invention are O-acetylated. In an embodiment, the degree of O-acetylation of type 5 capsular polysaccharide or oligosaccharide is 10-100%, 20-100%, 30-100%, 40-100%, 50-100%. 60-100%, 70-100%, 80-100%, 90-100%, 50-90%, 60-90%, 70-90% or 80-90%. In an embodiment, the degree of O-acetylation of type 8 capsular polysaccharide or oligosaccharide is 10-100%, 20-100%, 30-100%, 40-100%, 50-100%. 60-100%, 70-100%, 80-100%, 90-100%, 50-90%, 60-90%, 70-90% or 80-90%.

In an embodiment, the degree of O-acetylation of type 5 and type 8 capsular polysaccharides or oligosaccharides is 10-100%, 20-100%, 30-100%, 40-100%, 50-100%. 60-100%, 70-100%, 80-100%, 90-100%, 50-90%, 60-90%, 70-90% or 80-90%.

The degree of O-acetylation of the polysaccharide or oligosaccharide can be determined by any method known in the art, for example, by proton NMR (Lemercinier and Jones 1996, Carbohydrate Research 296; 83-96, Jones and Lemercinier 2002, J Pharmaceutical and Biomedical analysis 30; 1233-1247, WO 05/033148 or WO 00/56357). A further commonly used method is that described by Hestrin (1949) J. Biol. Chem. 180; 249-261.

O-acetyl groups can be removed by hydrolysis, for example by treatment with a base such as anhydrous hydrazine (Konadu et al 1994; Infect. Immun. 62; 5048-5054) or treatment with 0.1 N NaOH for 1-8 hours. In order to maintain high levels of O-acetylation on type 5 and/or 8 polysaccharide or oligosaccharide, treatments which would lead to hydrolysis of the O-acetyl groups are minimised. For example treatment at extremes of pH are minimised.

The type 5 and 8 polysaccharides included in the immunogenic composition of the invention are optionally conjugated to a carrier protein as described below or are alternatively unconjugated.

The immunogenic compositions of the invention alternatively contains either type 5 or type 8 polysaccharide.

S. aureus 336 Antigen

In an embodiment, the immunogenic composition of the invention comprises the S. aureus 336 antigen described in U.S. Pat. No. 6,294,177.

The 336 antigen comprises β-linked hexosamine, contains no O-acetyl groups and specifically binds to antibodies to S. aureus Type 336 deposited under ATCC 55804.

In an embodiment, the 336 antigen is a polysaccharide which is of native size or alternatively may be sized, for instance by microfluidisation, ultrasonic irradiation or by chemical treatment. The invention also covers oligosaccharides derived from the 336 antigen.

The 336 antigen, where included in the immunogenic composition of the invention is optionally conjugated to a carrier protein as described below or are alternatively unconjugated.

Type I, II and III Polysaccharides from S. epidermidis

Strains ATCC-31432, SE-360 and SE-10 of S. epidermidis are characteristic of three different capsular types, I, II and III respectively (Ichiman and Yoshida 1981, J. Appl. Bacteriol. 51; 229). Capsular polysaccharides extracted from each serotype of S. epidermidis constitute Type I, II and III polysaccharides. Polysaccharides may be extracted by several methods including the method described in U.S. Pat. No. 4,197,290 or as described in Ichiman et al 1991, J. Appl. Bacteriol. 71; 176.

In one embodiment of the invention, the immunogenic composition comprises type I and/or II and/or III polysaccharides or oligosaccharides from S. epidermidis.

Polysaccharides are of native size or alternatively may be sized, for instance by microfluidisation, ultrasonic irradiation or chemical cleavage. The invention also covers oligosaccharides extracted from S. epidermidis strains.

These polysaccharides are unconjugated or are optionally conjugated as described below.

Conjugation of Polysaccharides

Amongst the problems associated with the use of polysaccharides in vaccination, is the fact that polysaccharides per se are poor immunogens. Strategies, which have been designed to overcome this lack of immunogenicity, include the linking of the polysaccharide to large protein carriers, which provide bystander T-cell help. In an embodiment, the polysaccharides utilised in the invention are linked to a protein carrier which provide bystander T-cell help. Examples of these carriers which may be used for coupling to polysaccharide or oligosaccharide immunogens include the Diphtheria and Tetanus toxoids (DT, DT Crm197 and TT), Keyhole Limpet Haemocyanin (KLH), Pseudomonas aeruginosa exoprotein A (rEPA) and the purified protein derivative of Tuberculin (PPD), protein D from Haemophilus influenzae, pneumolysin or fragments of any of the above. Fragments suitable for use include fragments encompassing T-helper epitopes. In particular protein D fragment will optionally contain the N-terminal ⅓ of the protein. Protein D is an IgD-binding protein from Haemophilus influenzae (EP 0 594 610 B1).

An alternative carrier protein to use in the immunogenic composition of the invention is a single staphylococcal protein or fragment thereof or a fusion protein comprising at least or exactly 1, 2, 3 or 4 or more of the staphylococcal proteins or fragments thereof listed in the section below.

A new carrier protein that would be particularly advantageous to use in the context of a staphylococcal vaccine is staphylococcal alpha toxoid. The native form may be conjugated to a polysaccharide since the process of conjugation reduces toxicity. Optionally a genetically detoxified alpha toxin such as the His35Leu or His 35 Arg variants are used as carriers since residual toxicity is lower. Alternatively the alpha toxin is chemically detoxified by treatment with a cross-linking reagent, formaldehyde or glutaraldehyde. A genetically detoxified alpha toxin is optionally chemically detoxified, optionally by treatment with a cross-linking reagent, formaldehyde or glutaraldehyde to further reduce toxicity.

The polysaccharides may be linked to the carrier protein(s) by any known method (for example, by Likhite, U.S. Pat. No. 4,372,945 by Armor et al., U.S. Pat. No. 4,474,757, WO and Jennings et al., U.S. Pat. No. 4,356,170). Optionally, CDAP conjugation chemistry is carried out (see WO95/08348).

In CDAP, the cyanylating reagent 1-cyano-dimethylaminopyridinium tetrafluoroborate (CDAP) is optionally used for the synthesis of polysaccharide-protein conjugates. The cyanilation reaction can be performed under relatively mild conditions, which avoids hydrolysis of the alkaline sensitive polysaccharides. This synthesis allows direct coupling to a carrier protein.

The polysaccharide may be solubilized in water or a saline solution. CDAP may be dissolved in acetonitrile and added immediately to the polysaccharide solution. The CDAP reacts with the hydroxyl groups of the polysaccharide to form a cyanate ester. After the activation step, the carrier protein is added. Amino groups of lysine react with the activated polysaccharide to form an isourea covalent link. After the coupling reaction, a large excess of glycine is then added to quench residual activated functional groups. The product is then passed through a gel permeation column to remove unreacted carrier protein and residual reagents.

Proteins

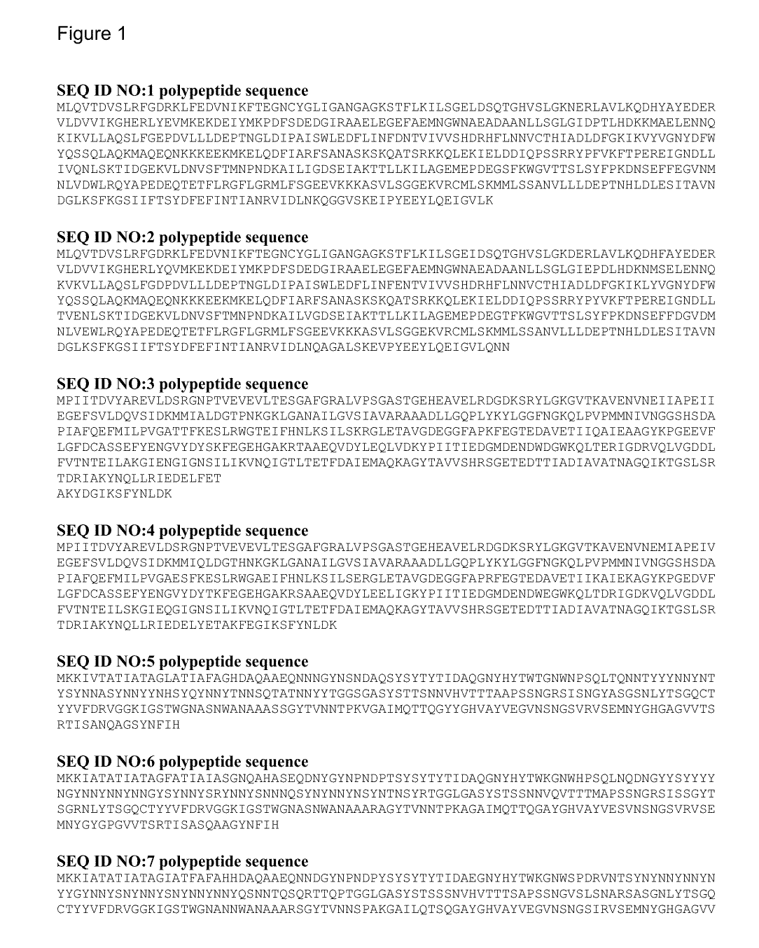

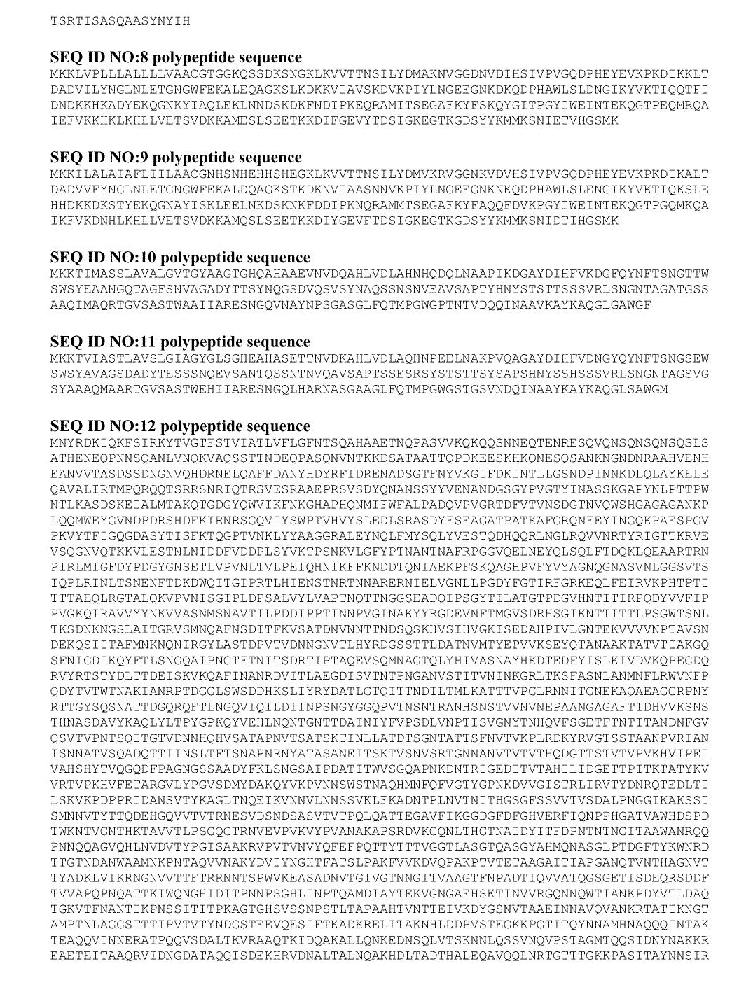

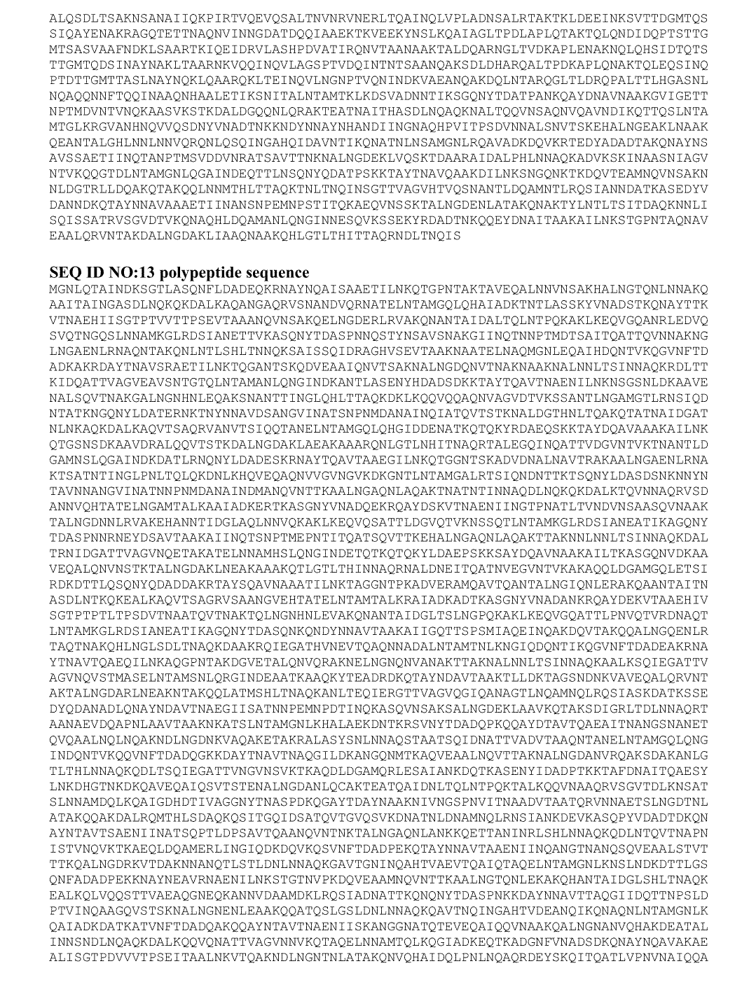

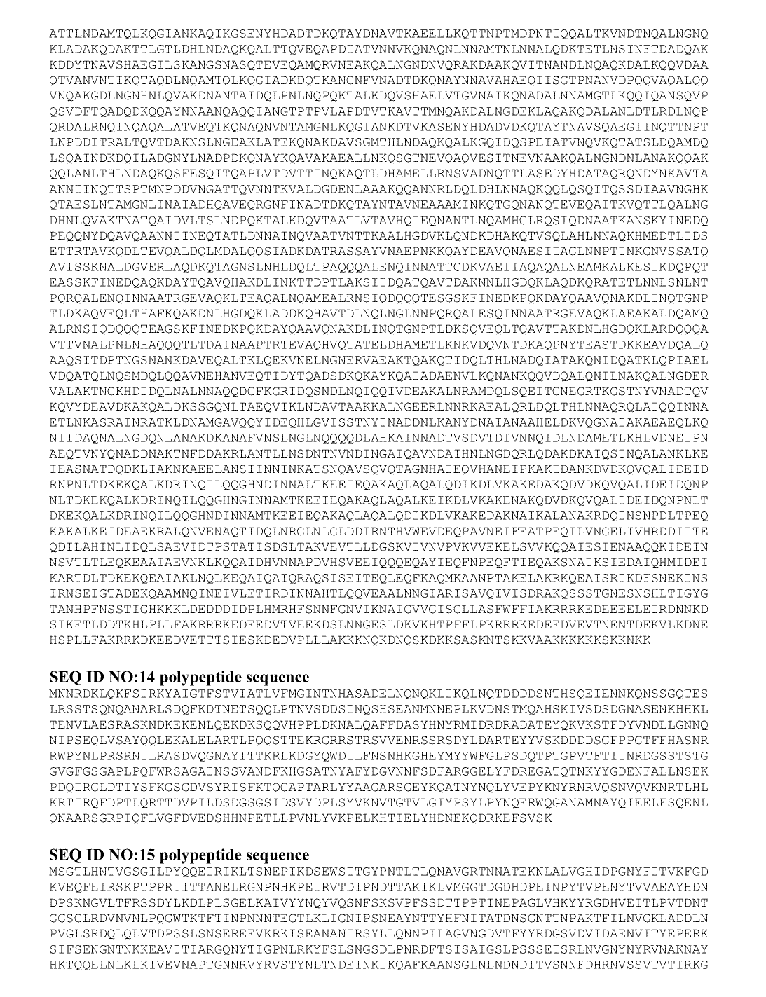

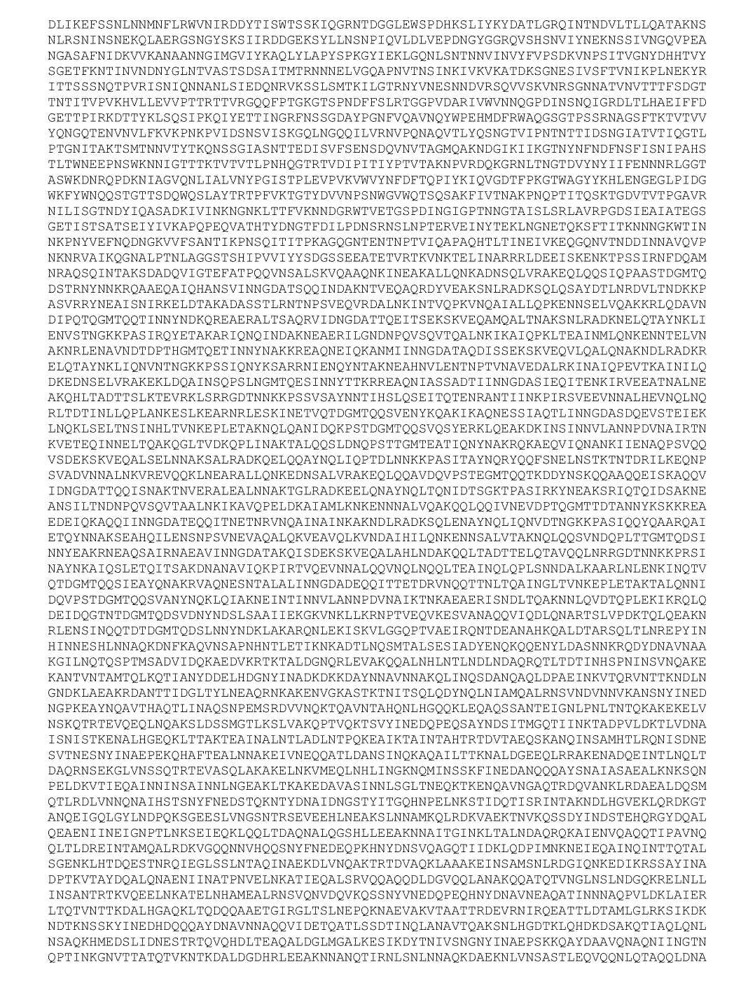

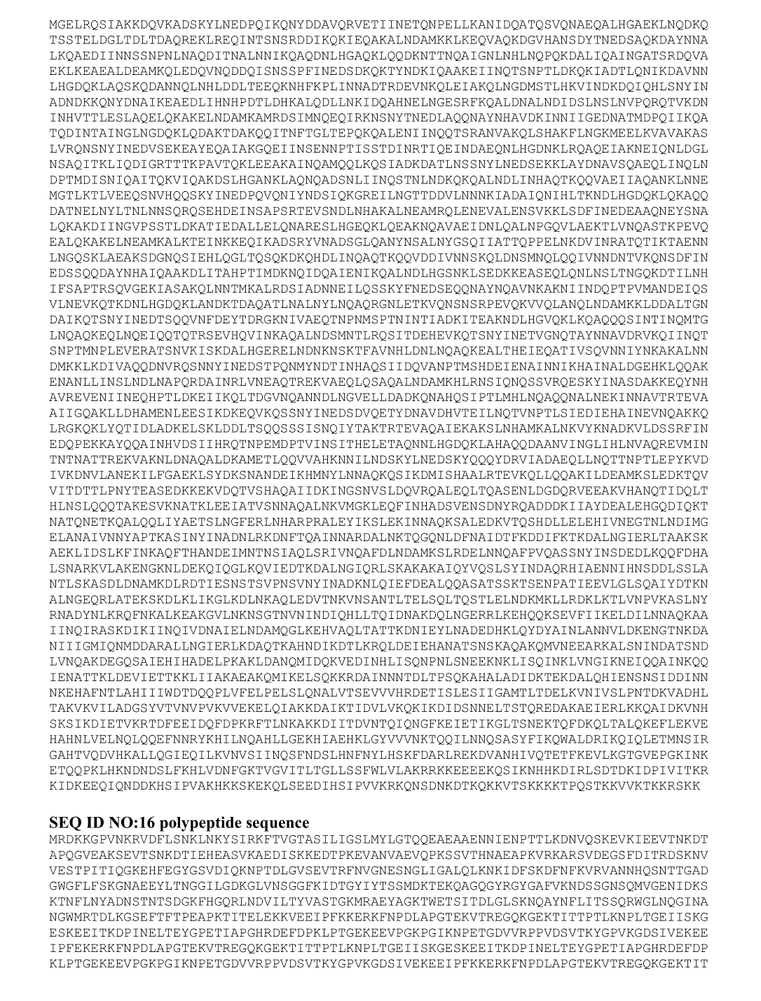

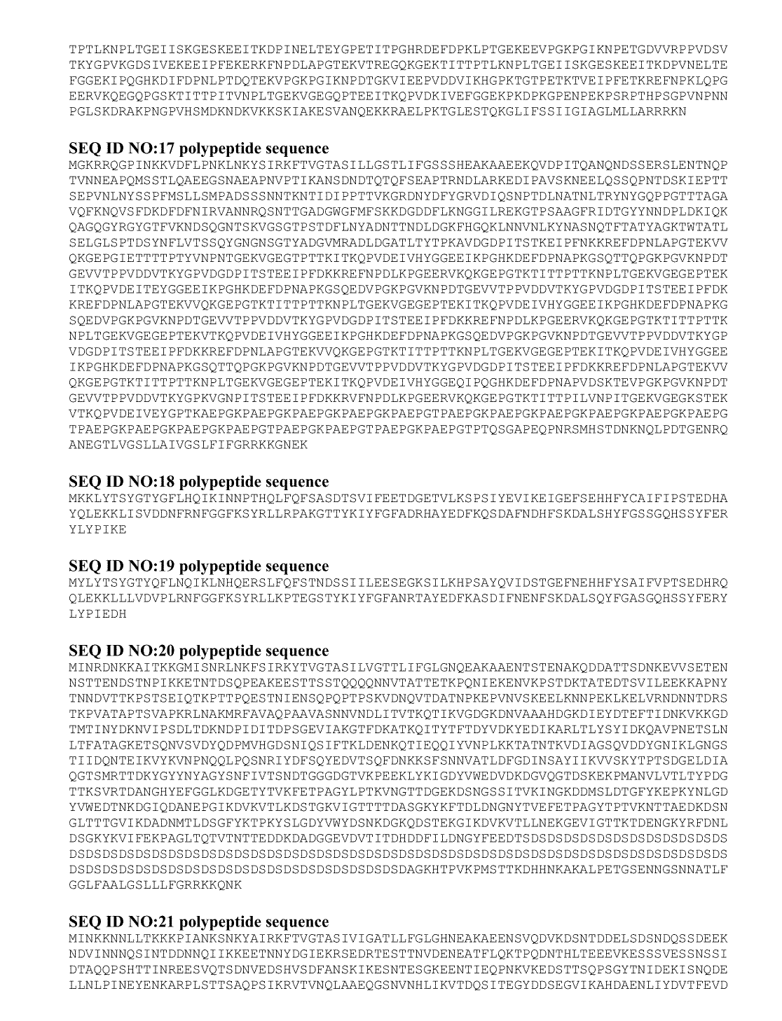

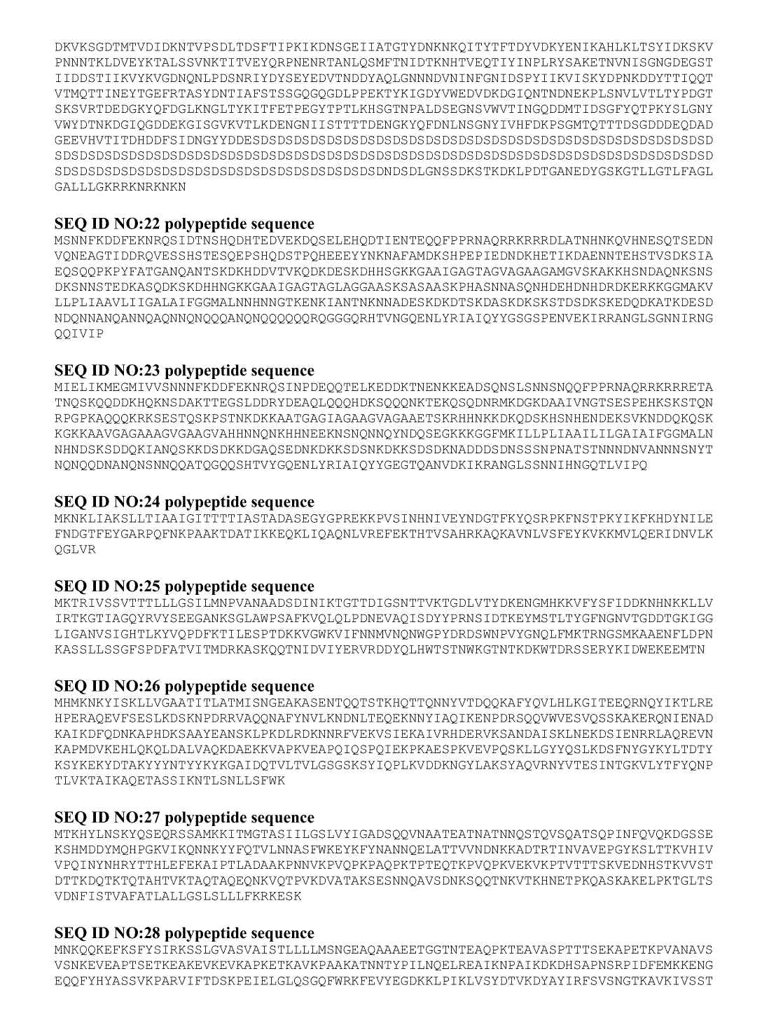

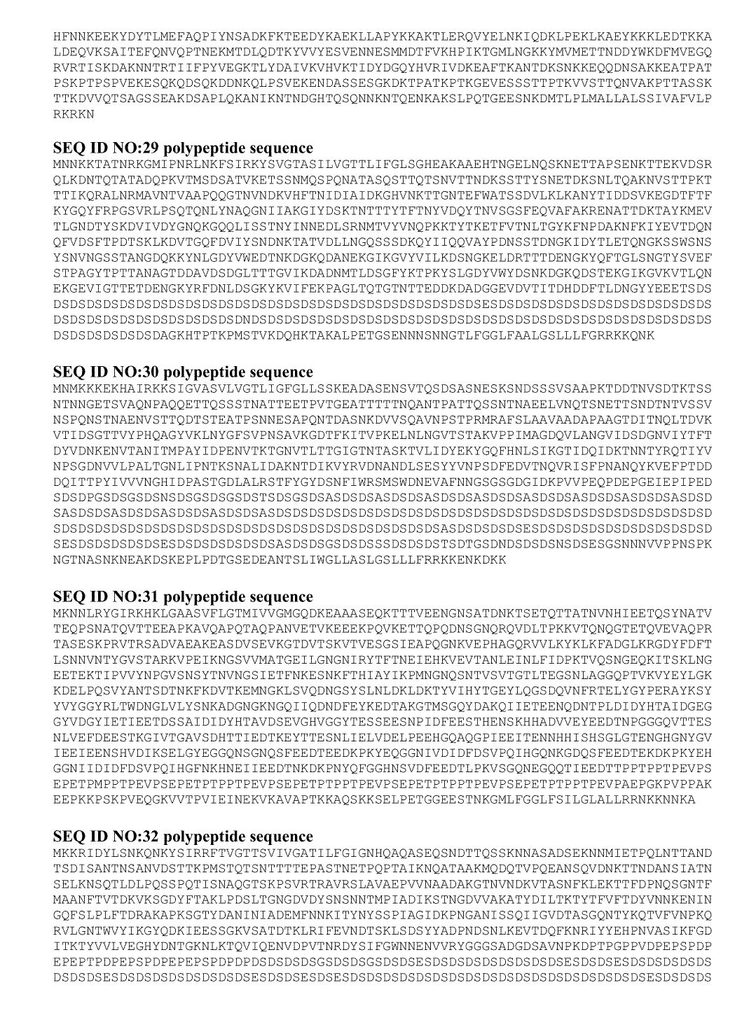

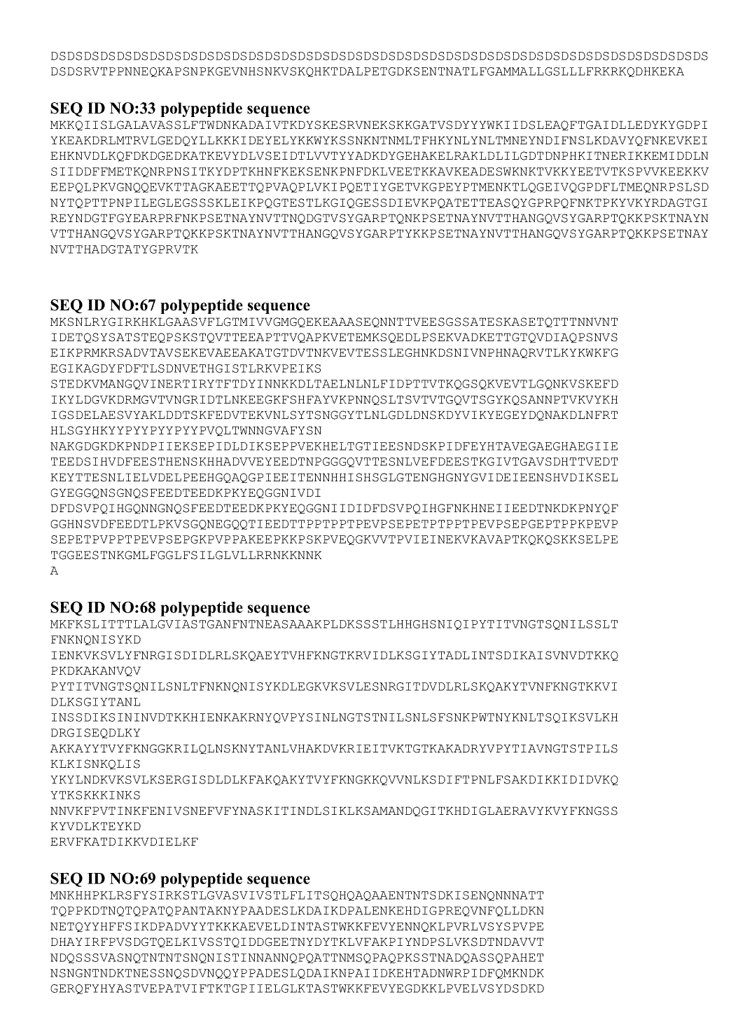

The immunogenic composition of the invention optionally further comprises a staphylococcal protein, for example a protein from S. aureus or S. epidermidis. Some embodiments of the invention contain proteins from both S. aureus and S. epidermidis. Immunogenic compositions of the invention comprise an isolated protein which comprises an amino acid sequence which has at least 85% identity, optionally at least 90% identity, at least 95% identity, at least 97-99% or exact identity, to that of any sequence of FIG. 1.

Where a protein is specifically mentioned herein, it is optionally a reference to a native or recombinant, full-length protein or optionally a mature protein in which any signal sequence has been removed. The protein may be isolated directly from the staphylococcal strain or produced by recombinant DNA techniques. Immunogenic fragments of the protein may be incorporated into the immunogenic composition of the invention. These are fragments comprising at least 10 amino acids, at least 20 amino acids, at least 30 amino acids, at least 40 amino acids, at least 50 amino acids or at least 100 amino acids, taken contiguously from the amino acid sequence of the protein. In addition, such immunogenic fragments are typically immunologically reactive with antibodies generated against the Staphylococcal proteins or with antibodies generated by infection of a mammalian host with Staphylococci or contain T cell epitopes. In an embodiment, immunogenic fragments also includes fragments that when administered at an effective dose, (either alone or as a hapten bound to a carrier), elicit a protective immune response against Staphylococcal infection, optionally it is protective against S. aureus and/or S. epidermidis infection. Such an immunogenic fragment may include, for example, the protein lacking an N-terminal leader sequence, and/or a transmembrane domain and/or a C-terminal anchor domain. In an embodiment, the immunogenic fragment according to the invention comprises substantially all of the extracellular domain of a protein which has at least 85%, 90%, 95%, 97% or 99% identity, to that a sequence selected from FIG. 1 over the entire length of the fragment sequence.

In an embodiment, immunogenic compositions of the invention may contain fusion proteins of Staphylococcal proteins, or fragments of staphylococcal proteins. Such fusion proteins may be made recombinantly and may comprise one portion of at least 2, 3, 4, 5 or 6 staphylococcal proteins, for example the combinations of staphylococcal proteins listed below. Alternatively, a fusion protein may comprise multiple portions of at least 2, 3, 4 or 5 staphylococcal proteins. These may combine different Staphylococcal proteins or fragments thereof in the same protein. Alternatively, the invention also includes individual fusion proteins of Staphylococcal proteins or fragments thereof, as a fusion protein with heterologous sequences such as a provider of T-cell epitopes or purification tags, for example: β-galactosidase, glutathione-S-transferase, green fluorescent proteins (GFP), epitope tags such as FLAG, myc tag, poly histidine, or viral surface proteins such as influenza virus haemagglutinin, or bacterial proteins such as tetanus toxoid, diphtheria toxoid, CRM197. The fusion protein may be present in the immunogenic composition of the invention as a free protein or it may be a carrier protein linked to a saccharide.

Proteins

In an embodiment, the immunogenic composition of the invention further comprises one or more of the proteins mentioned below or immunogenic fragments thereof. Many of the proteins fall into the categories of extracellular component binding proteins, transporter proteins or toxins and regulators of virulence. The immunogenic composition of the invention optionally further comprises a staphylococcal extracellular component binding protein or a staphylococcal transporter protein or a staphylococcal toxin or regulator of virulence. The immunogenic composition of the invention optionally comprises at least or exactly 1, 2, 3, 4, 5 or 6 staphylococcal proteins.

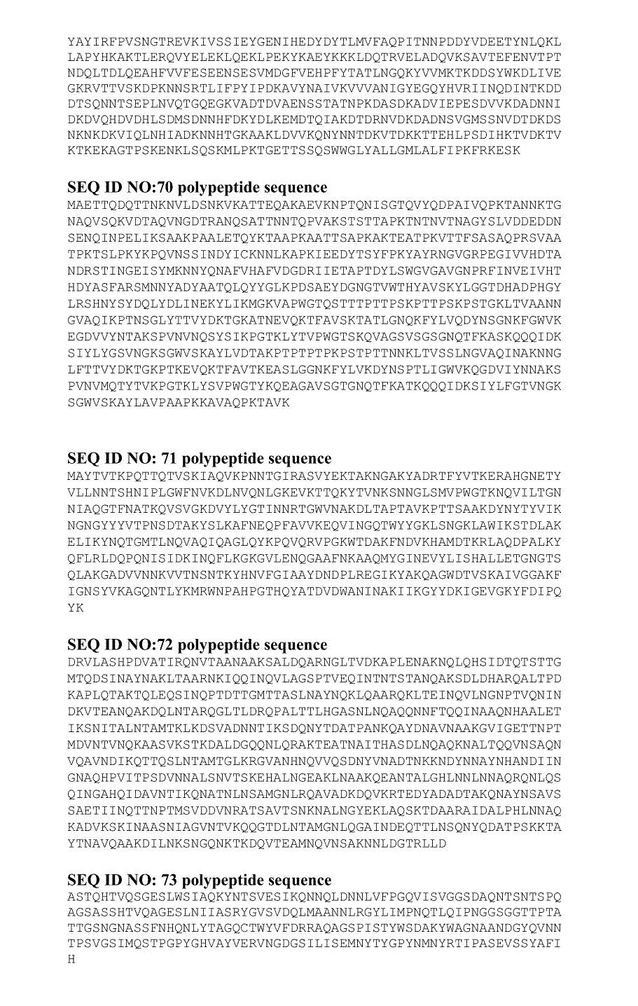

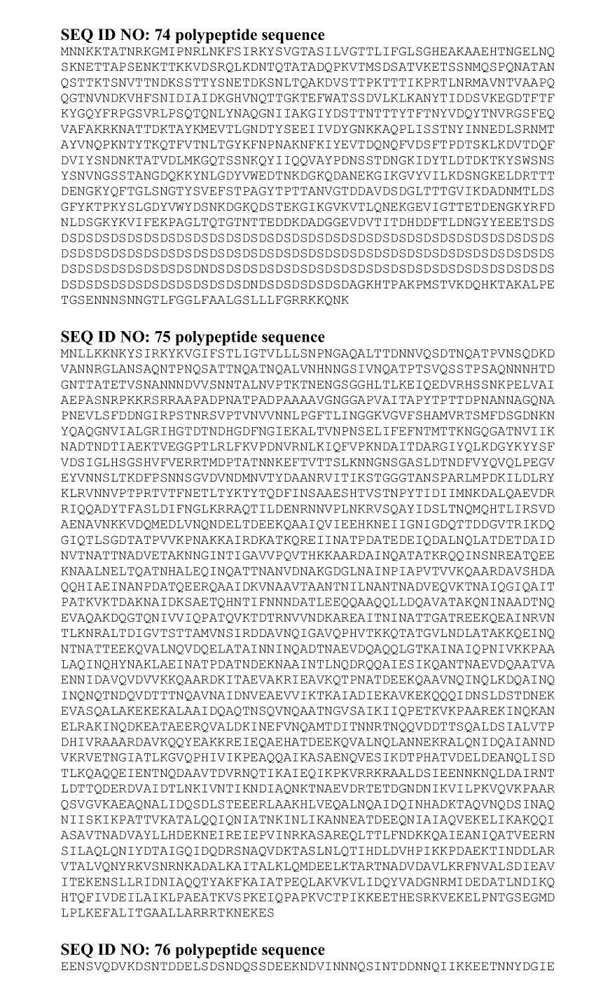

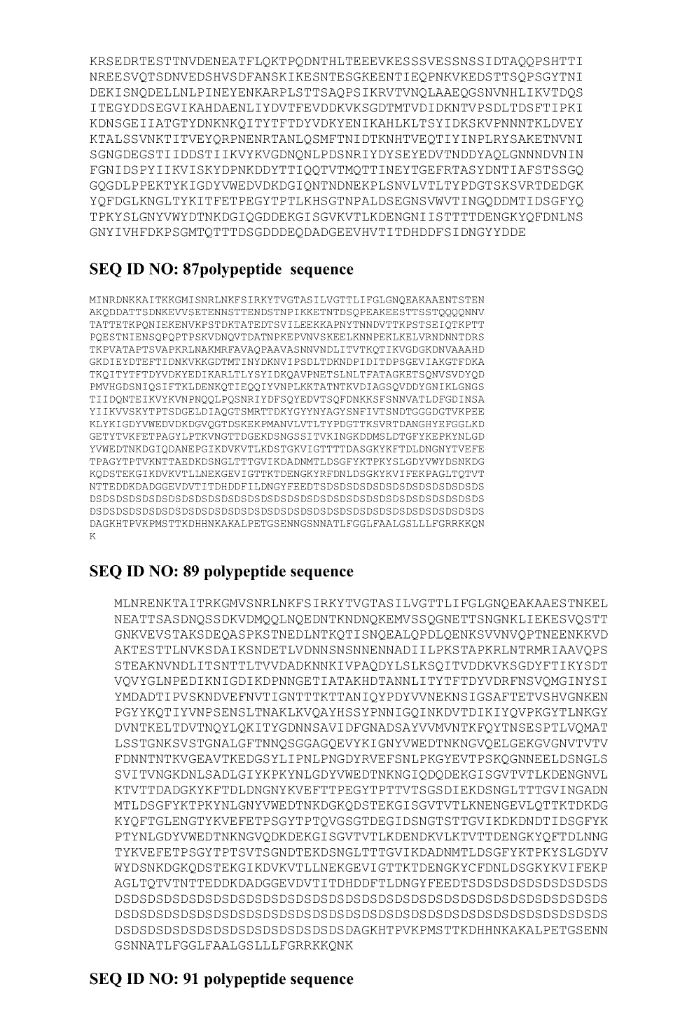

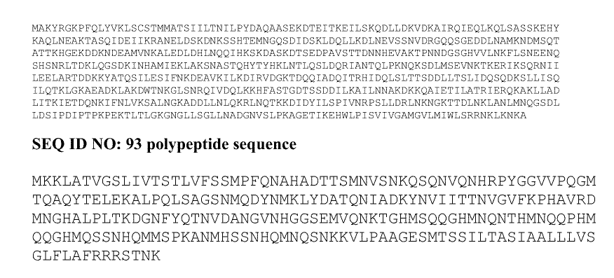

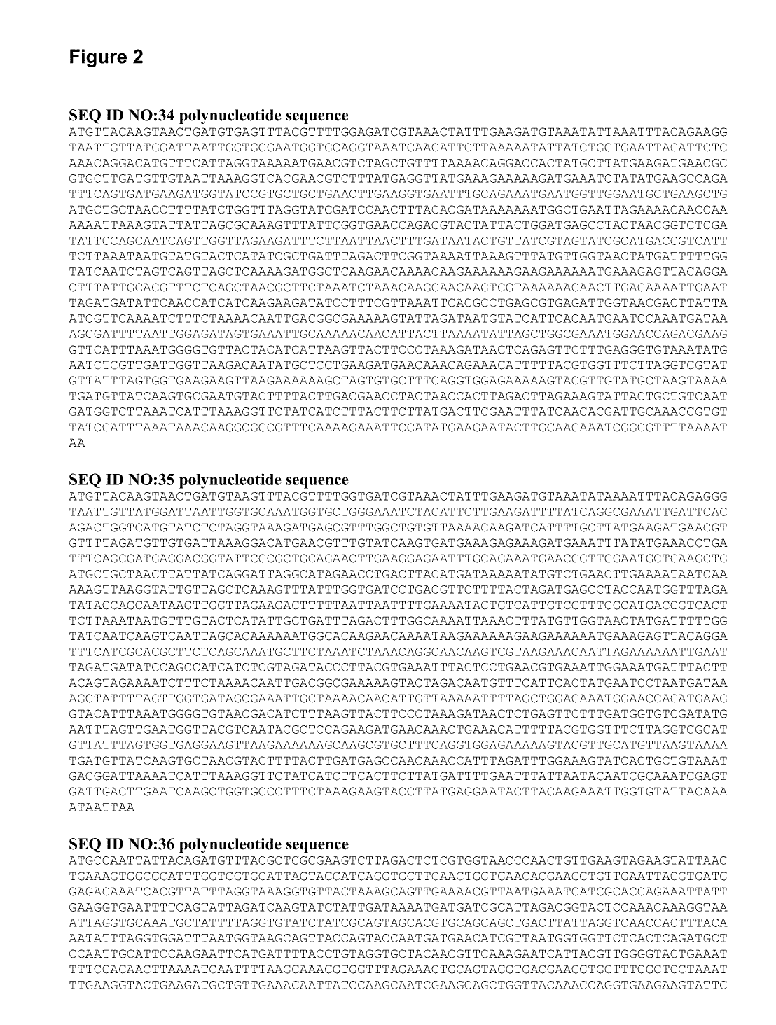

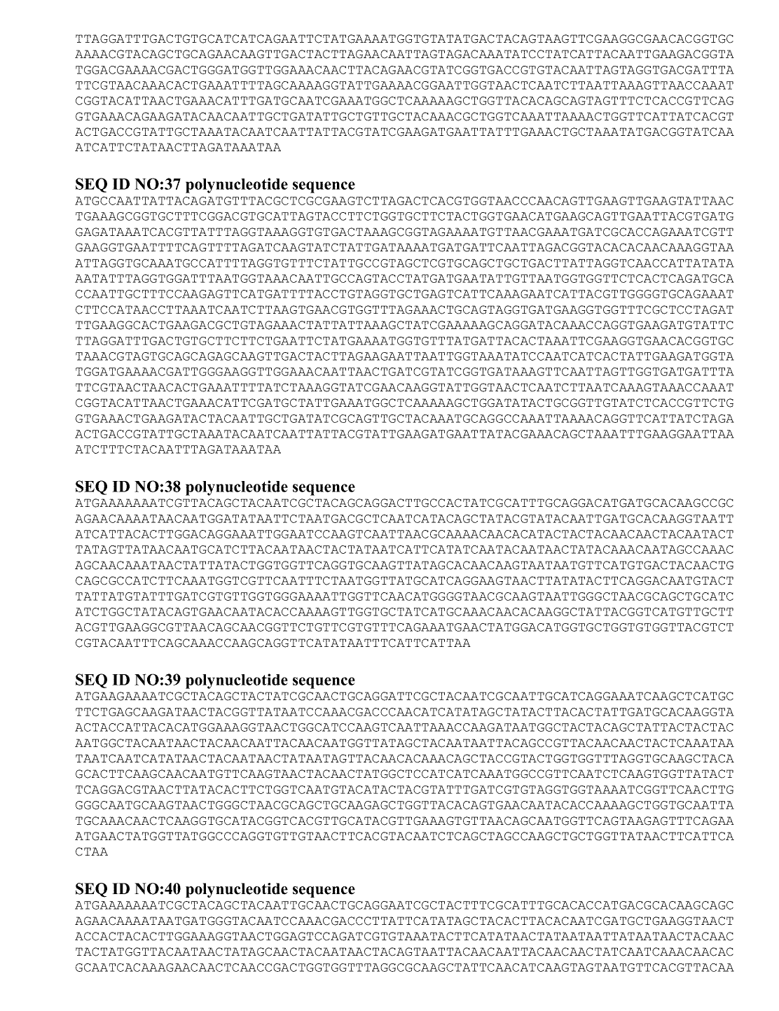

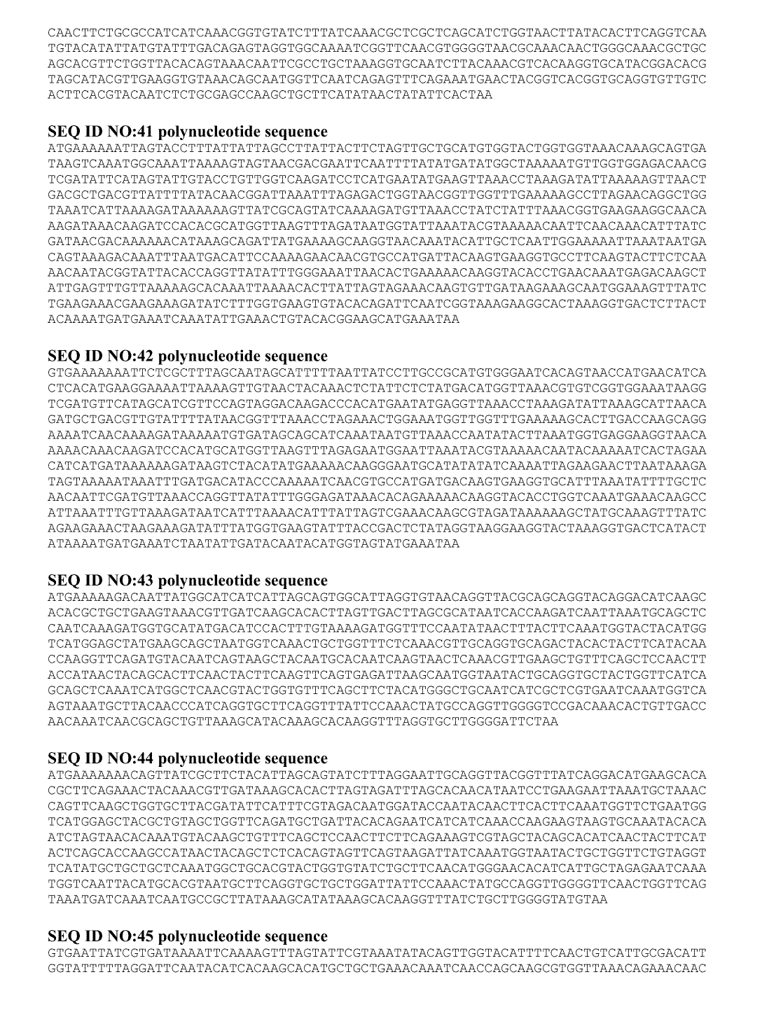

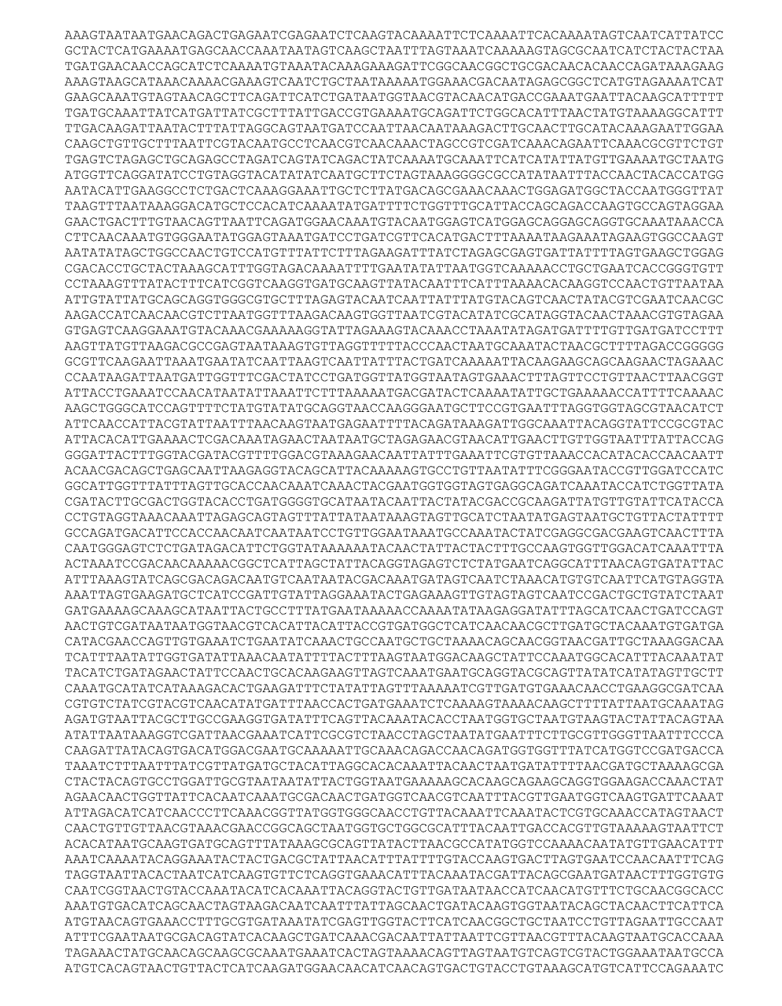

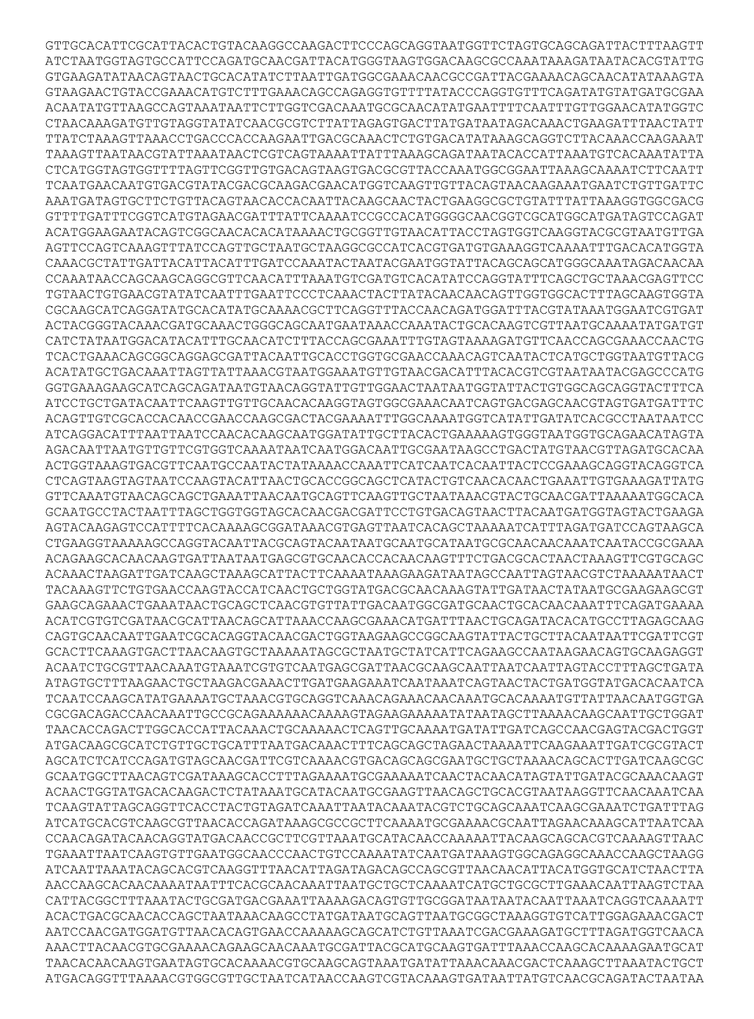

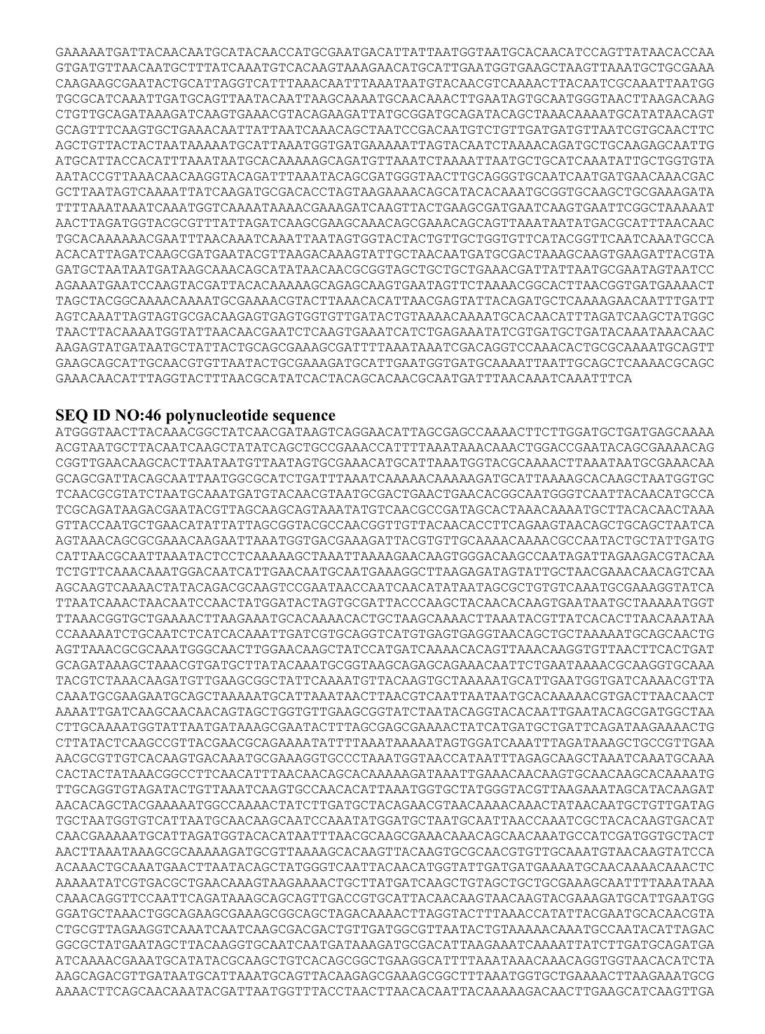

TABLE 1
The following table sets out the SEQ ID numbers of preferred
protein sequences and DNA sequences that are found in FIG. 1
and FIG. 2 respectively. SA indicates a sequence from S. aureus
and SE indicates a sequence from S. epidermidis.

| Name | Protein sequence | DNA sequence |
|---|---|---|
| Immunodominant ABC transporter | | |
| SA | SEQ ID 1 | SEQ ID 34 |
| SE | SEQ ID 2 | SEQ ID 35 |
| Laminin receptor | | |
| SA | SEQ ID 3 | SEQ ID 36 |
| SE | SEQ ID 4 | SEQ ID 37 |
| Secretory Antigen A SsaA | | |
| SA 1 | SEQ ID 5 | SEQ ID 38 |
| SA 2 | SEQ ID 6 | SEQ ID 39 |
| SE | SEQ ID 7 | SEQ ID 40 |
| SitC | | |
| SA | SEQ ID 8 | SEQ ID 41 |
| SE | SEQ ID 9 | SEQ ID 42 |
| IsaA/PisA (IssA) | | |
| SA | SEQ ID 10 | SEQ ID 43 |
| SE | SEQ ID 11 | SEQ ID 44 |
| EbhA/B | | |
| SA EbhA | SEQ ID 12 | SEQ ID 45 |
| SA EbhB | SEQ ID 13 | SEQ ID 46 |
| SE EbhA | SEQ ID 14 | SEQ ID 47 |
| SE EbhB | SEQ ID 15 | SEQ ID 48 |
| Accumulation-assoc pro Aap | | |
| SA | SEQ ID 16 | SEQ ID 49 |
| SE | SEQ ID 17 | SEQ ID 50 |
| RNA III activating protein RAP | | |
| SA | SEQ ID 18 | SEQ ID 51 |
| SE | SEQ ID 19 | SEQ ID 52 |
| FIG/SdrG | | |
| SA | SEQ ID 20 | SEQ ID 53 |
| SE | SEQ ID 21 | SEQ ID 54 |
| Elastin binding protein EbpS | | |
| SA | SEQ ID 22 | SEQ ID 55 |
| SE | SEQ ID 23 | SEQ ID 56 |
| Extracellular protein EFB SA | SEQ ID 24 | SEQ ID 57 |
| alpha toxin SA | SEQ ID 25 | SEQ ID 58 |
| SBI SA | SEQ ID 26 | SEQ ID 59 |
| IsdA SA | SEQ ID 27 | SEQ ID 60 |
| IsdB SA | SEQ ID 28 | SEQ ID 61 |
| SdrC SA | SEQ ID 29 | SEQ ID 62 |
| ClfA SA | SEQ ID 30 | SEQ ID 63 |
| FnbA SA | SEQ ID 31 | SEQ ID 64 |
| ClfB SA | SEQ ID 32 | SEQ ID 65 |
| Coagulase SA | SEQ ID 33 | SEQ ID 66 |
| FnbB SA | SEQ ID 67 | SEQ ID 77 |
| MAP SA | SEQ ID 68 | SEQ ID 78 |
| HarA SA | SEQ ID 69 | SEQ ID 79 |
| Autolysin glucosaminidase SA | SEQ ID 70 | SEQ ID 80 |
| Autolysin amidase SA | SEQ ID 71 | SEQ ID 81 |
| Ebh fragment SA | SEQ ID 72 | SEQ ID 82 |
| Autolysin Ant SA | SEQ ID 73 | SEQ ID 83 |
| SdrC SA | SEQ ID 74 | SEQ ID 84 |
| MRPII SA | SEQ ID 75 | SEQ ID 85 |
| SdrG SA | SEQ ID 76 | SEQ ID 86 |
| SdrE SA | SEQ ID 87 | SEQ ID 88 |
| SdrD SA | SEQ ID 89 | SEQ ID 90 |
| SasF SA | SEQ ID 91 | SEQ ID 92 |

Extracellular Component Binding Proteins

Extracellular component binding proteins are proteins that bind to host extracellular components. The term includes, but is not limited to adhesins.

Examples of extracellular component binding proteins include laminin receptor (Naidu et al J. Med. Microbiol. 1992, 36; 177), SitC/MntC/saliva binding protein (U.S. Pat. No. 5,801,234, Wiltshire and Foster Infec. Immun. 2001, 69; 5198), EbhA (Williams et al Infect. Immun. 2002, 70; 6805), EbhB, Elastin binding protein (EbpS) (Park et al 1999, J. Biol. Chem. 274; 2845), EFB (FIB) (Wastfelt and Flock 1995, J. Clin. Microbiol. 33; 2347), SBI (Zhang et al FEMS Immun. Med. Microbiol. 2000, 28; 211), autolysin (Rupp et al 2001, J. Infect. Dis. 183; 1038), ClfA (U.S. Pat. No. 6,008,341, McDevitt et al Mol. Microbiol. 1994, 11; 237), SdrC, SdrG (McCrea et al Microbiology 2000, 146; 1535), SdrH (McCrea et al Microbiology 2000, 146; 1535), Lipase GehD (US2002/0169288), SasA (Roche et al Microbiology 2003, 149; 643), SasC (Roche et al Microbiology 2003, 149; 643), SasK (Roche et al Microbiology 2003, 149; 643), FnbA (Flock et al Mol Microbiol. 1994, 12; 599, U.S. Pat. No. 6,054,572), FnbB (WO 97/14799, Booth et al 2001 Infec. Immun. 69; 345), collagen binding protein Cna (Visai et al 2000, J. Biol. Chem. 275; 39837), ClfB (WO 99/27109), SdrD (WO 99/27109), SdrE (WO 99/27109), FbpA (Phonimdaeng et al 1988 J. Gen Microbiol. 134; 75), Npase (Flock 2001 J. Bacteriol. 183; 3999), IsaA/PisA (Lonenz et al FEMS Immuno. Med. Microbiol. 2000, 29; 145), SsaA (Lang et al FEMS Immunol. Med. Microbiol. 2000, 29; 213), EPB (Hussain and Hermann symposium on Staph Denmark 14-17^{th} 2000), SSP-1 (Veenstra et al 1996, J. Bacteriol. 178; 537), SSP-2 (Veenstra et al 1996, J. Bacteriol. 178; 537), 17 kDa heparin binding protein HBP (Fallgren et al 2001, J. Med. Microbiol. 50; 547), Vitronectin binding protein (Li et al 2001, Curr. Microbiol. 42; 361), fibrinogen binding protein, coagulase, Fig (WO 97/48727) and MAP (U.S. Pat. No. 5,648,240)

SitC/MntC/Saliva Binding Protein

This is an ABC transporter protein which is a homologue of adhesin PsaA in S. pneumoniae. It is a highly immunogenic 32 kDa lipoprotein which is distributed through the bacterial cell wall (Cockayne et al Infect. Immun. 1998 66; 3767). It is expressed in S. aureus and S. epidermidis as a 32 kDa lipoprotein and a 40 kDa homologue is present in S. hominis. In S. epidermidis, it is a component of an iron-regulated operon. It shows considerable homology to both adhesins including FimA of Streptococcus parasanguis, and with lipoproteins of a family of ABC transporters with proven or putative metal iron transport functions. Therefore SitC is included as an extracellular biding protein and as a metal ion transporter.

The saliva binding protein disclosed in U.S. Pat. No. 5,801,234 is also a form of SitC and can be included in an immunogenic composition of the invention.

ClfA and ClfB

Both these proteins have fibrinogen binding activity and trigger S. aureus to form clumps in the presence of plasma. They contain a LPXTG (SEQ ID NO: 95) motif common to wall associated proteins.

ClfA is described in U.S. Pat. No. 6,008,341 and ClfB is described in WO 99/27109.

Coagulase (FbpA)

This is a fibrinogen binding protein which triggers S. aureus to form clumps in the presence of plasma. It is described in references related to Coagulase: Phonimdaeng et al (J. Gen. Microbio. 1988, 134:75-83), Phonimdaeng et al. (Mol Microbiol 1990; 4:393-404), Cheung et al. (Infect Immun 1995; 63:1914-1920) and Shopsin et al. (J. CLin. Microbiol. 2000; 38:3453-3456). In an embodiment, fragments for inclusion in the immunogenic composition of the invention include the mature protein in which the signal peptide has been removed (amino acids 27 to the C-terminus).

Coagulase has three distinct domains. Amino acids 59-297 which is a coiled coil region, amino acids 326-505 which is a proline and glycine rich region and the C-terminal domain from amino acid 506 to 645 which has a beta sheet conformation. Each of these domains is a fragment which may be incorporated into the immunogenic composition of the invention.

SdrG

This protein is described in WO 00/12689. SdrG is found in coagulase negative staphylococci and is a cell wall associated protein containing a LPXTG (SEQ ID NO: 95) sequence.

SdrG contains a signal peptide (amino acids 1-51), a region containing fibrinogen binding sites and collagen binding sites (amino acids 51-825), two CnaB domains (amino acids 627-698 and 738-809), a SD repeat region (amino acids 825-1000) and an anchor domain (amino acids 1009-1056).

In an embodiment fragments of SdrG include polypeptides in which the signal peptide and/or the SD repeats and the anchor domain have been removed. These include polypeptides comprising or consisting of amino acids 50-825, amino acids 50-633, amino acids 50-597 (SEQ ID NO 2 of WO 03/76470), amino acids 273-597 (SEQ ID NO 4 of WO 03/76470), amino acids 273-577 (SEQ ID NO 6 of WO 03/76470) amino acids 1-549, amino acids 219-549, amino acids 225-549, amino acids 219-528, amino acids 225-528 of SEQ ID NO: 70 or 20 or 21.

Optionally, an SdrG polypeptide having a sequence at least 80%, 85%, 90%, 92%, 95%, 97%, 98%, 99% or 100% homologous to the sequence of SEQ ID NO: 70, 20 or 21 is incorporated into the immunogenic composition of the invention.

The compositions of the invention optionally comprise a fragment of the SdrG polypeptides described above.

In an embodiment fragments have the signal peptide and/or the SD repeat domain and/or the anchoring domain deleted. For example sequences corresponding to amino acids 1-713, 1-549, 225-549, 225-529, 24-717, 1-707, 1-690, 1-680, 1-670, 1-660, 1-650, 1-640, 1-630, 1-620, 1-610, 1-600, 34-707, 44-697, 36-689 of SEQ ID 70 or sequences having 85%, 90%, 92%, 95%, 97%, 98%, 99% or 100% identity to SEQ ID 70 or 20 or 21.

In an embodiment, fragments with the signal peptide deleted have a methionine residue at the N-terminus of the fragment to ensure correct translation.

In an embodiment, the fragment has the following sequence:—

(SEQ ID NO: 96)
MEENSVQDVKDSNTDDELSDSNDQSSDEEKNDVINNNQSINTDDNNQIIK
KEETNNYDGIEKRSEDRTESTTNVDENEATFLQKTPQDNTHLTEEEVKES
SSVESSNSSIDTAQQPSHTTINREESVQTSDNVEDSHVSDFANSKIKESN
TESGKEENTIEQPNKVKEDSTTSQPSGYTNIDEKISNQDE
LLNLPINEYENKARPLSTTSAQPSIKRVTVNQLAAEQGSNVNHLIKVTDQ
SITEGYDDSEGVIKAHDAENLIYDVTFEVDDKVKSGDTMTVDIDKNTVPS
DLTDSFTIPKIKDNSGEIIATGTYDNKNKQITYTFTDYVDKYENIKAHLK
LTSYIDKSKVPNNNTKLDVEYKTALSSVNKTITVEYQRPNENRTANLQSM
FTNIDTKNHTVEQTIYINPLRYSAKETNVNISGNGDEGST
IIDDSTIIKVYKVGDNQNLPDSNRIYDYSEYEDVTNDDYAQLGNNNDVNI
NFGNIDSPYIIKVISKYDPNKDDYTTIQQTVTMQTTINEYTGEFRTASYD
NTIAFSTSSGQGQGDLPPEKTYKIGDYVWEDVDKDGIQNTNDNEKPLSNV
LVTLTYPDGTSKSVRTDEDGKYQFDGLKNGLTYKITFETPEGYTPTLKHS
GTNPALDSEGNSVWVTINGQDDMTIDSGFYQTPKYSLGNY
VWYDTNKDGIQGDDEKGISGVKVTLKDENGNIISTTTTDENGKYQFDNLN
SGNYIVHFDKPSGMTQTTTDSGDDDEQDADGEEVHVTITDHDDFSIDNGY
YDDE

EbhA and EbhB

EbhA and EbhB are proteins that are expressed in both S. aureus and S. epidermidis (Clarke and Foster Infect. Immun. 2002, 70; 6680, Williams et al Infect. Immun. 2002, 20; 6805) and which bind to fibronectin. Since fibronectin is an important component of extracellular matrix, EbhA and EbhB have an important function in adhering staphylococci to host extracellular matrix.

The Ebh proteins are large, having a molecular weight of 1.1 megadaltons. It is advantageous to use a fragment of the Ebh protein rather than the complete sequence due to ease of production and formulation. The central region of the protein contains imperfect repeats which contain fibronectin binding sites. Fragments containing one or more of the repeat domains described below are some fragments suitable for incorporation into the immunogenic composition of the invention.

Ebh proteins contain imperfect repeats units of 127 amino acids in length which are characterised by containing the consensus sequence:—

L.G.{10}A.{13}Q.{26}L...M..L.{33}A

or

.{19}L.G.{10}A.{13}Q.{26}L...M..L.{33}A.{12}

or

.....I/V..A...I/V..AK.ALN/DG..NL..AK..A.{6}L..LN.AQK..L..QI/V..A..V.. V.{6}A..LN/D.AM..L...I/V.D/E...TK.S.NY/F.N/DAD..K..AY/F..AV..A..I/V.N/D.......

Where ‘.’ means any amino acid and ‘.{10}’ means any 10 amino acids and I/V indicates alternative choices of amino acid.

By reference to the sequence disclosed in Kuroda et al (2001) Lancet 357; 1225-1240, and Table 2, the repeat sequences within Ebh proteins are readily deduced.

In an embodiment, fragments to be included in the immunogenic composition of the invention include proteins containing of one, two, three, four, five, six, seven, eight, nine, ten or more than 10 of the 127 amino acid repeat units. Such fragments may consist of 1, 2, 3, 4, 5, 6, 7, 8, 9, 10 or more repeats of the 127 amino acid repeat region or may consist of 1, 2, 3, 4, 5, 6, 7, 8, 9, 10 or more repeats with additional amino acid residues present at either or both ends of the fragment. Optionally the fragment is the H2 polypeptide of about 44 kDa spanning three repeats (amino acids 3202-3595) as described in Clarke et al Infection and Immunity 70, 6680-6687, 2002. Such fragments will optionally be able to bind fibronectin and/or to elicit antibodies that are reactive against the whole Ebh protein.

The Ebh proteins are capable of binding to fibronectin. In an embodiment, fragments of these polypeptides sequences retain the ability to bind to fibronectin. Binding to fibronectin can be assessed by ELISA as described by Clarke et al (Infection and Immunity 70; 6680-6687 2002).

In an embodiment, the fragment is one which comprises a B-cell or T-helper epitope, for example those fragments/peptides described in Tables 3 and 4.

TABLE 2
Repeat sequences in the full-length sequence of Ebh.
The full-length sequence of Ebh is disclosed in Kuroda et
al (2001) Lancet 357; 1225-1240. The following table shows
the amino acid residues at which the 127 amino acid
repeats begin and end within the full length sequence.

| | Begin | End |
|---|---|---|
| 1 | 3204 | 3330 |
| 2 | 3331 | 3457 |
| 3 | 3457 | 3583 |
| 4 | 3583 | 3709 |
| 5 | 3709 | 3835 |
| 6 | 3835 | 3961 |
| 7 | 3961 | 4087 |
| 8 | 4200 | 4326 |
| 9 | 4326 | 4452 |
| 10 | 4452 | 4578 |
| 11 | 4578 | 4704 |
| 12 | 4704 | 4830 |
| 13 | 4830 | 4956 |
| 14 | 4956 | 5082 |
| 15 | 5082 | 5208 |
| 16 | 5208 | 5334 |
| 17 | 5334 | 5460 |
| 18 | 5460 | 5586 |
| 19 | 5585 | 5711 |
| 20 | 5711 | 5837 |
| 21 | 5837 | 5963 |
| 22 | 5963 | 6089 |
| 23 | 6089 | 6215 |
| 24 | 6215 | 6341 |
| 25 | 6341 | 6467 |
| 26 | 6467 | 6593 |
| 27 | 6593 | 6719 |
| 28 | 6719 | 6845 |
| 29 | 6845 | 6971 |
| 30 | 6971 | 7097 |
| 31 | 7097 | 7223 |
| 32 | 7223 | 7349 |
| 33 | 7349 | 7475 |
| 34 | 7475 | 7601 |
| 35 | 7601 | 7727 |
| 36 | 7727 | 7853 |
| 37 | 7852 | 7978 |
| 38 | 7978 | 8104 |
| 39 | 8104 | 8230 |
| 40 | 8230 | 8356 |
| 41 | 8356 | 8482 |
| 42 | 8482 | 8608 |
| 43 | 8604 | 8730 |
| 44 | 8858 | 8984 |

TABLE 3
B-cell epitope prediction for a 127 amino acid
repeat:
The full-length sequence is disclosed in Kuroda
et al (2001) Lancet 357; 1225-1240. One of these
repeats, encoded by amino acids 3204-3331 of the
full-length sequence was chosen to carry out an
epitope prediction:-
MDVNTVNQKAASVKSTKDALDGQQNLQRAKTEATNAITHASDLNQAQ
KNALTQQVNSAQNVHAVNDIKQTTQSLNTAMTGLKRGVANHNQVVQS
DNYVNADTNKKNDYNNAYNHANDIINGNAQHPVI
(SEQ ID NO: 97)

| Begin | End | Start | Stop |
|---|---|---|---|
| 5 | 10 | 3208 | 3213 |
| 14 | 19 | 3217 | 3222 |
| 21 | 33 | 3224 | 3236 |
| 42 | 51 | 3245 | 3254 |
| 66 | 74 | 3269 | 3277 |
| 100 | 112 | 3303 | 3315 |
| 117 | 123 | 3320 | 3326 |

The “Begin” and “End” columns present the position of the predicted B-cell epitopes in the 127 amino acid repeat
The “Start” and “Stop” columns present the position of the predicted B-cell epitopes in the Ebh full length sequence

TABLE 4
T-helper cell epitope prediction in Ebh:
The full-length sequence is disclosed in TrEMBL
database, sequence reference Q8NWQ6. One of
these repeats, encoded by amino acids 3204-3331
of the full-length sequence was chosen to carry
out an epitope prediction:-
MDVNTVNQKAASVKSTKDALDGQQNLQRAKTEATNAITHASDLNQAQ
KNALTQQVNSAQNVHAVNDIKQTTQSLNTAMTGLKRGVANHNQVVQS
DNYVNADTNKKNDYNNAYNHANDIINGNAQHPVI
(SEQ ID NO: 98)

| Corresponding Amino Acids | |
|---|---|
| Numbers of SEQ ID NO: 99 | |
| (the repeat encoded by amino | |
| acids 3204-3331 of sequence | Position |
| reference Q8NWQ6) | sequence |
| 1-9 | 3204 |
| 3-11 | 3206 |
| 6-14 | 3209 |
| 26-34 | 3229 |
| 37-45 | 3240 |
| 43-50 | 3246 |
| 51-59 | 3254 |
| 55-63 | 3258 |
| 61-69 | 3264 |
| 64-72 | 3267 |
| 67-75 | 3270 |
| 74-82 | 3277 |
| 78-86 | 3281 |
| 81-89 | 3284 |
| 85-93 | 3288 |
| 91-99 | 3294 |
| 92-100 | 3295 |
| 97-105 | 3301 |
| 98-106 | 3302 |
| 108-116 | 3311 |
| 112-120 | 3315 |
| 118-126 | 3321 |
| 119-127 | 3322 |

The “Position repeat” column presents the position of the predicted T-cell epitopes in the repeat
The “Position sequence” column presents the position of the predicted T-cell epitopes in the Ebh full length sequence

Fragments of the proteins of the invention may be employed for producing the corresponding full-length polypeptide by peptide synthesis; therefore, these fragments may be employed as intermediates for producing the full-length proteins of the invention.

In an embodiment, variants are used in which several, 5-10, 1-5, 1-3, 1-2 or 1 amino acids are substituted, deleted, or added in any combination.

Elastin Binding Protein (EbpS)

EbpS is a protein containing 486 amino acids with a molecular weight of 83 kDa. It is associated with the cytoplasmic membrane of S. aureus and has three hydrophobic regions which hold the protein in the membrane (Downer et al 2002, J. Biol. Chem. 277; 243; Park et al 1996, J. Biol. Chem. 271; 15803).

Two regions between amino acids 1-205 and 343-486 are surface exposed on the outer face of the cytoplasmic membrane. The ligand binding domain of EbpS is located between residues 14-34 at the N-terminus (Park et al 1999, J. Biol. Chem. 274; 2845).

In an embodiment, the fragment to be incorporated into the immunogenic composition of the invention is the surface exposed fragment containing the elastin binding region (amino acids 1-205). Optionally the fragments do not contain the entire exposed loop but should contain the elastin binding region (amino acids 14-34). An alternative fragment which could be used consists of amino acids forming the second surface exposed loop (amino acids 343-486). Alternative fragments containing up to 1, 2, 5, 10, 20, 50 amino acids less at one or both ends are also possible.

Laminin Receptors

The laminin receptor of S. aureus μlays an important role in pathogenicity. A characteristic feature of infection is bloodstream invasion which allows widespread metastatic abscess formation. Bloodstream invasion requires the ability to extravasate across the vascular basement membrane. This is achieved through binding to laminin through the laminin receptor (Lopes et al Science 1985, 229; 275).

Laminin receptors are surface exposed and are present in many strains of staphylococci including S. aureus and S. epidermidis.

SBI

Sbi is a second IgG binding protein in addition to protein A and it is expressed in most strains of S. aureus (Zhang et al 1998, Microbiology 144; 985).

The N-terminus of the sequence of Sbi has a typical signal sequence with a cleavage site after amino acid 29. Therefore a fragment of Sbi which could be used in an immunogenic composition of the invention starts at amino acid residue 30, 31, 32 or 33 and continues to the C-terminus of Sbi, for example of SEQ ID NO: 26.

The IgG binding domain of Sbi has been identified as a region towards the N-terminus of the protein from amino acids 41-92. This domain is homologous to the IgG binding domains of protein A.

The minimal IgG binding domain of Sbi contains the following sequence (SEQ ID NO: 99):—

```
QTTQNNYVTDQQKAFYQVLHLKGITEEQRNQYIKTLREHPERAQEVFSES
                    ** *** * *** * * * *
LK
  *
* - denotes amino acids which are similar between
IgG binding domains
```

In an embodiment, a fragment of Sbi to be included in the immunogenic composition of the invention contains an IgG binding domain. This fragment contains the consensus sequence for an IgG binding domain as designated by * as shown in the above sequence. Optionally the fragment contains or consists of the complete sequence shown above. Optionally, the fragment contains or consists of amino acids 30-92, 33-92, 30-94, 33-94, 30-146, 33-146, 30-150, 33-150, 30-160, 33-160, 33-170, 33-180, 33-190, 33-200, 33-205 or 33-210 of Sbi, for example of SEQ ID NO:26.

A fragment may contain 1, 2, 3, 4, 5, 6, 7, 8, 9, 10 amino acid substitutions from the sequences indicated.

A fragments may contain multiple repeats (2, 3, 4, 5, 6, 7, 8, 9 or 10) of the IgG binding domain.

EFB-FIB

Fib is a 19 kDa fibrinogen binding protein which is secreted into the extracellular medium by S. aureus. It is produced by all S aureus isolates tested (Wastfelt and Flock 1995, J. Clin. Microbiol. 33; 2347).

S. aureus clumps in the presence of fibrinogen and binds to fibrinogen coated surfaces. This ability facilitates staphylococcal colonisation of catheters and endothelial cells.

Fib contains a signal sequence at the N-terminus of the protein with a putative cleavage site at about amino acid 30. In an embodiment, the immunogenic composition of the invention comprises or consists of the sequence of the mature protein (from about amino acid 30 to the C-terminus of the protein).

Fbe-EfB/FIG

Fbe is a fibrinogen binding protein that is found in many isolates of S. epidermidis and has a deduced molecular weight of 119 kDa (Nilsson et al 1998. Infect. Immun. 66; 2666). Its sequence is related to that of clumping factor from S. aureus (ClfA). Antibodies against Fbe can block the binding of S. epidermidis to fibrinogen coated plates and to catheters (Pei and Flock 2001, J. Infect. Dis. 184; 52).

Fbe has a putative signal sequence with a cleavage site between amino acids 51 and 52. Therefore a potential fragment of Fbe contains the mature form of Fbe extending from amino acid 52 to the C-terminus (amino acid 1,092).

The domain of Fbe from amino acid 52 to amino acid 825 is responsible for fibrinogen binding. In an embodiment, the fragment of Fbe consists of or contains amino acids 52-825.

The region between amino acid 373 and 516 of Fbe shows the most conservation between Fbe and ClfA. In an embodiment, the fragment contains amino acids 373-516 of Fbe.

Amino acids 825-1041 of Fbe contains a highly repetitive region composed of tandemly repeated aspartic acid and serine residues.

IsaA/PisA

IsaA is a 29 kDa protein, also known as PisA has been shown to be a immunodominant staphylococcal protein during sepsis in hospital patients (Lorenz et al 2000, FEMS Immunol. Med. Microb. 29; 145).

The first 29 amino acids of the IsaA sequence are thought to be a signal sequence. In an embodiment, the fragment of IsaA to be included in an immunogenic composition of the invention contains amino acid residues 30 onwards, to the end of the coded sequence.

Fibronectin Binding Protein

Fibronectin binding protein A contains several domains that are involved in binding to fibronectin (WO 94/18327). These are called D1, D2, D3 and D4. In an embodiment fragments of fibronectin binding protein A or B comprise or consist of D1, D2, D3, D4, D1-D2, D2-D3, D3-D4, D1-D3, D2-D4 or D1-D4.

Fibronectin binding protein contains a 36 amino acid signal sequence. For example:

(SEQ ID NO: 100)
VKNNLRYGIRKHKLGAASVFLGTMIVVGMGQDKEAA

Optionally, the mature protein omitting this signal sequence is included in the immunogenic composition of the invention.

Transporter Proteins

The cell wall of Gram positive bacteria acts as a barrier preventing free diffusion of metabolites into the bacterium. A family of proteins orchestrates the passage of essential nutrients into the bacterium and are therefore essential for the viability of the bacterium. The term transporter protein covers proteins involved in the initial step of binding to metabolites such as iron as well as those involved in actually transporting the metabolite into the bacterium.

Molecular iron is an essential co-factor for bacterial growth. Siderophores are secreted that bind free iron and then are captured by bacterial surface receptors that deliver iron for transport across the cytoplasmic membrane. Iron acquisition is critical for the establishment of human infections so that the generation of an immune response against this class of proteins leads to a loss of staphylococcal viability.

Examples of transporter proteins include Immunodominant ABC transporter (Burnie et al 2000 Infect. Imun. 68; 3200), IsdA (Mazmanian et al 2002 PNAS 99; 2293), IsdB (Mazmanian et al 2002 PNAS 99; 2293), IsdC (WO 06/59247), Mg2+ transporter, SitC (Wiltshire and Foster 2001 Infect. Immun. 69; 5198) and Ni ABC transporter.

Immunodominant ABC Transporter

Immunodominant ABC transporter is a well conserved protein which may be capable of generating an immune response that is cross-protective against different staphylococcal strains (Mei et al 1997, Mol. Microbiol. 26; 399). Antibodies against this protein have been found in patients with septicaemia (Burnie et al 2000, Infect. Immun. 68; 3200).

Optional fragments of imunodominant ABC transporter will include the peptides DRHFLN (SEQ ID NO: 101), GNYD (SEQ ID NO: 102), RRYPF (SEQ ID NO: 103), KTTLLK (SEQ ID NO: 104), GVTTSLS (SEQ ID NO: 105), VDWLR (SEQ ID NO: 106), RGFL (SEQ ID NO: 107), KIKVYVGNYDFWYQS (SEQ ID NO: 108), TVIVVSHDRHFLYNNV (SEQ ID NO: 109) and/or TETFLRGFLGRMLFS (SEQ ID NO: 110) since these sequences contain epitopes that are recognised by the human immune system.

IsdA-IsdB

The isd genes (iron-regulated surface determinant) of S. aureus encode proteins responsible for haemoglobin binding and passage of haem iron to the cytoplasm, where it acts as an essential nutrient. IsdA and IsdB are located in the cell wall of staphylococci.

IsdA appear to be exposed on the surface of bacterium since it is susceptible to proteinase K digestion. IsdB was partially digested suggesting that it is partially exposed on the surface of the bacterium (Mazmanian et al 2003 Science 299; 906).

IsdA and IsdB are both 29 kDa proteins which bind heme. Their expression is regulated by the availability of iron via the Fur repressor. Their expression will be high during infection in a host where the concentration of iron will be low.

They are also known as FrpA and FrpB (Morrissey et al 2002, Infect. Immun. 70; 2399). FrpA and FrpB are major surface proteins with a high charge. They have been shown to provide a major contribution to adhesion to plastic.

In an embodiment, the immunogenic composition of the invention comprises a fragment of IsdA and/or IsdB which is described in WO 01/98499 or WO 03/11899.

Toxins and Regulators of Virulence

Members of this family of proteins include toxin such as alpha toxin, hemolysin, enterotoxin B and TSST-1 as well as proteins that regulate the production of toxins such as RAP.

Alpha toxin (HIa)

Alpha toxin is an important virulence determinant produced by most strains of S. aureus. It is a pore forming toxin with haemolytic activity. Antibodies against alpha toxin have been shown to neutralise the detrimental and lethal effects of alpha toxin in animal models (Adlam et al 1977 Infect. Immun. 17; 250). Human platelets, endothelial cells and mononuclear cells are susceptible to the effects of alpha toxin.

The high toxicity of alpha toxin requires that it should be detoxified before being used as an immunogen. This can be achieved by chemical treatment, for instance by treating with formaldehyde, glutaraldehyde of other cross-linking reagents or by chemically conjugating it to bacterial polysaccharides as described below.

A further way of removing toxicity is to introduce point mutations that remove toxicity while retaining the antigenicity of the toxin. The introduction of a point mutation at amino acid 35 of alpha toxin where a histidine residue is replaced with a leucine residue results in the removal of toxicity whilst retaining immunogenicity (Menzies and Kernodle 1996; Infect. Immun. 64; 1839). Histidine 35 appears to be critical for the proper oligomerization required for pore formation and mutation of this residue leads to loss of toxicity.

When incorporated into immunogenic compositions of the invention, alpha toxin is optionally detoxified by mutation of His 35, for example by replacing His 35 with Leu or Arg. In an alternative embodiment, alpha toxin is detoxified by conjugation to other components of the immunogenic composition, for example capsular polysaccharides or PNAG, optionally to S. aureus type 5 polysaccharide and/or S. aureus Type 8 polysaccharide and/or PNAG.

RNA III Activating Protein (RAP)

RAP is not itself a toxin, but is a regulator of the expression of virulence factors. RAP is produced and secreted by staphylococci. It activates the agr regulatory system of other staphylococci and activates the expression and subsequent release of virulence factors such as hemolysin, enterotoxin B and TSST-1.

Other Immunodominant Proteins

Accumulation-Associated Protein (Aap)

Aap is a 140 kDa protein which is essential for the accumulation of S. epidermidis strains on surfaces (Hussain et al Infect. Immun. 1997, 65; 519). Strains expressing this protein produced significantly larger amounts of biofilm and Aap appear to be involved in biofilm formation. Antibodies against Aap are able to inhibit biofilm formation and inhibit the accumulation of S. epidermidis.

Staphylococcal Secretory Antigen SsaA

SsaA is a strongly immunogenic protein of 30 kDa found in both S. aureus and S. epidermidis (Lang et al 2000 FEMS Immunol. Med. Microbiol. 29; 213). Its expression during endocarditis suggested a virulence role specific to the pathogenesis of the infectious disease.

SsaA contains an N-terminal leader sequence and a signal peptidase cleavage site. The leader peptide is followed by a hydrophilic region of approximately 100 amino acids from residue 30 to residue 130.

An optional fragment of SsaA to be incorporated into the immunogenic composition of the invention is made up of the mature protein (amino acids 27 to the C-terminus or amino acids 30 to the C-terminus).

A further optional fragments contains the hydrophilic area of SsaA from amino acid 30 to amino acid 130.

Combinations

Staphylococcal infections progress through several different stages. For example, the staphylococcal life cycle involves commensal colonisation, initiation of infection by accessing adjoining tissues or the bloodstream, anaerobic multiplication in the blood, interplay between S. aureus virulence determinants and the host defence mechanisms and induction of complications including endocarditis, metastatic abscess formation and sepsis syndrome. Different molecules on the surface of the bacterium will be involved in different steps of the infection cycle. By targeting the immune response against a combination of particular antigens involved in different processes of Staphylococcal infection, multiple aspects of staphylococcal function are affected and this can result in good vaccine efficacy.

In particular, combinations of certain antigens from different classes, some of which are involved in adhesion to host cells, some of which are involved in iron acquisition or other transporter functions, some of which are toxins or regulators of virulence and immunodominant antigens can elicit an immune response which protects against multiple stages of infection.

Some combinations of antigens are particularly effective at inducing an immune response. This can be measured either in animal model assays as described in the examples and/or using an opsonophagocytic assay as described in the examples. Without wishing to be bound by theory, such effective combinations of antigens are thought to be enabled by a number of characteristics of the immune response to the antigen combination. The antigens themselves are usually exposed on the surface of Staphylococcal cells, they tend to be conserved but also tend not to be present in sufficient quantity on the surface cell for an optimal bactericidal response to take place using antibodies elicited against the single antigen. Combining the antigens of the invention can result in a formulation eliciting an advantageous combination of antibodies which interact with the Staphylococcal cell beyond a critical threshold. At this critical level, sufficient antibodies of sufficient quality bind to the surface of the bacterium to allow either efficient killing by complement or neutralisation of the bacterium. This can be measured in either an animal challenge model or an opsonisation assay as described in the examples.

Preferred immunogenic compositions of the invention comprise a plurality of proteins selected from at least two different categories of protein, having different functions within Staphylococci. Examples of such categories of proteins are extracellular binding proteins, transporter proteins such as Fe acquisition proteins, toxins or regulators of virulence and other immunodominant proteins.

In a preferred embodiment, immunogenic composition of the invention further comprises a number of proteins equal to or greater than 2, 3, 4, 5 or 6 selected from 2, 3 or 4 different groups selected from;
Group a) extracelular component binding proteins;
Group b) transporter proteins;
Group c) toxins or regulators of virulence
Group d) structural proteins.

In a preferred embodiment, immunogenic composition of the invention further comprises a number of proteins equal to or greater than 2, 3, 4, 5 or 6 selected from 2, 3 or 4 of the following groups:
group a)—at least one staphylococcal extracellular component binding protein or fragment thereof selected from the group consisting of laminin receptor, SitC/MntC/saliva binding protein, EbhA, EbhB, Elastin binding protein (EbpS), EFB (FIB), SBI, ClfA, SdrC, SdrD, SdrE, SdrG, SdrH, SasF, lipase GehD, SasA, SasB, SasC, SasD, SasK, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig and MAP;
group b)—at least one staphylococcal transporter protein or fragment thereof selected from the group consisting of Immunodominant ABC transporter, IsdA, IsdB, IsdC, Mg2+ transporter, HarA, SitC and Ni ABC transporter;
group c)—at least one staphylococcal regulator of virulence, toxin or fragment thereof selected from the group consisting of alpha toxin (Hla), alpha toxin H35R mutant, RNA III activating protein (RAP);
group d)—at least one staphylococcal structural protein or immunogenic fragment thereof selected from the group consisting of MRPII and autolysin.

In a preferred embodiment, the immunogenic composition of the invention comprises a number of proteins equal to or greater than 2, 3, 4, 5 or 6 selected from 2 or 3 of the following groups:
group a)—at least one staphylococcal extracellular component binding protein or immunogenic fragment thereof selected from the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, SasF, Lipase GehD, SasA, SasB, SasC, SasD, SasK, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig and MAP;
group b)—at least one staphylococcal transporter protein or immunogenic fragment thereof selected from the group consisting of Immunodominant ABC transporter, IsdA, IsdB, IsdC, HarA, Mg2+ transporter, SitC and Ni ABC transporter;
group c)—at least one staphylococcal regulator of virulence, toxin or immunogenic fragment thereof selected from the group consisting of alpha toxin (Hla), alpha toxin H35R mutant, RNA III activating protein (RAP).

In a preferred embodiment, the immunogenic composition of the invention contains at least one protein selected from group a) and an additional protein selected from group b) and/or group c).

In a further embodiment, the immunogenic composition of the invention contains at least one antigen selected from group b) and an additional protein selected from group c) and/or group a).

In a further embodiment, the immunogenic composition of the invention contains at least one antigen selected from group c) and an additional protein selected from group a) and/or group b).

An optional combination of proteins in the immunogenic composition of the invention comprises laminin receptor and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises SitC and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises EbhA and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises EbhB and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises EbpS and 1, 2, 3, 4 or 5 further antigens selected from the group consisting of immunodominant ABC transporter, IsdA, IsdB, IsdA, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises EFB(FIB) and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises SBI and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises autolysin and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises ClfA and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises SdrC and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises SdrD and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises SdrE and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises SdrG and 1, 2, 3, 4 or 5 further antigens selected from the group consisting of immunodominant ABC transporter, IsdA, IsdB, IsdC. HarA, Mg2+ transporter, SitC, Ni ABC transporter, alpha toxin, alpha toxin H35L or H35R mutant and RAP.

A further combination of proteins in the immunogenic composition of the invention comprises SdrH and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises SasF and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises Lipase GehD and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises SasF and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises FnbA and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises FnbB and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises Cna and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises ClfB and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises FbpA and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises Npase and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises IsaA/PisA and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises SsaA and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises EPB and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises SSP-1 and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises SSP-2 and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises HPB and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises vitronectin binding protein and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises fibrinogen binding protein and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises coagulase and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises Fig and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of proteins in the immunogenic composition of the invention comprises MAP and 1, 2, 3, 4 or 5 further antigens selected from the group consisting of immunodominant ABC transporter, IsdA, IsdB, IsdC, HarA, Mg2+ transporter, SitC, Ni ABC transporter, alpha toxin, alpha toxin H35L or H35R mutant, RAP, Aap and SsaA.

A further combination of protein in the immunogenic composition of the invention comprises immunodominant ABC transporter and 1, 2, 3, 4 or 5 further antigens selected from the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, Lipase GehD, SasA, FnbA, FnbB, Cna, ClfA, ClfB, FbpA, Npase, IsaA/PisA, SsaA, EPB, SSP-1, SSP-2, HBP, Vitronectin binding protein, fibrinogen binding protein, coagulase, Fig, MAP, alpha toxin, alpha toxin H35L or H35R mutant, RAP, Aap and SsaA.

A further combination of protein in the immunogenic composition of the invention comprises IsdA and 1, 2, 3, 4 or 5 further antigens selected from the group consisting of laminin receptor, SitC/MntC/saliva binding protein, EbhA, EbhB, Elastin binding protein (EbpS), EFB (FIB), SBI, autolysin, ClfA, SdrC, SdrC, SdrE, SdrG, SdrH, SasF, Lipase GehD, SasA, FnbA, FnbB, Cna, ClfA, ClfB, FbpA, Npase, IsaA/PisA, SsaA, EPB, SSP-1, SSP-2, HBP, Vitronectin binding protein, fibrinogen binding protein, coagulase, Fig, MAP, alpha toxin, alpha toxin H35L or H35R mutant, RAP, Aap and SsaA.

A further combination of protein in the immunogenic composition of the invention comprises IsdB and 1, 2, 3, 4 or 5 further antigens selected from the group consisting of laminin receptor, SitC/MntC/saliva binding protein, EbhA, EbhB, Elastin binding protein (EbpS), EFB (FIB), SBI, autolysin, ClfA, SdrC, SdrG, SdrH, SasF, Lipase GehD, SasA, FnbA, FnbB, Cna, ClfA, ClfB, FbpA, Npase, IsaA/PisA, SsaA, EPB, SSP-1, SSP-2, HBP, Vitronectin binding protein, fibrinogen binding protein, coagulase, Fig, MAP, alpha toxin, alpha toxin H35L or H35R mutant, RAP, Aap and SsaA.

A further combination of protein in the immunogenic composition of the invention comprises SitC and 1, 2, 3, 4 or 5 further antigens selected from the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, SasF, Lipase GehD, SasA, FnbA, FnbB, Cna, ClfA, ClfB, FbpA, Npase, IsaA/PisA, SsaA, EPB, SSP-1, SSP-2, HBP, Vitronectin binding protein, fibrinogen binding protein, coagulase, Fig, MAP, alpha toxin, alpha toxin H35L or H35R mutant, RAP, Aap and SsaA.

A further combination of protein in the immunogenic composition of the invention comprises alpha toxin and 1, 2, 3, 4 or 5 further antigens selected from the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, SasF, Lipase GehD, SasA, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig, MAP, immunodominant ABC transporter, IsdA, IsdB, IsdC, HarA, Mg2+ transporter, SitC, Ni ABC transporter, Aap and SsaA.

A further combination of protein in the immunogenic composition of the invention comprises alpha toxin H35L OR H35R variant and 1, 2, 3, 4 or 5 further antigens selected from the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, SasF, Lipase GehD, SasA, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig, MAP, immunodominant ABC transporter, IsdA, IsdB, IsdC, HarA, Mg2+ transporter, SitC, Ni ABC transporter, Aap and SsaA.

A further combination of protein in the immunogenic composition of the invention comprises RAP and 1, 2, 3, 4 or 5 further antigens selected from the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, SasF, Lipase GehD, SasA, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig, MAP, immunodominant ABC transporter, IsdA, IsdB, IsdC, HarA, Mg2+ transporter, SitC, Ni ABC transporter, Aap and SsaA.

A further combinations of protein in the immunogenic composition of the invention comprises IsdA and IsdB; IsdA and ClfA; IsdA and ClfB; IsdA and SdrC; IsdA and SdrD; IsdA and SdrE; IsdA and SdrG; IsdA and SasF;IsdB and ClfA; IsdB and ClfB; IsdB and SdrC; IsdB and SdrD; IsdB and SdrE; IsdB and SdrG; IsdB and SasF; ClfA and ClfB; ClfA and SdrC; ClfA and SdrD; ClfA and SdrE; ClfA and SasF; ClfB and SdrC; ClfB and SdrD; ClfB and SdrE; ClfB and SasF; SdrC and SdrD; SdrC and SdrE; SdrC and SasF; SdrD and SdrE; SdrD and SasF; SdrE and SasF.

In the above and below combinations, the specified proteins may optionally be present in the immunogenic composition of the invention as a fragment or fusion protein as described above.

Combinations of Three Proteins

In an embodiment, the immunogenic composition of the invention further comprises three protein components in a combination of alpha-toxin, an extracellular component binding protein (for example an adhesin) and a transporter protein (for example an iron-binding protein).

In such a combination, the alpha toxin may be chemically detoxified or genetically detoxified by introduction of point mutation(s), for example the His35Leu point mutation.

The alpha toxin is present as a free protein or alternatively is conjugated to a polysaccharide or PNAG component of the immunogenic composition.

Examples of Combinations Include:—

An immunogenic composition comprising alpha toxin, IsdA and an extracellular component binding protein selected from the group consisting of laminin receptor, SitC/MntC/saliva binding protein, EbhA, EbhB, Elastin binding protein (EbpS), EFB (FIB), SBI, autolysin, ClfA, SdrC, SdrG, SdrH, Lipase GehD, SasA, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig and MAP.

An immunogenic composition comprising alpha toxin, IsdB and an extracellular component binding protein selected from the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, Lipase GehD, SasA, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig and MAP.

An immunogenic composition comprising alpha toxin, IsdA and an adhesin selected from the group consisting of laminin receptor, EbhA, EbhB, Elastin binding protein (EbpS), EFB (FIB), ClfA, SdrC, SdrD, SdrE, SdrG, SdrH, autolysin, FnbA, FnbB, Cna, ClfB, FbpA, Npase, SSP-1, SSP-2, Vitronectin binding protein, fibrinogen binding protein, coagulase, Fig and MAP.

An immunogenic composition comprising alpha toxin, IsdB and an adhesin selected from the group consisting of laminin receptor, EbhA, EbhB, Elastin binding protein (EbpS), EFB (FIB), autolysin, ClfA, SdrC, SdrD, SdrE, SdrG, SdrH, FnbA, FnbB, Cna, ClfB, FbpA, Npase, SSP-1, SSP-2, Vitronectin binding protein, fibrinogen binding protein, coagulase, Fig and MAP.

An immunogenic composition comprising alpha toxin, IsdA and laminin receptor.

An immunogenic composition comprising alpha toxin, IsdA and EbhA.

An immunogenic composition comprising alpha toxin, IsdA and EbhB.

An immunogenic composition comprising alpha toxin, IsdA and EbpS.

An immunogenic composition comprising alpha toxin, IsdA and EFB (FIB).

An immunogenic composition comprising alpha toxin, IsdA and SdrG.

An immunogenic composition comprising alpha toxin, IsdA and ClfA.

An immunogenic composition comprising alpha toxin, IsdA and ClfB.

An immunogenic composition comprising alpha toxin, IsdA and FnbA.

An immunogenic composition comprising alpha toxin, IsdA and coagulase.

An immunogenic composition comprising alpha toxin, IsdA and Fig.

An immunogenic composition comprising alpha toxin, IsdA and SdrH.

An immunogenic composition comprising alpha toxin, IsdA and SdrC.

An immunogenic composition comprising alpha toxin, IsdA and SdrD.

An immunogenic composition comprising alpha toxin, IsdA and SdrE.

An immunogenic composition comprising alpha toxin, IsdA and MAP.

An immunogenic composition comprising IsaA and Sbi.

An immunogenic composition comprising IsaA and IsdB.

An immunogenic composition comprising IsaA and IsdA.

An immunogenic composition comprising IsaA and SdrC.

An immunogenic composition comprising IsaA and Ebh or fragment thereof as described above.

An immunogenic composition comprising Sbi and SdrC.

An immunogenic composition comprising Sbi and Ebh or fragment thereof as described above.

An immunogenic composition of the invention comprising IsaA, Sbi or SdrC

Selection of antigens expressed in different clonal lineages

Analysis of the occurrence of virulence factors in relation with the population structure of

Staphylococcus aureus showed variable presence of virulence genes in natural populations of S. aureus.

Among clinical isolates of Staphylococcus aureus, at least five clonal lineages were shown to be highly prevalent (Booth et al., 2001 Infect Immun. 69(1):345-52). Alpha-hemolysin (hla), fibronectin-binding protein A (fnbA) and clumping factor A (clfA) were shown to be present in most of the isolates, regardless of lineage identity, suggesting an important role of these proteins in the survival of S. aureus (Booth et al., 2001 Infect Immun. 69(1):345-52). Moreover, according to Peacock et al. 2002 the distributions of fnbA, clfA, coagulase, spa, map, pvl (Panton-Valentine leukocidin), hlg (gamma-toxin), alpha-toxin and ica appeared to be unrelated to the underlying clonal structure suggesting considerable horizontal transfer of these genes.

In contrary, other virulence genes such as fibronectin binding protein B (fnbB), beta-hemolysin (hlb), collagen binding protein (cna), TSST-1 (tst) and methicillin resistance gene (mecA) are strongly associated with specific lineages (Booth et al., 2001 Infect Immun. 69(1):345-52). Similarly, Peacock et al. 2002 (Infect Immun. 70(9):4987-96) showed that the distributions of the enterotoxins, tst, the exfolatins (eta and etb), beta- and delta-toxins, the sdr genes (sdrD, sdrE and bbp), cna, ebpS and efb within the population are all highly significantly related to MLST-derived clonal complexes.

MLST data provide no evidence that strains responsible for nosocomial disease represent a distinct subpopulation from strains causing community-acquired disease or strains recovered from asymptomatic carriers (Feil et al., 2003 J Bacteriol. 185(11):3307-16).

In an embodiment, immunogenic compositions of the invention are effective against staphylococci from different clonal lineages.

In an embodiment, the immunogenic composition comprises 1, 2, 3, 4, or at least 1 protein that is expressed in most isolates of staphylococci. Examples of such proteins include alpha-hemolysin (hla), fibronectin-binding protein A (fnbA) and clumping factor A (clfA), coagulase, spa, map, pvl (Panton-Valentine leukocidin), hlg (gamma-toxin), ica, immunodominant ABC transporter, RAP, autolysin (Rupp et al 2001, J. Infect. Dis. 183; 1038), laminin receptors, SitC, IsaA/PisA, SPOIIIE ( ), SsaA, EbpS, SasF (Roche et al 2003, Microbiology 149; 643), EFB(FIB), SBI, ClfB, IsdA, IsdB, FnbB, Npase, EBP, Bone sialo binding protein II, IsaB/PisB (Lorenz et al FEMS Immuno. Med. Microb. 2000, 29; 145), SasH (Roche et al 2003, Microbiology 149; 643), MRPI, SasD (Roche et al 2003, Microbiology 149; 643), SasH (Roche et al 2003, Microbiology 149; 643), aureolysin precursor (AUR)/Sepp1 and novel autolysin.

In an alternative embodiment, 2 or more proteins which are expressed in different sets of clonal strains are included in the immunogenic composition of the invention. Optionally the combination of antigens will allow an immune response to be generated that is effective against multiple clonal strains, or against all clonal stains. For example combinations include FnbB and betahemolysin, FnbB and Cna, FnbB and TSST-1, FnbB and mecA, FnbB and SdrD, FnbB and SdrF, FnbB and EbpS, FnbB and Efb, beta-haemolysin and Cna, beta-haemolysin and TSST-1, beta-haemolysin and mecA, beta-haemolysin and SdrD, beta-haemolysin and SdrF, beta-haemolysin and EbpS, beta-haemolysin and Efb, Cna and TSST-1, Cna and mecA, Cna and SdrD, Cna and SdrF, Cna and EbpS, Cna and Efb, TSST-1 and mecA, TSST-1 and SdrD, TSST-1 and SdrF, TSST-1 and EbpS, TssT-1 and Efb, MecA and SdrD, MecA and SdrF, MecA and EbpS, MecA and Efb, SdrD and SdrF, SdrD and EbpS, SdeD and Efb, SdrF and EbpS, SdrF and Efb, and, EbpS and Efb.

The combinations described above may be combined with additional components described above.

Protection Against S. aureus and S. epidermidis

In an embodiment of the invention the immunogenic composition provides an effective immune response against more than one strain of staphylococci, for example against strains from both S. aureus and S. epidermidis. For example, a protective immune response is generated against type 5 and 8 serotypes of S. aureus.

One use of the immunogenic composition of the invention is to prevent nosocomial infections, for instance in elective surgery patients, by inoculating prior to hospital treatment. At this stage, it is difficult to accurately predict which staphylococcal strains the patient will be exposed to. It is therefore advantageous to inoculate with a vaccine that is capable of generating an effective immune response against various strains of staphylococci.

An effective immune response is defined as an immune response that gives significant protection in a mouse challenge model or opsonophagocytosis assay as described in the examples. Significant protection in a mouse challenge model, for instance that of example 5, is defined as an increase in the LD50 in comparison with carrier inoculated mice of at least 10%, 20%, 50%, 100% or 200%. Significant protection in a cotton rat challenge model, for instance that of example 8, is defined as a decrease in the mean observed Log CFU/nose of at least 10%, 20%, 50%, 70% or 90%. The presence of opsonising antibodies is known to correlate with protection, therefore significant protection is indicated by a decrease in the bacterial count of at least 10%, 20%, 50%, 70% or 90% in an opsonophagocytosis assay, for instance that of example 7.

Several of the proteins including immunodominant ABC transporter, RNA III activating protein, Laminin receptors, SitC, IsaA/PisA, SsaA, EbhA/EbhB, EbpS and Aap are well conserved between S. aureus and S. epidermidis and example 8 shows that IsaA, ClfA, IsdB, SdrG, HarA, FnbpA and Sbi can generate a cross-reactive immune response (for example cross-reactive between at least one S. aureus and at least one S. epidermidis strain). PIA is also well conserved between S. aureus and S. epidermidis.

Therefore in an embodiment, the immunogenic composition of the invention will comprise PNAG and type 5 and 8 polysaccharides and one, two, three or four of the above proteins.

Vaccines

In an embodiment, the immunogenic composition of the invention is mixed with a pharmaceutically acceptable excipient, and optionally with an adjuvant to form a vaccine.

The vaccines of the present invention may be adjuvanted, particularly when intended for use in an elderly population but also for use in infant populations. Suitable adjuvants include an aluminum salt such as aluminum hydroxide gel or aluminum phosphate or alum, but may also be other metal salts such as those of calcium, magnesium, iron or zinc, or may be an insoluble suspension of acylated tyrosine, or acylated sugars, cationically or anionically derivatized saccharides, or polyphosphazenes.

It is preferred that the adjuvant be selected to be a preferential inducer of a TH1 type of response. Such high levels of Th1-type cytokines tend to favour the induction of cell mediated immune responses to a given antigen, whilst high levels of Th2-type cytokines tend to favour the induction of humoral immune responses to the antigen.

The distinction of Th1 and Th2-type immune response is not absolute. In reality an individual will support an immune response which is described as being predominantly Th1 or predominantly Th2. However, it is often convenient to consider the families of cytokines in terms of that described in murine CD4 +ve T cell clones by Mosmann and Coffman (Mosmann, T. R. and Coffman, R.L. (1989) TH1 and TH2 cells: different patterns of lymphokine secretion lead to different functional properties. (Annual Review of Immunology, 7, p145-173). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of II-4, IL-5, IL-6, IL-10. Suitable adjuvant systems which promote a predominantly Th1 response include: Monophosphoryl lipid A or a derivative thereof (or detoxified lipid A in general—see for instance WO2005107798), particularly 3-de-O-acylated monophosphoryl lipid A (3D-MPL) (for its preparation see GB 2220211 A); and a combination of monophosphoryl lipid A, preferably 3-de-O-acylated monophosphoryl lipid A, together with either an aluminum salt (for instance aluminum phosphate or aluminum hydroxide) or an oil-in-water emulsion. In such combinations, antigen and 3D-MPL™ are contained in the same particulate structures, allowing for more efficient delivery of antigenic and immunostimulatory signals. Studies have shown that 3D-MPL™ is able to further enhance the immunogenicity of an alum-adsorbed antigen [Thoelen et al. Vaccine (1998) 16:708-14; EP 689454-B1].

An enhanced system involves the combination of a monophosphoryl lipid A and a saponin derivative, particularly the combination of QS21 and 3D-MPL™ as disclosed in WO 94/00153, or a less reactogenic composition where the QS21 is quenched with cholesterol as disclosed in WO 96/33739. A particularly potent adjuvant formulation involving QS21, 3D-MPL™ and tocopherol in an oil in water emulsion is described in WO 95/17210. In one embodiment the immunogenic composition additionally comprises a saponin, which may be QS21. The formulation may also comprise an oil in water emulsion and tocopherol (WO 95/17210). Unmethylated CpG containing oligonucleotides (WO 96/02555) and other immunomodulatory oligonucleotides (WO0226757 and WO03507822) are also preferential inducers of a TH1 response and are suitable for use in the present invention.

Particular adjuvants are those selected from the group of metal Salts, oil in water emulsions, Toll like receptors agonist, (in particular Toll like receptor 2 agonist, Toll like receptor 3 agonist, Toll like receptor 4 agonist, Toll like receptor 7 agonist, Toll like receptor 8 agonist and Toll like receptor 9 agonist), saponins or combinations thereof.

An adjuvant that can be used with the vaccine compositions of the invention are bleb or outer membrane vesicle preparations from Gram negative bacterial strains such as those taught by WO02/09746—particularly N. meningitidis blebs. Adjuvant properties of blebs can be improved by retaining LOS (lipooligosacccharide) on its surface (e.g. through extraction with low concentrations of detergent [for instance 0-0.1% deoxycholate]). LOS can be detoxified through the msbB(−) or htrB(−) mutations discussed in WO02/09746. Adjuvant properties can also be improved by retaining PorB (and optionally removing PorA) from meningococcal blebs. Adjuvant properties can also be improved by truncating the outer core saccharide structure of LOS on meningococcal blebs—for instance via the IgtB(−) mutation discussed in WO2004/014417. Alternatively, the aforementioned LOS (e.g. isolated from a msbB(−) and/or IgtB(−) strain) can be purified and used as an adjuvant in the compositions of the invention.

A further adjuvant which may be used with the compositions of the invention may be selected from the group: a saponin, lipid A or a derivative thereof, an immunostimulatory oligonucleotide, an alkyl glucosaminide phosphate, an oil in water emulsion or combinations thereof. A further preferred adjuvant is a metal salt in combination with another adjuvant. It is preferred that the adjuvant is a Toll like receptor agonist in particular an agonist of a Toll like receptor 2, 3, 4, 7, 8 or 9, or a saponin, in particular Qs21. It is further preferred that the adjuvant system comprises two or more adjuvants from the above list. In particular the combinations preferably contain a saponin (in particular Qs21) adjuvant and/or a Toll like receptor 9 agonist such as a CpG containing immunostimulatory oligonucleotide. Other preferred combinations comprise a saponin (in particular QS21) and a Toll like receptor 4 agonist such as monophosphoryl lipid A or its 3 deacylated derivative, 3 D—MPL, or a saponin (in particular QS21) and a Toll like receptor 4 ligand such as an alkyl glucosaminide phosphate.

Particularly preferred adjuvants are combinations of 3D-MPL™ and QS21 (EP 0 671 948 B1), oil in water emulsions comprising 3D-MPL™ and QS21 (WO 95/17210, WO 98/56414), or 3D-MPL™ formulated with other carriers (EP 0 689 454 B1). Other preferred adjuvant systems comprise a combination of 3D-MPL™, Q521 and a CpG oligonucleotide as described in U.S. Pat. No. 6,558,670, U.S. Pat. No. 6,544,518.

In an embodiment the adjuvant is a Toll like receptor (TLR) 4 ligand, preferably an agonist such as a lipid A derivative particularly monophosphoryl lipid A or more particularly 3 Deacylated monophoshoryl lipid A (3 D—MPL).

3D-MPL™ is available from GlaxoSmithKline Biologicals North America and primarily promotes CD4+ T cell responses with an IFN-g (Th1) phenotype. It can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3D-MPL™ is used. Small particle 3D-MPL™ has a particle size such that it may be sterile-filtered through a 0.22 μm filter. Such preparations are described in International Patent Application No. WO 94/21292.

Synthetic derivatives of lipid A are known and thought to be TLR 4 agonists including, but not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)

OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO99/64301 and WO 00/0462)

OM 197 MP-Ac DP (3S-, 9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which may be used are alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants.

Another preferred immunostimulant for use in the present invention is Quil A and its derivatives. Quil A is a saponin preparation isolated from the South American tree Quilaja Saponaria Molina and was first described as having adjuvant activity by Dalsgaard et al. in 1974 (“Saponin adjuvants”, Archiv. für die gesamte Virusforschung, Vol. 44, Springer Verlag, Berlin, p243-254). Purified fragments of Quil A have been isolated by HPLC which retain adjuvant activity without the toxicity associated with Quil A (EP 0 362 278), for example QS7 and QS21 (also known as QA7 and QA21). QS-21 is a natural saponin derived from the bark of Quillaja saponaria Molina which induces CD8+ cytotoxic T cells (CTLs), Th1 cells and a predominant IgG2a antibody response and is a preferred saponin in the context of the present invention.

Particular formulations of QS21 have been described which are particularly preferred, these formulations further comprise a sterol (WO96/33739). The saponins forming part of the present invention may be separate in the form of micelles, mixed micelles (preferentially, but not exclusively with bile salts) or may be in the form of ISCOM matrices (EP 0 109 942 B1), liposomes or related colloidal structures such as worm-like or ring-like multimeric complexes or lipidic/layered structures and lamellae when formulated with cholesterol and lipid, or in the form of an oil in water emulsion (for example as in WO 95/17210). The saponins may preferably be associated with a metallic salt, such as aluminium hydroxide or aluminium phosphate (WO 98/15287).

Preferably, the saponin is presented in the form of a liposome, ISCOM or an oil in water emulsion.

An enhanced system involves the combination of a monophosphoryl lipid A (or detoxified lipid A) and a saponin derivative, particularly the combination of QS21 and 3D-MPL™ as disclosed in WO 94/00153, or a less reactogenic composition where the QS21 is quenched with cholesterol as disclosed in WO 96/33739. A particularly potent adjuvant formulation involving tocopherol with or without QS21 and/or 30-MPL™ in an oil in water emulsion is described in WO 95/17210. In one embodiment the immunogenic composition additionally comprises a saponin, which may be QS21.

Immunostimulatory oligonucleotides or any other Toll-like receptor (TLR) 9 agonist may also be used. The preferred oligonucleotides for use in adjuvants or vaccines of the present invention are CpG containing oligonucleotides, preferably containing two or more dinucleotide CpG motifs separated by at least three, more preferably at least six or more nucleotides. A CpG motif is a Cytosine nucleotide followed by a Guanine nucleotide. The CpG oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204.

Examples of preferred oligonucleotides have the following sequences. The sequences preferably contain phosphorothioate modified internucleotide linkages.

OLIGO 1(SEQ ID NO: 111):
TCC ATG ACG TTC CTG ACG TT (CpG 1826)
OLIGO 2(SEQ ID NO: 112):
TCT CCC AGC GTG CGC CAT (CpG 1758)
OLIGO 3(SEQ ID NO: 113):
ACC GAT GAC GTC GCC GGT GAC GGC ACC ACG
OLIGO 4(SEQ ID NO: 114):
TCG TCG TTT TGT CGT TTT GTC GTT (CpG 2006)
OLIGO 5(SEQ ID NO: 115):
TCC ATG ACG TTC CTG ATG CT (CpG 1668)
OLIGO 6(SEQ ID NO: 116):
TCG ACG TTT TCG GCG CGC GCC G (CpG 5456)

Alternative CpG oligonucleotides may comprise the preferred sequences above in that they have inconsequential deletions or additions thereto.

The CpG oligonucleotides utilised in the present invention may be synthesized by any method known in the art (for example see EP 468520). Conveniently, such oligonucleotides may be synthesized utilising an automated synthesizer.

The adjuvant may be an oil in water emulsion or may comprise an oil in water emulsion in combination with other adjuvants. The oil phase of the emulsion system preferably comprises a metabolisable oil. The meaning of the term metabolisable oil is well known in the art. Metabolisable can be defined as “being capable of being transformed by metabolism” (Dorland's Illustrated Medical Dictionary, W.B. Sanders Company, 25^{th} edition (1974)). The oil may be any vegetable oil, fish, oil, animal or synthetic oil, which is not toxic to the recipient and is capable of being transformed by metabolism. Nuts, seeds, and grains are common sources of vegetable oils. Synthetic oils are also part of this invention and can include commercially available oils such as NEOBEE® and others. Squalene (2,6,10,15,19, 23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in this invention. Squalene is a metabolisable oil by virtue of the fact that it is an intermediate in the biosynthesis of cholesterol (Merck index, 10^{th} Edition, entry no.8619).

Tocols (e.g. vitamin E) are also often used in oil emulsions adjuvants (EP 0 382 271 B1; U.S. Pat. No. 5,667,784; WO 95/17210). Tocols used in the oil emulsions (preferably oil in water emulsions) of the invention may be formulated as described in EP 0 382 271 B1, in that the tocols may be dispersions of tocol droplets, optionally comprising an emulsifier, of preferably less than 1 micron in diameter. Alternatively, the tocols may be used in combination with another oil, to form the oil phase of an oil emulsion. Examples of oil emulsions which may be used in combination with the tocol are described herein, such as the metabolisable oils described above.

Oil in water emulsion adjuvants per se have been suggested to be useful as adjuvant compositions (EP 0 399 843B), also combinations of oil in water emulsions and other active agents have been described as adjuvants for vaccines (WO 95/17210; WO 98/56414; WO 99/12565; WO 99/11241). Other oil emulsion adjuvants have been described, such as water in oil emulsions (U.S. Pat. No. 5,422,109; EP 0 480 982 B2) and water in oil in water emulsions (U.S. Pat. No. 5,424,067; EP 0 480 981 B). All of which form preferred oil emulsion systems (in particular when incorporating tocols) to form adjuvants and compositions of the present invention.

Most preferably the oil emulsion (for instance oil in water emulsions) further comprises an emulsifier such as TWEEN™ 80 and/or a sterol such as cholesterol.

A preferred oil emulsion (preferably oil-in-water emulsion) comprises a metabolisible, non-toxic oil, such as squalane, squalene or a tocopherol such as alpha tocopherol (and preferably both squalene and alpha tocopherol) and optionally an emulsifier (or surfactant) such as TWEEN™ 80. A sterol (preferably cholesterol) may also be included. The method of producing oil in water emulsions is well known to the man skilled in the art. Commonly, the method comprises mixing the tocol-containing oil phase with a surfactant such as a PBS/TWEEN™80 solution, followed by homogenisation using a homogenizer, it would be clear to a man skilled in the art that a method comprising passing the mixture twice through a syringe needle would be suitable for homogenising small volumes of liquid. Equally, the emulsification process in MICROFLUIDISER® (M110S Microfluidics machine, maximum of 50 passes, for a period of 2 minutes at maximum pressure input of 6 bar (output pressure of about 850 bar)) could be adapted by the man skilled in the art to produce smaller or larger volumes of emulsion. The adaptation could be achieved by routine experimentation comprising the measurement of the resultant emulsion until a preparation was achieved with oil droplets of the required diameter.

In an oil in water emulsion, the oil and emulsifier should be in an aqueous carrier. The aqueous carrier may be, for example, phosphate buffered saline.

The size of the oil droplets found within the stable oil in water emulsion are preferably less than 1 micron, may be in the range of substantially 30-600 nm, preferably substantially around 30-500 nm in diameter, and most preferably substantially 150-500 nm in diameter, and in particular about 150 nm in diameter as measured by photon correlation spectroscopy. In this regard, 80% of the oil droplets by number should be within the preferred ranges, more preferably more than 90% and most preferably more than 95% of the oil droplets by number are within the defined size ranges. The amounts of the components present in the oil emulsions of the present invention are conventionally in the range of from 0.5-20% or 2 to 10% oil (of the total dose volume), such as squalene; and when present, from 2 to 10% alpha tocopherol; and from 0.3 to 3% surfactant, such as polyoxyethylene sorbitan monooleate. Preferably the ratio of oil (preferably squalene): tocol (preferably α-tocopherol) is equal or less than 1 as this provides a more stable emulsion. An emulsifier, such as TWEEN™80 or SPAN™ 85 may also be present at a level of about 1%. In some cases it may be advantageous that the vaccines of the present invention will further contain a stabiliser.

Examples of preferred emulsion systems are described in WO 95/17210, WO 99/11241 and WO 99/12565 which disclose emulsion adjuvants based on squalene, α-tocopherol, and TWEEN™ 80, optionally formulated with the immunostimulants QS21 and/or 3D-MPL.

Thus in a particularly, preferred embodiment of the present invention, the adjuvant of the invention may additionally comprise further immunostimulants, such as LPS or derivatives thereof, and/or saponins. Examples of further immunostimulants are described herein and in “Vaccine Design—The Subunit and Adjuvant Approach” 1995, Pharmaceutical Biotechnology, Volume 6, Eds. Powell, M. F., and Newman, M.J., Plenum Press, New York and London, ISBN 0-306-44867-X.

In a preferred aspect the adjuvant and immunogenic compositions according to the invention comprise a saponin (preferably QS21) and/or an LPS derivative (preferably 3D-MPL) in an oil emulsion described above, optionally with a sterol (preferably cholesterol). Additionally the oil emulsion (preferably oil in water emulsion) may contain SPAN™ 85 and/or lecithin and/or tricaprylin. Adjuvants comprising an oil-in-water emulsion, a sterol and a saponin are described in WO 99/12565.

Typically for human administration the saponin (preferably QS21) and/or LPS derivative (preferably 3D-MPL™) will be present in a human dose of immunogenic composition in the range of 1 μg-200 μg, such as 10-100 μg, preferably 10 μg-50 μg per dose. Typically the oil emulsion (preferably oil in water emulsion) will comprise from 2 to 10% metabolisible oil. Preferably it will comprise from 2 to 10% squalene, from 2 to 10% alpha tocopherol and from 0.3 to 3% (preferably 0.4-2%) emulsifier (preferably TWEEN™80 [polyoxyethylene sorbitan monooleate]). Where both squalene and alpha tocopherol are present, preferably the ratio of squalene: alpha tocopherol is equal to or less than 1 as this provides a more stable emulsion. SPAN™85 (Sorbitan trioleate) may also be present at a level of 0.5 to 1% in the emulsions used in the invention. In some cases it may be advantageous that the immunogenic compositions and vaccines of the present invention will further contain a stabiliser, for example other emulsifiers/surfactants, including caprylic acid (THE MERCK INDEX® 10^{th} Edition, entry no. 1739), of which Tricaprylin is particularly preferred.

Where squalene and a saponin (preferably QS21) are included, it is of benefit to also include a sterol (preferably cholesterol) to the formulation as this allows a reduction in the total level of oil in the emulsion. This leads to a reduced cost of manufacture, improvement of the overall comfort of the vaccination, and also qualitative and quantitative improvements of the resultant immune responses, such as improved IFN-γ production. Accordingly, the adjuvant system of the present invention typically comprises a ratio of metabolisable oil:saponin (w/w) in the range of 200:1 to 300:1, also the present invention can be used in a “low oil” form the preferred range of which is 1:1 to 200:1, preferably 20:1 to 100:1, and most preferably substantially 48:1, this vaccine retains the beneficial adjuvant properties of all of the components, with a much reduced reactogenicity profile. Accordingly, the particularly preferred embodiments have a ratio of squalene:QS21 (w/w) in the range of 1:1 to 250:1, also a preferred range is 20:1 to 200:1, preferably 20:1 to 100:1, and most preferably substantially 48:1. Preferably a sterol (most preferably cholesterol) is also included present at a ratio of saponin:sterol as described herein.

The emulsion systems of the present invention preferably have a small oil droplet size in the sub-micron range. Most preferably the oil droplet sizes will be in the range 120 to 750 nm, and most preferably from 120-600 nm in diameter.

A particularly potent adjuvant formulation (for ultimate combination with AlPO4 in the immunogenic compositions of the invention) involves a saponin (preferably QS21), an LPS derivative (preferably 3D-MPL) and an oil emulsion (preferably squalene and alpha tocopherol in an oil in water emulsion) as described in WO 95/17210 or in WO 99/12565 (in particular adjuvant formulation 11 in Example 2, Table 1).

Examples of a TLR 2 agonist include peptidoglycan or lipoprotein. Imidazoquinolines, such as Imiquimod and Resiquimod are known TLR7 agonists. Single stranded RNA is also a known TLR agonist (TLR8 in humans and TLR7 in mice), whereas double stranded RNA and poly IC (polyinosinic-polycytidylic acid—a commercial synthetic mimetic of viral RNA). are exemplary of TLR 3 agonists. 3D-MPL™ is an example of a TLR4 agonist whilst CPG is an example of a TLR9 agonist.

The immunogenic composition may comprise an antigen and an immunostimulant adsorbed onto a metal salt. Aluminium based vaccine formulations wherein the antigen and the immunostimulant 3-de-O-acylated monophosphoryl lipid A (3D-MPL), are adsorbed onto the same particle are described in EP 0 576 478 B1, EP 0 689 454 B1, and EP 0 633 784 B1. In these cases then antigen is first adsorbed onto the aluminium salt followed by the adsorption of the immunostimulant 3D-MPL™ onto the same aluminium salt particles. Such processes first involve the suspension of 3D-MPL™ by sonication in a water bath until the particles reach a size of between 80 and 500 nm. The antigen is typically adsorbed onto aluminium salt for one hour at room temperature under agitation. The 3D-MPL™ suspension is then added to the adsorbed antigen and the formulation is incubated at room temperature for 1 hour, and then kept at 4° C. until use.

In another process, the immunostimulant and the antigen are on separate metal particles, as described in EP 1126876. The improved process comprises the adsorption of immunostimulant, onto a metallic salt particle, followed by the adsorption of the antigen onto another metallic salt particle, followed by the mixing of the discrete metallic particles to form a vaccine. The adjuvant for use in the present invention may be an adjuvant composition comprising an immunostimulant, adsorbed onto a metallic salt particle, characterised in that the metallic salt particle is substantially free of other antigen. Furthermore, vaccines are provided by the present invention and are characterised in that the immunostimulant is adsorbed onto particles of metallic salt which are substantially free from other antigen, and in that the particles of metallic salt which are adsorbed to the antigen are substantially free of other immunostimulant.

Accordingly, the present invention provides an adjuvant formulation comprising immunostimulant which has been adsorbed onto a particle of a metallic salt, characterised in the composition is substantially free of other antigen. Moreover, this adjuvant formulation can be an intermediate which, if such an adjuvant is used, is required for the manufacture of a vaccine. Accordingly there is provided a process for the manufacture of a vaccine comprising admixing an adjuvant composition which is one or more immunostimulants adsorbed onto a metal particle with an antigen. Preferably, the antigen has been pre-adsorbed onto a metallic salt. Said metallic salt may be identical or similar to the metallic salt which is adsorbed onto the immunostimulant. Preferably the metal salt is an aluminium salt, for example Aluminium phosphate or Aluminium hydroxide.

The present invention further provides for a vaccine composition comprising immunostimulant adsorbed onto a first particle of a metallic salt, and antigen adsorbed onto a metallic salt, characterised in that first and second particles of metallic salt are separate particles.

LPS or LOS derivatives or mutations or lipid A derivatives described herein are designed to be less toxic (e.g. 3D-MPL) than native lipopolysaccharides and are interchangeable equivalents with respect to any uses of these moieties described herein.

In one embodiment the adjuvant used for the compositions of the invention comprises a liposome carrier (made by known techniques from a phospholipids (such as dioleoyl phosphatidyl choline [DOPC]) and optionally a sterol [such as cholesterol]). Such liposome carriers may carry lipid A derivatives [such as 3D-MPL™—see above] and/or saponins (such as QS21—see above). In one embodiment the adjuvant comprises (per 0.5 mL dose) 0.1-10 mg, 0.2-7, 0.3-5, 0.4-2, or 0.5-1 mg (e.g. 0.4-0.6, 0.9-1.1, 0.5 or 1 mg) phospholipid (for instance DOPC), 0.025-2.5, 0.05-1.5, 0.075-0.75, 0.1-0.3, or 0.125-0.25 mg (e.g. 0.2-0.3, 0.1-0.15, 0.25 or 0.125 mg) sterol (for instance cholesterol), 5-60, 10-50, or 20-30 μg (e.g. 5-15, 40-50, 10, 20, 30, 40 or 50 μg) lipid A derivative (for instance 3D-MPL), and 5-60, 10-50, or 20-30 μg (e.g. 5-15, 40-50, 10, 20, 30, 40 or 50 μg) saponin (for instance QS21).

In one embodiment the adjuvant used for the compositions of the invention comprises an oil in water emulsion made from a metabolisable oil (such as squalene), an emulsifier (such as TWEEN™80) and optionally a tocol (such as alpha tocopherol). In one embodiment the adjuvant comprises (per 0.5 mL dose) 0.5-15, 1-13, 2-11, 4-8, or 5-6 mg (e.g. 2-3, 5-6, or 10-11 mg) metabolisable oil (such as squalene), 0.1-10, 0.3-8, 0.6-6, 0.9-5, 1-4, or 2-3 mg (e.g. 0.9-1.1, 2-3 or 4-5 mg) emulsifier (such as TWEEN™80) and optionally 0.5-20, 1-15, 2-12, 4-10, 5-7 mg (e.g. 11-13, 5-6, or 2-3 mg) tocol (such as alpha tocopherol).

This adjuvant may optionally further comprise 5-60, 10-50, or 20-30 μg (e.g. 5-15, 40-50, 10, 20, 30, 40 or 50 μg) lipid A derivative (for instance 3D-MPL).

This adjuvant may optionally contain 0.025-2.5, 0.05-1.5, 0.075-0.75, 0.1-0.3, or 0.125-0.25 mg (e.g. 0.2-0.3, 0.1-0.15, 0.25 or 0.125 mg) sterol (for instance cholesterol), 5-60, 10-50, or 20-30 μg (e.g. 5-15, 40-50, 10, 20, 30, 40 or 50 μg) lipid A derivative (for instance 3D-MPL), and 5-60, 10-50, or 20-30 μg (e.g. 5-15, 40-50, 10, 20, 30, 40 or 50 μg) saponin (for instance QS21).

In one embodiment the adjuvant used for the compositions of the invention comprises aluminium phosphate and a lipid A derivative (such as 3D-MPL™). This adjuvant may comprise (per 0.5 mL dose) 100-750, 200-500, or 300-400 μg Al as aluminium phosphate, and 5-60, 10-50, or 20-30 μg (e.g. 5-15, 40-50, 10, 20, 30, 40 or 50 μg) lipid A derivative (for instance 3D-MPL).

The vaccine preparations of the present invention may be used to protect or treat a mammal susceptible to infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. Intranasal administration of vaccines for the treatment of pneumonia or otitis media is preferred (as nasopharyngeal carriage of pneumococci can be more effectively prevented, thus attenuating infection at its earliest stage). Although the vaccine of the invention may be administered as a single dose, components thereof may also be co-administered together at the same time or at different times (for instance pneumococcal polysaccharides could be administered separately, at the same time or 1-2 weeks after the administration of any bacterial protein component of the vaccine for optimal coordination of the immune responses with respect to each other). For co-administration, the optional Th1 adjuvant may be present in any or all of the different administrations, for example, it may be present in combination with the bacterial protein component of the vaccine. In addition to a single route of administration, 2 different routes of administration may be used. For example, polysaccharides may be administered IM (or ID) and bacterial proteins may be administered IN (or ID). In addition, the vaccines of the invention may be administered IM for priming doses and IN for booster doses.

The amount of conjugate antigen in each vaccine dose is selected as an amount which induces an immunoprotective response without significant, adverse side effects in typical vaccines. Such amount will vary depending upon which specific immunogen is employed and how it is presented. Generally, it is expected that each dose will comprise 0.1-100 μg of polysaccharide, typically 0.1-50 μg, 0.1-10 μg 1-10 μg or 1-5 μg for polysaccharide conjugates.

The content of protein antigens in the vaccine will typically be in the range 1-100 μg, 5-50 μg or 5-25 μg. Following an initial vaccination, subjects may receive one or several booster immunizations adequately spaced.

Vaccine preparation is generally described in Vaccine Design (“The subunit and adjuvant approach” (eds Powell M.F. & Newman M.J.) (1995) Plenum Press New York). Encapsulation within liposomes is described by Fullerton, U.S. Pat. No. 4,235,877.

The vaccines of the present invention may be stored in solution or lyophilized. Optionally the solution is lyophilized in the presence of a sugar such as sucrose, trehalose or lactose. It is typical that they are lyophilized and extemporaneously reconstituted prior to use. Lyophilizing may result in a more stable composition (vaccine).

Methods

The invention also encompasses method of making the immunogenic compositions and vaccines of the invention.

In an embodiment, the process of the invention, is a method to make a vaccine comprising the steps of mixing antigens to make the immunogenic composition of the invention and adding a pharmaceutically acceptable excipient.

Methods of Treatment

The invention also encompasses method of treatment or staphylococcal infection, particularly hospital acquired nosocomial infections.

This immunogenic composition or vaccine of the invention is particularly advantageous to use in cases of elective surgery. Such patients will know the date of surgery in advance and could be inoculated in advance. Since it is not know whether the patient will be exposed to S. aureus or S. epidermidis infection, it is preferred to inoculate with a vaccine of the invention that protects against both, as described above. Typically adults over 16 awaiting elective surgery are treated with the immunogenic compositions and vaccines of the invention. Alternatively children aged 3-16 awaiting elective surgery are treated with the immunogenic compositions and vaccines of the invention.

It is also possible to inoculate health care workers with the vaccine of the invention.

The vaccine preparations of the present invention may be used to protect or treat a mammal susceptible to infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts.

The amount of antigen in each vaccine dose is selected as an amount which induces an immunoprotective response without significant, adverse side effects in typical vaccines. Such amount will vary depending upon which specific immunogen is employed and how it is presented. The protein content of the vaccine will typically be in the range 1-100 μg, 5-50 μg, typically in the range 10-25 μg. An optimal amount for a particular vaccine can be ascertained by standard studies involving observation of appropriate immune responses in subjects. Following an initial vaccination, subjects may receive one or several booster immunisations adequately spaced.

Although the vaccines of the present invention may be administered by any route, administration of the described vaccines into the skin (ID) forms one embodiment of the present invention. Human skin comprises an outer “horny” cuticle, called the stratum corneum, which overlays the epidermis. Underneath this epidermis is a layer called the dermis, which in turn overlays the subcutaneous tissue. Researchers have shown that injection of a vaccine into the skin, and in particular the dermis, stimulates an immune response, which may also be associated with a number of additional advantages. Intradermal vaccination with the vaccines described herein forms an optional feature of the present invention.

The conventional technique of intradermal injection, the “Mantoux procedure”, comprises steps of cleaning the skin, and then stretching with one hand, and with the bevel of a narrow gauge needle (26-31 gauge) facing upwards the needle is inserted at an angle of between 10-15°. Once the bevel of the needle is inserted, the barrel of the needle is lowered and further advanced whilst providing a slight pressure to elevate it under the skin. The liquid is then injected very slowly thereby forming a bleb or bump on the skin surface, followed by slow withdrawal of the needle.

More recently, devices that are specifically designed to administer liquid agents into or across the skin have been described, for example the devices described in WO 99/34850 and EP 1092444, also the jet injection devices described for example in WO 01/13977; U.S. Pat. No. 5,480,381, U.S. Pat. No. 5,599,302, U.S. Pat. No. 5,334,144, U.S. Pat. No. 5,993,412, U.S. Pat. No. 5,649,912, U.S. Pat. No. 5,569,189, U.S. Pat. No. 5,704,911, U.S. Pat. No. 5,383,851, U.S. Pat. No. 5,893,397, U.S. Pat. No. 5,466,220, U.S. Pat. No. 5,339,163, U.S. Pat. No. 5,312,335, U.S. Pat. No. 5,503,627, U.S. Pat. No. 5,064,413, U.S. Pat. No. 5,520,639, U.S. Pat. No. 4,596,556, U.S. Pat. No. 4,790,824, U.S. Pat. No. 4,941,880, U.S. Pat. No. 4,940,460, WO 97/37705 and WO 97/13537. Alternative methods of intradermal administration of the vaccine preparations may include conventional syringes and needles, or devices designed for ballistic delivery of solid vaccines (WO 99/27961), or transdermal patches (WO 97/48440; WO 98/28037); or applied to the surface of the skin (transdermal or transcutaneous delivery WO 98/20734; WO 98/28037).

When the vaccines of the present invention are to be administered to the skin, or more specifically into the dermis, the vaccine is in a low liquid volume, particularly a volume of between about 0.05 ml and 0.2 ml.

The content of antigens in the skin or intradermal vaccines of the present invention may be similar to conventional doses as found in intramuscular vaccines (see above). However, it is a feature of skin or intradermal vaccines that the formulations may be “low dose”. Accordingly the protein antigens in “low dose” vaccines are optionally present in as little as 0.1 to 10 μg, optionally 0.1 to 5 μg per dose; and the polysaccharide (optionally conjugated) antigens may be present in the range of 0.01-1 μg, and optionally between 0.01 to 0.5 μg of polysaccharide per dose.

As used herein, the term “intradermal delivery” means delivery of the vaccine to the region of the dermis in the skin. However, the vaccine will not necessarily be located exclusively in the dermis. The dermis is the layer in the skin located between about 1.0 and about 2.0 mm from the surface in human skin, but there is a certain amount of variation between individuals and in different parts of the body. In general, it can be expected to reach the dermis by going 1.5 mm below the surface of the skin. The dermis is located between the stratum corneum and the epidermis at the surface and the subcutaneous layer below. Depending on the mode of delivery, the vaccine may ultimately be located solely or primarily within the dermis, or it may ultimately be distributed within the epidermis and the dermis.

An embodiment of the invention is a method of preventing or treating staphylococcal infection or disease comprising the step of administering the immunogenic composition or vaccine of the invention to a patient in need thereof.

A further embodiment of the invention is a use of the immunogenic composition of the invention in the manufacture of a vaccine for treatment or prevention of staphylococcal infection or disease, optionally post-surgery staphylococcal infection.

The term ‘staphylococcal infection’ encompasses infection caused by S. aureus and/or S. epidermidis and other staphylococcal strains capable of causing infection in a mammalina, optionally human host.

The terms “comprising”, “comprise” and “comprises” herein are intended by the inventors to be optionally substitutable with the terms “consisting of”, “consist of” and “consists of”, respectively, in every instance.

All references or patent applications cited within this patent specification are incorporated by reference herein.

In order that this invention may be better understood, the following examples are set forth. These examples are for purposes of illustration only, and are not to be construed as limiting the scope of the invention in any manner.

### EXAMPLES

### Example 1 Construction of Plasmid to Express Recombinant Proteins

A: Cloning.

Appropriate restriction sites engineered into oligonucleotides specific for the staphylococcal gene permitted directional cloning of the PCR product into the E. coli expression plasmid pET24d or pQE-30 such that a protein could be expressed as a fusion protein containing a (His)6 affinity chromatography tag at the N- or C-terminus.

The primers used were:

Alpha toxin -
(SEQ ID NO: 117)
5′-CGCGGATCCGCAGATTCTGATATTAATATTAAAAC-3′
and
(SEQ ID NO: 118)
5′CCCAAGCTTTTAATTTGTCATTTCTTCTTTTTC-3′
EbpS -
(SEQ ID NO: 119)
5′-CGCGGATCCGCTGGGTCTAATAATTTTAAAGATG-3′
and
(SEQ ID NO: 120)
5′CCCAAGCTTTTATGGAATAACGATTTGTTG-3′
ClfA -
(SEQ ID NO: 121)
5′-CGCGGATCCAGTGAAAATAGTGTTACGCAATC-3′
and
(SEQ ID NO: 122)
5′CCCAAGCTTTTACTCTGGAATTGGTTCAATTTC-3′
FnbpA -
(SEQ ID NO: 123)
5′-CGCGGATCCACACAAACAACTGCAACTAACG-3′
and
(SEQ ID NO: 124)
5′CCCAAGCTTTTATGCTTTGTGATTCTTTTTCAAAC3′
Sbi -
(SEQ ID NO: 125)
5′-CGCGGATCCAACACGCAACAAACTTC-3′
and
(SEQ ID NO: 126)
5′GGAACTGCAGTTATTTCCAGAATGATAATAAATTAC-3′
SdrC -
(SEQ ID NO: 127)
5′-CGCGGATCCGCAGAACATACGAATGGAG-3′
and
(SEQ ID NO: 128)
5′CCCAAGCTTTTATGTTTCTTCTTCGTAGTAGC-3′
SdrG -
(SEQ ID NO: 129)
5′-CGCGGATCCGAGGAGAATTCAGTACAAG-3′
and
(SEQ ID NO: 130)
5′CCCAAGCTTTTATTCGTCATCATAGTATCCG-3′
Ebh -
(SEQ ID NO: 131)
5′-AAAAGTACTCACCACCACCACCACC-3′
and
(SEQ ID NO: 132)
5′AAAAGTACTCACTTGATTCATCGCTTCAG-3′
Aaa -
(SEQ ID NO: 133)
5′-GCGCGCCATGGCACAAGCTTCTACACAACATAC-3′
and
(SEQ ID NO: 134)
5′GCGCGCTCGAGATGGATGAATGCATAGCTAGA-3′
IsaA -
(SEQ ID NO: 135)
5′-GCATCCATGGCACCATCACCATCACCACGAAGTAAACGTTGATCAAG
C-3′
and
(SEQ ID NO: 136)
5′-AGCACTCGAGTTAGAATCCCCAAGCACCTAAACC-3′
HarA -
(SEQ ID NO: 137)
5′-GCACCCATGGCAGAAAATACAAATACTTC-3′
and
(SEQ ID NO: 138)
5′TTTTCTCGAGCATTTTAGATTGACTAAGTTG-3′
Autolysin glucosaminidase -
(SEQ ID NO: 139)
5′-CAAGTCCCATGGCTGAGACGACACAAGATCAAC-3′
and
(SEQ ID NO: 140)
5′-CAGTCTCGAGTTTTACAGCTGTTTTTGGTTG-3′
Autolysin amidase -
(SEQ ID NO: 141)
5′-AGCTCATATGGCTTATACTGTTACTAAACC-3′
and
(SEQ ID NO: 142)
5′GCGCCTCGAGTTTATATTGTGGGATGTCG-3′
IsdA -
(SEQ ID NO: 143)
5′-CAAGTCCCATGGCAACAGAAGCTACGAACGCAAC-3′
and
(SEQ ID NO: 144)
5′ACCAGTCTCGAGTAATTCTTTAGCTTTAGAGCTTG-3′
IsdB -
(SEQ ID NO: 145)
5′-TATTCTCGAGGCTTTGAGTGTGTCCATCATTTG-3′
and
(SEQ ID NO: 146)
5′GAAGCCATGGCAGCAGCTGAAGAAACAGGTGG-3′
MRPII -
(SEQ ID NO: 147)
5′-GATTACACCATGGTTAAACCTCAAGCGAAA-3′
and
(SEQ ID NO: 148)
5′AGGTGTCTCGAGTGCGATTGTAGCTTCATT-3′

The PCR products were first introduced into the pGEM®-T cloning vector (NOVAGEN™ using Top10 bacterial cells, according to the manufacturer's instructions. This intermediate construct was made to facilitate further cloning into an expression vector. Transformants containing the DNA insert were selected by restriction enzyme analysis. Following digestion, a ˜20 μl aliquot of the reaction was analyzed by agarose gel electrophoresis (0.8% agarose in a Tris-acetate-EDTA (TAE) buffer). DNA fragments were visualized by UV illumination after gel electrophoresis and ethidium bromide staining. A DNA molecular size standard (1 Kb ladder, Life Technologies) was electrophoresed in parallel with the test samples and was used to estimate the size of the DNA fragments. Plasmid purified from selected transformants for each cloning was then sequentially digested to completion with appropriate restriction enzymes as recommended by the manufacturer (Life Technologies). The digested DNA fragment was then purified using silica gel-based spin columns prior to ligation with the pET24d or pQE-30 μlasmid. Cloning of Ebh (H2 fragment), AaA, IsdA, IsdB, HarA, Atl-amidase, Atl-glucosamine, MRPII, IsaA was carried out using the pET24d plasmid and cloning of ClfA, SdrC, SdrE, FnbpA, SdrG/Fbe, alpha toxin and Sbi were carried out using the pQE-30 μlasmid.

B: Production of Expression Vector.

To prepare the expression plasmid pET24d or pQE-30 for ligation, it was similarly digested to completion with appropriate restriction enzymes. An approximately 5-fold molar excess of the digested fragments to the prepared vector was used to program the ligation reaction. A standard ˜20 μl ligation reaction (˜16° C., ˜16 hours), using methods well known in the art, was performed using T4 DNA ligase (˜2.0 units/reaction, Life Technologies). An aliquot of the ligation (˜5 μl) was used to transform M15(pREP4) or BT21::DE3 electro-competent cells according to methods well known in the art. Following a ˜2-3 hour outgrowth period at 37° C. in ˜1.0 ml of LB broth, transformed cells were plated on LB agar plates containing ampicillin (100 μg/ml) and/or kanamycin (30 μg/ml). Antibiotics were included in the selection. Plates were incubated overnight at 37° C. for ˜16 hours. Individual ApR/KanR colonies were picked with sterile toothpicks and used to “patch” inoculate fresh LB ApR/KanR plates as well as a ˜1.0 ml LB Ap/Kan broth culture. Both the patch plates and the broth culture were incubated overnight at 37° C. in either a standard incubator (plates) or a shaking water bath. A whole cell-based PCR analysis was employed to verify that transformants contained the DNA insert. Here, the ˜1.0 ml overnight LB Ap/Kan broth culture was transferred to a 1.5 ml polypropylene tube and the cells collected by centrifugation in a Beckmann microcentrifuge (˜3 min., room temperature, ˜12,000×g). The cell pellet was suspended in ˜200 μl of sterile water and a ˜10 μl aliquot used to program a ˜50 μl final volume PCR reaction containing both forward and reverse amplification primers. The initial 95° C. denaturation step was increased to 3 minutes to ensure thermal disruption of the bacterial cells and liberation of plasmid DNA. An ABI Model 9700 thermal cycler and a 32 cycle, three-step thermal amplification profile, i.e. 95° C., 45 sec; 55-58° C., 45 sec, 72° C., 1 min., were used to amplify the BASB203 fragment from the lysed transformant samples. Following thermal amplification, a ˜20 μl aliquot of the reaction was analyzed by agarose gel electrophoresis (0.8% agarose in a Tris-acetate-EDTA (TAE) buffer). DNA fragments were visualised by UV illumination after gel electrophoresis and ethidium bromide staining. A DNA molecular size standard (1 Kb ladder, Life Technologies) was electrophoresed in parallel with the test samples and was used to estimate the size of the PCR products. Transformants that produced the expected size PCR product were identified as strains containing a protein expression construct. Expression plasmid containing strains were then analyzed for the inducible expression of recombinant protein.

C: Expression Analysis of PCR-Positive Transformants.

An aliquot of the overnight seed culture (˜1.0 ml) was inoculated into a 125 ml Erlenmeyer flask containing −25 ml of LB Ap/Kan broth and was grown at 37° C. with shaking (˜250 rpm) until the culture turbidity reached O.D.600 of ˜0.5, i.e. mid-log phase (usually about 1.5-2.0 hours). At this time approximately half of the culture (˜12.5 ml) was transferred to a second 125 ml flask and expression of recombinant protein induced by the addition of IPTG (1.0 M stock prepared in sterile water, Sigma) to a final concentration of 1.0 mM. Incubation of both the IPTG-induced and non-induced cultures continued for an additional ˜4 hours at 37° C. with shaking. Samples (˜1.0 ml) of both induced and non-induced cultures were removed after the induction period and the cells collected by centrifugation in a microcentrifuge at room temperature for ˜3 minutes. Individual cell pellets were suspended in ˜50 μl of sterile water, then mixed with an equal volume of 2× Laemelli SDS-PAGE sample buffer containing 2-mercaptoethanol, and placed in boiling water bath for ˜3 min to denature protein. Equal volumes (˜15 μl) of both the crude IPTG-induced and the non-induced cell lysates were loaded onto duplicate 12% Tris/glycine polyacrylamide gel (1 mm thick Mini-gels, NOVEX™, Inc.). The induced and non-induced lysate samples were electrophoresed together with prestained molecular weight markers (SEEBLUE™, NOVEX™, Inc.) under conventional conditions using a standard SDS/Tris/glycine running buffer (BIO-RAD™ Laboratories, Inc.). Following electrophoresis, one gel was stained with COOMASSIE™ brilliant blue R250 (BIO-RAD™ Laboratories, Inc.) and then destained to visualize novel IPTG-inducible protein(s).The second gel was electroblotted onto a PVDF membrane (0.45 micron pore size, NOVEX™ Inc.) for ˜2 hrs at 4° C. using a BIO-RAD™ Laboratories, Inc. MINI-PROTEAN™ 11 blotting apparatus and Towbin's methanol (20%) transfer buffer. Blocking of the membrane and antibody incubations were performed according to methods well known in the art. A monoclonal anti-RGS (His)3 antibody, followed by a second rabbit anti-mouse antibody conjugated to HRP (QIAGEN™), were used to confirm the expression and identity of the recombinant protein. Visualization of the anti-His antibody reactive pattern was achieved using either an ABT insoluble substrate or using Hyperfilm with the Amersham ECL chemiluminescence system.

### Example 2: Production of Recombinant Protein

Bacterial Strain

A recombinant expression strain of E. coli M15(pREP4) containing a plasmid (pQE30) or BL21::DE3 containing plasmid pET24d encoding staphylococcal protein was used to produce cell mass for purification of recombinant protein.

Media

The fermentation medium used for the production of recombinant protein consisted of 2×YT broth (DIFCO™) containing 100 μg/ml Ap and/or 30 μg/ml Km. Antifoam was added to medium for the fermentor at 0.25 ml/L (Antifoam 204, Sigma). To induce expression of the recombinant protein, IPTG (Isopropyl R-D-Thiogalactopyranoside) was added to the fermentor (1 mM, final).

Production of Recombinant Proteins

Under Native Conditions

IPTG was added at a final concentration of 1 mM and the culture was grown for 4 additional hours. The culture was then centrifuged at 6,000 rpm for 10 minutes and the pellet was resuspended in phosphate buffer (50 mM K2HPO4, KH2PO4 pH 7) including a protease inhibitor cocktail. This sample was subjected to French pressure lysis using 1500 bar pressure (2 runs). After centrifugation for 30 minutes at 15,000 rpm, the supernatant was reserved for further purification and NaCl was added to 0.5M. The sample was then loaded on a Ni-NTA resin (XK 16 column Pharmacia, Ni-NTA resin QIAGEN™) conditioned in 50 mM K2HPO4, KH2PO4 pH 7. After loading the sample, the column was washed with Buffer A (0.2M NaH2PO4 pH7, 0.3M NaCl, 10% glycerol). To elute bound protein, a step gradient was used where different proportions of buffer B (0.2M NaH2PO4 pH7, 0.3M NaCl, 10% glycerol and 200 mM imidazole) were added to buffer A. The proportion of buffer B was gradually increased from 10% to 100%. After purification, eluted fraction containing the protein were pooled, concentrated and dialysed against 0.002M KH2PO4/K2HPO4 pH7, 0.15M NaCl.

This method was used to purify ClfA, SdrG, IsdA, IsaB, HarA, Atl-glucosamine and alpha toxin.

Under Denaturing Conditions

IPTG was added at a final concentration of 1 mM and the culture was grown for 4 additional hours. The culture was then centrifuged at 6,000 rpm for 10 minutes and the pellet was resuspended in phosphate buffer (50 mM K2HPO4, KH2PO4 pH 7) including a protease inhibitor cocktail. This sample was subjected to French pressure lysis using 1500 bar pressure (2 runs). After centrifugation for 30 minutes at 15,000 rpm, the pellet was washed with phosphate buffer including 1M urea. The sample was centrifuged for 30 mins at 15000 rpm and the pellet was resuspended in 8M urea, 0.1M NaH2PO4, 0.5M NaCl, 0.01 M Tris-Hcl pH8 and kept overnight at room temperature. The sample was centrifuged fro 20 minutes at 15000 rpm and the supernatant was collected for further purification. The sample was then loaded on a Ni-NTA resin (XK 16 column Pharmacia, Ni-NTA resin QIAGEN™) conditioned in 8M urea, 0.1M NaH2PO4, 0.5M NaCl, 0.01M Tris-Hcl pH8. After passage of the flowthrough, the column was washed successively with buffer A (8M Urea, 0.1MNaH2PO4, 0,5M NaCl, 0.01M Tris, pH 8.0), buffer C (8M Urea, 0.1MNaH2PO4, 0.5M NaCl, 0.01M Tris, pH 6.3), buffer D (8M Urea, 0.1MNaH2PO4, 0.5M NaCl, 0.01M Tris, pH 5.9) and buffer E (8M Urea, 0.1MNaH2PO4, 0.5M NaCl, 0.01M Tris, pH 4.5). The recombinant protein was eluted from the column during washes with buffer D and E. The denatured, recombinant protein could be solubilized in a solution devoid of urea. For this purpose, denatured protein contained in 8M urea was successively dialyzed against 4M urea, 0.1MNa2PO4, 0.01M Tris-HCl, pH7.1, 2M urea, 0.1 M NaH2PO4, 0.01M Tris-HCl, pH 7.1, 0.5M arginine and 0.002M KH2PO4/K2HPO4 pH7.1, 0.15M NaCl, 0.5M arginine.

This method was used to purify Ebh (H2 fragment), AaA, SdrC, FnbpA, Sbi, Atl-amidase and IsaA.

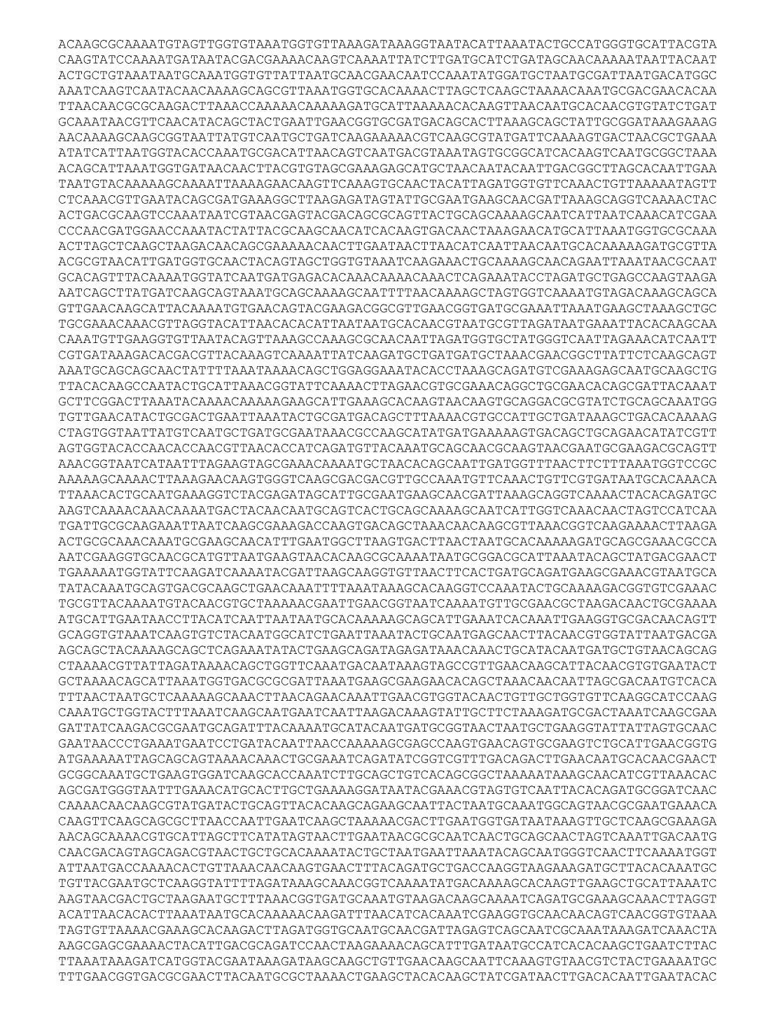

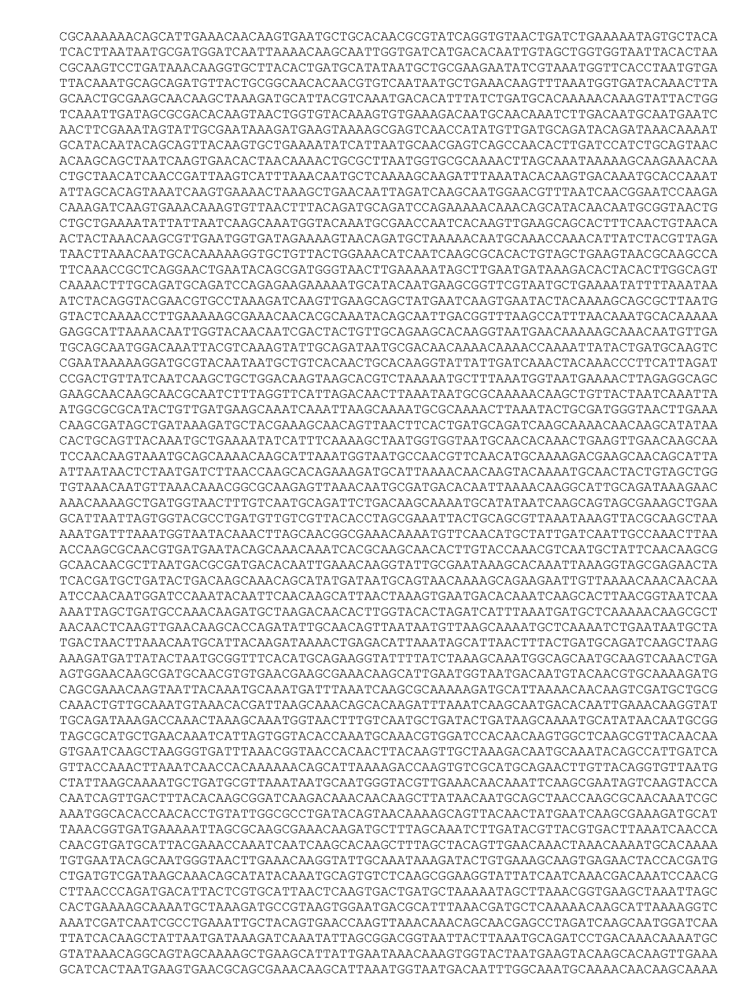

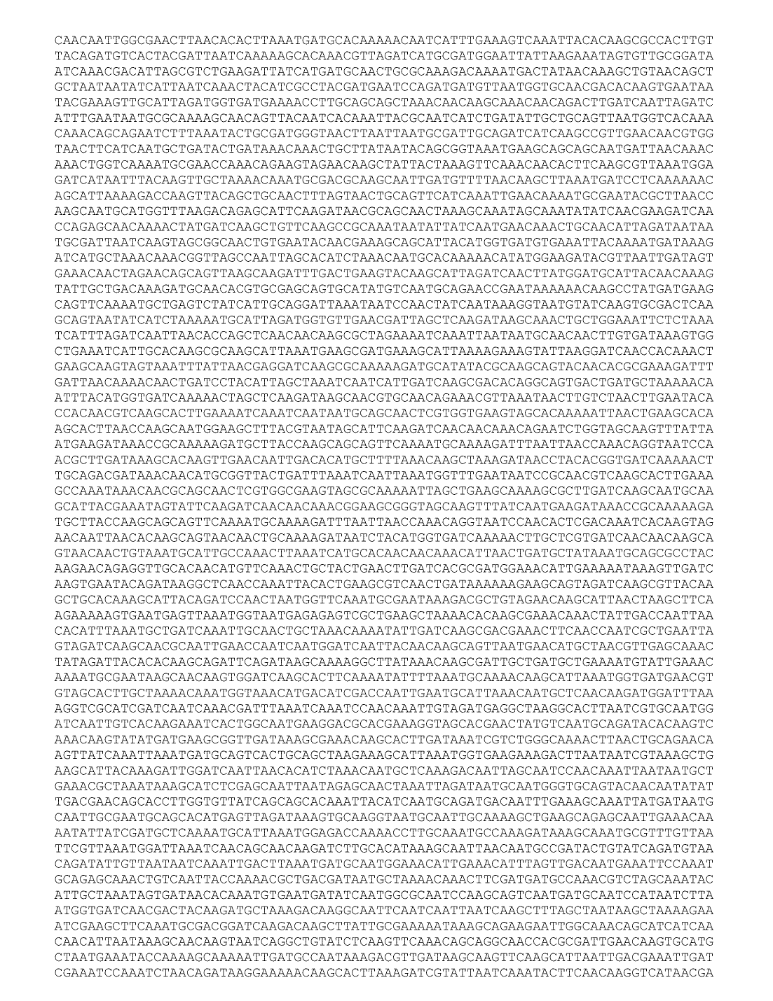

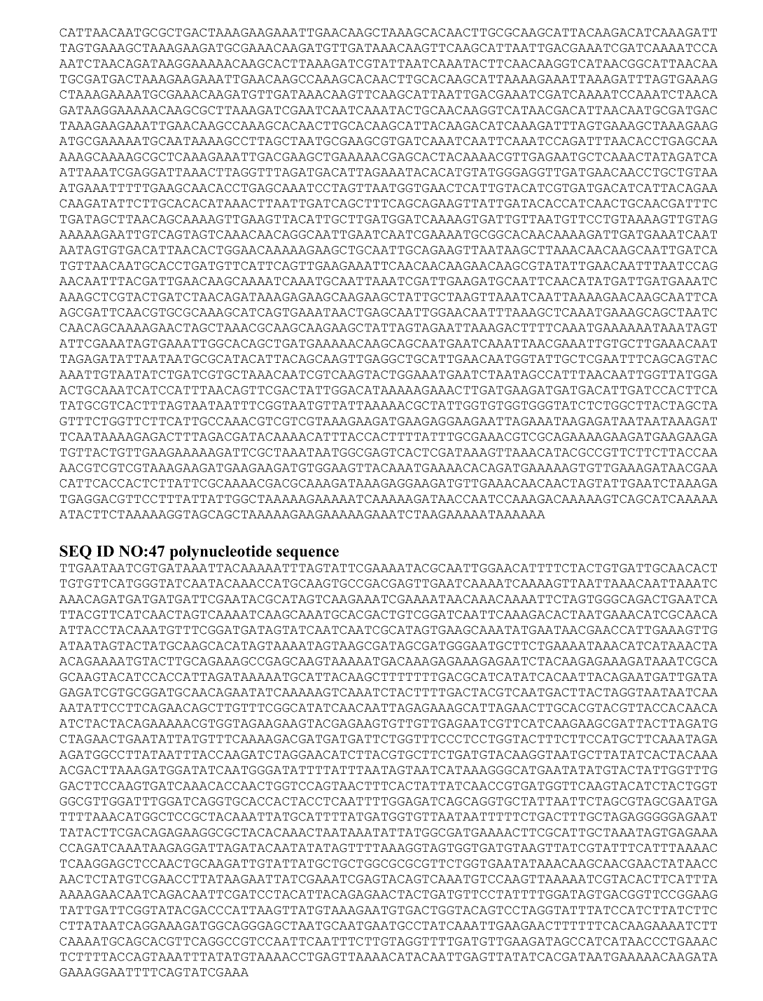

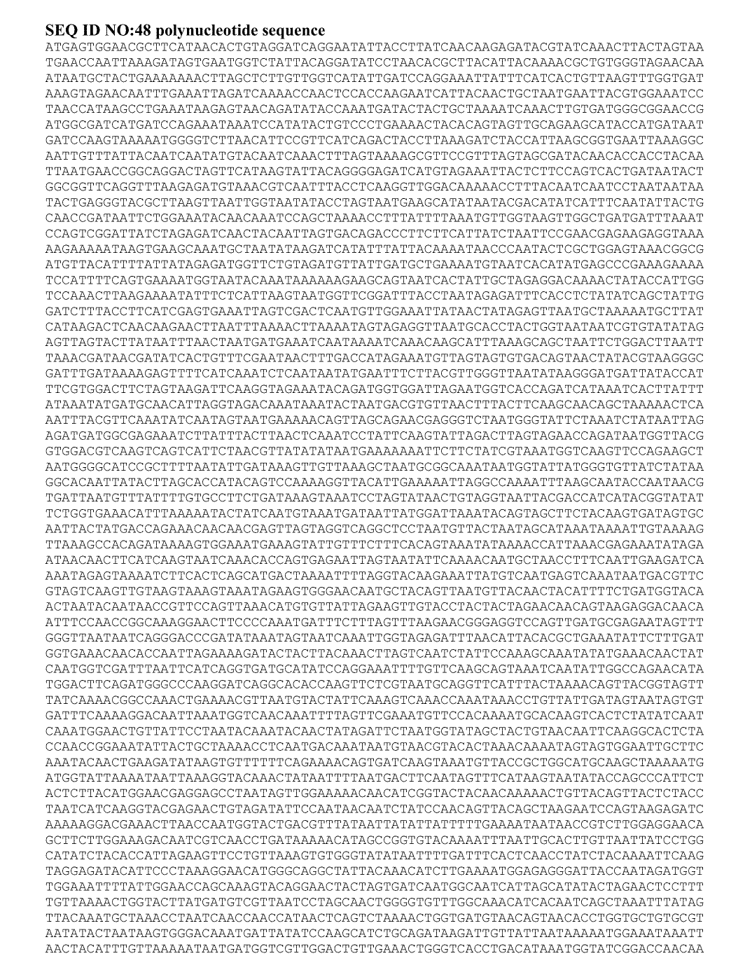

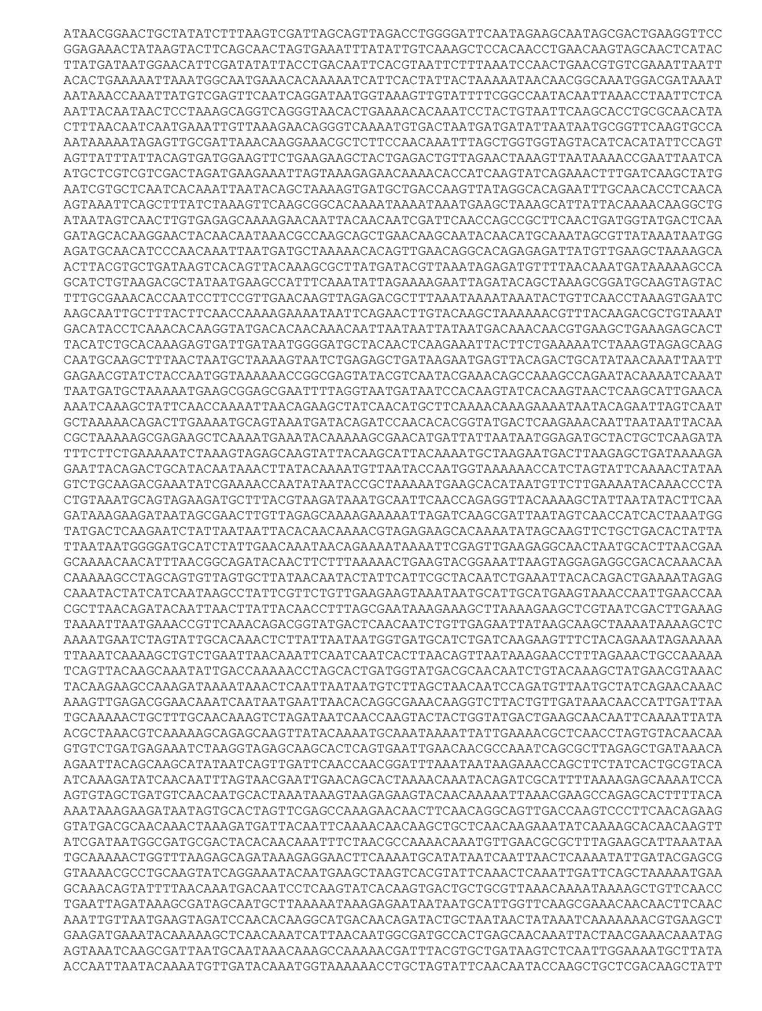

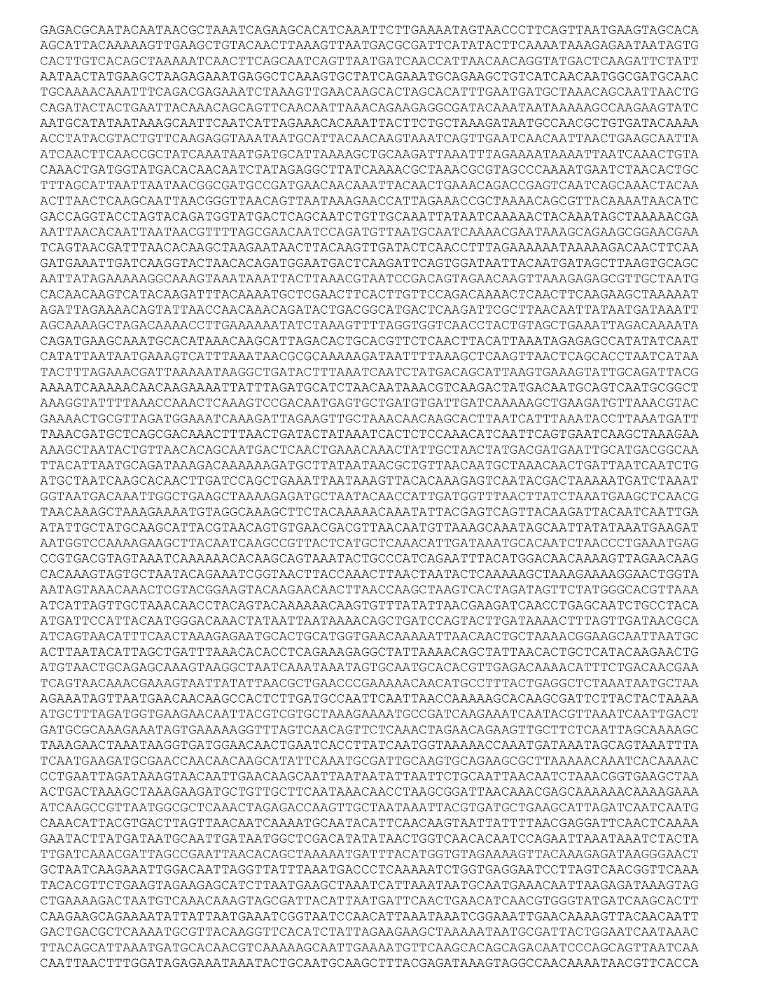

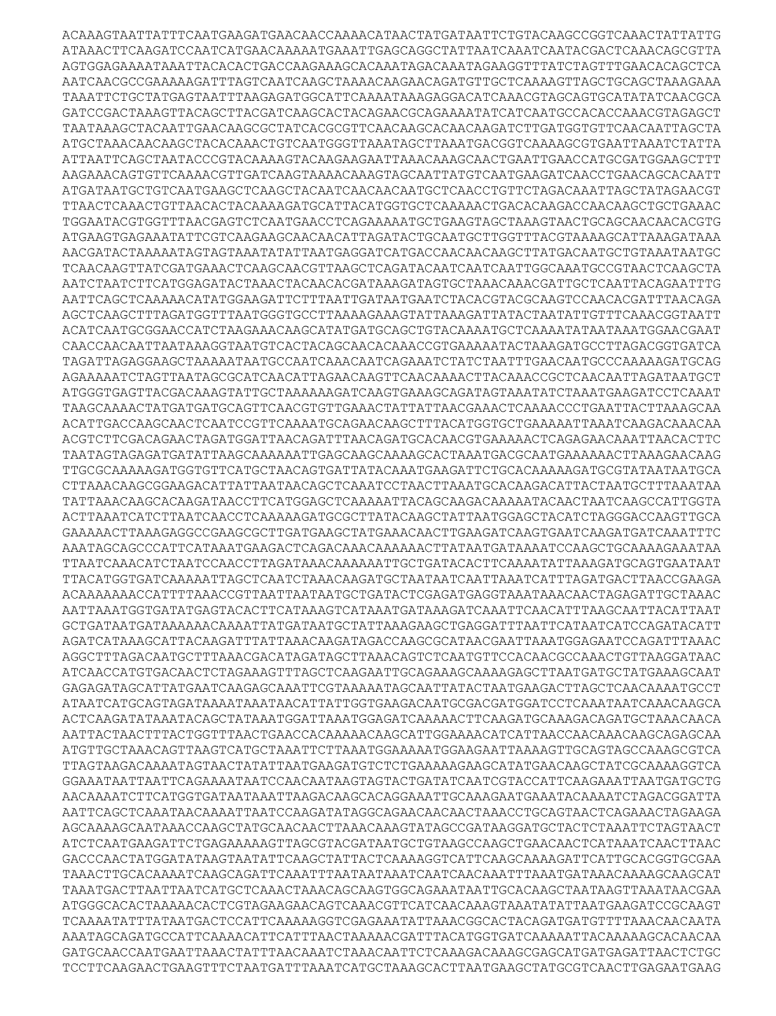

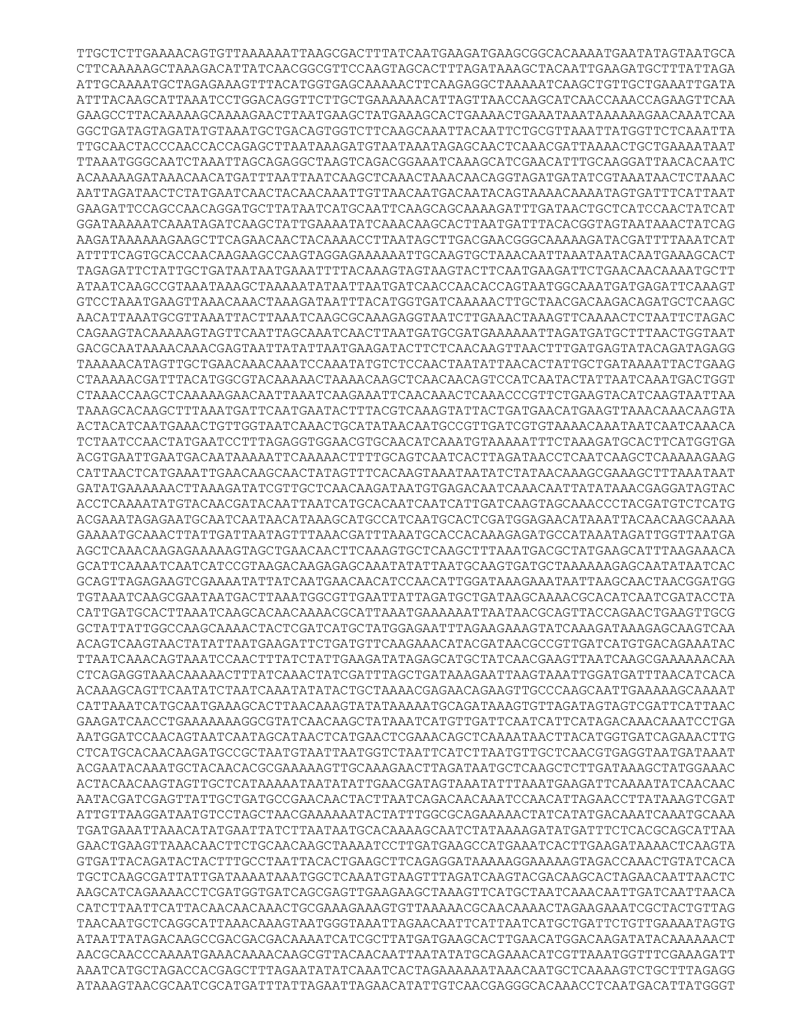

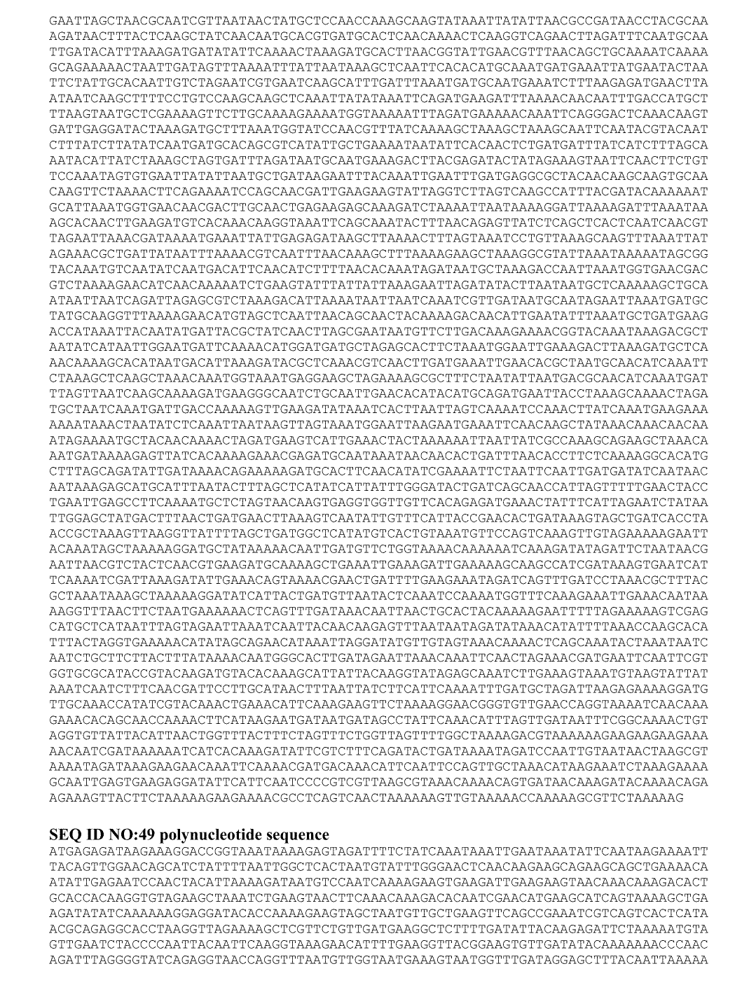

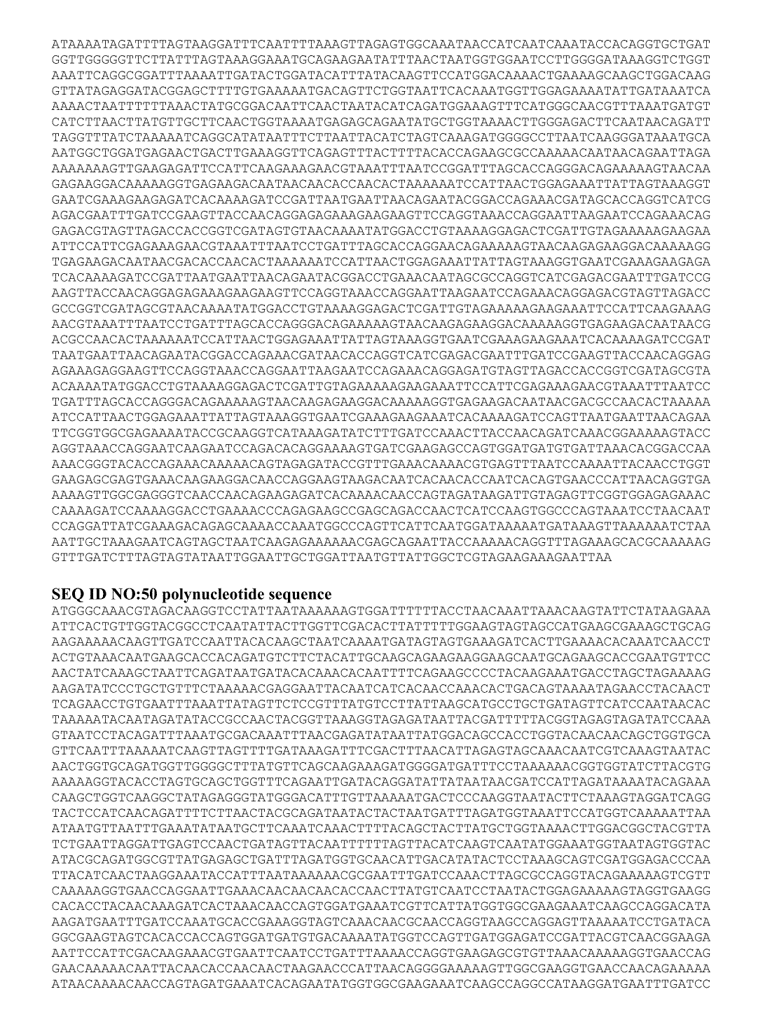

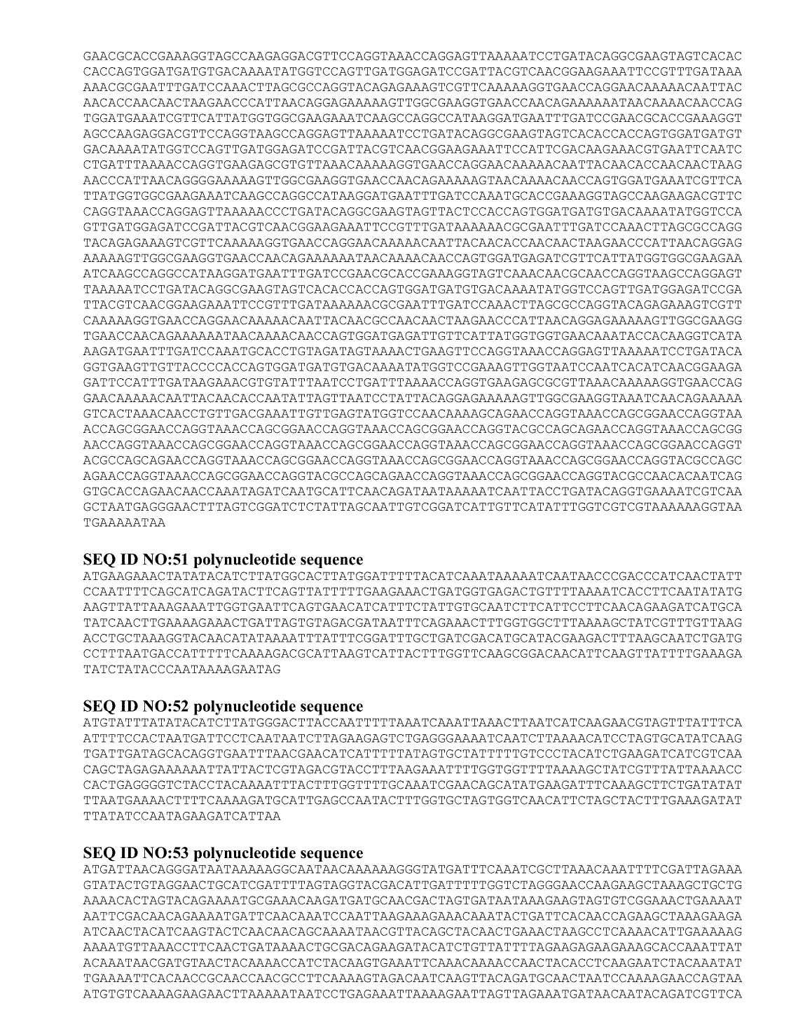

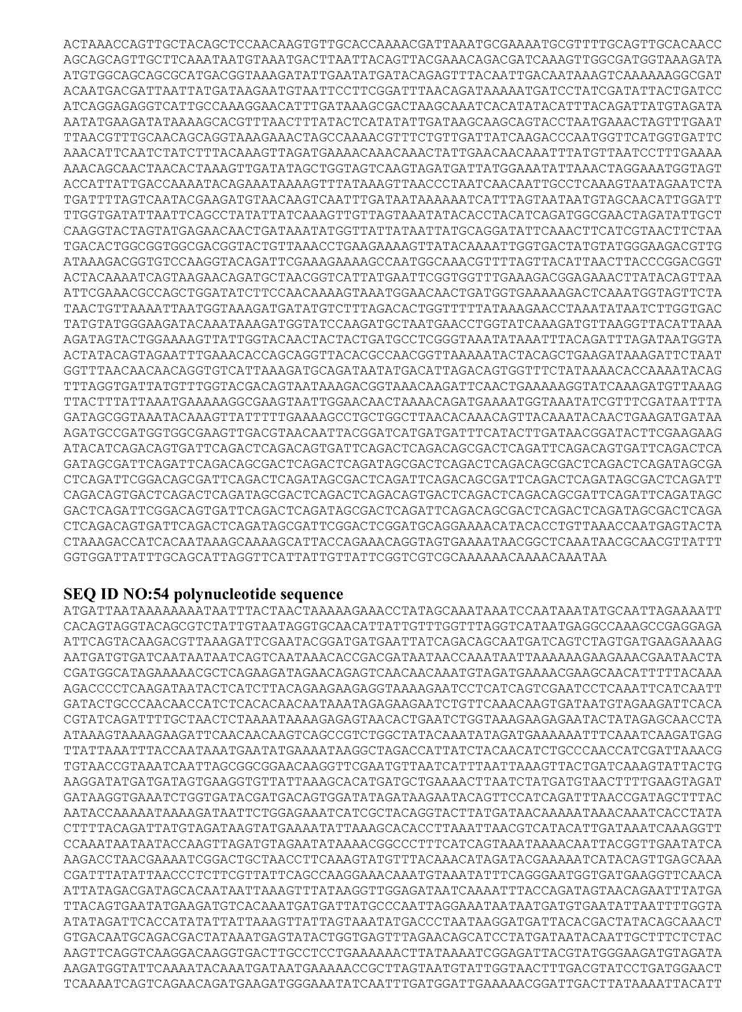

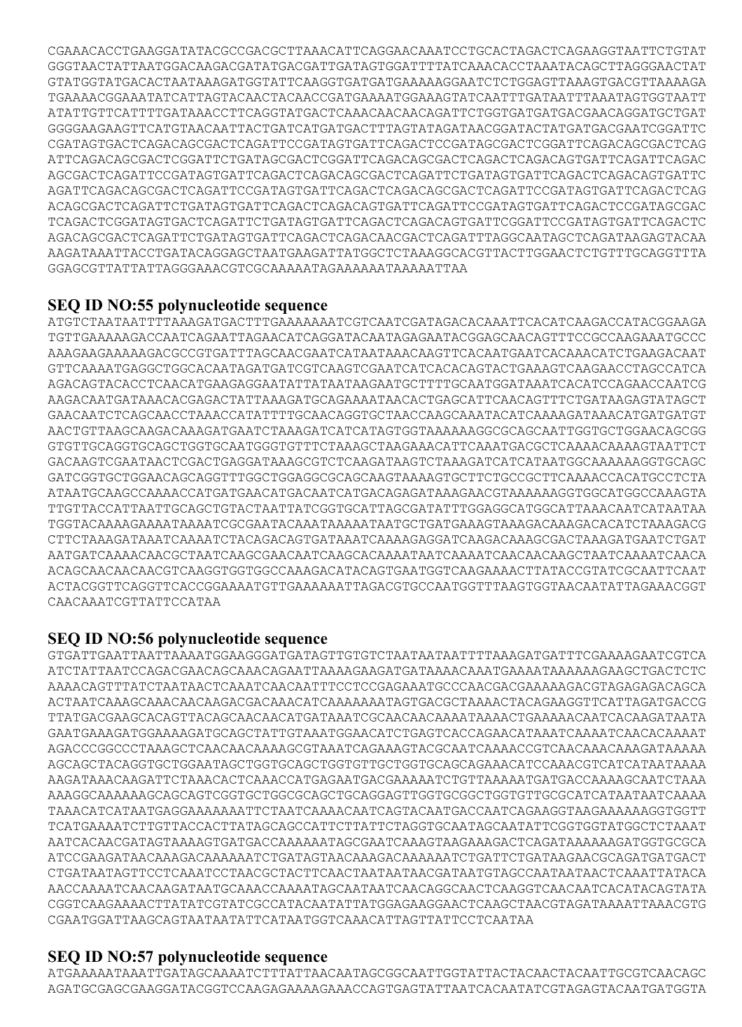

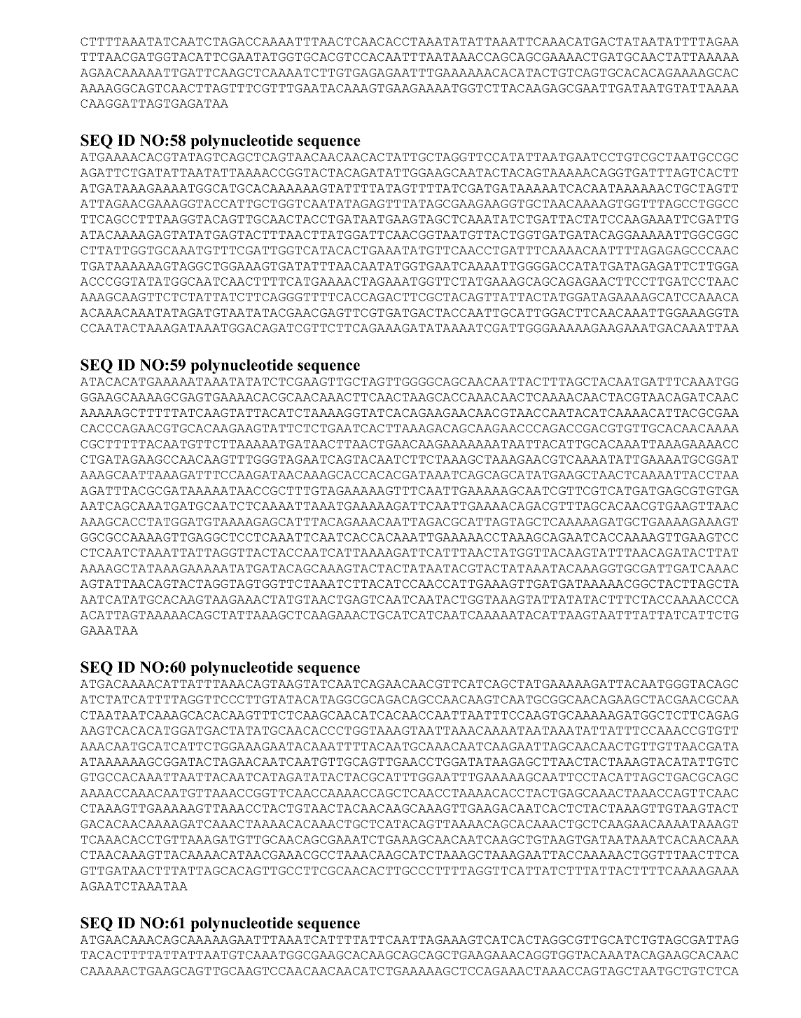

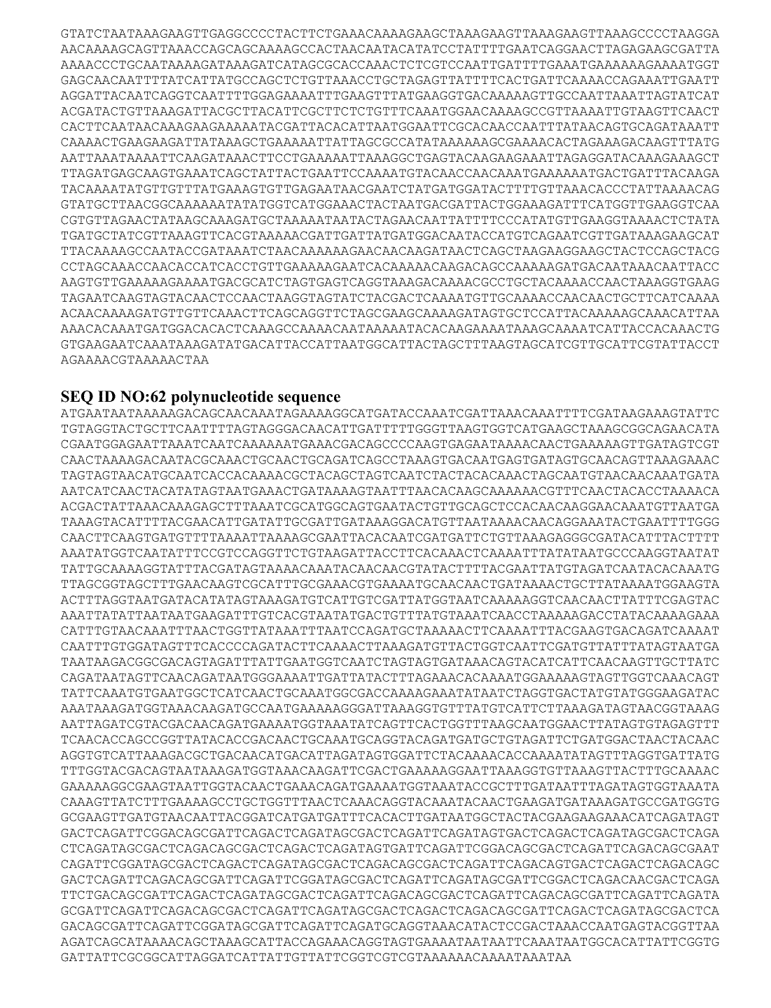

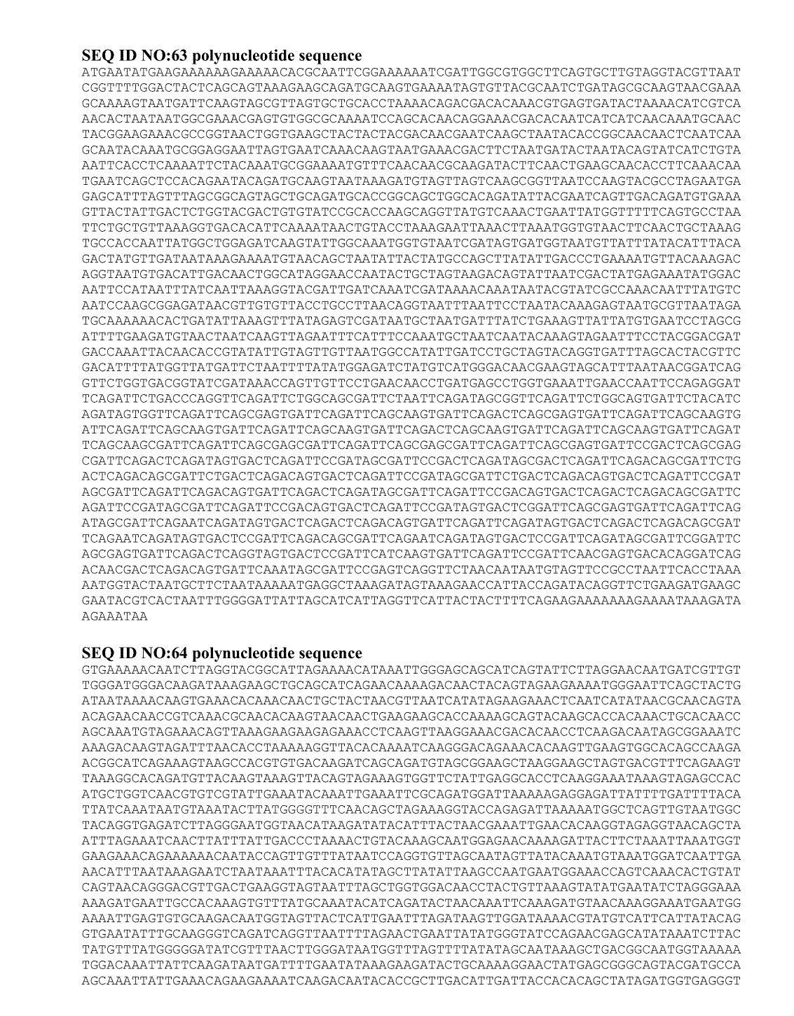

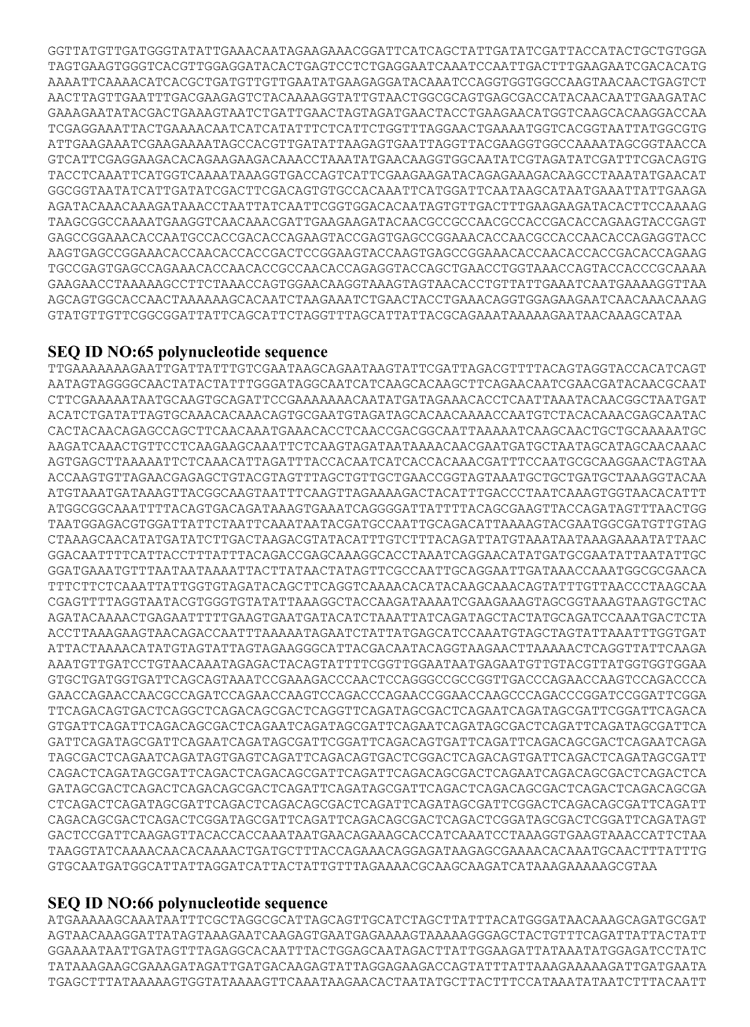

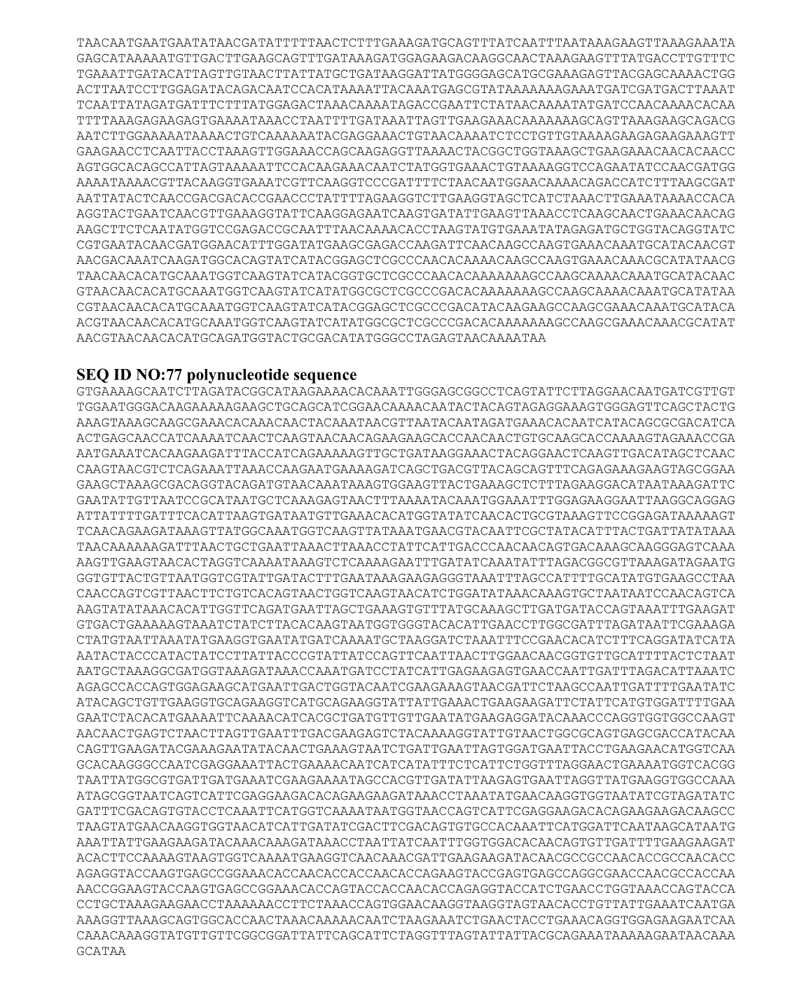

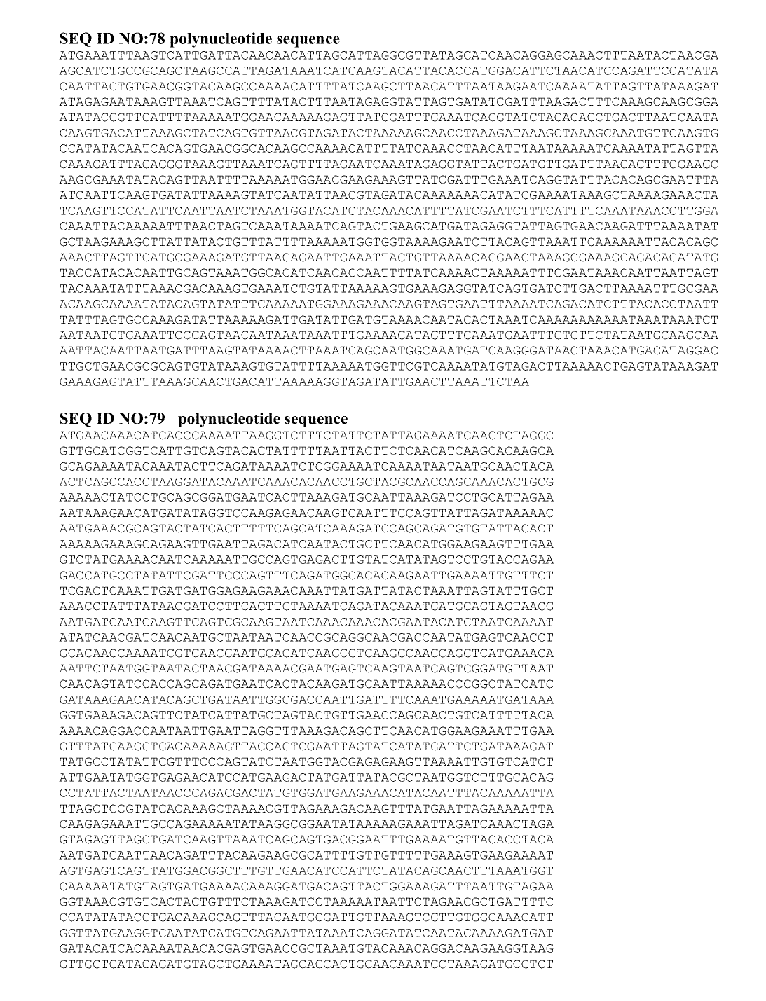

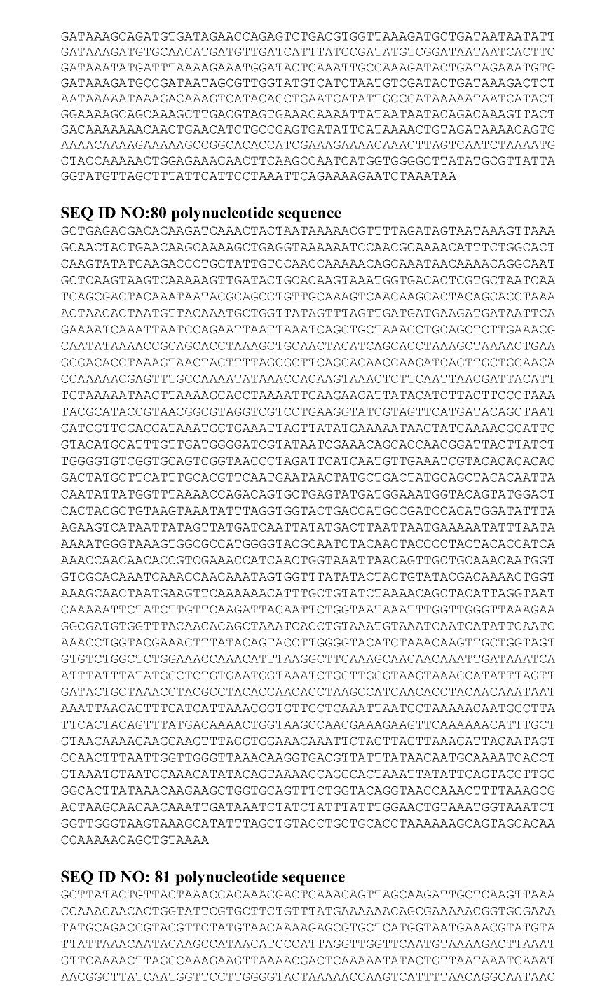

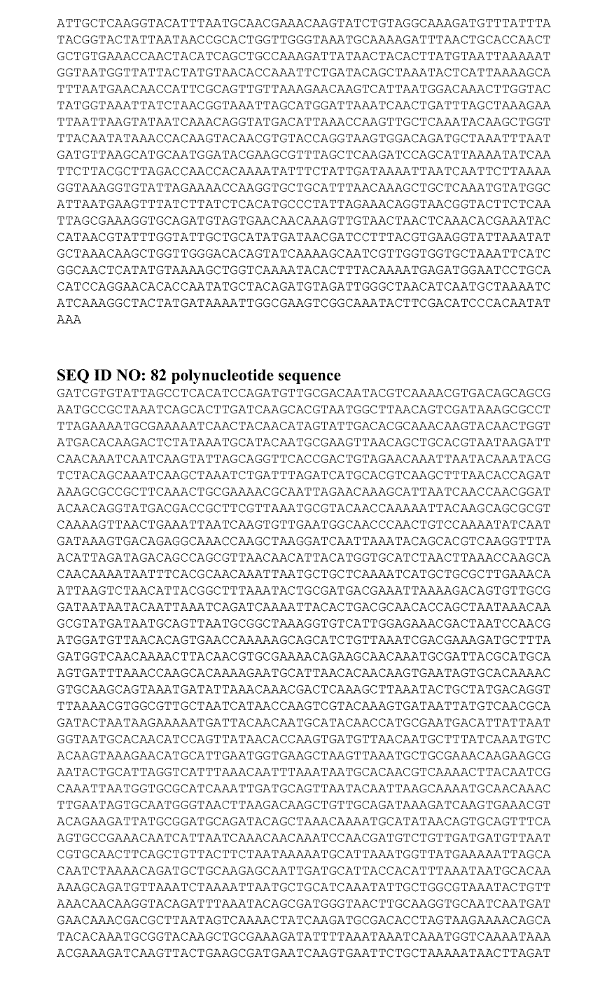

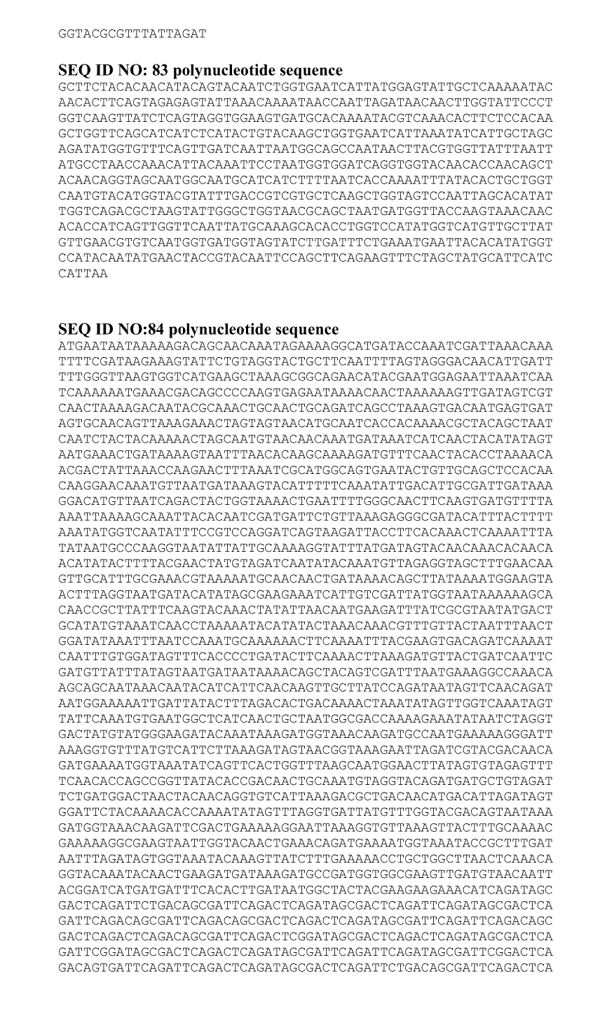

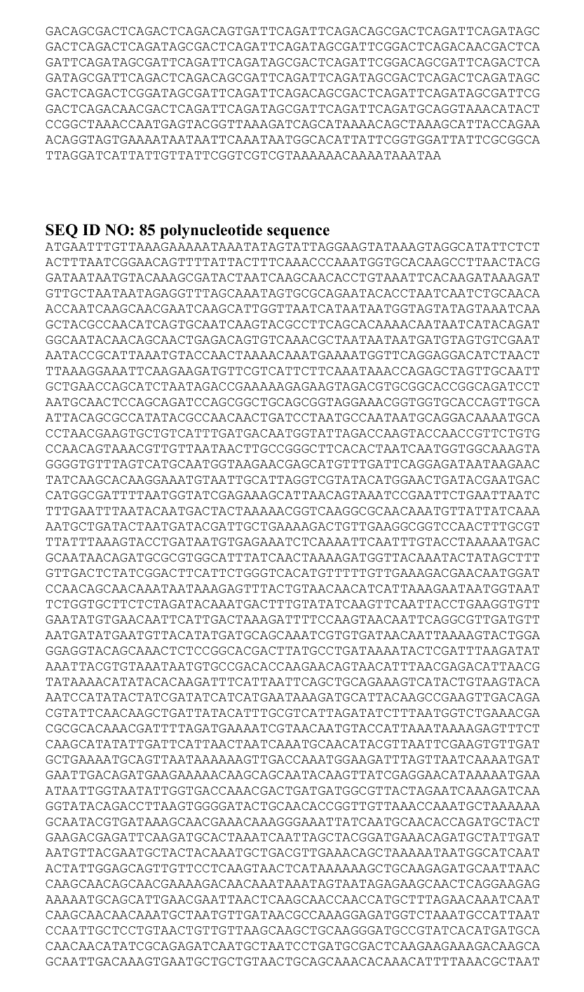

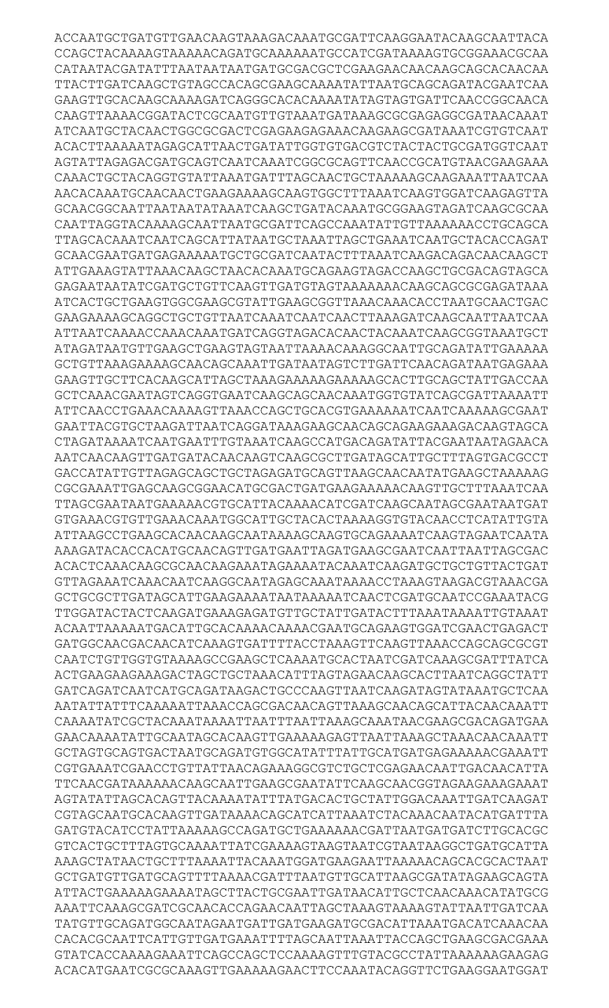

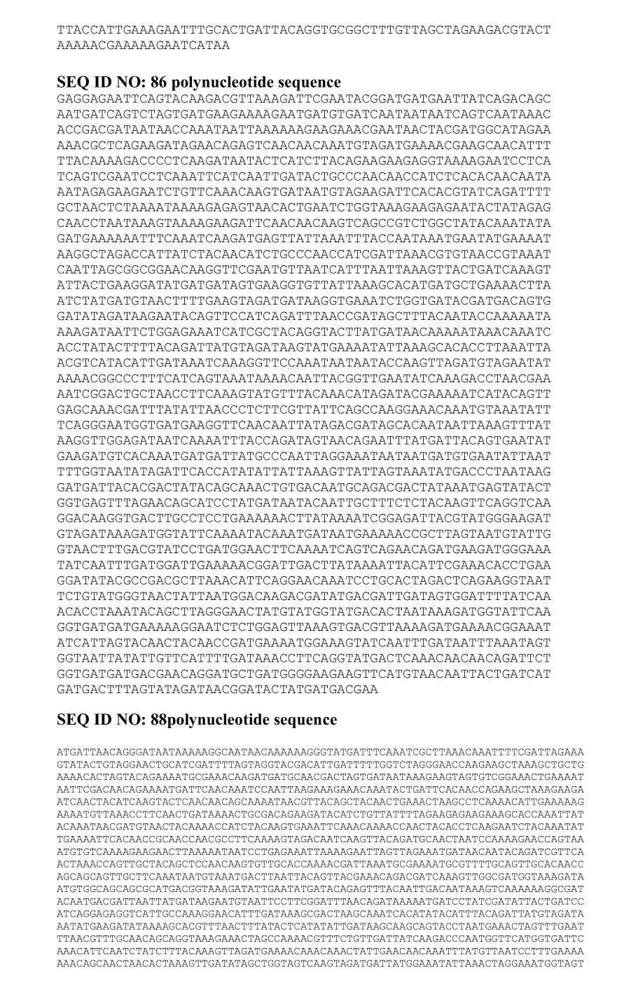

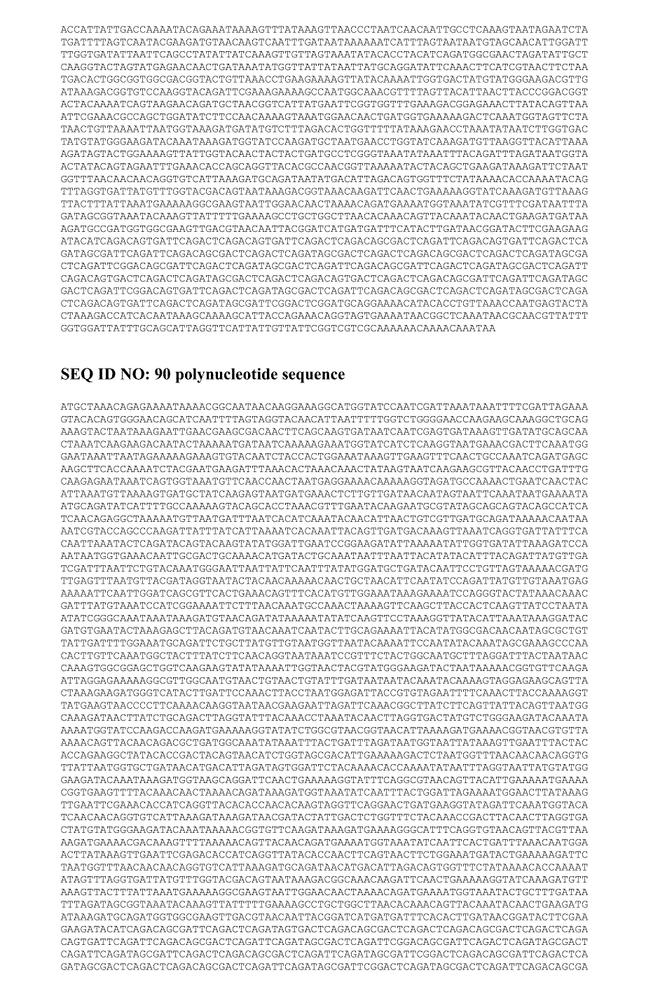

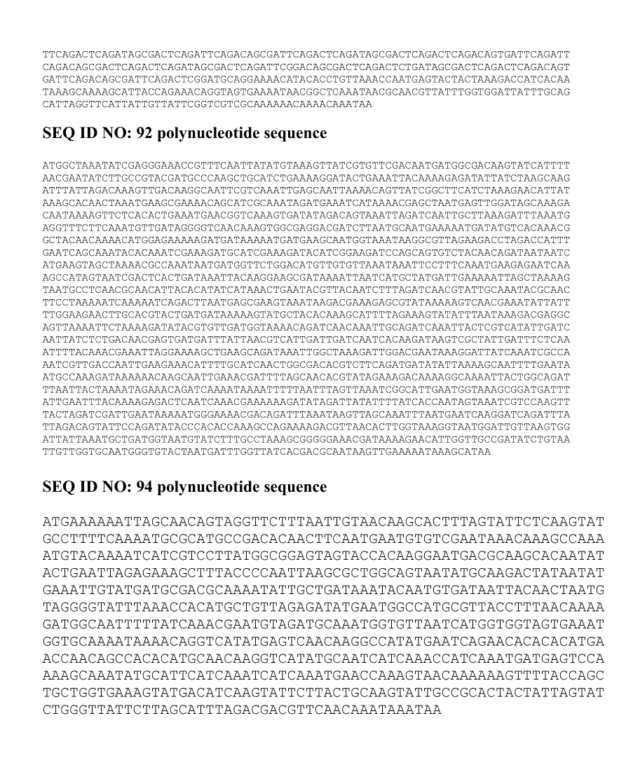

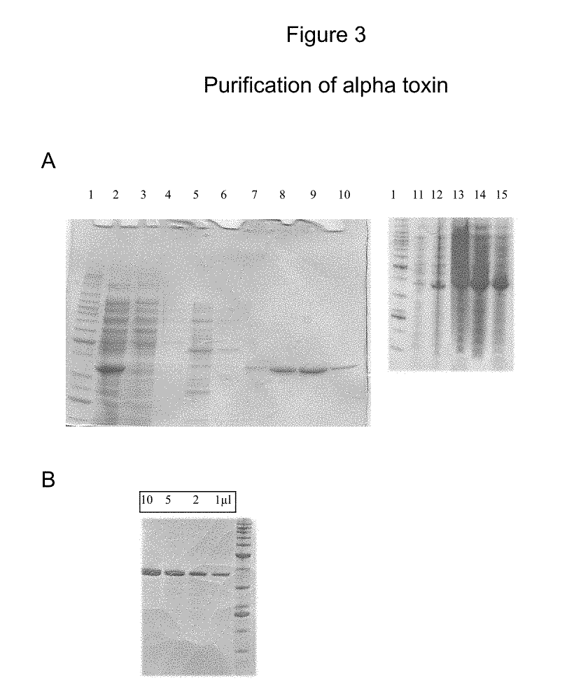

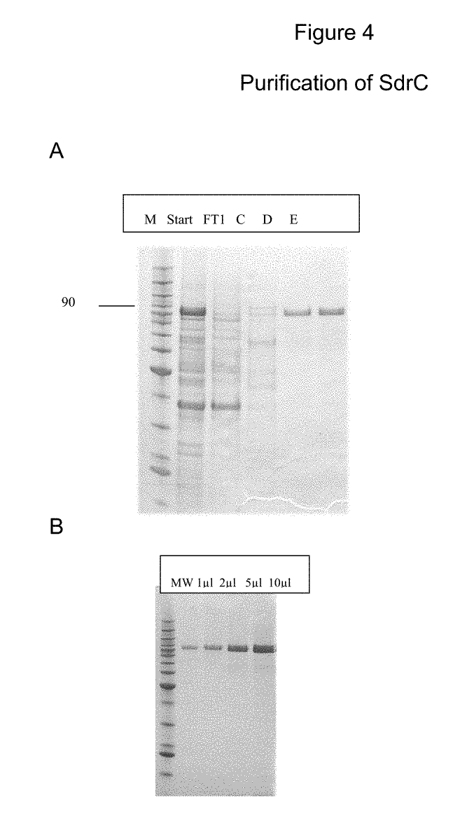

The purified proteins were analysed by SDS-PAGE. The results for one protein purified under native conditions (alpha toxin) and one protein purified under denaturing conditions (SdrC) are shown in FIGS. 3 and 4.

### Example 3 Preparation of S. aureus Capsular Polysaccharide Conjugates Using CDAP

Activation and Coupling Chemistry for Native PS8 Using CDAP:

SA08-TT004

Activation and coupling were performed at room temperature under continuous stirring. 10 mg of native polysaccharide were dissolved to obtain a final PS concentration of 2.5 mg/ml in 0.2M NaCl. The solution was then adjusted to pH 6.0+/−0.2 before the activation step.

At time 0, 50 μl of a CDAP solution (100 mg/ml freshly prepared in acetonitrile/WFI, 50/50) were added manually to reach the appropriate CDAP/PS (0.5/1) ratio.

After 1.5 minutes the pH was raised to pH 9.00+/−0.05 by addition of 0.5M NaOH.

NaOH addition takes about 1 minutes and pH is stabilised at pH 9.00+/−0.05 up to carrier addition.

At time 4.5 minutes, 1.5 ml of TT (10 mg/ml in 0.2M NaCl) was added to reach the appropriate Protein/PS ratio (1.5/1); pH was immediately adjusted to coupling pH 9.00+/−0.05. The solution is left for one hour under manual pH regulation.

After the coupling step, 0.5 ml of 2M glycine (ratio gly/PS (w/w): 7.5/1) were added; pH was immediately adjusted to 9.00+/−0.05. The solution was left for 30 minutes under manual pH regulation. Then the conjugate was clarified using a 5 μm Minisart filter and injected on SEPHACRYL™ S400HR (XK16/100). The flow-rate was fixed at 30 ml/h, using 150 mM NaCl.

The elution fractions were analysed by resorcinol and by μBCA. Interesting fractions were pooled and filtered on 0.22 μm STERIVEX™

The resulting conjugate had a final TT/PS ratio (w/w) of 1.05 as assessed by resorcinol and Lowry assays.

### Example 4 Preparation of S. aureus Capsular Polysaccharide Conjugates Using CDAP on Sized Polysaccharides

Activation and coupling chemistry for sized PS8 using CDAP

PS is weighted on the basis of 10% theoretical moisture content. 2 g of native, humid PS was dissolved overnight in WFI at an initial concentration of 10 mg/ml. Before the sizing, the solution of native PS was clarified on 5 μm cut-off filter.

An EMULSIFLEX™ C-50 homogenizer apparatus, in which the homogenizing cell was replaced with a Microfluidics F20Y-0.75 μm interaction chamber, was used to reduce the molecular weight and the viscosity of the polysaccharide before the activation step

The size reduction was realized at 10000 psi during the 10 first cycles and then at 15000 psi for the following 60 cycles. The progress of the size reduction was followed in-process by measuring viscosity. The sizing was stopped after 70 cycles when the target of 2.74±0.2 cp was reached.

Activation and coupling were performed at room temperature under continuous stirring. 50 mg of sized polysaccharide 8 were diluted to obtain a final PS concentration of 5 mg/ml in 0.2M NaCl.

At time 0, 375 μl of a CDAP solution (100 mg/ml freshly prepared in acetonitrile/WFI, 50/50) were added manually to reach the appropriate CDAP/PS (0.75/1) ratio. After 1 minute the pH was raised to pH 9.00+/−0.05 by addition of 0.5M NaOH.

At time 2.5 minutes, 10 ml of TT at 10 mg/ml in 0.2M NaCl were added to reach the appropriate Protein/PS ratio (2/1); pH was immediately adjusted to coupling pH 9.00+/−0.05. The solution was left for 55 minutes under manual pH regulation.

After the coupling step, 2.5 ml of 2M glycine (ratio gly/PS (w/w): 7.5/1) were added; pH was immediately adjusted to 9.00+/−0.05 by the regulator. The solution was left for 30 minutes under manual pH regulation.

Then the conjugate was clarified using a 5 μm Minisart filter and injected on SEPHACRYL™ S400HR (XK26/100). The flow-rate was fixed at 60 ml/h.

The elution fractions were analysed by resorcinol and by protein dosage. Interesting fractions were pooled and filtered on 0.22 μm MILLIPAK™20.

The resulting conjugate has a final TT/PS ratio of 1.94.

### Example 5 Preparation of S. aureus Capsular Polysaccharide Conjugates Using EDAC

Activation and Coupling Chemistry Using EDAC:

S. aureus Capsular Polysaccharide Type 8-TT Conjugate:

PS Derivatization

Activation and coupling were performed at room temperature under continuous stirring. 30 mg of native polysaccharide were diluted to obtain a final polysaccharide concentration of 5 mg/ml in water. The solution was adjusted to pH 4.5-5.0 with 0.5N HCl and then 66 μg of ADH were added (2.2 mg/mg PS). After complete dissolution, 60 mg of EDAC were added (2 mg/mg PS). After 70 min the pH was raised to pH 7.5 with 1N NaOH to stop the reaction. Free ADH was removed by purification on SEPHACRYL™ S100HR (XK 16/40). The flow-rate was fixed at 60 ml/h using 0.2 M NaCl as elution buffer. A size reduction was done by sonication of 15 min allowing a sterile filtration on MILLEX™ filter (0.22 μm).

Coupling

Tetanus toxoid was added to 5 to 10 mg of derivatized polysaccharide in 0.2M NaCl and the pH was adjusted to pH 5.0 or pH 6.0 by addition of 0.5N HCl. EDAC was dissolved in 0.1M Tris buffer pH 7.5 and then added over a period of 10 min (⅕ vol each 2 min). According to the conditions used (see Table 6), the reaction was stopped after between 30 and 180 minutes by addition of 1M Tris-HCl pH 7.5. Prior to purification on SEPHACRYL™ S400HR, the conjugate was clarified using a 5 μm Minisart filter. Alternatively, the conjugate was clarified by a 5 minute sonication step. The conjugate was then injected on SEPHACRYL™ S400HR (XK16/100). The flow-rate was fixed at 30 ml/h using 150 mM NaCl as elution buffer. The elution pool was selected on the basis of resorcinol and μBCA profiles (which measure polysaccharide and protein dosage respectively). The conjugate was filtered on a 0.22 μm sterilizing membrane (MILLIPAK™ 20) at 10 ml/min.

TABLE 5

| | Coupling | | [PS (AH)] | [TT (AH)] | [reagent EDAC] |
|---|---|---|---|---|---|
| Conjugate | time | | (mg/ml) | (mg/ml) | (mg/mg PS) |
| SA08-TT011 | 40 | min | 3.58 | 6.45 | 0.5/1 |
| SA08-TT015* | 180 | min | 2 | 4.0 | 0.25/1 |
| SA08-TT017 | 30 | min | 3.75 | 7.5 | 0.25/1 |
| SA08-TT018 | 50 | min | 3.75 | 7.5 | 0.10/1 |

Table 5:
*coupling done at pH 6.0

The resulting conjugates have the following characteristics shown in Table 6:

TABLE 6

| | In. TT/PS | F. TT/PS | Y. PS | Filtr. |
|---|---|---|---|---|
| | ratio | ratio | rec | Yield |
| Conjugate | (w/w) | (w/w) | (%) | (%) |
| SA08-TT011 | 2/1 | 2.43/1 | 48 | 99 |
| SA08-TT015 | 2/1 | 2.40/1 | 53 | 104 |
| SA08-TT017 | 2/1 | 2.41/1 | 44 | 107 |
| SA08-TT018 | 2/1 | 2.40/1 | 42 | 106 |

S. aureus polysaccharide type 8 was also treated by microfluidization before derivatization with ADH

PS Derivatization

Activation and coupling are performed at room temperature under continuous stirring. 200 mg of sized polysaccharide are diluted to obtain a final PS concentration of 10 mg/ml in water. Then 440 mg of ADH were added (2.2 mg/mg PS). The solution was adjusted to pH 4.7 with 1N HCl before the addition of 400 mg of EDAC (2 mg/mg PS). After 60 min the pH was raised to pH 7.5 with 5M NaOH to stop the reaction. The mixture was concentrated on Amicon Ultra (cut-off 10.000 MWCO). Prior to purification on SEPHACRYL™ S200HR (XK16/100), the conjugate was clarified using a 5 μm Minisart filter. The flow-rate was fixed at 30 ml/h using 0.150 M NaCl as elution buffer.

Coupling

100 mg of TT was added to 50 mg of derivatized polysaccharide in 0.2M NaCl. The pH was adjusted to pH 5.0±0.02 by addition of 0.3N HCl. EDAC was dissolved in 0.1 M Tris buffer pH 7.5 and then added over a period of 10 min ( 1/10 vol each minute). According to the conditions used (see Table 8), the reaction was stopped after between 30 and 180 minutes by addition of 1M Tris-HCl pH 7.5. Prior to purification on SEPHACRYL™ S400HR, the conjugate was clarified using a 5 μm Minisart filter. The conjugate was then injected on SEPHACRYL™ S400HR (XK50/100). The flow-rate was fixed at 60 ml/h using 150 mM NaCl as elution buffer. The elution pool was selected on the basis of resorcinol and μBCA profiles (which measure polysaccharide and protein dosage respectively). Then, the conjugate was filtered on a 0.22 μm sterilizing membrane (MILLIPAK™20) at 10 ml/min.

TABLE 7

| | Coupling | | [PS-AH] | [TT] | [EDAC] |
|---|---|---|---|---|---|
| Conjugate | time | | (mg/ml) | (mg/ml) | (mg/mg PS) |
| SA08-TT045 | 65 | min | 3.83 | 7.66 | 0.1 |
| SA08-TT046 | 45 | min | 3.75 | 7.5 | 0.2 |
| SA08-TT047 | 30 | min | 5.0 | 15.0 | 0.2 |
| SA08-TT048 | 120 | min | 5.0 | 10.0 | 0.05 |
| SA08-TT049* | 50 | min | 5.0 | 10.0 | 0.1 |

*EDAC added in “one time”

TABLE 8

| | In. TT/PS | F. TT/PS | Y. PS | Filtr. |
|---|---|---|---|---|
| | ratio | ratio | rec | Yield |
| Conjugate | (w/w) | (w/w) | (%) | (%) |
| SA08-TT045 | 2/1 | 2.20/1 | 57 | 101 |
| SA08-TT046 | 2/1 | 2.80/1 | | |
| SA08-TT047 | 3/1 | Gel- Not | — | — |
| | | purified | | |
| SA08-TT048 | 2/1 | 3.35 | 30 | 101 |
| SA08-TT049 | 2/1 | 3.5 | 24 | 106 |

### Example 6 Preparation of S. aureus Capsular Polysaccharide Conjugates Using EDAC on De-O-Acetylated S. aureus Polysaccharide 8

De-O-acetylation

0.1N NaOH was added to 16 ml of sized PS (10 mg/ml) to target a final PS concentration of 9 mg/ml and a final NaOH concentration of 0.1N. After a treatment of 1 or 2 h at 37° C., the PS had a level of O-acetylation of 35 and 12% (Hestrin dosage) respectively in comparison to the untreated PS.

0.1N NaOH was added to 19 ml of sized PS (10 mg/ml) to target a final PS concentration of 9.5 mg/ml and a final NaOH concentration of 0.05N. After a treatment of 1 or 2 h at 37° C., PS had a level of O-acetylation of 78 and 58% (Hestrin dosage) respectively in comparison to the untreated PS.

The derivatization step was done as shown previously for an untreated PS.

TABLE 9

| Conjugate | O-acetyl level % | ADH/PS w/w* % |
|---|---|---|
| SA08-TT056 | 35 | 9.3 |
| SA08-TT057 | 12 | 13.1 |
| SA08-TT058 | 78 | 5.3 |
| SA08-TT059 | 58 | 8.2 |

*TNBS assay

Removal of the O-acetyl groups resulted in an increased availability of reactive carboxylic groups. Indeed, the derivatization level of a PS having only 12% of O-acetyl groups was ±2.5-fold superior to the one having 78% of O-acetyl groups.

Coupling was done as shown previously for a untreated PS

TABLE 10

| | O-acetyl | Coupling | [PS-AH] | [TT] | [EDAC] |
|---|---|---|---|---|---|
| Conjugate | level % | time | (mg/ml) | (mg/ml) | (mg/mg PS) |
| SA08-TT056 | 35 | 45 min | 2.87 | 5.74 | 0.5 |
| SA08-TT057 | 12 | 30 min | 2.62 | 5.24 | 0.5 |
| SA08-TT058 | 78 | 50 min | 3.16 | 6.32 | 0.5 |
| SA08-TT059 | 58 | 40 min | 2.53 | 5 | 0.5 |

TABLE 11

| | In. TT/PS | F. TT/PS | Y. PS | Filtr. |
|---|---|---|---|---|
| | ratio | ratio | rec | Yield |
| Conjugate | (w/w) | (w/w) | (%) | (%) |
| SA08-TT056 | 2/1 | 1.70/1 | 51.3 | 100 |
| SA08-TT057 | 2/1 | 1.78/1 | 63.0 | 105.4 |
| SA08-TT058 | 2/1 | 2.08/1 | 46.3 | 99.6 |
| SA08-TT059 | 2/1 | 1.86/1 | 50.8 | 99.2 |

### Example 7 Conjugation of dPNAG

Activation and Coupling of dPNAG:

dPNAG-TT Conjugates

The following conjugates were produced using the approaches described herebelow:

dPNAG-TT010: dPNAG-S-GMBS+DTT treated TT-LC-SPDP

dPNAG-TT011: dPNAG-S-GMBS+DTT treated TT-LC-SPDP

dPNAG-TT012: dPNAG-S-GMBS+DTT treated TT-SPDP

dPNAG-TT014: dPNAG-SPDP+DTT treated TT-SPDP

dPNAG-TT017:DTT treated dPNAG-SPDP+TT-LC-SPDP

dPNAG-TT019: dPNAG-S-GMBS+DTT treated TT-SPDP

dPNAG-TT020: dPNAG-S-GMBS+DTT treated TT-SPDP

dPNAG

1 g of PNAG was dissolved in 5N HCl at a concentration of 20 mg/ml and was incubated for 1 hour. It was then neutralized with 5N NaOH. The solution was clarified on a 5 μm membrane and purified on SEPHACRYL™ S400HR. Interesting fractions, corresponding to the “medium molecular size” (see Infection and Immunity, 70: 4433-4440 (2002)), were pooled and concentrated prior to de-N-acetylation treatment.

The solution was adjusted at 1M NaOH and left 24 hours at 37° C. After neutralization, the product was subjected to dialysis and concentration.

dPNAG Activation

S-GMBS (N-(γ-Maleimidobutyryloxy) sulfosuccinimide, Pierce) was added to dPNAG in 0.2M NaCl (ratio S-GMBS/PS (w/w):1/1) and incubated during 2 h at room temperature at pH 7.0 (pH regulation using 1M NaOH). Excess GMBS and by-products were removed by purification on TOYOPEARL® HW-40F using PBS, 10 mM EDTA, 50 mM NaCl pH 7.2 as elution buffer with a flow-rate fixed at 60 ml/h. The elution pool was selected in function of the optical density (UV=206 nm) and then concentrated on VIVASPIN® tubes 3,000 MWCO or AMICON® Ultra 10,000 MWCO.

Coupling

GMBS-activated dPNAG and DTT reduced TT-SPDP were mixed and stirred at room temperature. According to the conditions used the reaction was quenched after 20-120 min by the addition of cysteine (4 mg/ml in Na phosphate buffer pH 8.0) for 30 minutes. The conjugate was clarified on 5 μm filter and injected on SEPHACRYL™ S300HR resin (XK16/100) for purification. Elution was realized in 200 mM NaCl with a flow-rate fixed at 30 ml/h. The elution fractions were analysed by hexosamine and by protein dosage. Interesting fractions were pooled and filtered on 0.22 μm STERIVEX™. The final conjugate was tested for polysaccharide (hexosamine dosage) and protein composition (Lowry dosage).

TABLE 12

| | | | | PS | Coupl. |
|---|---|---|---|---|---|
| | N-acetylation | [dPNAG] | [TT] | scale | time |
| Conjugate | level % | mg/ml | mg/ml | (mg) | (min) |
| dPNAG-TT 010 | 10* | 15 | 15 | 30 | 120 |
| dPNAG-TT 011 | 10* | 12 | 24 | 20 | 120 |
| dPNAG-TT 012 | 10* | 17.5 | 35 | 22 | 80 |
| dPNAG-TT 019 | 34 | 5 | 10 | 10 | 20 |
| dPNAG-TT 020 | 34 | 2 | 2 | 10 | 20 |

*Not done on the lot used in the conjugation but estimated on a previous lot by NMR using the same de-N-acetylation method.

TABLE 13

| | In. TT/PS | F. TT/PS | Yield PS | Filtration |
|---|---|---|---|---|
| | ratio | ratio | rec | yield |
| Conjugate | (w/w) | (w/w) | (%) | (%) |
| dPNAG-TT010 | 1/1 | 1.86/1 | 43 | 99 |
| dPNAG-TT011 | 2/1 | 2.86/1 | 56 | 99 |
| dPNAG-TT012 | 2/1 | 2.29/1 | 61 | 108 |
| dPNAG-TT019 | 2/1 | 1.45/1 | 81 | 97 |
| dPNAG-TT020 | 1/1 | 0.89/1 | 82 | 109 |

dPNAG-SPDP:

A 5-fold molar excess of SPDP (N-Succinimidyl-3-(2-Pyridyldithio) Propionate, MW: 312.4, Pierce) dissolved in DMSO (dimethylsulfoxid, Merck) was added to 100 mg of dPNAG at 5 mg/ml in 100 mM Na phosphate, pH 7.2) and incubated 1 h at room temperature. Before purification on SEPHACRYL™ S100HR (XK16/40) the reaction mixture was concentrated to ±6 ml on AMICON® Ultra 10,000 MWCO (centrifugation at 3000 rpm during 28 min). Elution was realized in phosphate buffer pH 7.4 with a flow-rate fixed at 60 ml/h. The interesting fractions (read at 206 nm) were pooled and concentrated to 1.1 ml on AMICON® Ultra 10,000 MWCO (centrifugation at 3000 rpm during 30 min).

TT-SPDP:

A 15-fold molar excess of SPDP (Pierce) dissolved in DMSO (dimethylsulfoxid, Merck) was added to 1 g of TT (50 mg/ml) in 100 mM Na phosphate, pH 7.2 and incubated 80 min at room temperature. Then the product was injected on SEPHACRYL™ S100HR (XK16/40) and eluted in 100 mM Na acetate pH 5.6, 100 mM NaCl, 1 mM EDTA with a flow-rate fixed at 60 ml/h. The elution pool was selected in function of the optical density (UV=280 nm) and then concentrated to 19.6 ml on AMICON® Ultra 10,000 MWCO (centrifugation at 3000 rpm during 75 min).

TT-LC-SPDP was produced as TT-SPDP but using LC-SPDP (Succinimidyl 6-[3-(2-pyridyldithio)-propionamido]hexanoate, Pierce) and an incubation time of 60 min.

TT-SH or TT-LC-SH

DTT was added to TT-SPDP or TT-LC-SPDP in a DTT/TT ratio (mg/mg) of 0.7/1. After 2 h at room temperature, the release of pyridine-2-thione was followed by its characteristic absorbance at 343 nm. The thiolated protein was purified from excess DTT by gel filtration (PD-10, Amersham). After concentration on AMICON® Ultra 10,000 MWCO, protein content was estimated by Lowry dosage.

dPNAG-SPDP+TT-SH or TT-LC-SH (dPNAG-TT014 and 016)

Coupling was performed at room temperature under continuous stirring and with an initial TT/PS ratio (w/w) of 2/1.

dPNAG and TT-SH were mixed in order to obtain a final PS concentration of 20 mg/ml and a final protein concentration of 40 mg/ml. After 30 min, unreacted sulfhydryl groups were quenched by addition of 2-Iodoacetamide (Merck). dPNAG and TT-LC-SH was mixed in order to obtain a final PS concentration of 10 mg/ml and a final protein concentration of 20 mg/ml. After 75 min, unreacted sulfhydryl groups were quenched by addition of 2-Iodoacetamide (Merck).

Then the conjugate is clarified using a 5 μm Minisart filter and injected on SEPHACRYL™ S300HR (XK16/100). Elution was realized in 200 mM NaCl with a flow-rate fixed at 30 ml/h.

The elution fractions were analysed by hexosamine and by protein dosage. Interesting fractions were pooled and filtered on 0.22 μm STERIVEX™.

The resulting conjugates have a final TT/PS ratio (w/w) of 2.18 (TT-SH) and 2.24 (TT-LC-SH).

Thiolation of dPNAG

11.6 mg of DTT (1, 4-Dithiothreitol, Boerhinger Mannheim, MW: 154.24) were added to 16.5 mg of dPNAG-SPDP. After 2 h at room temperature, the release of pyridine-2-thione was followed by its characteristic absorbance at 343 nm. The thiolated PS was purified from excess DTT by gel filtration (TOYOPEARL® HW40F) and then concentrated to 860 μl on AMICON® Ultra 10,000 MWCO.

dPNAG-SH+TT-SPDP (dPNAG-TT017)

Coupling was performed at room temperature under continuous stirring and with an initial TT/PS ratio (w/w) of 1.7/1.

dPNAG-SH and TT-SPDP were mixed in order to obtain a final PS concentration of 7.73 mg/ml and a final protein concentration of 13.3 mg/ml. After 90 min, unreacted sulfhydryl groups were quenched by addition of 2-Iodoacetamide (Merck).

Then the conjugate was clarified using a 5 μm Minisart filter and injected on SEPHACRYL™ S300HR (XK16/100). Elution was realized in 200 mM NaCl with a flow-rate fixed at 30 ml/h.

The elution fractions are analysed by hexosamine and by protein dosage. Interesting fractions were pooled and filtered on 0.22 μm STERIVEX™.

The resulting conjugate has a final TT/PS ratio (w/w) of 2.74.

### Example 8 Formulation

Adjuvant Compositions

The conjugates were inoculated either unadjuvanted or adjuvanted with adjuvant A, having the following composition:

Composition of Adjuvant A

Qualitative Quantitative (per 0.5 mL dose)

Liposomes:
DOPC 1 mg
cholesterol 0.25 mg

3DMPL™ 50 μg

QS21 50 μg

KH₂PO₄₁ 3.124 mg Buffer

Na₂HPO₄₁ 0.290 mg Buffer

NaCl 2.922 mg

(100 mM)

WFI q.s. ad 0.5 ml Solvent

pH 6.1

1. Total PO4 concentration=50 mM

### Example 9

Animal Experiments.

Female CD-1 mice, 8 to 10 weeks old, are obtained from Charles River Laboratories, Kingston, Mass. For lethality studies, five groups of 9 to 11 CD-1 mice are challenged intraperitoneally (i.p.) with serial dilutions of S. aureus grown on CSA plates. The inocular sizes range from ˜10¹⁰ to 10⁸ CFU/mouse. Mortality is assessed on a daily basis for 3 days. The 50% lethal doses (LD₅₀s) is estimated by using a probit model of the dose-response relationship. The null hypothesis of common LD₅₀s was tested by the likelihood ratio test. Sublethal bacteremia is initiated by challenging groups of 8 to 20 mice by the intravenous (i.v.) route with ˜2×10⁶ CFU/mouse or by the i.p. route with ˜2×10⁷ CFU/mouse. After inoculation separate groups of animals are bled from the tail at specified times, and the bacteremia levels are estimated by quantitative plate counts performed in duplicate on tryptic soy agar plates with 5% sheep blood (Becton Dickinson Microbiology Systems). Statistical significance is determined with the Welch modification of the unpaired Student's t test.

### Example 10

Immunogenicity of S. aureus PS8-TT and dPNAG-TT Conjugates

Groups of 30 mice were inoculated subcutaneously with S. aureus PS8-TT conjugate at a saccharide dose of 3 μg, either unadjuvanted or combined with adjuvant A, on days 0, 14, 28 and 42. On day 0, the mice received a first saccharide dose including between 0.001 and 0.013 μg. The further three immunisations were done with a dose of 0.3 μg in saline. On day 55 serum was collected from the mice and each serum sample was tested by ELISA to assess the immune response against PS8. Groups of 10 mice were used in the control groups and these were inoculated with either saline or saline containing adjuvant A.

The purified PS8 was coated at 2 μg/ml in phosphate buffered saline (PBS) on high binding microtitre plates (NUNC™ MAXISORP™) overnight at 4° C. The plates were blocked with PBS-BSA 1% for 30 min at room temperature with agitation. The mice antisera were prediluted 1/100, then further twofold dilutions were made in microplates which were incubated at 37° C. for 1 hour. After washing, bound murine antibody was detected using Jackson ImmunoLaboratories Inc. peroxidase-conjugated AFFINIPURE™ Goat Anti-Mouse IgG (H+L) (ref: 115-035-003) diluted 1:5000 in PBS-TWEEN™ 0.05%. The detection antibodies were incubated for 30 minutes at room temperature with agitation. The color was developed using 4 mg OPD (Sigma)+5 μl H2O2 per 10 ml pH 4.5 0.1M citrate buffer for 15 minutes in the dark at room temperature. The reaction was stopped with 50 μl HCl, and the optical density was read at 490 nm relative to 650 nm.

The results were expressed in mid-point titers and the GMT was calculated for the 30 samples (10 for controls). The results are shown in Table 14 below.

TABLE 14

| | Anti-PS8 titre (GMT) | Anti-PS8 titre (GMT) |
|---|---|---|
| Conjugate | nonadsorbed | Adjuvant A |
| SA08-TT011 | 4714 | 2109 |
| SA08-TT015 | 2806 | 5631 |
| SA08-TT017 | 3770 | 4396 |
| SA08-TT018 | 5349 | 4748 |
| Control | 50 | 50 |

Groups of 30 mice were inoculated subcutaneously with S. aureus dPNAG-TT conjugates (containing dPNAG which was between 10% and 30% N-acetylated) at a saccharide dose of 0.34 in 200 mM NaCl, either unadjuvanted or combined with adjuvant A. The mice received three inoculations on days 0, 14 and 28. On day 41 or 42 serum was collected from the mice and each serum sample was tested by ELISA to assess the immune response against PNAG. Groups of 10 mice were used in the control groups and these were inoculated with saline or with adjuvant alone.

Anti-PNAG ELISA:

Purified PNAG (2.5 μg/ml) mixed with methylated HSA (2.5 μg/ml) diluted in phosphate buffered saline (PBS) was coated on high binding microtitre plates (NUNC™ MAXISORP™) overnight at 4° C.

The plates were blocked with PBS-BSA 1%, 30 min at RT with agitation. The mice antisera were prediluted 1/100, then further twofold dilutions were made in microplates and incubated at room temperature with agitation for 1 hour. After washing, bound murine antibody was detected using Jackson ImmunoLaboratories Inc. peroxidase-conjugated AFFINIPURE™ Goat Anti-Mouse IgG (H+L) (ref: 115-035-003) diluted 1:5000 in PBS-BSA 0.2%-TWEEN™ 0.05%. The detection antibodies were incubated for 30 min. at room temperature with agitation. The color was developed using 4 mg OPD (Sigma)+5 μl H2O2 per 10 ml pH 4.5 0.1M citrate buffer for 15 minutes in the dark at room temperature. The reaction was stopped with 50 μl HCl, and the optical density was read at 490 nm relative to 650 nm.

A GMT was calculated on the mid-point titers of the 30 samples (10 for the controls).

TABLE 15

| | Anti-PNAG GMT | Anti-PNAG GMT |
|---|---|---|
| Conjugate | Non-adsorbed | Adjuvant A |
| dPNAG-TT010 | 1371 | 28465 |
| dPNAG-TT011 | 1133 | 40899 |
| dPNAG-TT019 | 425 | 13429 |
| dPNAG-TT020 | 656 | 10080 |
| dPNAG-TT014 | 342 | 9806 |
| dPNAG-TT017 | 203 | 8094 |
| dPNAG-TT012 | 398 | 40509 |
| dPNAG-TT016 | 719 | 7937 |
| Control | 50 | 50 |

### Example 11 Immunogenicity of PS*-TT Conjugates Made by the CDAP Method

Results

TABLE 16

| | Anti PS8 GMT post | Anti-PS8 GMT post |
|---|---|---|
| | three inoculations | two inoculations |
| Conjugate | in mice | in mice |
| SAPS8-TT-04 Specol | 207068 | 41326 |
| SAPS8-TT-04 Adjuvant A | 47405 | 15577 |
| SAPS8-TT-04 AlPO4 | 7380 | 4510 |
| Specol | 50 | |
| Adjuvant A | 50 | |
| AlPO4 | 50 | |

### Example 12

Opsonophagocytosis Assay.

The in vitro opsonophagocytosic killing of S.aureus by human polymorphonuclear leykocytes (PMNs) is performed as described in Xu et al 1992 Infect. Immun. 60; 1358. Human PMNs are prepared from heparinized blood by sedimentation in 3% dextran T-250. The opsonic reaction mixture (1 ml) contains ˜10⁶ PMNs in RPMI 1640 medium supplemented with 10% heat-inactivated fetal calf serum, ˜10⁸ CFU of S-aureus, and 0.1 ml of the test serum or IgG preparation. Hyperimmunized rabbit serum is used as a positive control, and 0.1 ml of nonimmune rabbit serum was used as a complete source for the IgG samples. The reaction mixtures are incubated at 37° C., and bacterial samples are transferred at 0, 60, and 120 min into water and subsequently diluted, spread on tryptic soy agar plates, and incubated at 37° C. for bacterial count after overnight incubation.

### Example 13

Immunogenicity of Staphylococcal Proteins in Mice and Rabbits

Animals were immunized with purified staphylococcal proteins in order to generate hyperimmune sera. Mice were immunized three times (days 0, 14 and 28) with 10 μg of each proteins adjuvanted in Specol. Rabbits were immunized three times (days 0, 21 and 42) with 20 μg of each proteins adjuvanted in Specol. Immune sera were collected and evaluated in anti-protein and anti-killed whole cells ELISA.

Anti-Protein ELISA:

The purified protein was coated at 1 μg/ml in phosphate buffered saline (PBS) on high binding microtitre plates (NUNC™ MAXISORP™) overnight at 4° C. The plates were blocked with PBS-BSA 1%, for 30 min at RT with agitation. The test samples were then diluted 1/1000 and incubated at room temperature for 1 hour with agitation. After washing, bound murine or rabbit antibody was detected using Jackson ImmunoLaboratories Inc. peroxidase-conjugated AFFINIPURE™ Goat Anti-Mouse IgG (H+L) (ref: 115-035-003) or AFFINIPURE™ Goat Anti-Rabbit IgG (H+L) (ref: 11-035-003) diluted 1:5000 in PBS-TWEEN™ 0.05%. The detection antibodies were incubated for 30 min. at room temperature with agitation. The color was developed using 4 mg OPD (Sigma)+5 μl H2O2 per 10 ml pH 4.5 0.1M citrate buffer for 15 minutes in the dark at room temperature. The reaction was stopped with 50 μl HCl, and the optical density was read at 490 nm relative to 650 nm.

The O.D. for a 1/1000 dilution of Post III was compared to the O.D. obtained with the same dilution of Pre-immune sera.

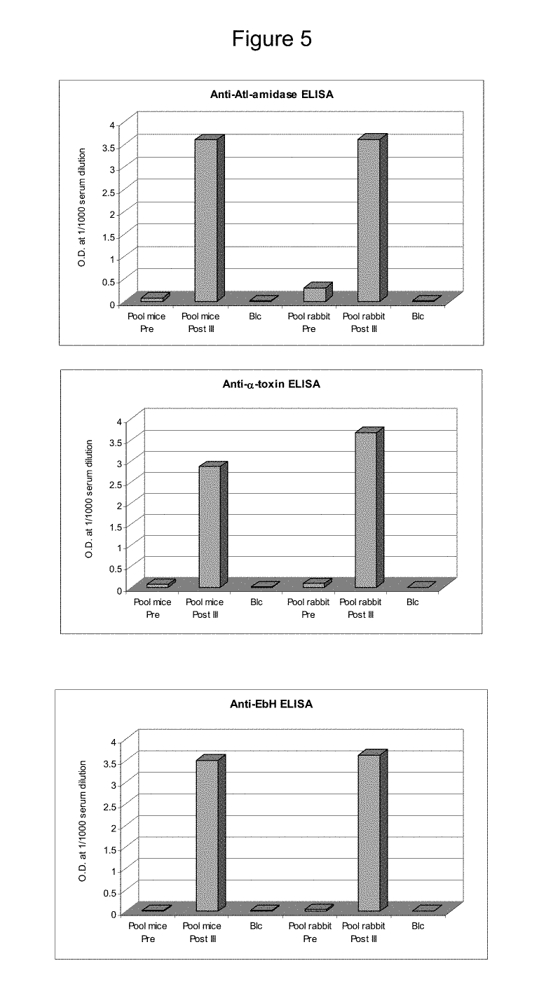

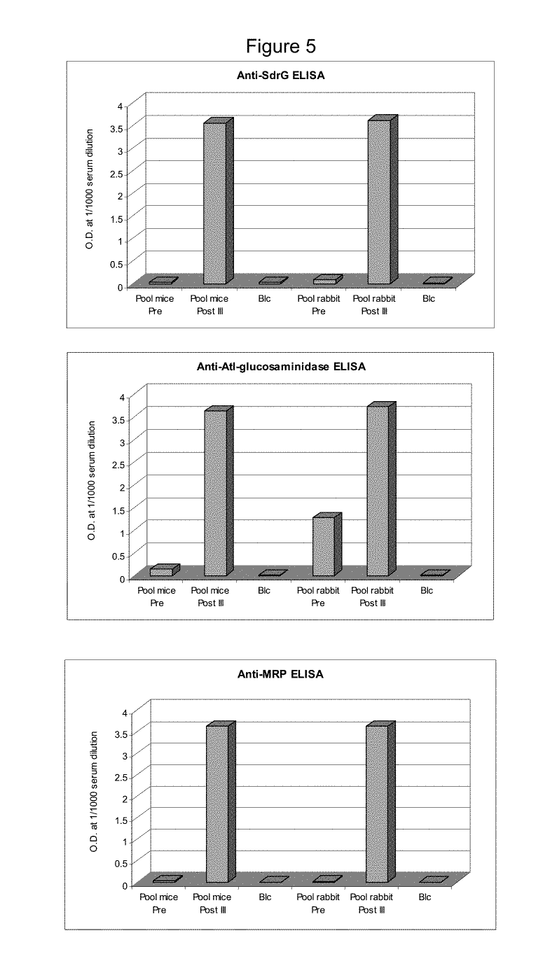

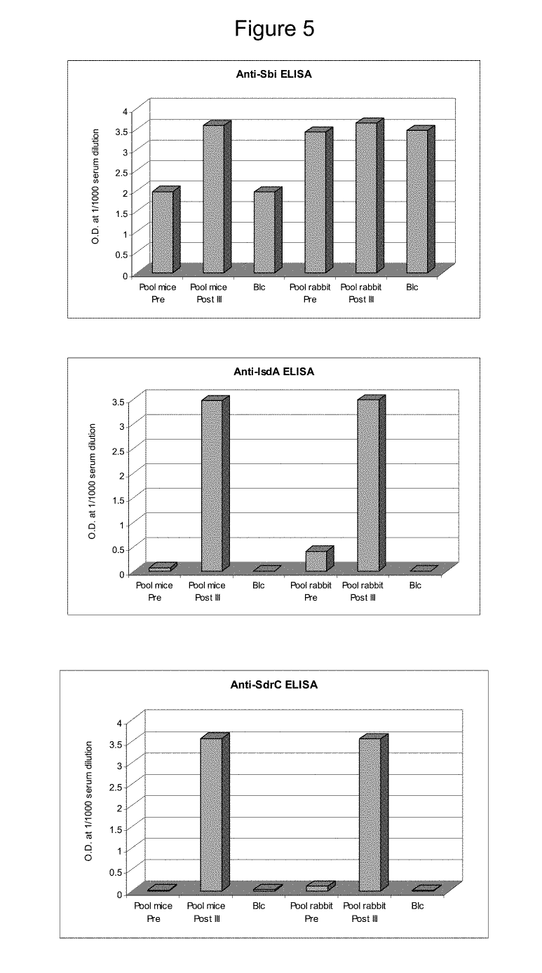

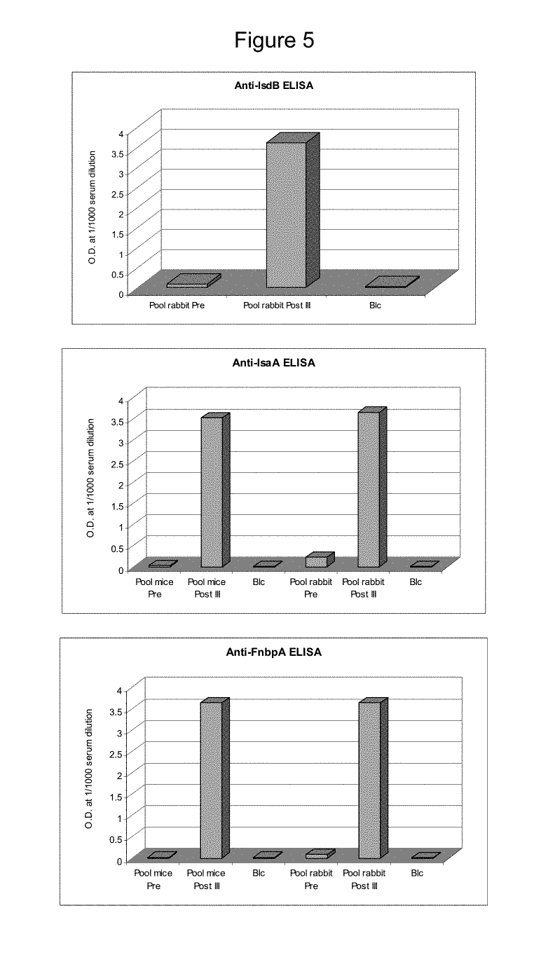

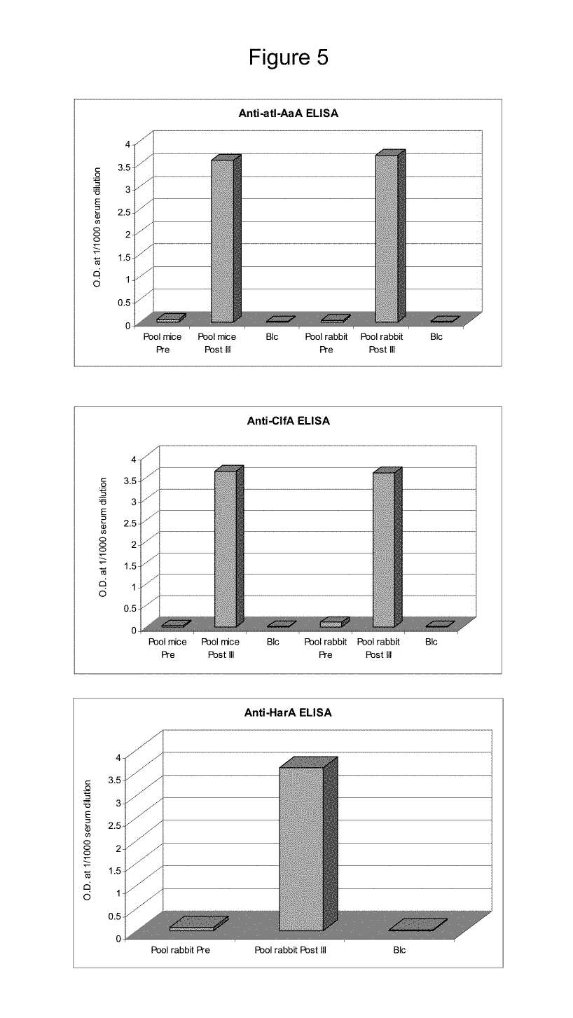

Results generated with mice and rabbit sera are presented in FIG. 5. A good seroconversion against each antigen was observed. Evaluation of sera directed against SBI was impaired due to the Ig binding activity of this protein.

Anti-Killed Whole Cells ELISA:

Killed whole cells (heat or formaldehyde inactivated) from S. aureus type 5 and 8 or S. epidermidis strain Hay were coated at 20 μg/ml in phosphate buffered saline (PBS) on high binding microtitre plates (NUNC™ MAXISORP™) overnight at 4° C. with evaporation. The plates were blocked with PBS-BSA 1% 30 min at room temperature with agitation. Protein A was neutralised by addition of 10 μg/ml of Affinity Purified Chicken anti-Protein A (ICL ref: CPA-65A-2) diluted in PBS-TWEEN™ 0.05% followed by incubation for 1 hour at room temperature. The test samples were then diluted two-fold on the microplate in PBS-0.05% from a starting dilution at 1/10 and incubated 1 hour at room temperature with agitation. After washing, bound murine or rabbit antibody was detected using Jackson ImmunoLaboratories Inc. peroxidase-conjugated AFFINIPURE™ Goat Anti-Mouse IgG (H+L) (ref: 115-035-003) or AFFINIPURE™ Goat Anti-Rabbit IgG (H+L) (ref: 11-035-003) diluted 1:5000 in PBS-TWEEN™ 0.05%. This detection antibodies were incubated for 30 min. at room temperature with agitation. The color was developed using 4 mg OPD (Sigma)+5 μl H2O2 per 10 ml pH 4.5 0.1M citrate buffer for 15 minutes in the dark, at room temperature. The reaction was stopped with 50 μl HCl, and the optical density was read at 490 nm relative to 650 nm.

It should be noted that expression levels of proteins in staphylococci will vary depending on culture conditions. Therefore a negative result may reflect the choice of incorrect culture conditions rather than a lack of immunogenicity.

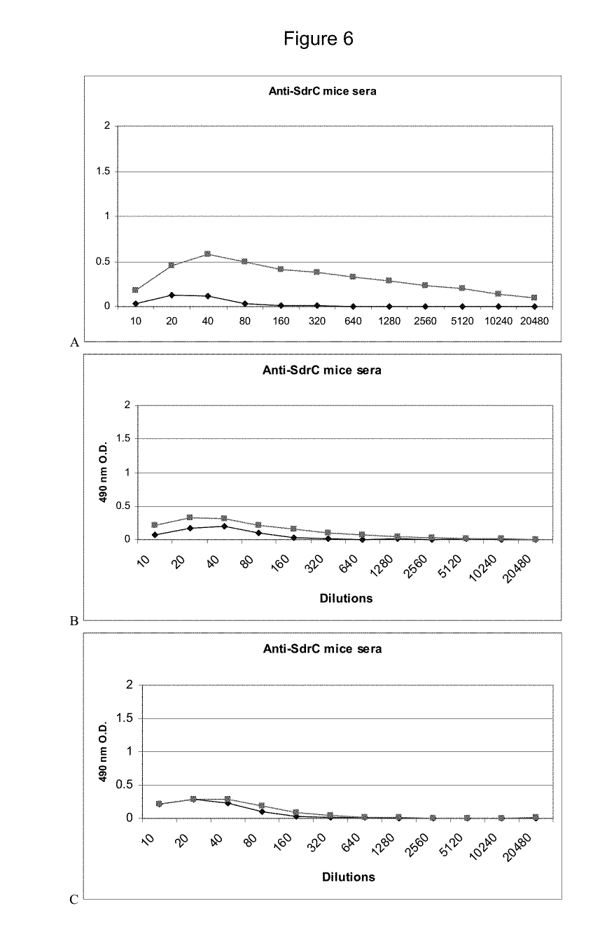

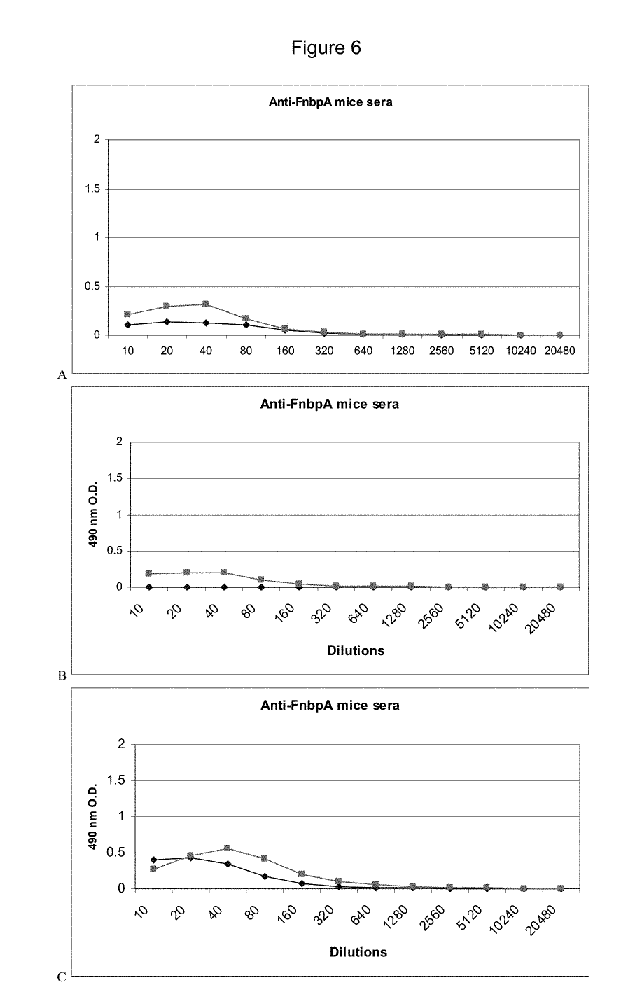

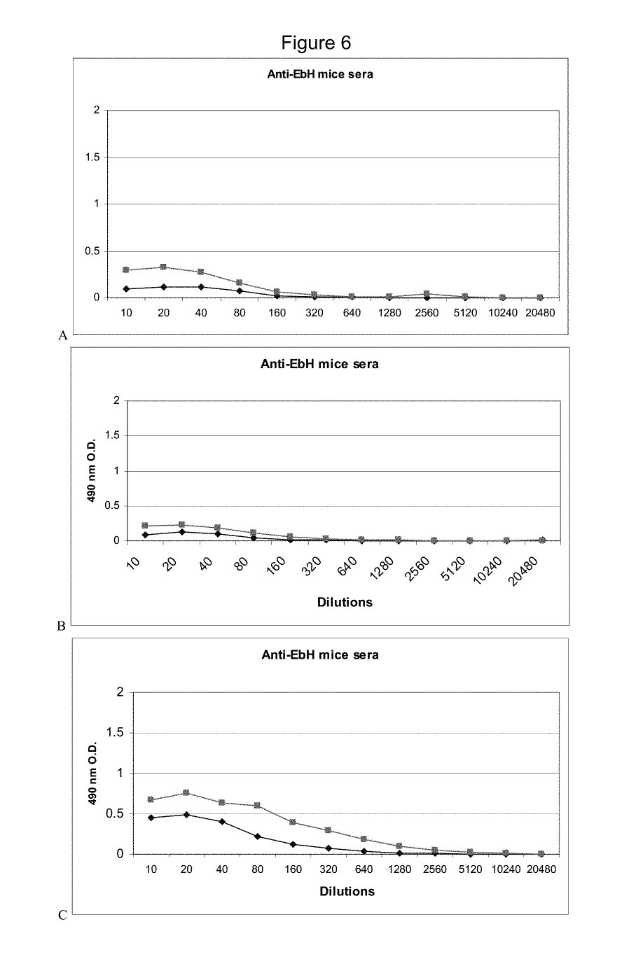

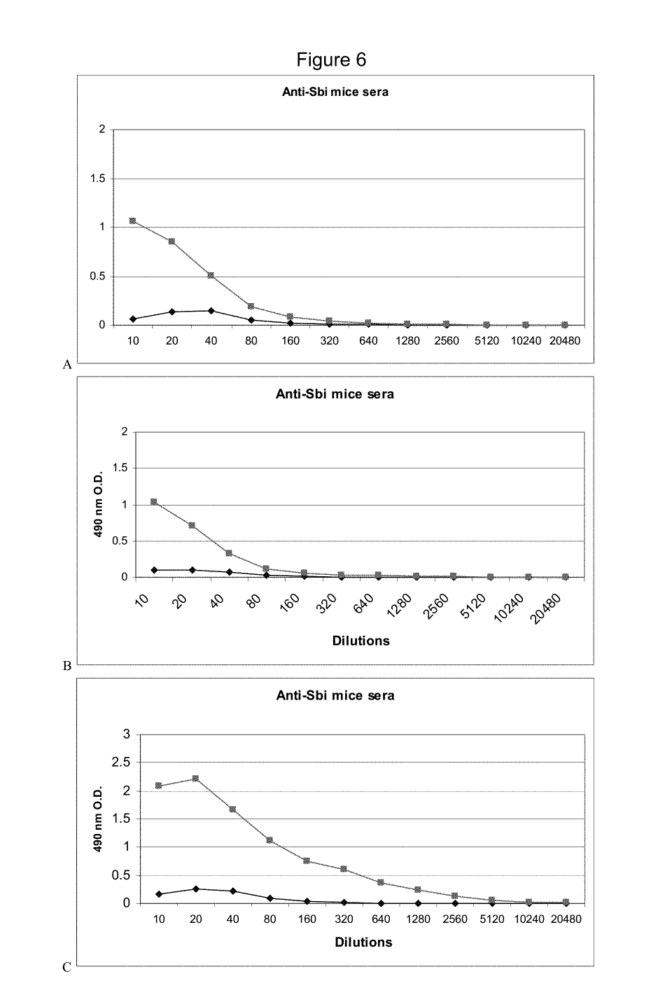

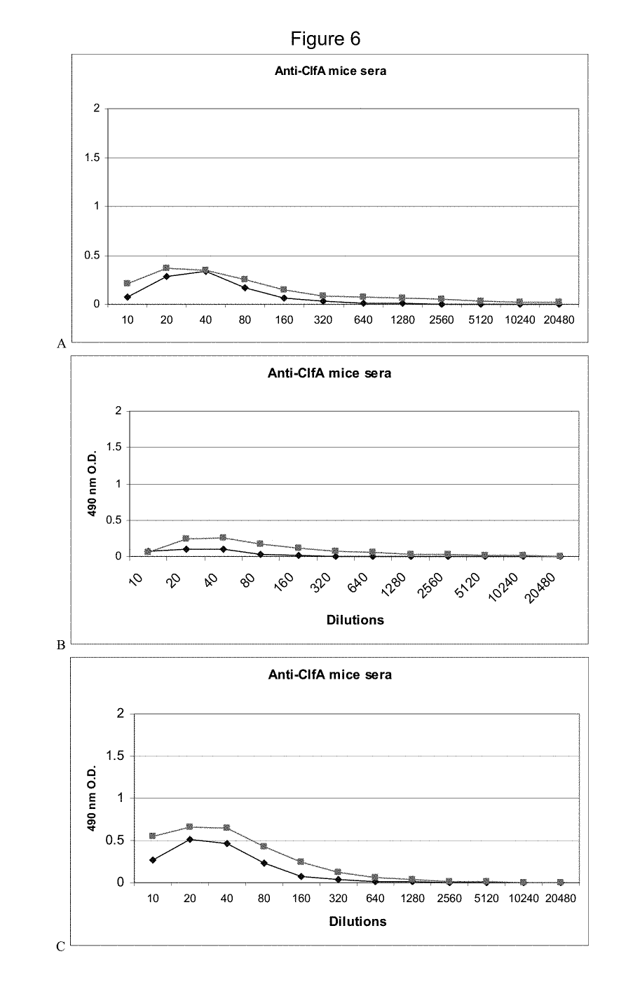

The results using mice sera are shown in Table 17 and some of the graphs are shown in FIG. 6. A weak recognition of S. aureus strain 5 is observed with sera directed against SdrC, FnbpA, Ebh, Sbi and IsaA. Recognition of S. aureus strain 8 is only observed with the serum directed against Sbi. Weak recognition of S. epidermidis Hay is observed with sera directed against Atl amidase, MRPII, IsdA, IsaA, Ebh, Aaa and Sbi.

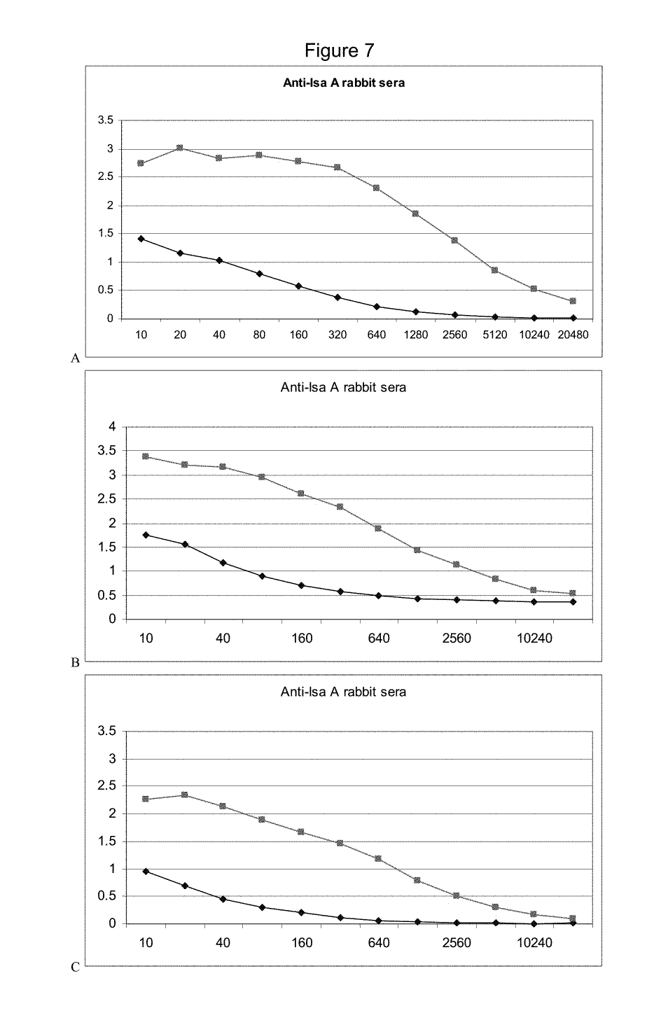

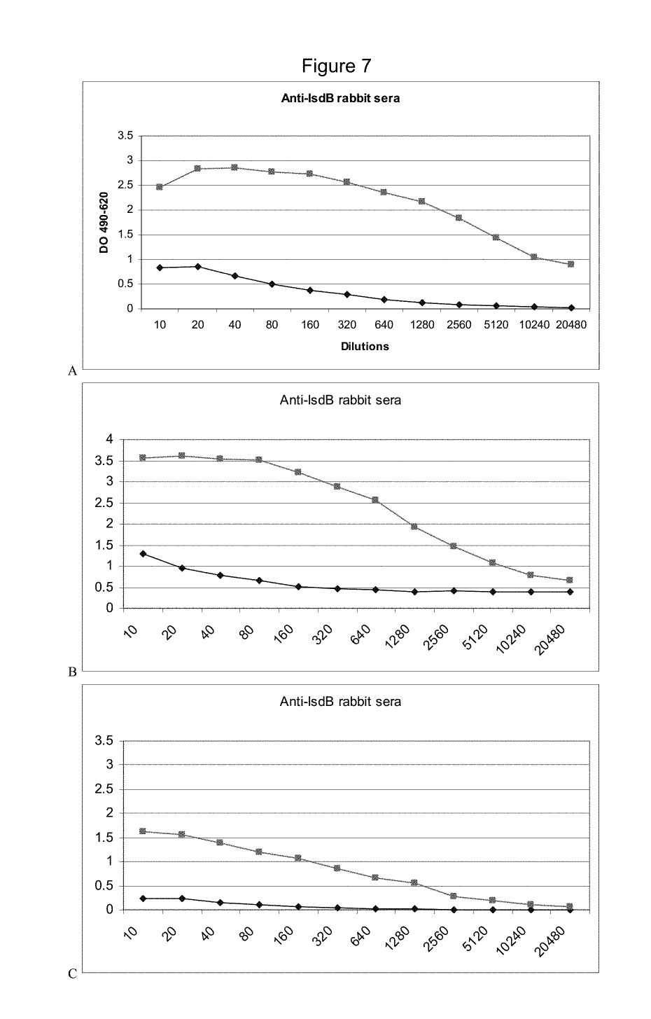

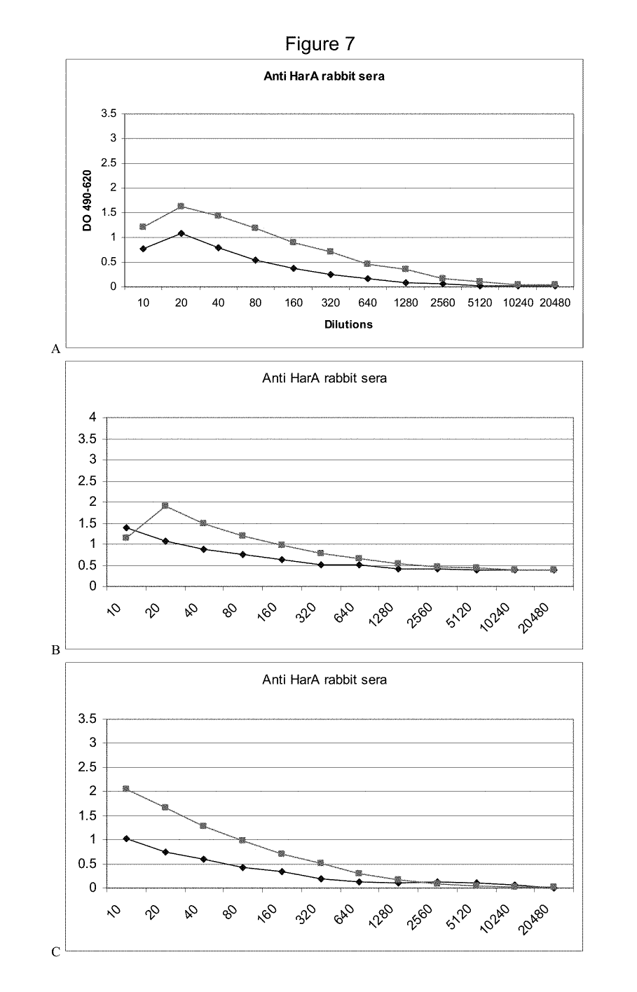

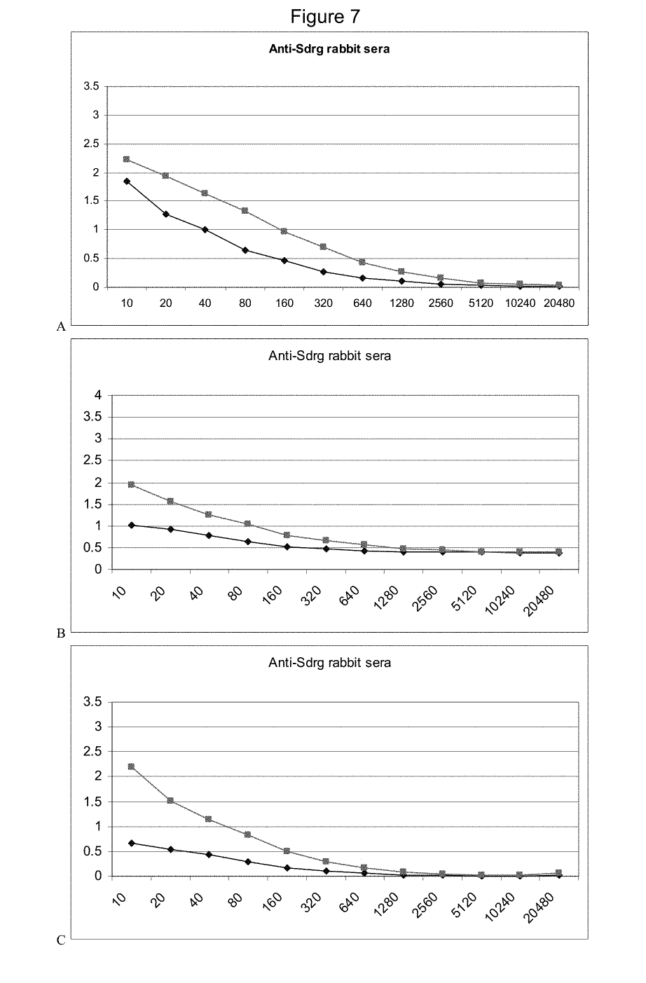

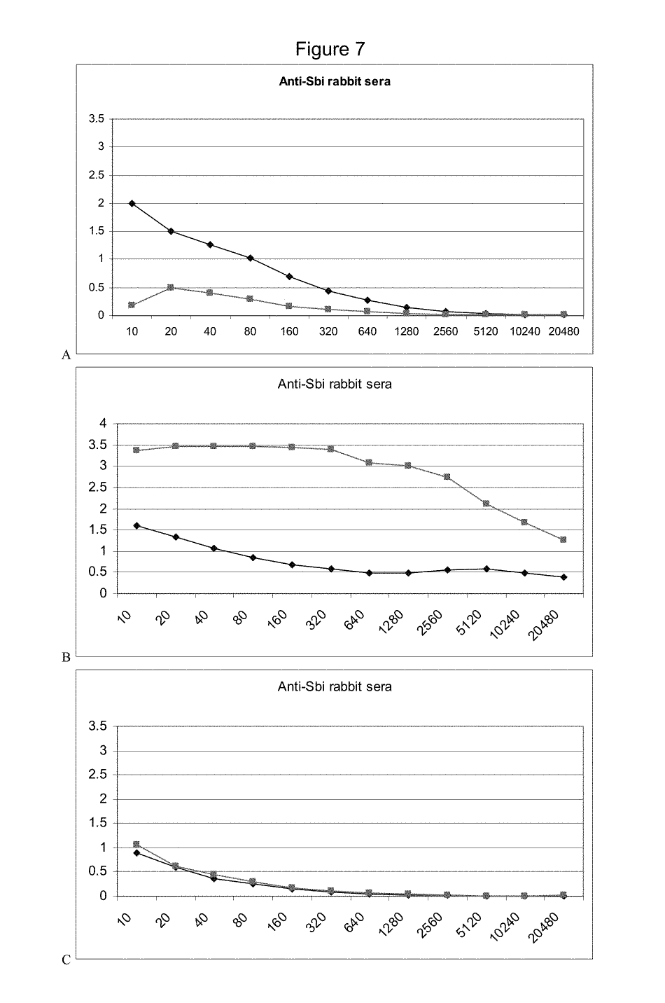

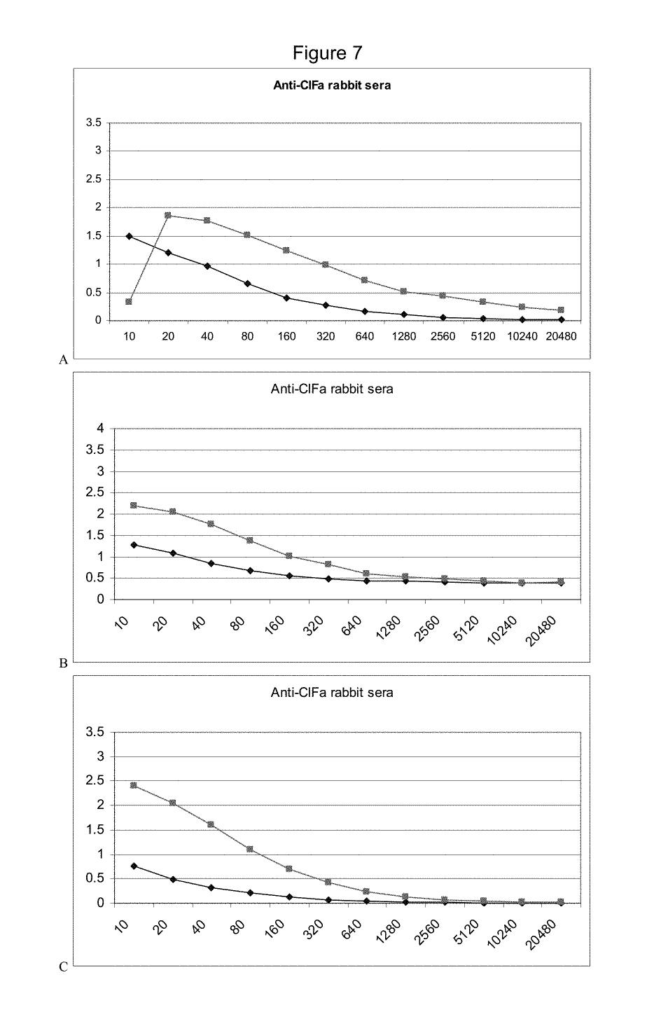

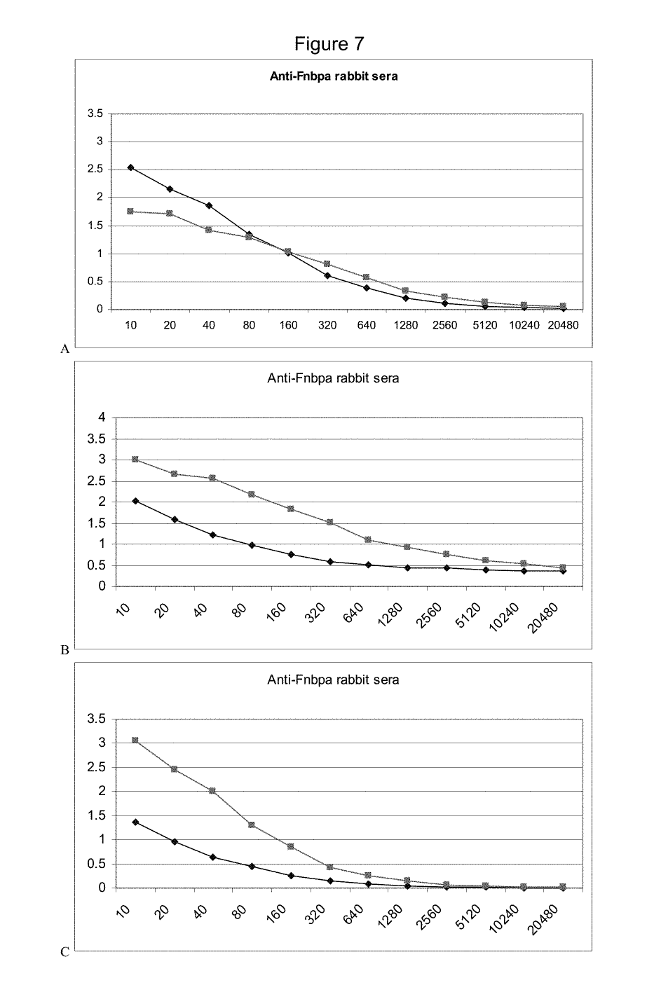

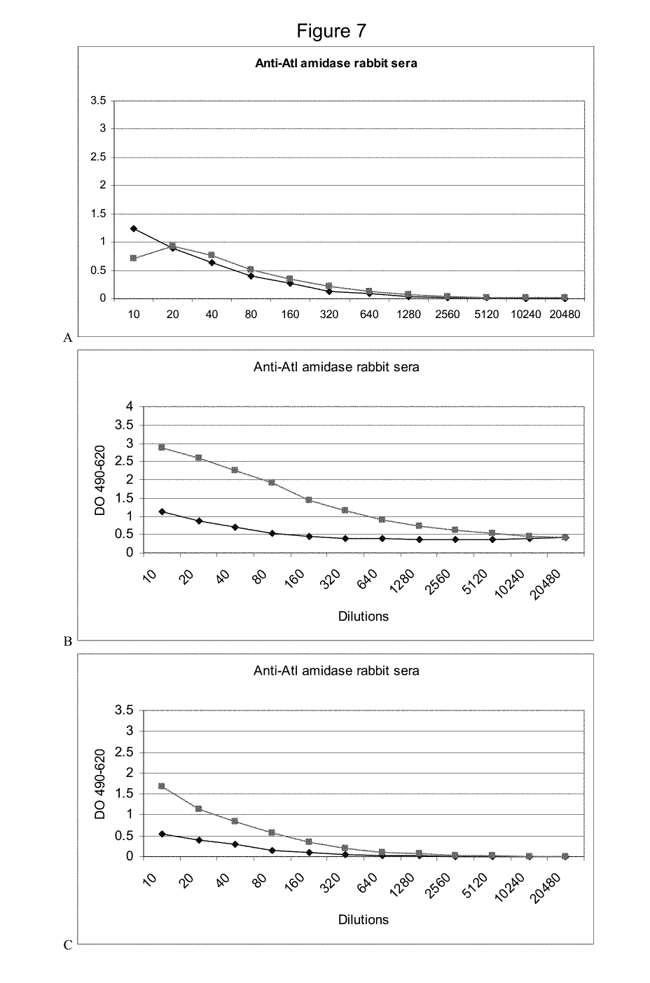

A selection of results generated using rabbit sera are shown in FIG. 7 and summarized in Table 18. Very good recognition of the three strains was observed with IsaA and IsdB. A weak recognition of the three stains was observed with HarA although animals only received one injection rather than the three injections used for the other proteins.

TABLE 17

| Protein name | React on SA5 | React on SA8 | React on SE Hay |
|---|---|---|---|
| IsaA | (+) | (+) | (+) |
| ClfA | − | (+) | (+) |
| Atl amidase | − | − | ++ |
| SdrG | − | − | − |
| Glucosamidase | − | − | − |
| IsdA | − | − | ++ |
| Alpha toxin | − | − | − |
| SrdC | ++ | (+) | − |
| Ebh | + | − | + |
| AaA | − | − | ++ |
| MRPII | − | − | ++ |
| Sbi | ++ | ++ | +++ |
| FnbpA | + | + | (+) |

TABLE 18

| Protein name | React on SA5 | React on SA8 | React on SE Hay |
|---|---|---|---|
| IsaA | +++ | +++ | +++ |
| ClfA | + | ++ | ++ |
| Atl amidase | − | ++ | + |
| IsdB | +++ | +++ | +++ |
| SdrG | + | + | + |
| Glucosamidase | − | − | − |
| HarA (1 inject.) | + | + | + |
| IsdA | − | − | − |
| Alpha toxin | − | − | + |
| SrdC | − | − | − |
| Ebh | − | + | − |
| AaA | − | − | − |
| MRPII | − | − | ++ |
| Sbi | − | +++ | − |
| FnbpA | − | ++ | ++ |

### Example 14

Efficacy of Combinations of Staphylococcal Proteins in a Nasal Colonization Model.

Fifteen groups of three cotton rats were inoculated with combinations of eight staphylococcal antigens and five cotton rats which acted as controls were treated with no antigen. These sixteen groups are as follows:

Group 1—Atl-glucosamine, Atl-amidase, AAA, alpha toxin, SdrC, SdrG, Ebh, Sbi

Group 2—Atl-glucosamine, Atl-amidase, IsdA, IsdB, ClfA, SdrC, Ebh, FnbpA

Group 3—Atl-glucosamine, Atl-amidase, HarA, IsdA, MRPII, IsdB, AAA, alpha toxin

Group 4—Atl-glucosamine, HarA, IsdA, AAA, ClfA, IsaA, Ebh, Sbi

Group 5—HarA, MRPII, AAA, alpha toxin, ClfA, SdrC, Ebh, FnbpA

Group 6—IsdA, IsdB, AAA, alpha toxin, ClfA, SdrG, Sbi, FnbpA

Group 7—Atl-aminidase, IsdA, MRPII, AAA, IsaA, SdrG, Ebh, FnbpA

Group 8—Control

Group 9—Atl-glucosamine, IsdA, MRPII, alpha toxin, IsaA, SdrC, Sbi, FnbpA

Group 10—Atl-glucosamine, MRPII, IsdB, AAA, ClfA, IsaA, SdrC, SdrG

Group 11—Atl-amindase, MRPII, IsdB, alpha toxin, ClfA, IsaA, Ebh, Sbi

Group 12—Atl-glucosamine, HarA, IsdB, alpha toxin, IsaA, SdrG, Ebh, FnbpA

Group 13—Atl-amidase, HarA, IsdB, AAA, IsaA, SdrC, Sbi, FnbpA

Group 14—Atl-glucosamine, Atl-amidase, HarA, MRPII, ClfA, SdrG, Sbi, FnbpA

Group 15—Atl-amidase, HarA, IsdA, alpha toxin, ClfA, IsaA, SdfC, SdrG

Group 16—HarA, IsdA, MRPII, IsdB, SdrC, SdrG, Ebh, Sbi

Each mix of antigens contained 3 μg of each antigen mixed with an adjuvant made of liposomes containing MPL and QS21. The cotton rats were inoculated three times on days 1, 14 and 28 of the experiment. Two weeks after inoculation, the efficacy of the immunisations were assessed using a nasal colonisation assay as described in Kokai-Kun et al (2003) Antimicrob.Agents.Chemother. 47; 1589-1597.

Classical multiple linear regression analysis was carried out on the data using “Design Expert 6” software. The presence of an antigen was coded as +1 and the absence of an antigen by −1. Using the equation of the model it was possible to determine which antigens were the key antigens which produced a large decrease in the number of colonies per nose.

Results

The results of the nasal colonisation assay are shown in Table 19. The control group had a mean log CFU/nose of 3.51335 and a decrease in nasal colonisation could be see for all the groups of cotton rats inoculated with staphylococcal proteins. Groups 4, 9 and 13 showed the greatest decrease in nasal colonisation with a decrease of over 2 logs in CFU/nose. Groups 12 and 16 also gave good results, showing a decease of about 2 logs in CFU/nose.

TABLE 19

| Group | Mean observed LogCFU/nose | Predicted LogCFU/nose |
|---|---|---|
| 1 | 1.77527 | 2.03560 |
| 2 | 2.90435 | 2.52684 |
| 3 | 1.96556 | 2.23033 |
| 4 | 1.27748 | 1.21872 |
| 5 | 1.67304 | 1.93128 |
| 6 | 2.79745 | 2.98193 |
| 7 | 2.21481 | 2.30705 |
| 8 | 3.51355 | 3.47317 |
| 9 | 1.22480 | 1.44080 |
| 10 | 2.03085 | 1.93204 |
| 11 | 2.02522 | 1.81581 |
| 12 | 1.5302 | 1.70996 |
| 13 | 1.36063 | 1.49100 |
| 14 | 2.31201 | 1.73909 |
| 15 | 2.22979 | 1.98223 |
| 16 | 1.58109 | 1.44004 |

The contribution of specific antigens within the antigen mix was calculated using multiple regression analysis of the nasal colonisation data. The final model contains the seven best antigens. Results for these antigens are shown in Table 20. Within the context of the protein mix, the inclusion of HarA gave the greatest decrease in nasal colonisation, followed by IsaA, Sbi, SdrC, autolysin-glucosamine, MRPII and Ebh.

TABLE 20
Effects in difference of logCFU/nose and ratio of CFU/nose for the
seven best antigens in the model and corresponding p-values.

| | | Effect | Reduction | Cumulative | Cumulative |
|---|---|---|---|---|---|
| antigen | prob > F | estimate | ratio | effect | ratio |
| HarA | 0.033 | −0.596 | 3.9 | −0.596 | 3.9 |
| IsaA | 0.046 | −0.558 | 3.6 | −1.154 | 14.3 |
| Sbi | 0.077 | −0.491 | 3.1 | −1.645 | 44.2 |
| SdrC | 0.22 | −0.337 | 2.2 | −1.982 | 96.0 |
| Atl-glucos | 0.238 | −0.324 | 2.1 | −2.306 | 202.2 |
| MRPII | 0.239 | −0.323 | 2.1 | −2.629 | 425.3 |
| Ebh | 0.297 | −0.286 | 1.9 | −2.914 | 821.0 |

1. An immunogenic composition comprising Type 5 and/or 8 capsular polysaccharide or oligosaccharide from S. aureus wherein the Type 5 capsular polysaccharide or oligosaccharide is between 30% and 100% O-acetylated and comprising a staphylococcal protein or fragment thereof which is an extracellular component binding protein selected form the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, Lipase GehD, SasA, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig and MAP.

2. An immunogenic composition comprising Type 5 and/or 8 capsular polysaccharide or oligosaccharide from S. aureus wherein the Type 8 capsular polysaccharide or oligosaccharide is between 30% and 100% O-acetylated and comprising a staphylococcal protein or fragment thereof which is an extracellular component binding protein selected form the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, Lipase GehD, SasA, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig and MAP.

3. The immunogenic composition of claim 2 wherein the Type 5 capsular polysaccharide or oligosaccharide is between 30% and 100% O-acetylated.

4. The immunogenic composition of claim 1 comprising staphylococcal PNAG.

5. The immunogenic composition of claim 4 wherein the PNAG is less than 40% N acetylated.

6. The immunogenic composition of claim 1 further comprising Type I, and/or Type II and/or Type III capsular polysaccharide or oligosaccharide from S. epidermidis.

7. The immunogenic composition of claim 1 further comprising a S. aureus 336 antigen.

8. The immunogenic composition of claim 1 further comprising a staphylococcal protein or fragment thereof.

9. The immunogenic composition of claim 8 comprising 2 or more staphylococcal proteins selected from at least 2 different groups selected from;
a) at least one staphylococcal extracellular component binding protein or fragment thereof selected from the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, Lipase GehD, SasA, FnbA, FnbB, Cna, ClfB, FbpA, Npase, IsaA/PisA, SsaA, EPB, SSP-1, SSP-2, Vitronectin binding protein, fibrinogen binding protein, coagulase, Fig and MAP;
b) at least one staphylococcal transporter protein or fragment thereof selected from the group consisting of Immunodominant ABC transporter, IsdA, IsdB, IsdC, HarA, Mg2+ transporter, SitC and Ni ABC transporter;
c) at least one staphylococcal regulator of virulence, toxin or fragment thereof selected from the group consisting of alpha toxin (Hla), alpha toxin H35R mutant, RNA III activating protein (RAP).

10. The immunogenic composition of claim 1 wherein a staphylococcal polysaccharide is conjugated to a protein carrier.

11. The immunogenic composition of claim 4 wherein the PNAG is conjugated to a carrier protein.

12. The immunogenic composition of claim 10 wherein the carrier protein comprises a staphylococcal protein or fragment thereof selected from the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, Lipase GehD, SasA, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig, MAP, Immunodominant ABC transporter, IsdA, IsdB, IsdC, Mg2+ transporter, SitC and Ni ABC transporter, alpha toxin (Hla), alpha toxin H35R mutant and RNA III activating protein (RAP).

13. The immunogenic composition of claim 10 wherein the carrier protein is selected from the group consisting of tetanus toxoid, diphtheria toxoid, CRM197, Haemophilus influenzae protein D, Pseudomonas aeruginosa exoprotein A, pneumococcal pneumolysin and alpha toxoid.

14. The immunogenic composition of claim 1 wherein an effective immune response is generated against both S. aureus and S. epidermidis.

15. A vaccine comprising the immunogenic composition of claim 1 and a pharmaceutically acceptable excipient.

16. A method of making a vaccine comprising the steps of mixing antigens to make the immunogenic composition of claim 1 and adding a pharmaceutically acceptable excipient.

17. A method of preventing or treating staphylococcal infection comprising the step of administering the vaccine of claim 15 to a patient in need thereof.

18. A use of the immunogenic composition of claim 1 in the manufacture of a vaccine for treatment or prevention of staphylococcal infection.

19. A process for conjugating Type 5 or 8 capsular polysaccharide or oligosaccharide from S. aureus comprising the steps of:
a) dissolving the Type 5 or 8 polysaccharide or oligosaccharide in water or a saline solution;
b) adding a cyanylating agent (for example CDAP) to form an activated polysaccharide or oligosaccharide;
c) adding carrier protein so that amino groups react with the activated polysaccharide to form an isourea covalent link

20. The process of claim 19 wherein the Type 5 capsular polysaccharide or oligosaccharide is between 30% and 100% O-acetylated.
